(19) United States
(12) Patent Application Publication
(10) Pub. No.: US 2020/0231974 A1
(43) Pub. Date: Jul. 23, 2020

(54) A TWO-COMPONENT VECTOR LIBRARY SYSTEM FOR RAPID ASSEMBLY AND DIVERSIFICATION OF FULL-LENGTH T-CELL RECEPTOR OPEN READING FRAMES

(71) Applicant: GENOVIE AB, Karlskrona (SE)

(72) Inventors: Reagan Micheal Jarvis, Karlskrona (SE); Ryan Edward HILL, Karlskrona (SE); Luke Benjamin PASE, Karlskrona (SE)

(73) Assignee: GENOVIE AB, Karlskrona (SE)

(21) Appl. No.: 16/632,301

(22) PCT Filed: Jul. 17, 2018
(86) PCT No.: PCT/EP2018/069333

§ 371 Date: Jan. 17, 2020

(30) Foreign Application Priority Data
Jul. 18, 2017 (EP) 17181798.4

(51) Int. Cl. C12N 15/64; C12N 15/10; C07K 14/725; C12P 19/34

(52) U.S. Cl. CPC C12N 15/64; C12P 19/34; C07K 14/7051; C12N 15/1093

(57) ABSTRACT

A combined system comprising two separate components, wherein a first component is a vector carrying variable and constant (V-C) T-cell receptor (TCR) gene segments, and a second component is a vector carrying joining (J) TCR gene segments. The combined system may be modified so that said first component is a modified V-C entry vector encoding a first TCR chain, the system further comprises a fourth and a fifth component, wherein the fourth component comprises a Bidirectional Terminator (BiT) donor vector, and the fifth component comprises a modified V-C entry vector encoding a second TCR chain complimentary to said first TCR chain.

### FIELD OF THE INVENTION

The heterodimeric T-cell receptor (TCR) is central to adaptive immunity. Unique TCRs are continually generated during T-cell genesis whereby an array of gene segments are recombined into a single contiguous TCR open reading frame (ORF). Due to the large degree of diversity generated in this process of recombination, it is challenging to capture sequence data from TCR pairs that are expressed in single cells. Moreover, this diversity also makes it a challenging to provide these TCR open reading frames (ORFs) within genetic constructs on a high-throughput basis for testing and manipulation of TCR function. The present invention in the first aspect provides a pre-assembled two-component vector library system consisting of Variable-Constant entry vectors (V-C entry) and Joining donor (J donor) vectors comprising portions of TCR gene segments. The two component system is designed in such a way that when a V-C entry vector selected from the V-C entry vector library is combined with a J donor vector selected from the J donor vector library, along with a synthetic DNA oligonucleotide duplex encoding TCR complementarity determining region 3 (odeCDR3) in a restriction enzyme digestion/ligase cycle reaction, a single vector is created reconstituting the full-length TCR ORF. Such a vector library system enables PCR-independent methods for rapid and cost effective generation of TCR ORFs in selected vector contexts. In addition, this system permits novel workflows for generating synthetic TCR sequences for affinity and/or functional maturation workflows. This TCR ORF Reconstitution and Engineering System (TORES) is thus a strong tool for TCR functional analysis and engineering. In the second aspect, a TORES with modified V-C entry vectors is provided along with a third component bidirectional terminator donor vector (BiT donor) as to enable a second step to adjoin two TCR ORF chain pairs, in an antiparallel coding sense, into a single product vector. This bidirectional TORES² system enables alternative workflows to deliver paired TCR ORF constructs for TCR manipulation and characterisation.

### BACKGROUND OF THE INVENTION

Immune surveillance by T lymphocytes (I-cells) is a central function in the adaptive immunity of all jawed vertebrates. Immune surveillance by T-cells is achieved through a rich functional diversity across T-cell subtypes, which serve to eliminate pathogen-infected and neoplastic cells and orchestrate adaptive immune responses to invading pathogens, commensal microorganisms, commensal non-self factors such as molecular components of foodstuffs, and even maintain immune tolerance of self. In order to respond to various foreign and self factors, T-cells must be able to specifically detect molecular constituents of these foreign and self factors. Thus T-cells must be able to detect a large cross-section of the self and non-self molecules that an individual encounters, with sufficient specificity to mount efficient responses against pathogenic organisms and diseased self, while avoiding the mounting of such responses against health self. The highly complex nature of this task becomes clear when considering the practically unlimited diversity of both foreign and self molecules, and that pathogenic organisms are under evolutionary pressure to evade detection by T-cells.

### The T-Cell Receptor (TCR)

T-cells are primarily defined by the expression of a T-cell receptor (TCR). The TCR is the component of the T-cell that is responsible for interacting with and sensing the targets of T-cell adaptive immunity. In general terms, the TCR is comprised of a heterodimeric protein complex presented on the cell surface. Each of the two TCR chains are composed of two extracellular domains, being the variable (V)-region and the constant (C)-region, both of the immunoglobulin superfamily (IgSF) domain, forming antiparallel β-sheets. These are anchored in the cell membrane by a type-I transmembrane domain, which adjoins a short cytoplasmic tail. The quality of the T-cells to adapt and detect diverse molecular constituents arises from variation in the TCR chains that is generated during T-cell genesis. This variation is generated by somatic recombination in a similar manner to antibody genesis in B-cells.

### TCR Chain Diversity

The T cell pool consists of several functionally and phenotypically heterogeneous subpopulations. However, T cells may be broadly classified as αβ or γδ according to the somatically rearranged TCR form they express at their surface. There exist two TCR chain pair forms; TCR alpha (TRA) and TCR beta (TRB) pairs; and TRC gamma (TRG) and TCR delta (TRD) pairs. T-cells expressing TRA:TRB pairs are referred to as αβ T-cells, while T-cells expressing TRG:TRD pairs are often referred to as γδ T-cells.

TCRs of both αβ and γδ forms are responsible for recognition of diverse ligands, or ‘antigens’, and each T-cell generates αβ or γδ receptor chains de novo during T-cell maturation. These de novo TCR chain pairs achieve diversity of recognition through generation of receptor sequence diversity in a process called somatic V(D)J recombination after which each T-cell expresses copies of a single distinctly rearranged TCR. At the TRA and TRG loci, a number of discrete variable (V) and functional (J) gene segments are available for recombination and juxtaposed to a constant (C) gene segments, thus referred to as VJ recombination. Recombination at the TRB and TRD loci additionally includes a diversity (D) gene segment, and is referred to as VDJ recombination.

Each recombined TCR possess potential for unique ligand specificity, determined by the structure of the ligand-binding site formed by the α and β chains in the case of αβ T-cells or γ and δ chains in the case of γδ T-cells. The structural diversity of TCRs is largely confined to three short hairpin loops on each chain, called complementarity-determining regions (CDR). Three CDRs are contributed from each chain of the receptor chain pair, and collectively these six CDR loops sit at the membrane-distal end of the TCR extracellular domain to form the antigen-binding site.

Sequence diversity in each TCR chain is achieved in two modes. First, the random selection of gene segments for recombination provides basal sequence diversity. For example, TRB recombination occurs between 47 unique V, 2 unique D and 13 unique J germline gene segments. In general, the V gene segment contributes both the CDR1 and CDR2 loops, and are thus germline encoded. The second mode to generate sequence diversity occurs within the hypervariable CDR3 loops, which are generated by random deletion of template nucleotides and addition of non-template nucleotides, at the junctions between recombining V, (D) and J gene segments.

### TCR:CD3 Complex

Mature αβ and γδ TCR chain pairs are presented at the cell surface in a complex with a number of accessory CD3 subunits, denoted ε, γ, δ and ζ. These subunits associate with αβ or γδ TCRs as three dimers (εγ, εδ, ζζ). This TCR:CD3 complex forms the unit for initiation of cellular signalling responses upon engagement of a αβ or γδ TCR with cognate antigen. The CD3 accessories associated as a TCR:CD3 complex contribute signalling motifs called immunoreceptor tyrosine-based activation motifs (ITAMs). CD3ε, CD3γ and CD3δ each contribute a single ITAM while the CD3ζ homodimer contains 3 ITAMs. The three CD3 dimers (εγ, εδ, ζζ) that assemble with the TCR thus contribute 10 ITAMs. Upon TCR ligation with cognate antigen, phosphorylation of the tandem tyrosine residues creates paired docking sites for proteins that contain Src homology 2 (SH2) domains, such as the critical ζ-chain-associated protein of 70 kDa (ZAP70). Recruitment of such proteins initiate the formation of TCR:CD3 signalling complexes that are ultimately responsible for T-cell activation and differentiation.

### αβ T-Cells

αβ T-cells are generally more abundant in humans than their γδ T-cell counterparts. A majority of αβ T-cells interact with peptide antigens that are presented by HLA complexes on the cell surface. These peptide-HLA (pHLA)-recognising T-cells were the first to be described and are by far the best characterised. More rare forms of αβ T-cells have also been described. Mucosal-associated invariant T (MAIT) cells appear to have a relatively limited α and β chain diversity, and recognise bacterial metabolites rather than protein fragments. The invariant natural killer T-cells (iNK T-cells) and germline-encoded mycolyl-reactive T-cells (GEM T-cells) are restricted to recognition of glycolipids that are cross-presented by non-HLA molecules. iNK T-cells are largely considered to interact with CD1d-presented glycolipids, whereas GEM T-cells interact with CD1b-presented glycolipids. Further forms of T-cells are thought to interact with glycolipids in the context of CD1a and CD1c, however, such cells are yet to be characterised in significant detail.

### Conventional αβ T-Cells

The key feature of most αβ T-cells is the recognition of peptide antigens in the context of HLA molecules. These are often referred to as ‘conventional’ αβ T-cells. Within an individual, self-HLA molecules present peptides from self and foreign proteins to T-cells, providing the essential basis for adaptive immunity against malignancies and foreign pathogens, adaptive tolerance towards commensal organisms, foodstuffs and self. The HLA locus that encodes HLA proteins is the most gene-dense and polymorphic region of the human genome, and there are in excess of 12,000 alleles described in humans. The high degree of polymorphism in the HLA locus ensures a diversity of peptide antigen presentation between individuals, which is important for immunity at the population level.

### HLA Class I and II

There are two forms of classical HLA complexes: HLA class I (HLAI) and HLA class II (HLAII). There are three classical HLAI genes: HLA-A, HLA-B, HLA-C. These genes encode a membrane-spanning α-chain, which associates with an invariant β2-microglobulin (β2M) chain. The HLAI α-chain is composed of three domains with an immunoglobulin fold: α1, α2 and α3. The α3 domain is membrane-proximal and largely invariant, while the α1 and α2 domains together form the polymorphic membrane-distal antigen-binding cleft. There are six classical HLAII genes: HLA-DPA1, HLA-DPB1, HLA-DQA1, HLA-DQB1, HLA-DRA, and HLA-DRB1. These genes encode paired DP, DQ and DR heterodimeric HLA complexes comprising a α-chain and a β-chain. Each chain has two major structural domains with an immunoglobulin fold, where the α2 and β2 domain comprise membrane-proximal and largely invariant modules similar to that of HLAI α3 domain. The HLAII α2 and β2 domains together form the membrane-distal antigen-binding cleft and are regions of high polymorphism.

The antigen-binding cleft of HLAI and HLAII comprises two anti-parallel α-helices on a platform of eight anti-parallel β-sheets. In this cleft the peptide antigen is bound and presented in an extended conformation. The peptide-contacting residues in HLAI and HLAII are the location of most of the sequence polymorphism, which constitutes the molecular basis of the diverse peptide repertoires presented by different HLA alleles. The peptide makes extensive contacts with the antigen-binding cleft and as a result each HLA allele imposes distinct sequence constraints and preferences on the presented peptides. A given peptide will thus only bind a limited number of HLAs, and reciprocally each allele only accommodates a particular fraction of the peptide collection from a given protein. The set of HLAI and HLAII alleles that is present in each individual is called the HLA haplotype. The polymorphism of HLAI and HLAII genes and the co-dominant expression of inherited alleles drives very large diversity of HLA haplotype across the human population, which when coupled to the enormous sequence diversity of αβ TCRs, presents high obstacles to standardisation of analysis of these HLA-antigen-TCR interactions.

### αβ TCR Engagement of HLAI and HLAII

αβ TCRs recognize peptides as part of a mixed pHLA binding interface formed by residues of both the HLA and the peptide antigen (altered self). HLAI complexes are presented on the surface of nearly all nucleated cells and are generally considered to present peptides derived from endogenous proteins. T-cells can thus interrogate the endogenous cellular proteome of an HLAI-presenting cell by sampling pHLAI complexes of an interacting cell. Engagement of HLAI requires the expression of the TCR co-receptor CD8 by the interacting T-cell, thus HLAI sampling is restricted to CD8⁺ αβ T-cells. In contrast, the surface presentation of HLAII complexes is largely restricted to professional APC, and are generally considered to present peptides derived from proteins exogenous to the presenting cell. An interacting T-cell can therefore interrogate the proteome of the extracellular microenvironment in which the presenting cell resides.

The engagement of HLAII requires the expression of the TCR co-receptor CD4 by the interacting T-cell, thus HLAII sampling is restricted to CD4⁺ αβ T-cells.

### Thymic Selection of αβ TCRs

The role of αβ TCRs as described above is the detection of pHLA complexes, such that the TCR-presenting T-cell can raise responses germane to the role of that T-cell in establishing immunity. It should be considered that the αβ TCR repertoire generated within an individual must account for the immense and unforeseen diversity of all foreign antigens likely to be encountered in the context of a specific haplotype and prior to their actual occurrence. This outcome is achieved on a background where extremely diverse and numerous αβ TCRs are generated in a quasi-randomised manner with the potential to recognise unspecified pHLA complexes while only being specifically instructed to avoid strong interactions with self pHLA. This is carefully orchestrated during T-cell maturation in a process call thymic selection.

During the first step of T-cell maturation in the thymus, T-cells bearing αβ TCRs that are incapable of interacting with self-pHLA complexes with sufficient affinity, are deprived of a survival signal and eliminated. This step called positive selection assures that the surviving T-cells carry a TCR repertoire that is at least potentially capable of recognizing foreign or altered peptides presented in the right HLA context. Subsequently, αβ TCR that strongly interact with self-pHLA and thus have the potential to drive autoimmunity are actively removed through a process of negative selection. This combination of positive and negative selection results in only T-cells bearing αβ TCRs with low affinity for self-pHLA populating the periphery. This establishes an αβ T-cell repertoire that is self-restricted but not self-reactive. This highly individualised nature of T-cell genesis against HLA haplotype underscores the challenges in standardised analysis αβ TCR-antigen-HLA interactions. Moreover, it forms the basis of both graft rejection and graft versus host disease and the general principle that αβ TCRs identified in one individual may have completely different effect in a second individual, which has clear implications for TCR-based and T-cell based therapeutic and diagnostic strategies emerging in clinical practice.

### Unconventional αβ T-Cells

The non-HLA-restricted, or ‘unconventional’, forms of αβ T-cells have very different molecular antigen targets. These unconventional αβ T-cells do not engage classical HLA complexes, but rather engage conserved HLA-like proteins such as the CD1 family or MR1. The CD1 family comprises four forms involved in antigen cross-presentation (CD1a,b,c and d). These cell surface complexes have an α-chain resembling HLAI, which forms heterodimers with β2-M. A small hydrophobic pocket presented at the membrane distal surface of the α-chain forms a binding site for pathogen-derived lipid-based antigens. Innate like NK T-cells (iNK T-cells) form the best-understood example of lipid/CD1 family recognition with GEM T-cells representing another prominent example. ‘Type I’ iNK T-cells are known to interact strongly with the lipid α-GalCer in the context of CD1d. These iNK T-cells display very limited TCR diversity with a fixed TCR α-chain (Vα10/Jα18) and a limited number of β-chains (with restricted vβ usage) and they have been likened to innate pathogen-associated molecular patterns (PAMPS) recognition receptors such as Toll-like and Nod-like receptors. In contrast, ‘type II’ NK T-cells present a more diverse TCR repertoire, and appear to have a more diverse mode of CD1d-lipid complex engagement. GEM T-cells recognize mycobacteria-derived glycolipids presented by CD1b, however, the molecular details of antigen presentation by CD1a, b and c as well as their T-cell recognition are only beginning to be understood.

MAIT cells largely express an invariant TCR α-chain (TRAV1-2 ligated to TRAJ33, TRAJ20, or TRAJ12), which is capable of pairing with an array of TCR β-chains. Instead of peptides or lipids MAIT TCRs can bind pathogen-derived folate- and riboflavin-based metabolites presented by the HLAI-like molecule, MR1. The limited but significant diversity in the TCRs observed on MAIT TCRs appear to enable the recognition of diverse but related metabolites in the context of the conserved MR1.

It is not well-understood how non-classical HLA-restricted αβ T-cell TCRs are selected in the thymus during maturation. However, it appears likely that the fundamental process of negative and positive selection outlined above still applies and some evidence suggests that this occurs in specialized niches within the thymus.

### γδ T-Cells

In contrast to the detailed mechanistic understanding of αβ TCR genesis and pHLA engagement, relatively little is known about the antigen targets and context of their γδ T-cell counterparts. This is in part due to their relatively low abundance in the circulating T-cell compartment. However, it is broadly considered that γδ T-cells are not strictly HLA restricted and appear to recognize surface antigen more freely not unlike antibodies. Additionally, more recently it has become appreciated that γδ T-cells can dominate the resident T-cell compartment of epithelial tissues, the main interaction site of the immune system with foreign antigen. In addition, various mechanisms for γδ T-cell tumour immunuosurveillance and surveillance of other forms of dysregulated-self are beginning to emerge in the literature. The specific antigen targets of both innate-like and adaptive γδ T-cells remain poorly defined but the tissue distribution and fast recognition of PAMPs suggests a fundamental role for γδ T-cells both early in responses to foreign antigens as well as early in life when the adaptive immune system is still maturing.

The diverse functions of γδ T-cells appear to be based on different Vγ Vδ gene segment usage and can be broadly understood in two main categories in which γδ T-cells with largely invariant TCRs mediate innate-like recognition of PAMPs very early during infection. Beyond PAMPs these type of γδ T-cells are furthermore believed to recognize self-molecules, including phosphoantigens that could provide very early signatures of cellular stress, infection and potentially neoplastic development. Recognition of PAMPs and such so-called danger associated molecular patterns (DAMPS) as well as the large numbers of tissue-restricted innate-like γδ T-cells strongly suggests that these cells are suited to respond rapidly to antigenic challenge without the need for prior activation, homing and clonal expansion.

A second form of γδ T-cells are considered to be more adaptive in nature, with a highly diverse γδ TCR repertoire and the ability to peripherally circulate and access lymphoid tissues directly. Such antigen-specific γδ T-cells to common human pathogens such as CMV have been described and they appear to form a memory response. However, it has also been observed that γδ T-cells show only relatively limited clonal proliferation after activation and little data is available on the extent of TCR diversity and specific responses of γδ T-cells in peripheral circulation, or in tissues. Furthermore, while it is generally considered that γδ TCRs do not interact with pHLA complexes, and thus do not engage with peptide antigens in this context only few antigen targets of γδ T-cells have been characterised and the underlying molecular framework is only poorly understood.

The low frequency of peripheral γδ T-cells and the difficulty to study tissue-resident T-cells in humans has limited our knowledge of how this important and diverse type of T-cells participate in adaptive immune responses. This emerging area of research would require more reliable technologies with which to capture and characterise rare γδ T-cells, isolate their TCR pairs, and to identify their cognate antigens.

### Antigens and Antigen Presenting Cells

In the context of T-cells and TCRs, antigens may be defined as any molecule that may be engaged by a TCR and resulting in a signal being transduced within the T-cell. The most well characterised T-cell antigens are peptides presented in an HLAI and HLAII complex, and which are engaged by conventional αβ T-cells. However, in recent years it has become apparent that non-conventional αβ T-cells and γδ T-cells are able to engage a wide range of biomolecules as antigens, including lipids, lipopeptides, glycopeptides, glycolipds and a range of metabolites and catabolites. In addition, it has emerged that γδ T-cells may be able to engage fully folded proteins directly in an antibody-like fashion. Therefore, the view of T-cell antigens being largely restricted to HLA-presented peptides has expanded over the past two decades to include almost any biomolecule. With this concept in mind, it is relevant to define what may be considered an antigen-presenting cell (APC).

As defined in the above sections, HLAI and HLAII have a disparate expression profiles across cell types. It is widely accepted that nearly all nucleated cells present HLAI complexes on the cell surface, and are thus competent to present peptide antigens for T-cell sampling. In contrast, HLAII has a restricted expression profile, and at least in steady state conditions is only expressed on the surface of cells that have a specialist role in antigen presentation, including dendritic cells (DC), macrophage and B-cells. These specialist cell types are often referred to as professional APC. For the purposes of this document, the term APC is used to describe any nucleated cell that is capable of presenting an antigen for sampling by αβ or γδ T-cells. Such antigens are not restricted to those presented as ‘cargo’ in specific antigen-presenting complexes such as HLA and HLA-like molecules, but may also include any cell-surface presented moiety that is able to engage a αβ or γδ TCR-bearing cell.

### Therapeutic Use of TCRs

Adoptive transfer of primary T-cells was first trialled in a clinical setting in the early 1990s, starting with ex vivo expanded T-cells polarised towards viral antigens to confer viral immunity in immunocompromised patients. Similar approaches using primary T-cells expanded ex vivo against specific cancer antigens were soon after trialled in treatment of malignancies. One limitation in these early approaches that continues to be a challenge today is a lack of understanding of the nature and diversity of T-cells clashing with the need to finely-optimize their composition in the therapeutic product. At present, the use of ex vivo expanded primary T-cells has largely been abandoned by the pharmaceutical industry with the exception of a handful of initiatives using primary T-cells with specificity for viral antigens.

In recent years the ability to reliably introduce genetic material into primary human cells has seen a variety of experimental genetically modified T-cell therapeutics arise. Such therapeutic cell products aim to harness the power of T-cell responses and redirect T-cell specificity towards a disease-associated antigen target, for example, an antigen uniquely expressed by malignant cells. These have largely relied on the transfer of a chimeric antigen receptor (CAR) into recipient T-cells, rather than actual TCR chain pairs. A CAR represents a targeting moiety (most often a single-chain antibody element targeting a surface expressed protein of malignant cells) grafted to signal receptor elements such as the ζ-chain of the CD3 complex, to produce a synthetic chimeric receptor that mimics CD3-TCR function. These so-called CAR T-cell (CAR-T) products have met mixed success in clinical trials to date and despite their potential are not easy to translate beyond tumours with inherent unique molecular targets such as B-cell malignancies. Alternatively, the transfer of full-length TCR chain pair ORFs into T-cells is of emerging interest. Such TCR-engineered T-cell therapeutics are at present limited by challenging manufacturing processes, and like the CAR-T products, a dearth of validated antigen targets and targeting constructs. To date this has been focused on the use of αβ TCRs for recognition of peptide antigens presented by HLAI on malignant cells and a fundamental challenge of this approach is the need for antigens that are specific to malignant cells.

It has been considered that since the TCR-pHLA interaction is of relatively low-affinity, native TCRs are likely to be suboptimal for TCR-engineered T-cell therapies. Several approaches have been devised to affinity-mature TCRs in vitro, in much the same manner as single-chain antibody affinity maturation. These TCR affinity maturation approaches generally also utilise a single-chain formats, wherein the V-region of one chain is fused to V-region of another chain to make a single polypeptide construct. Such single polypeptides may then be used in phage- or yeast-display systems adapted from antibody engineering workflows, and passed through rounds of selection based on target binding. Two inherent limitations exist in such a single-chain TCR approach in terms of yielding functional TCR chain pairs. Firstly, the selection is based on binding affinity to the target. However, it has been well documented that TCR affinity does not always correlate to the strength or competency of TCR signalling output. Secondly, the selection of single-chain constructs based on affinity does not always translate to equivalent affinities once they are reconstituted as full-length receptors.

In a therapeutic context, there exists an additional limitation in affinity-matured TCR pairs. That is, considering their sequences have been altered, the resulting constructs by definition have no longer been subject to thymic selection, wherein TCRs that react strongly to self-antigens are deleted from the repertoire. Therefore, these modified TCRs carry an inherent risk of being auto-reactive, which is very difficult to rule out in vitro using current methods. For the same reason, any selected or engineered TCR for therapeutic application needs to be individualised. If TCRs are artificially engineered or native TCRs applied across individuals, cross-reactivities have to be ruled out on the basis of the HLA haplotype and presented peptide repertoire of each specific individual in order to avoid potentially catastrophic autoimmunity. This is due to the fact that thymic selection is conducted on a background of all available HLA molecules specific only to that given individual. The likelihood of such cross-reactivity is unclear. However, the ability of our TCR repertoire to recognize pHLA complexes of other individuals of the same species as foreign is a fundamental property of adaptive immunity and underpins graft rejection and graft versus host disease. Recent clinical trials using a matured TCR chain pair against the cancer-specific melanoma associated antigen (MAGE) highlighted the potential problem of bypassing thymic selection. When autologous T-cells harbouring the matured TCRs were infused back to two cancer patients, these patients rapidly developed a fatal heart disease. Subsequent studies determined that the MAGE-specific matured TCRs were cross-reactive with an HLAI-presented peptide from the heart protein titin. This strongly suggests that cross-reactivity is a distinct possibility in therapeutic use of TCRs.

A distinct avenue of utilising TCRs for therapeutic purposes is in their use as affinity reagents in much the same manner as antibody therapeutic substances. Single-chain TCR molecules have been trialled for delivery of conjugated drug substances to specific HLA-antigen expressing cell populations. Such an approach is generally considered safer than CAR-T or TCR engineered T-cell therapeutics, as administration of the drug substance may simply be withdrawn. However, the potential for cross-reactivity and off target effects that are difficult to predict remains a potential limitation in this setting.

### TCR Repertoire Detection in Clinical Diagnostics

In a related aspect, there is an emerging interest in using the detection of the abundance of specific TCR sequences for clinical diagnostic purposes. With the rise of deep-sequencing methods in particular, it is possible to capture the full TCR diversity within an individual globally and for matched αβ pairs in specific contexts. This potentially represents a means to diagnose specific conditions and disease states simply by detecting the abundance of expanded T-cell clones, as proxy readout for established immune response against a disease-associated antigen in the patient. However, such global approaches are currently limited to very strong immune responses with established clinical time-points and suffer from the underlying difficulty in identifying the specific antigen target of any particular TCR identified via sequencing.

### Therapeutic and Diagnostic Use of T-Cell Antigens

The fundamental strength of harnessing adaptive immune responses translates into a central technical challenge in that the exquisite specificity of the TCR-antigen interaction requires detailed knowledge of the antigens specifically associated with each pathogen, cancer cell or autoimmune disease. Furthermore, each antigen may be presented by a specific antigen presenting complex, or allele thereof, such that antigen discovery has be performed for each relevant HLA gene and allele. For several infectious diseases like HIV, influenza and CMV that are associated with strong adaptive immune responses and generally display conserved epitope response hierarchies, the most important epitopes have been mapped in context of some common HLA. Similarly, the fields of cancer, allergy and autoimmunity have seen increased and systematic efforts to map the associated T-cell antigens. However, these are challenging procedures and the efforts to systematically describe T-cell antigens associated with different clinical contexts are hindered by the absence of efficient, robust, fast and scalable protocols.

Specifically, cancer cells represent a challenging and important aspect as most of the peptides presented on the surface of malignant cells are self antigens or very similar to self antigens. Therefore, thymic selection will have deleted TCRs that could strongly recognize these peptides, while at the same time the tumour has evolved to evade immune recognition. This means that potent immune responses against established tumours are relatively rare and targets difficult to predict or discover. However, these responses do exist and, importantly, are generally associated with better outcome. The target of such responses, tumour-associated-antigens (TAA), will in most cases have distinguishing characteristics from self and be derived from proteins that are overexpressed during cancer development, otherwise absent from the cell type at this stage of development or specifically altered through genetic mutation or post-translational modifications such as phosphorylation.

When available, the knowledge of such epitopes makes it possible to interrogate the associated T-cell response for fundamental discovery, diagnostic purposes and for example as a test of vaccine efficacy. Importantly, they also provide highly specific targets for T-cell tolerization in allergy and autoimmunity and, crucially, point towards valuable targets for specific immunotherapy and against malignant cells. Malignancies represent a particularly valuable target as the promise of cellular immunotherapies and the progress in the T-cell manipulations are slowed by a lack of validated target TAAs that go beyond the few cases where specific markers for the type of cancer happen to be available.

In the light of the potential of cellular therapy and lack of validated targets the identification of promising TCR antigens remains one of the most pressing bottlenecks of TCR-based immunotherapy, particularly in the effort to treat cancer.

### Obtaining Full-Length TCR ORFs

Primarily due to the diversity of usage in V gene segments in naturally occurring TCRs, it is challenging to capture sequence data of paired TCR chains from pools of T-cells. In general, in order to reliably capture sequence with high reliability it is necessary to amplify TCR using reverse transcription and/or PCR methods, using a pool of primers that cover the full V region diversity of the target pool. This process generates ORF fragments from which sequence data may be obtained, however, make unsuitable starting material for readily cloning full-length TCR ORFs. Therefore, high-efficiency strategies for mining paired sequence data from pools of T-cells is generally incompatible with cloning of full-length TCR ORFs for downstream applications. Time consuming and expensive synthesis of TCR ORFs is thus required for any functional testing or application of the sequenced TCR pairs.

### Diversification of Full-Length TCR ORFs

The emerging therapeutic use of TCRs has seen a rise in interest to diversify TCR sequences within affinity and/or functional maturation workflows to derive synthetic TCR pairs of defined specificity and function. Generally, this has been achieved through linking TCR chain pairs into a single-chain construct for phage-display methodologies, and introduction of sequence diversity into these single chain constructs. There is currently a lack of technologies that can rapidly diversify single TCR ORFs in a systematic manner, and where these full-length TCR ORFs are applicable to immediate testing and/or selection in a context of surface expression on viable mammalian cells. Such a technology would be of great value in the maturation of TCR pairs, with highly defined specificity and signalling capacity, for therapeutic use.

### Technological Aspects of TCR and T-Cell Antigen Analyses

Overall, the development of TCR-based therapies is still in its early stages, and success has been limited. Diagnostic approaches, while of immense potential, have seldom been deployed into controlled clinical studies that aim to assess patient disease states or response to therapy. Underdeveloped techniques for the reliable capture of native TCR chain pairs, and the systematic analysis of TCR-antigen interactions at high-throughput and in a functional context of cell-cell communication, have been the main hurdles to the development of TCR-based therapies and diagnostics.

Deep sequencing approaches have led to an improved understanding of T-cell receptor diversity in heath and disease. However, these approaches have generally focused on short stretches spanning the CDR3 regions, mainly of the TCR β-chain. Most studies have ignored the contribution of the TCR α-chain, and few have sought to analyse paired αβ chains as well as the antigen specificity of TCRs determined to be of interest. Recent workflows using single cell encapsulation and genetic barcoding has enabled the pairing of native TCR αβ or γδ chain pairs and analysis of full-length sequences, however, such workflows remain experimental.

Isolated TCR chain pairs may be analysed in terms of antigen specificity in either biophysical or functional modes. Biophysical analysis requires the recombinant production of both the TCR as well as the analyte antigen in soluble form. In the case of HLA-restricted TCRs this would thus require the generation of all individual TCRs as well as the cognate pHLA complexes. This is technically highly challenging, slow and very low-throughput. Furthermore, such analysis would only provide interaction affinities, which are not well-correlated with functional characteristics in predictable ways.

Until recently, the detailed functional analysis of isolated TCR sequences in a cellular context has been limited to laborious protocols of transfection of analyte TCR chain pairs into primary T-cells or immortal T-cell lines, and detection of cellular responses by traditional flow cytometric analysis of cell activation, or detection of secreted factors from the transfected cells upon antigen challenge. In a recent publication by Guo et al, rapid cloning, expression, and functional characterization of paired TCR chains from single-cells was reported (Molecular Therapy—Methods and clinical development (2016) 3:15054). In this study, analyte human αβ TCR pairs were expressed in a reporter cell line that lacked αβ TCR expression, and which contained a green fluorescent protein (GFP) reporter system linked to the Nur77 promoter that is activated upon TCR stimulation. This system remains inefficient due to the lack of standardised TCR integration into the reporter cell line genome, and does not provide a systematic manner for cell-bound antigen challenge by an APC element.

Similar to workflows for identification of TCRs against known T-cell antigens, the de novo discovery of novel T-cell antigens in health and disease remains highly challenging. Most approaches remain biophysical in nature, and aim to produce candidate antigens that may be tested in immunisation protocols, or through identifying cognate TCRs as addressed above. Little or no standardisation exists in the field of T-cell antigen discovery, and the field is largely restricted to academic study.

With the accumulating interest in TCRs and their cognate antigens in both therapeutic and diagnostic use, and the emergence of means to capture significant numbers of native TCR αβ and γδ chain pairs, there remains a lack of reliable high-throughput and standardised technologies for the systematic analysis of TCR-antigen interactions. Importantly, there is a lack of standardised systems for rapid reconstitution and/or systematic diversification of full-length TCR ORFs, such that these ORFs may be directly applied to functional analysis of TCR chain pairs in the native context of an analyte TCR being presented on the surface of a viable cell in a native context. Such capability is important for achieving high-throughput analyses of native TCR chain pairs, but also affinity and/or functional maturation of TCR chain pairs, for therapeutic and diagnostic uses.

There is a clear need for rapid and systematic methods for TCR chain reconstitution, and their systematic diversification, in high-throughput methods that will enable the use of TCR diagnostics on an informatics and reagent basis, and also personalised TCR-based immunotherapies.

### DETAILED DESCRIPTION OF THE INVENTION

The present invention addresses the above-mentioned needs. The present invention provides in a first aspect, a two-component vector system comprising pre-assembled libraries consisting of vectors harbouring variable (V), joining (J) and constant (C) sequences for TCR chains. The first component of such a system comprises a V-C entry vector containing V and C sequences. The second component of the system comprises a J donor vector containing J sequence. The two-component vector system is preassembled into libraries of V-C entry vectors and J donor vectors with all desirable V-C sequence combinations and J sequences, respectively. The two-component vector system is designed in such a manner that a single V-C entry vector and a single J donor vector with desired sequences can be combined with a short DNA oligonucleotide duplex encoding CDR3 (odeCDR3) sequence to reconstitute a full-length TCR ORF in vitro, in a single-tube reaction in a restriction enzyme and ligase dependent and PCR independent manner. In addition, the modular two-component system is ideally suited to rapidly generate large libraries of synthetic or mutant full-length TCR ORFs for affinity or functional maturation workflows. In a second aspect, modified V-C entry vectors for a reciprocal pair of TCR chains (i.e. TRA/TRB or TRD/TRG) are compiled into a 5-component vector system to provide a system in which reconstituted TCR chain pairs may be adjoined within a single vector. This second system utilises both modified V-C entry vectors, the same J donor vectors as the first system, and also a Bidirectional Terminator Donor Vector (BiT Donor) as to achieve adjoined TCR chain pairs encoded in antiparallel sense orientation in a final construct, with each TCR chain interposing a ‘bidirectional terminator’ element.

### TCR ORF Reconstitution and Engineering System (TORES)

The present invention first provides a two-component vector system with unique characteristics suitable for the above-mentioned uses. This TCR ORF reconstitution and engineering system (TORES) is used in conjunction with a third component, an oligonucleotide duplex encoding CDR3 (odeCDR3), to de novo assemble full-length TCR ORFs within a defined vector context and/or generate formulaic sequence diversity within a given TCR ORF.

The present invention is summarised in FIG. 1A. A selected V-C entry vector containing V and C TCR gene segments required for a target full-length TCR ORF is combined with a J donor vector that contains the required J TCR gene segment. The full-length TCR ORF is completed by the addition of an oligonucleotide duplex encoding CDR3 (odeCDR3), which accounts for unfixed non-germline sequence generated during V(D)J recombination and interposed by fixed germline encoded V and J sequence encoded by the V-C entry vector and J donor vector, respectively. The two-component vector system, and the third odeCDR3 component, is designed such that when combined into a restriction enzyme and ligase cycle reaction, the full V-CDR3-J-C TCR ORF is reconstituted. This is achieved via a Type IIS restriction enzyme(s) that are used to perform ‘scarless’ assembly of the genetic elements in a standardised manner. The two-component vector system is assembled into a library containing all required V, C and J gene segments for reconstitution of target full-length TCR ORFs (FIG. 1B). For instance, a library can be constructed to contain all gene segments encoding native protein sequences of the human TRA repertoire as described in Examples 1 and 2, and the human TRB repertoire as described in Example 3.

To reconstitute a full-length TCR ORF, from sequence information that is sufficient to define V, J and C gene segment usage, along with unfixed CDR3 sequence interposed by fixed V and J segments, the V-C entry vector and J donor vector that correspond to the V/C and J usage of the target TCR ORF are first selected. An odeCDR3 corresponding the unfixed CDR3 sequence that is needed to complete the full-length TCR ORF is also generated. These three components are combined with a Type IIS restriction enzyme and DNA ligase enzyme in a cycle reaction to generate the target full-length TCR ORF as described in FIG. 4 and Example 7. The resulting reconstituted full-length TCR is contained within the V-C entry vector backbone, thus contains all vector features contained within this parent construct.

The action of the Type IIS restriction enzyme of the three combined components (FIG. 4 a, b, c) within a restriction enzyme/ligase cycle reaction, results in two reaction by-products and two reaction intermediates. The V-C entry vector derived reaction by-product is the excised native selection marker and Type IIS binding sites (FIG. 4d). The J donor vector backbone from which the J segment part has been excised represents a second reaction by-product (FIG. 4e). The excised J segment part from the J donor vector represents a reaction intermediate, and contains both a J segment part, a small C part from the C segment, and single stranded overhangs required for ligation (FIG. 4f). The second reaction intermediate is the parental V-C entry backbone containing the V and C segments, and single stranded overhangs required for ligation (FIG. 4g). The final product of reaction represents a full-length TCR ORF reconstituted within the parental V-C entry vector backbone, comprised of ligation of the odeCDR3 (FIG. 4c), the excised J segment part (FIG. 4f) and the V-C entry backbone carrying the V and C gene segments (FIG. 4g).

### The V-C Entry Vector and J Donor Vector Components

In the present context, a combined two-component system includes one or more V-C entry vector/s containing
a. origin of replication,
b. a first positive selection marker,
c. 5′ genetic element, or elements,
d. Kozak Sequence,
e. TCR variable gene segment,
f. a first Type IIS sequence, for site specific recognition and cleavage by a Type IIS restriction enzyme,
g. a negative selection marker,
h. a second Type IIS sequence,
i. TCR constant gene segment, and
j. 3′ genetic element, or elements
   wherein, a) and b) are used for propagation and selection of both parental V-C entry vector and the reconstituted TCR-containing vector in a bacterial host; c) and j) are used to define the downstream application of the reconstituted full-length TCR ORF; d) ensures efficient initiation of translation in eukaryotic cells, which could alternatively represent a Shine-Dalgarno sequence for transitional regulation in prokaryotes and archaea; e) represents the variable (V) gene segment from the start codon to a motif at the 5′ edge of the CDR3 region conserved across all V segments in a given two-component vector system; f) represents a Type IIS recognition sequence that directs a Type IIS restriction enzyme to cut in the 5′ direction as to create a standardised single stranded overhang at the 3′ end of the V gene segment; g) represents a negative selection marker to eliminate parental V-C entry vector during operation of the system to reconstitute a full-length TCR ORF; h) represents a Type IIS recognition sequence that directs a Type IIS restriction enzyme to cut in the 3′ direction as to create a standardised single stranded overhang at the 5′ end of the C gene segment; i) represents the constant (C) gene segment from a motif at the 5′ end of the C gene segment conserved across all C segments in a given two-component vector system, and which defines the boundary with the J segment (see FIGS. 2 and 4).

A V-C entry vector that is used for genetic reconstitution of the full-length TCR ORF without the need for downstream biological application, for example, to be used as a template for molecular biology workflows, the minimal V-C entry vector would comprise elements a), b), e), f), h) and i), lacking regulatory elements.

A V-C entry vector can also contain one or more transcriptional units for the expression of additional ORFs suitable for downstream applications, for example, a mammalian antibiotic resistance gene or reporter construct.

The combined two-component system includes one or more J-donor vector containing
a. origin of replication,
b. a second positive selection marker,
c. a third Type IIS sequence,
d. TCR Joining gene segment,
e. a C part, corresponding to a small 5′ portion of a constant gene segment, and
f. a fourth Type IIS sequence.
   wherein, a) and b) are used for propagation and selection of the J donor vector; c) represents a Type IIS recognition sequence that directs a Type IIS restriction enzyme to cut in the 3′ direction as to create a standardised single stranded overhang at the 5′ end of the J gene segment; d) represents the Joining (J) gene segment starting from a 5′ from the motif defining the 3′ edge of the CDR3 region conserved across all J segments in a given two-component vector system, to a 3′ sequence that incorporates C part, representing a 5′ portion of the C segment encoded by V-C entry vector(s) contained within the two-component system; represents a Type IIS recognition sequence that directs a Type IIS restriction enzyme to cut in the 5′ direction as to create a standardised single stranded overhang at the 3′ end of the J gene segment, and contained within the C part portion of the sequence (see FIGS. 3 and 4).

A J-donor vector does not strictly need to carry a C part sequence, encoding a small 5′ portion of the C gene segment. This C part is used to optimise and standardise overhangs for the reconstitution reaction during operation of a TORES. This is because of the higher sequence variation found at the 3′ end of J gene segments, such that inclusion of a C part allows standardisation by generation of overhangs within the less diverse C gene segment. In the instance of constructing a TORES for a TCR loci from other organism that does not have 3′ J segment diversity, or using synthetic J gene segments, this C-part may be omitted in favour of standardisation of overhangs within said J segments. This would reduce the complexity of the J donor library construction.

Each of first, second, third and fourth Type IIS sequences may be the same or different. Preferably, they are the same. This ensures that each of the restriction sites within the two-component vector system is compatible with the same Type IIS enzyme, and only a single enzyme is needed for the restriction enzyme/ligase cycle reaction during reconstitution of full-length TCR ORF using the system. Type IIS enzymes do not cut within their recognition sequence, and thus the single-stranded overhangs are generated extrinsic to the recognition sequence. Therefore, the nature of the overhang generated upon Type IIS restriction enzyme action is dependent on both the orientation of the recognition sequence, and indeed the adjacent sequence (see examples 1 to 4).

Alternatively, each of the Type IIS restriction sequences may be different from one another. However, with the addition of each unique recognition sequence, an additional Type IIS enzyme must be incorporated into the restriction enzyme/ligase cycle reaction. This would increase the complexity and cost of a reconstitution reaction for assembling a full-length TCR ORF.

The first and second positive selection markers within the V-C entry vector and J donor vector, respectively, are normally different. This is to ensure that the V-C entry vector, which provides the backbone of the final full-length TCR ORF product, can be selected for independently of the J donor vector, and thus eliminate transformants that carry undigested or re-circularised J donor vectors that would otherwise contribute background to the reconstitution reaction (see FIGS. 2 and 3 and Example 7).

The positive selection markers can be selected from
a. an antibiotic resistance gene,
b. an auxotroph complementing gene,
c. a reporter gene
   wherein the choice, formatting and application of such positive selection markers are well known to those skilled in the art.

The 5′ genetic element incorporated into a V-C entry vector comprises one or more elements selected from
a. gene cis/acting element,
b. heterospecific recognition site for recombinase enzymes,
c. a 5′ homologous recombination arm for a genomic site of interest′
d. a mRNA splice acceptor site,
e. an internal ribosomal entry site, and
f. epigenetic insulator sequence
   wherein, a) drives expression of the transcript encoded by the full-length TCR ORF product reconstituted within the V-C entry vector backbone; b) represents a sequence that directs site-directed recombination in the presence of recombinase enzymes to insert the full-length TCR ORF product reconstitute within the V-C entry vector backbone into a specific genetic context; c) represents a sequence that directs site-directed homologous recombination to insert the full-length TCR ORF product reconstituted within the V-C entry vector backbone into a specific genetic context; d) permits engineered domain-fusion approaches to manipulate the form of the protein expressed from the full-length TCR ORF reconstituted in the V-C entry vector backbone e) permits cap-independent initiation of translation of the mRNA expressed from the full-length TCR ORF reconstituted in the V-C entry vector backbone f) permits insulation of transcriptional activity otherwise affected by enhancer elements in a genomic context of where the full-length TCR ORF reconstituted in the V-C entry vector backbone may be inserted.

A cis/acting element may be used to drive transient expression of TCRs reconstituted into a V-C entry vector backbone provided in Examples 1 and 3, when said vector containing a reconstituted TCR ORF is transfected into mammalian cells.

A heterospecific recognition site for recombinase enzymes may be used to permit recombinase mediated cassette exchange of TCRs reconstituted into a V-C entry vector backbone provided in Example 4, when said vector containing a reconstituted TCR ORF is transfected into mammalian cells in the presence of appropriate recombinase enzyme.

A first Type IIS recognition sequence that is included in the V-C entry vector is oriented to cleave 5′ of said recognition sequence and within the TCR variable gene segment (FIG. 4a) to produce a single-stranded DNA overhang at the 3′ end of the variable gene segment (FIG. 4g) that is complementary to that at the 5′ end of the synthesised odeCDR3 (FIG. 4c) For details on how this first Type IIS recognition sequence is designed, see Examples 1, 3, 5 and 6.

A V-C entry vector contains a negative selection marker between the first Type IIS recognition sequence, and the second Type IIS recognition sequence (infra vide, Figure 2). This negative selection marker is selected from
a. a restriction enzyme recognition site not contained elsewhere in the first component or within the TCR joining gene segment,
b. a bacterial suicide gene, and
c. a reporter element.
   wherein, the negative selection marker is used to eliminate host cells transformed with parental V-C entry vector, and thus reduce the background of a reconstitution reaction when using the first positive selection marker to select for transformants containing the target TCR ORF within the V-C entry vector backbone (see Example 7).

With the exception of the negative selection marker itself, all other sequences in the two-part system must be devoid of said sequence as to not confer undue negative selection on the basis of the inclusion of this sequence elsewhere in the system.

In the present context, a second Type IIS recognition sequence that is included in the V-C entry vector is orientated to cleave 3′ of said recognition sequence and within the TCR constant gene segment (FIG. 4a) to produce a single-stranded DNA overhang at the 5′ end of the constant gene segment (FIG. 4g) that is complementary to that at the 3′ end of the J donor fragment reaction intermediate (FIG. 4f). For details on how this second Type IIS recognition sequence is designed, see Examples 1, 2, 3 and 5.

The 3′ genetic element incorporated into a V-C entry vector comprises one or more elements selected from
a. a terminator element,
b. heterospecific recognition site for recombinase enzymes,
c. a 3′ homologous recombination arm for a genomic site of interest,
d. a mRNA splice donor site,
e. an internal ribosomal entry site, and
f. epigenetic insulator sequence.
   wherein a) represents a sequence that directs transcriptional termination for effective mRNA production of the TCR ORF in situ and may encode a poly-A signal; b) represents a sequence that directs site-directed homologous recombination to insert the full-length TCR ORF product reconstituted within the V-C entry vector backbone into a specific genetic context; c) permits the fusion of a TCR ORF to a transcriptional unit after integration into a genomic locus encoding an downstream mRNA splice acceptor site to manipulate the strength of TCR expression levels or form of the protein expressed from the full-length TCR ORF reconstituted in the V-C entry vector backbone e) permits cap-independent initiation of translation of the mRNA expressed from the full-length TCR ORF reconstituted in the V-C entry vector backbone f) prevent inappropriate interaction between adjacent chromatin domains, thus insulating the full-length TCR ORF from adjacent transcriptional regulation or spread of heterochromatin in a genomic context of where the reconstituted TCR ORF in the V-C entry vector backbone may be inserted

A terminator element is used to ensure transcriptional termination during expression of TCRs reconstituted into a V-C entry vector backbone provided in Examples 1 and 3, when said vector containing a reconstituted TCR ORF is a transfected into mammalian cells.

A heterospecific recognition site for recombinase enzymes is used to permit recombinase mediated cassette exchange of TCRs reconstituted into V-C entry vector backbones provided in Examples 4, when said vector containing a reconstituted TCR ORF is transfected into mammalian cells in the presence of appropriate recombinase enzyme.

A J donor vector contains a J gene segment with a C-part sequence, representing a 5′ fragment of the C gene segment, to the 3′ of the J gene segment (FIG. 3).

The C-part sequence is designed to standardise the single stranded overhangs generated by Type IIS enzyme action within the at the 3′ end of the J donor vector-derived J fragment reaction intermediate (FIG. 4f), and that at the 5′ end of the C gene segment within the Type IIS digested open V-C entry vector reaction intermediate (FIG. 4g).

A third Type IIS recognition sequence that is included in the J donor vector is oriented to cleave 3′ of said recognition sequence and within the TCR joining gene segment (FIG. 4b) to produce a single-stranded DNA overhang at the 5′ end of the joining gene segment (FIG. 4f) that is complementary to that at the 5′ end of the synthesised odeCDR3 (FIG. 4c) For details on how this third Type IIS recognition sequence is designed, see Examples 2, 3, 5 and 6.

A fourth Type IIS recognition sequence that is included in the J donor vector is orientated to cleave 5′ of said recognition sequence and within the TCR C-part (FIG. 4b) to produce a single-stranded DNA overhang at the 3′ end of the C-part (FIG. 4f) that is complementary to that at the 5′ Type IIS digested open V-C entry vector reaction intermediate (FIG. 4g). For details on how this third Type IIS recognition sequence is designed, see Examples 1, 2, 3, 5 and 6.

The two-part vector system, all encoded TCR gene segments and parts should not contain Type IIS recognition sequences that are used for operation, or assembly, of the V-C entry vector or J donor vector. Inclusion of such sequences would result in Type IIS restriction enzyme action within the encoded gene segments or parts, and result in disruption of the TCR reconstitution process. Similarly, the Type IIS recognition sequences should not be included in the vector backbones, or in any 5′ and 3′ genetic elements within these vectors, nor the cloning fragments used to assemble the two-part vector system, nor the odeCDR3 representing a third system component (infra vide).

A two-component vector system of the TORES may be constructed for any collection of TCR chains. In examples 1 to 4 below, two-component vector systems are constructed for the human TRA and TRB loci, encoding the human TCR alpha and beta chains, respectively. The construction of such a TORES is equally applicable in the context of the TRD and TRG loci, encoding the TCR delta and gamma chain pair, respectively, or indeed for any TRA/TRB, TRD/TRG or variant TCR chain pair system found in jawed vertebrates.

The Third odeCDR3 Component

To reconstitute a full-length TCR ORF using any given TORES, a small ORF fragment not encoded by the two-component V-C entry vector and J donor vector system is required as a third component. This third component takes the form of an oligonucleotide duplex encoding CDR3 (odeCDR3).

Such a third component, odeCDR3, comprises
a. a first single strand overhang sequence complimentary to first Type IIS restriction enzyme recognition and cleavage site orientated to cleave 5′ of the recognition sequence and within the TCR variable gene segment of the V-C entry vector,
b. a double strand segment encoding a TCR CDR3 region and devoid of negative selection element, which negative selection element is as defined in item 10, and also devoid of any Type IIS restriction sequences of the first or second part, and
c. a second single strand overhang sequence complimentary to the third Type IIS restriction enzyme recognition and cleavage site orientated to cleave 3′ of the recognition sequence and within the TCR joining gene segment of the J donor vector.

Alternatively, the odeCDR3 can be comprised of a dsDNA molecule and/or plasmid DNA encoding the CDR3 flanked by two Type IIS enzymes consistent with the first (V-C entry vector) or second (J donor vector) component, oriented such that when digested a product comprising of a, b and c described previously is generated, and two by-products encoding short dsDNA fragments flanked by the Type IIS sites. This alternative dsDNA odeCDR3 is compatible the restriction enzyme/ligase reaction, not necessarily requiring prior digestion or processing.

As an alternative to the use of V-C entry vector and J donor vector configuration in a TORES, J-C entry vector and V donor vector configuration may also be used by applying the same conceptual framework.

### Methods to Use a TORES to Reconstitute Full-Length TCR ORFs

A TORES can be used to reconstitute a full-length TCR ORF in a genetic vector context, from sequence information, as is presented for a human TRA/TRB chain pair in Example 7.

To operate a TORES to reconstitute a full-length TCR ORF from sequence information, given the resource of a two-component vector system for a given TCR chain, the method comprises
a. selecting a V-C entry vector,
b. selecting a J donor vector,
c. selecting an odeCDR3,
d. combining a, b and c to react with i) Type IIS restriction enzyme(s) to cleave all Type IIS restriction enzyme recognition and cleavage sites present in the V-C entry vector and in the J donor vector and ii) DNA ligase enzyme and iii) subjecting the combined mix to a thermocycling reaction,
e. transforming the reaction product obtained from step d to a selectable host organism competent for DNA vector propagation, and
f. performing a selection of host organism to obtain full length reconstituted TCR open reading frame in the V-C entry vector backbone.
   wherein, a) and b) are selected on the basis of the selected vector encoding the V,J and C gene segments in the target full-length TCR ORF; c) is selected on the basis of completing the full-length TCR ORF sequence not encoded by the V-C entry or J donor vectors selected in a) and b), and bounded by the Variable and Joining segments encoded therein; d) combining the three selected components into a reaction mixture along with a restriction enzyme that will cut the first, second, third and fourth Type IIS restriction enzyme recognition sequences within the V-C entry and J donor vectors; e) generally represents transformation-competent bacteria; f) selection of host is on the basis of the first positive selection marker provided by the V-C entry vector backbone.

Generally, a workflow to select and define the genetic elements of a full-length TCR ORF for reconstitution entails de novo sequencing of TCR chains from target organism tissues. Example 8 below presents the de novo identification of a set of TRA/TRB chain pairs specific for a HCMV antigen in a HLA-B*07:02 restricted context. The workflow described in FIG. 13, incorporates reverse transcription and PCR based amplification of TCR chain pairs from sorted single cells with subsequent Sanger sequencing. There exists a requirement for high-quality sequence information spanning V, CDR3, J and C segments of the TCR ORF, which dictates the specific sequencing approach(es) taken.

A method for selecting and reconstituting a TCR open reading frame thus comprises
a. Obtaining a TCR open reading frame sequence wherein said sequence information is sufficient to identify i) variable gene segment usage ii) constant gene segment usage iii) joining gene segment usage iv) a full CDR3 sequence spanning the variable gene segment border to the joining gene segment border, and
b. selecting a V-C entry vector corresponding to the variable and constant gene segments identified in step a. i) and a. ii), respectively, and
c. selecting a J donor vector corresponding to the joining gene segment identified in step a, iii), and
d. generating an odeCDR3 corresponding to CDR3 sequence identified in step a. iv), and
e. combining b, c and d to react with i) Type IIS restriction enzyme(s) to cleave all Type IIS restriction enzyme recognition and cleavage sites present in the V-C entry vector and in the J donor vector and ii) DNA ligase enzyme, iii) subjecting the combined mix to a thermocycling reaction, and
f. transforming the reaction product obtained from step e. to a selectable host organism competent for plasmid replication, and
g. performing a selection of host organism to obtain full length reconstituted TCR open reading frame in the V-C entry vector backbone.
   wherein, a) is conducted by sequencing methods well known to one skilled in the art, able to obtain sufficient sequence length and quality to identify all four required genetic elements; b) and c) are selected from a TORES library containing required vectors; d) is synthesised de novo or selected from an odeCDR3 library; e) is conducted in a single reaction vessel.

In order to select the appropriate V-C entry vector, J donor vector and odeCDR3, target TCR sequences were aligned against a library of V, C and J gene segments for their corresponding TCR chains to determine the V, C and J segment usage of the target chain. This sequence alignment and analysis step must also permit the definition of the CDR3 coding sequence, and thus the definition of odeCDR3 sequence. Thus, overall such sequence analysis permits the selection of V-C entry vectors and J donor vectors for TCR chain reconstitution. The analysis also permits the synthesis of odeCDR3 for each chain reconstitution reaction. This process is well described in Example 8 and summarised as part of FIG. 13.

It is desirable to conduct the Type IIS digestion and DNA ligase-dependent ligation (step e) in a single cycle reaction. This minimises processing steps and is made possible by the design of the system, with Type IIS restriction enzymes cutting outside their recognitions sequences, such that a number of unique overhangs may be generated with a single enzyme, thus maintaining efficient directional cloning of the J donor vector reaction intermediate and odeCDR3 into the V-C entry vector backbone.

Alternatively, the Type IIS restriction digest and DNA ligation may be performed in sequential procedures.

In Example 8, the application of the TORES is exemplified in the context of single-cell fluorescence-activated cell sorting (FACS) of antigen-specific CD8 T-cells from human peripheral blood for reverse transcription and PCR based amplification of TRA/TRB TCR chain pairs, followed by Sanger sequencing. This is a generally applicable workflow, wherein any tissue may be the source of T-cells from any jawed vertebrate, and cells may be sorted based on any phenotypic characteristic. Importantly, the single-sorted cells need not be stained for antigen specificity using HLA-multimer reagents.

The TCR sequencing approach used is not restricted to any particular method or technology, provided sufficient high-quality sequence information is obtained such that the above-defined genetic characteristics of the TCR ORF(s) can be defined based on said sequence information.

The use of FACS for partitioning single cells such that native TCR chain pairs may be sequenced and identified is a powerful method due to the accurate and rich phenotypic information that may be collected with multi-specificity antibody panels. However, other methods exist to partition cells, including; emulsion PCR; digital PCR approaches using microfluidic cell encapsulation, digital PCR using physical partitioning substrates.

It is generally desirable to obtain native TCR pairs from a source material, as both chains of a TCR pair contribute to HLA-antigen engagement and recognition. However, there are instances where recovery of just a single chain may be desirable, such as high-throughput screening of a single chain against a set specificity. In such a case, TCRs may be amplified and/or sequenced from non-partitioned cells.

Methods to Use a TORES to Generate Full-Length TCR ORFs with Diversified Sequence

A TORES system is ideally suited to generate diversified full-length TCR ORFs in several systematic modes. Such systematic diversification may be applied to affinity and/or functional maturation workflows for TCR chains. Such diversification of target TCR chain sequences is well described in Examples 9 and 10.

Such TCR ORF sequence diversification methods follow the same general scheme as for a reconstitution reaction. Diversification can be conducted in multiple parallel reconstitution reactions, whereby a single variant TCR ORF is generated per reaction. However, in most scenarios it is desirable to generate a pool of variant TCR ORFs in a single reaction. Each of these approaches is achieved by providing multiple variants of one or more of each genetic component(s) (V-C entry vector, J donor vector, odeCDR3) to a reconstitution reaction.

As described in Example 9, a TCR ORF can be systematically diversified at the CDR3 region by adding a pool of odeCDR3 with defined positional sequence diversity.

A method for selecting and reconstituting a TCR open reading frame to achieve TCR ORF diversity in the CDR3 region, thus comprises
a. Obtaining a TCR open reading frame sequence wherein said sequence information is sufficient to identify i) variable gene segment usage ii) constant gene segment usage iii) joining gene segment usage iv) a full CDR3 sequence spanning the variable gene segment border to the joining gene segment border, and
b. selecting a V-C entry vector corresponding to the variable and constant gene segments identified in step a. i) and a. ii), respectively, and
c. selecting a J donor vector corresponding to the joining gene segment identified in step a, iii), and
d. generating two or more odeCDR3 corresponding to CDR3 sequence identified in step a. iv), with variant sequence composition, and
e. combining b, c and d to react with i) Type IIS restriction enzyme(s) to cleave all Type IIS restriction enzyme recognition and cleavage sites present in the V-C entry vector and in the J donor vector and ii) DNA ligase enzyme, iii) subjecting the combined mix to a thermocycling reaction, and
f. transforming the reaction product obtained from step e. to a selectable host organism competent for plasmid replication, and
g. performing a selection of host organism to obtain full length reconstituted TCR open reading frame in the V-C entry vector backbone,
   wherein, a) is conducted by sequencing methods well known to one skilled in the art, able to obtain sufficient sequence length and quality to identify all four required genetic elements; b) and c) are selected from a TORES library containing required vectors; d) is synthesised de novo, or selected from an odeCDR3 library; e) is conducted in a single reaction vessel.

Such a method can be achieved by pooling all odeCDR3 variants to a single reaction to generate a pool of sequence-diversified but may be equally achieved by proving each odeCDR3 variant to a parallel reaction.

Variant odeCDR3 can be generated via a variety of methods well known to those skilled in the art. The selection of position and extent of odeCDR3 degeneracy/diversity can range from a single residue change at a single position, to completely degenerate sequence to the length of the odeCDR3.

As described in Example 10, a TCR ORF can be systematically diversified by maintaining the CDR3 region via provision of odeCDR3, but diversifying V, C and J segment usage by providing two or more of the V-C entry vector and/or J donor vector to the reconstitution reaction.

A method for selecting and reconstituting a TCR open reading frame with diversified V, C and/or J segment usage, thus comprises
a. Obtaining a TCR open reading frame sequence wherein said sequence information is sufficient to identify i) variable gene segment usage ii) constant gene segment usage iii) joining gene segment usage iv) a full CDR3 sequence spanning the variable gene segment border to the joining gene segment border, and
b. selecting two or more V-C entry vectors not corresponding to the variable and constant gene segments identified in step a. i) and a. ii), respectively, and
c. selecting two or more J donor vectors not corresponding to the joining gene segment identified in step a, iii), and
d. generating an odeCDR3 corresponding to CDR3 sequence identified in step a. iv), and
e. combining b, c and d to react with i) Type IIS restriction enzyme(s) to cleave all Type IIS restriction enzyme recognition and cleavage sites present in the V-C entry vector and in the J donor vector and ii) DNA ligase enzyme, iii) subjecting the combined mix to a thermocycling reaction, and
f. transforming the reaction product obtained from step e. to a selectable host organism competent for plasmid replication, and
g. performing a selection of host organism to obtain full length reconstituted TCR open reading frame in the V-C entry vector backbone.
   wherein, a) is conducted by sequencing methods well known to one skilled in the art, able to obtain sufficient sequence length and quality to identify all four required genetic elements; b) and c) are selected from a TORES library containing required vectors; d) is synthesised de novo, or selected from an odeCDR3 library; e) is conducted in a single reaction vessel.

Such a method can be achieved by pooling all V-C entry vectors and/or J donor vector variants to a single reaction to generate a pool of sequence-diversified but may be equally achieved by proving each vector variant to a parallel reaction.

Each V-C entry and J donor vector from a given library could be selected to provide full coverage of V, C and J gene segments.

Any combination of CDR3 and V, C and J diversification describe above could be used to generate pools or libraries of diversified TCR ORFs.

This system can be used to generate entirely synthetic libraries of TCRs ORFs with full coverage of native V, C and J gene segment usage, and defined CDR3 characteristics.

Features of a TORES with Regard to Reconstitution/Diversification Methods

As mentioned above, it is desirable to conduct the assembly cycle reaction with a single Type IIS restriction enzyme. This economises the use of restriction enzyme and is made possible by the nature of Type IIS action, and the design of unique single stranded overhangs in the two-component vector system and odeCDR3.

Alternatively, up to four Type IIS restriction enzyme recognition sequences across the four Type IIS recognition sites of the V-C entry vector and J donor vector.

For efficient cloning of TCR ORF products, at least one step of negative selection is performed during the assembly of a full-length TCR ORF using the TORES, selected from
a. performing restriction enzyme digest of reaction product to eliminate parental V-C entry vector
b. performing a suicide gene selection to eliminate competent hosts transformed with parental V-C entry vector, and/or
c. performing selection of host cells transformed with parental V-C entry vector by way of reporter identification.
   wherein, the negative selection is used to eliminate parental V-C entry vector that have remained undigested by the Type IIS enzyme(s), or have re-ligated to the parental form after digestion.

Elimination of parental V-C entry vector is critical, considering that the V-C entry vector backbone, and thus the positive selection marker carried in this backbone, is used for positive selection of the vector containing the full-length TCR ORF reaction product.

In the present context, negative selection is performed using a restriction enzyme site has been designed within the reaction by-product excised from the V-C entry vector (FIG. 4d). This negative selection procedure is described in examples 7 and 8.

Any one, or a combination of the above-mentioned negative selection methods can be employed to eliminate parental V-C entry vector from the final cloned products. Such a negative selection procedure may be omitted if the cloning efficiency is deemed high enough for efficient recovery of cloned reaction products.

The selection of the cloned full-length TCR ORF containing vectors in transformed host organism is required to obtain the final cloned product. Such selections are well-known to those skilled in the art.

A host organism represents a transformation-competent bacterium, and the selection of transformants containing the full-length TCR ORF contained in a V-C entry vector backbone comprises antibiotic selection. This entails adding antibiotic to the culture system in which the transformed cells are placed, and resistance to this antibiotic is encoded by the gene represented as the first positive selection marker in the V-C entry vector backbone.

Alternatively, removal of auxotrophic factors of the culture system in which transformats are placed can be a form of positive selection, wherein auxotrophic complementation is conferred by a gene product encoded in the V-C entry vector backbone. A combination of the above-described positive selections may be employed.

### V-C Entry Vector and J Donor Vector Libraries Comprising a TORES

For the efficient operation of a TORES to perform reconstitution or diversification of selected TCR ORFs, the pre-construction of a V-C entry vector and J donor vector library is required. It is from this library, which is specific for each TCR chain form that selections are made to fulfil the V/J/C usage of the target TCR ORF sequence, when complemented with the odeCDR3 sequence.

V-C entry and J donor vector libraries may be constructed to contain all germline TCR variable, constant and joining gene segments of an organism having such TCRs. Such a library may also include all V-C combinations in the V-C entry vector, as for the TRB locus specific TORES presented in Example 3, wherein the library is replicated with both Constant gene segments against each Variable segment.

A library of V-C entry and J donor vectors may contain V/J/C gene segments, such that translated amino acid sequence of the encoded protein is unmodified in relation to the protein sequence encoded by the germline gene segments.

Such a library permits change in the underlying nucleic acid sequence as to generate a library otherwise devoid of unwanted Type IIS recognition sequences, or positive and negative selection elements. Changes in the underlying nucleic acid sequence can also be used for codon optimisation, for expression reconstituted TCR chains in cells from different host organisms.

Alternatively, a library of V-C entry and J donor vectors may contain V/J/C gene segments, such that translated amino acid sequence of the encoded protein is modified in relation to the protein sequence encoded by the germline gene segments.

Such a library may be used to construct TCRs with characteristics that are optimised for different diagnostic or therapeutic uses. Changes in framework residues or regions within the V/J/C gene segments could be used to increase expression or stability in various scenarios, such as expression of TCRs as soluble reagents. Similarly, alterations in framework regions that are not involved in direct HLA-antigen contacts may be used to alter the signalling capacity of reconstituted TCRs produced by the TORES.

Affinity tags or immunogenic sequences may also be encoded within framework regions as to aid in purification and/or detection of the reconstituted TCRs in downstream applications.

V-C entry and J donor vector libraries may be assembled into kit comprising a combination of
a. one or more V-C entry vectors encoding combinations of Variable and Constant gene segments, and
b. one or more J donor vectors encoding J gene segments, and optionally
c. one or more standardised odeCDR3 with single stranded overhangs matched to V-C entry vector and J donor vector single strand overhangs as positive control odeCDR3, and optionally
d. A pre-assembled full-length TCR ORF as a reference
   wherein, a) and b) cover the required genetic diversity of gene segments from a target organism, with unmodified or modified amino acid sequence relevant for the intended application; c) is used as a positive control in reconstitution reactions d) is used as a positive control in downstream applications of full-length TCR ORFs reconstituted with the V-C entry vector and J donor vector libraries provided in said kit.

### Method to Construct V-C Entry Vectors

The assembly of V-C entry and J donor vector libraries may be achieved by a variety of molecular biology methods well known to those skilled in the art, including direct DNA synthesis of the required vectors. However, a rapid and cost-effective combinatorial approach using small gene segment-containing fragments is desirable. Such a method permits rapid cycling of V-C entry and J donor vector forms that are important for TCR engineering workflows. Similarly, a rapid expansion of a given V-C entry vector and/or J donor vector library can be used to account for single nucleotide polymorphism and other allelic differences of TCR gene segments between individuals of a given population, which may have functional significance or impact the immunogenicity of a TCR sequence in a pseudo-allogeneic therapeutic context. Systematic methods for assembling V-C entry vector and J donor vector libraries are well described in Examples 1, 2 and 3.

A method to construct a V-C entry comprises combining three DNA components selected from
a. a Variable gene segment cloning fragment
b. a Constant gene segment cloning fragment
c. a V-C entry vector backbone
   wherein, a) contains the Variable gene segment; b) contains the constant gene segment; c) represent the V-C entry vector backbone into which the Variable and Constant gene fragments are assembled.

In the present context, a Variable gene segment cloning fragment comprises
a. a 5′ primer bind sequence for polymerase chain reaction dependent propagation of the fragment
b. a fifth Type IIS sequence orientated to cut in the 3′ direction
c. a first overhang sequence that encodes a defined single stranded overhang upon Type IIs enzyme action on the fifth Type IIs sequence in b.
d. a Kozak sequence
e. a TCR variable gene segment
f. a first Type IIS sequence
g. a 5′ sequence segment of a negative selection marker
h. a sixth Type IIS sequence orientated to cut in the 5′ direction such that a single stranded overhang is generated within the 5′ sequence segment of the negative selection marker in g.
i. a 3′ primer bind sequence for polymerase chain reaction dependent propagation of the fragment
   wherein, b) and h) encode Type IIS sequences used in the assemble of the V-C entry vector; f) encodes a Type IIS sequences used in the operation of a reconstitution reaction; g) represents a fragment of the negative selection marker sequence that is completed by a complementary fragment provided in the Constant gene segment cloning fragment.

A schematic representation of the Variable gene segment cloning fragment is presented in FIG. 5. Examples 1 and 3 describe the format and use of these cloning fragments to assemble V-C entry vectors for the human TRA and TRB loci, respectively.

These examples define Human TRA Variable gene segment cloning fragments as SEQ0001 to SEQ0046, and Human TRB Variable gene segment cloning fragments as SEQ0435 to SEQ0481.

To assemble a V-C entry vector, a Variable gene segment cloning fragment must be combined with a Constant gene segment cloning fragment.

A Constant gene segment cloning fragment comprises
a. a 5′ primer bind sequence for polymerase chain reaction dependent propagation of the fragment
b. a seventh Type IIS sequence orientated to cut in the 3′ direction such that a single stranded overhang is generated within the 3′ sequence segment of the negative selection marker in c.
c. a 3′ sequence segment of a negative selection marker
d. a second Type IIS sequence
e. a TCR constant gene segment
f. a second overhang sequence that encodes a defined single stranded overhang upon Type IIs enzyme action on the eighth Type IIs sequence in g.
g. an eighth Type IIS sequence orientated to cut in the 5′ direction such that a single stranded overhang is generated in overhang sequence of f.
h. a 3′ primer bind sequence for polymerase chain reaction dependent propagation of the fragment
   wherein, b) and g) encode Type IIS sequences used in the assemble of the V-C entry vector; g) encodes a Type IIS sequences used in the operation of a reconstitution reaction; c) represents a fragment of the negative selection marker sequence that is completed by a complementary fragment provided in the Variable gene segment cloning fragment.

A schematic representation of the Variable gene segment cloning fragment is presented in FIG. 6. Examples 1 and 3 describe the format and use of these cloning fragments to assemble V-C entry vectors for the human TRA and TRB loci, respectively.

These examples define Human TRB Constant gene segment cloning fragment as SEQ0047, and Human TRB Constant gene segment cloning fragments as SEQ0482 and SEQ0483.

The Variable and Constant gene segment cloning fragments are combined into a V-C entry vector backbone to assemble a V-C entry vector,

A V-C entry vector backbone comprises
a. an origin or replication
b. a first positive selection marker
c. a 5′ genetic element
d. a first restriction enzyme recognition sequence permitting digestion of the backbone to create a single stranded overhang complimentary to the overhang created within the Variable gene segment cloning fragment by Type IIS action during the assemble reaction
e. a second restriction enzyme recognition sequence permitting digestion of the backbone to create the Constant gene segment cloning fragment by Type IIS action during the assemble reaction
f. a 3′ genetic element.
   wherein, c) and f) represent genetic elements used for application of reconstituted TCR ORFs in differing biological systems as mentioned above.

A schematic representation of the V-C entry vector backbone is presented in FIG. 7. Examples 1, 3 and 4 describe the format and use of different V-C entry vector backbone forms to assemble V-C entry vectors for TRA and TRB loci, respectively.

A V-C entry vector backbone used for downstream transient expression of reconstituted TCR ORFs in mammalian cells is defined as SEQ0048, whereas a pair of V-C entry vector backbones used for recombinase mediated cassette exchange of reconstituted TCR ORFs are defined as SEQ0688 and SEQ0689,

Rapid cycling of Variable and Constant gene cloning fragments into different V-C entry vector backbones is a cost-effective approach for altering the characteristics of the vector context of reconstituted TCR ORFs from a given V/J/C combination. This permits rapid replication of re-tasking of native or synthetic TCR gene segment libraries into different biological applications.

Within the examples of Variable and Constant gene segment cloning fragments and V-C entry vector backbones cited herein, the Type IIS enzyme used for vector assembly is BbsI, whereas the Type IIS enzyme used for TCR ORF reconstitution is BsaI. The V-C entry vector backbone contains restriction enzyme recognition sites for Acc65I and XbaI, to create compatible overhangs with the 5′ overhang and 3′ overhang generated in the Variable and Constant gene cloning fragments, respectively, by BbsI action.

Any other combination of restriction enzymes could be used for assembly and reconstitution reactions, provided they satisfy the above-described criteria.

Preferably, the Type IIS sequences used for assembly of the V-C entry vector, designated the fifth, sixth, seventh and eighth Type IIS sequences above, are the same.

Alternatively, up to four different Type IIS recognition sequences could be used for this procedure.

The Type IIS sequences used for assembly of the V-C entry vector, designated the fifth, sixth, seventh and eighth Type IIS sequences above, must be different from those used for the reconstitution reaction; designated first, second third and fourth Type IIS sequences above.

The method for combining of the Variable and Constant gene segment cloning fragments with the V-C entry vector backbone to assemble a V-C entry vector is well described in examples 1 and 3.

A method for assembly of a V-C entry vector comprises
a. Digestion of the V-C entry vector backbone with the two restriction enzymes specific for the recognition sequences contained within the V-C entry vector backbone
b. Combining the digested V-C entry vector backbone with the V cloning fragment and the C cloning fragment, along with DNA ligase enzyme and one or more Type IIS restriction enzyme(s) recognising the fifth, sixth, seventh and eighth Type IIS sequences, and subjecting the combined mix to a thermocycling reaction, and
c. Transformation of the resulting reaction product into competent host organism and positive selection using said first positive selection marker to obtain complete V-C entry vector
   wherein; a) creates single stranded overhangs complimentary with those generated in Variable and Constant cloning fragments by Type IIS enzyme action in b); b) represents the digestion of Variable and Constant fragments to generate overhangs that will ligate to the overhangs generated in a), and permit ligation of the complementary overhangs generated within the negative selection marker fragments to ligate the Variable and Constant fragments; c) represents selection and propagation of the V-C entry vector product.

### Generic Features of V-C Entry Vectors and Construction Elements

The above descriptions use the human HLA TRA and TRB loci as templates for definition of a TORES system, as also outlined in Examples 1 to 4. However, a gene segment family from any TCR loci can be assembled into a TORES. In the above description, guidelines is given on where there is flexibility in the design of a TORES system, both for the human TRA/TRB loci, but also applicable to any other loci, from any organism. In the current section, generic features of a system are described using the TRA/TRB design presented in Examples 1 to 4 as a template.

To achieve a TORES for any given TCR loci, four sequence elements are required specific for a TCR chain encoded by said TCR loci:
X—a variable (V) gene segment fragment
Y—a constant (C) genes segment fragment
Y′—a constant (C) gene segment part
Z—a joining (J) gene segment fragment

According to the above description and examples below, these four forms of TCR sequence element can be assembled into various vector contexts to construct and deploy a TORES for any given V-J-C combination for any given TCR chain. For example, systems for the native human TRG and TRD locus, variant and/or synthetic TCR chain forms, or native TCR chain forms of an organism other than humans.

The V and C gene segment fragments are those assembled into a V-C entry vector via a V cloning fragment and C cloning fragment, respectively. This process is well described in examples 1 and 3.

A generic Variable gene segment cloning fragment, wherein the first Type IIS sequence encodes recognition for the enzyme BsaI, and the fifth and sixth Type IIS sequences encode recognition for the enzyme BbsI, and the Variable gene segment cloning fragment is thus represented by the sequence SEQ0690. The encoded Variable gene segment fragment is denoted as XNₙ, wherein X is the designation for the Variable gene segment, N represents any nucleotide, and n represents the number of nucleotides in said sequence.

A generic Constant gene segment cloning fragment, wherein the first Type IIS sequence encodes recognition for the enzyme BsaI, and the seventh and eighth Type IIS sequences encode for recognition for the enzyme BbsI, and the Constant gene segment cloning fragment is thus represented by the sequence SEQ0691. The encoded Constant gene segment fragment is denoted as YNₙ, wherein Y is the designation for the Constant gene segment, N represents any nucleotide, and n represents the number of nucleotides in said sequence.

A V-C entry vector backbone represented by sequence SEQ0048, is used to construct V-C entry vectors suitable for transient expression or reconstituted full-length TCR open reading frames in a mammalian host cell, wherein the first and second overhangs are generated by Acc65I and XbaI enzyme action of the backbone, respectively, and the 5′ and 3′ genetic elements are represented by constitutive promoter and polyadenylation signal, respectively.

A V-C entry vector backbone represented by sequence SEQ0688, is used to construct

V-C entry vectors suitable for recombinase mediated cassette exchange with matched genetic targets with suitable heterospecific recombinase sequences, wherein the first and second overhangs are generated by Acc65I and XbaI enzyme action of the backbone, respectively, and the 5′ and 3′ genetic elements are represented by F14 and F15 heterospecific recombinase sequences directing flippase activity, respectively.

A V-C entry vector backbone represented by sequence SEQ0689, is used to construct V-C entry vectors suitable for recombinase mediated cassette exchange with genetic targets with matched heterospecific recombinase sequences, wherein the first and second overhangs are generated by Acc65I and XbaI enzyme action of the backbone, respectively, and the 5′ and 3′ genetic elements are represented by FRT and F3 heterospecific recombinase sequences directing flippase activity, respectively.

By combining the above-described generic V cloning fragment, C cloning fragment with a selected V-C entry vector backbone, Variant V-C entry vectors can be constructed for different downstream application of TCR ORFs reconstituted within these varying vector contexts.

A generic V-C entry vector constructed with the use of the V-C entry vector backbone having sequence SEQ0048, is represented by sequence SEQ0692. This resulting V-C entry vector is suitable for transient expression or reconstituted full-length TCR open reading frames in a mammalian host cell, wherein the first and second Type IIS sequences encode recognition for the enzyme BsaI, and the the Variable gene segment fragment is denoted XNₙ, and the Constant gene segment fragment is denoted YNₙ, wherein X and Y are designations for said sequences, N represents any nucleotide, and n represents the number of nucleotides in each sequence.

A generic V-C entry vector constructed with the use of the V-C entry vector backbone having sequence SEQ0688, is represented by sequence SEQ0693, containing F14 and F15 sequences suitable for recombinase mediated cassette exchange with genetic targets with matched heterospecific recombinase sequences, wherein the first and second Type IIS sequences encode recognition for the enzyme BsaI, and the the Variable gene segment fragment is denoted XNₙ, and the Constant gene segment fragment is denoted YNₙ, wherein X and Y are designations for said sequences, N represents any nucleotide, and n represents the number of nucleotides in each sequence.

A generic V-C entry vector constructed with the use of the V-C entry vector backbone having sequence SEQ0689, is represented by sequence SEQ0694, containing FRT and F3 sequences suitable for recombinase mediated cassette exchange with genetic targets with matched heterospecific recombinase sequences, wherein the first and second Type IIS sequences encode recognition for the enzyme BsaI, and the the Variable gene segment fragment is denoted XNₙ, and the Constant gene segment fragment is denoted YNₙ, wherein X and Y are designations for said sequences, N represents any nucleotide, and n represents the number of nucleotides in each sequence.

The use of pairs of V-C entry vectors with differing heterospecific recombinase sites may be used for each chain of a TCR chain pair, as presented in Example 4 for the human TRA and TRB chain pair. This means that in downstream application of TCR chains reconstituted in this paired TORES system, can be delivered into a genetic context with dual heterospecific recombinase receiver sites. For example, a cell line containing such dual heterospecific recombinase receiver sites for genomic integration of the TCR chain pair.

The above description of V-C entry vector, and the components from which they are assembled, is based on the use of Type IIS enzymes BbsI and BsaI for construction and operation, respectively. Based on the guidance given above, one or more alternative Type IIS enzymes may be used for each of these tasks.

### Method to Construct J Donor Vectors

As for the above-described combinatorial method for construction of V-C entry vectors, a combinatorial method may be used to construct J donor vectors. In the present method, a J donor vector is constructed in a two-step process involving the construction of an intermediate J receiving cassette vector in a first step, into which J segment parts are inserted in a second step to form the J donor vector. In this context, a J receiving cassette vector, as the J donor vector derived from it, contains a small fragment of a Constant gene segment, termed a C part. Thus a rapid combinatorial method or construction is desirable to iterate different J donor vector forms that require differential usage of Constant gene segments. This method is well described practically in Examples 2 and 3.

A method to construct J donor vector comprises combining four DNA components selected from
a. J receiving cassette fragment
b. J donor vector backbone
c. J receiving cassette vector
d. J segment part
   wherein, a) contains the above-mentioned C-part and four distinct Type IIS cloning sites for vector assemble and reconstitution reaction operation; b) is the vector backbone into which one is inserted to create c); d) is the J gene segment part that is combined with c) to create a J donor vector.

A J receiving cassette fragment comprises
a. a first single stranded overhang at the 5′ end complimentary to overhang sequence generated in the J donor vector backbone
b. a third Type IIS sequence orientated to cut in the 3′ direction, joined with a sequence that forms a single stranded overhang when acted upon by the enzyme directed by a ninth Type IIS sequence mentioned in c.
c. a ninth Type IIs sequence orientated to cut in the 5′ direction, and to create a single stranded overhang mentioned in b.
d. a negative selection marker
e. a tenth Type IIS sequence orientated to cut in the 3′ direction and create a single stranded overhang at the 5′ of the sequence described in f.
f. a C-part representing a 5′ portion of the Constant gene fragment, with an overhang sequence at the 5′ end generated by enzyme action directed by the tenth Type IIS sequence, and an overhang sequence at the 3′ end generated by enzyme action directed by the fourth Type IIS sequence mentioned in g.
g. a fourth Type IIS sequence orientated to cut in the 5′ direction such that a single stranded overhang is generated within the 5′ sequence containing the C-part mentioned in g
h. a second single stranded overhang at the 3′ end commentary to overhang sequence generated in the J donor vector backbone
   wherein; a) and h) are used for directional cloning into the J donor vector backbone; b) and g) encode Type IIS sequences used in the operation of a reconstitution reaction; c) and e) encode Type IIS sequences used in the assemble of the J donor vector; d) represents a negative selection marker for elimination of parental J receiving cassette vector during assembly of a J donor vector; f) represents the fragment of the Constant gene segment carried by the J donor vector.

A schematic representation of the J receiving cassette fragment is presented in FIG. 8. Examples 2 and 3 describe the format and use of J receiving cassette fragments to assemble J receiving cassette vectors for the human TRA and TRB loci, respectively.

In the present context, the J receiving cassette fragments are formed by annealing partially complimentary single stranded oligonucleotides resulting in a DNA duplex with single stranded overhangs at each termini.

A J receiving cassette fragment for the TRA locus are described as SEQ0098 and SEQ0099.

A J receiving cassette fragments for the TRB locus are described as SEQ0578 and SEQ0581, wherein two forms exist to account for the two Constant gene segments utilised at the human TRB locus.

In the provided examples, the J receiving cassette fragment, the Type IIS enzyme used for J donor vector assembly is BbsI, whereas the Type IIS enzyme used for TCR ORF reconstitution is BsaI.

A J donor vector backbone comprises
a. an origin or replication
b. a second positive selection marker
c. a first restriction enzyme recognition sequence permitting digestion of the backbone to create a single stranded overhang complimentary to an overhang in the J receiving cassette fragment
d. a second restriction enzyme recognition sequence permitting digestion of the backbone to create a single stranded overhang complimentary to an overhang in the J receiving cassette fragment
   wherein c) and d) permit the directional cloning of the J receiving cassette fragments through the use of unique complimentary overhang sequences.

A J donor vector backbone is represented schematically in FIG. 9 and described in detail in examples 2 and 3. A J donor vector backbone sequences is represented as SEQ0097.

The J donor vector backbone contains restriction enzyme recognition sites for EcoRI and XhoI, to create compatible overhangs with the 5′ overhang and 3′ overhangs provided in the J receiving cassette gene cloning fragments.

Any other combination of restriction enzymes could be used for assembly and reconstitution reactions, provided they satisfy the above-described criteria.

The method for generating a J receiving cassette vector by combining a J receiving cassette fragment and J donor vector backbone is well described in in examples 2 and 3.

A J receiving cassette vector is constructed by combining the TRAJ receiving cassette fragment and the TRAJ J donor vector backbone, wherein the method comprises
a. Digestion of the J donor vector backbone with the two restriction enzymes specific for the recognition sequences contained within the J donor vector backbone
b. Combining the digested J donor vector backbone with the J receiving cassette fragment with DNA ligase enzyme
c. Transformation of the resulting reaction product into competent host organism and positive selection using said the second positive selection marker to obtain complete J receiving cassette vector
   wherein; a) creates single stranded overhangs complimentary with those contained within the J receiving cassette fragments; b) represents ligation of complimentary overhangs to form the J receiving cassette vector; c) represents selection and propagation of the J receiving cassette vector product.

A schematic representation of a J receiving cassette vector is presented in FIG. 10. Examples 2 and 3 describe the construction of J receiving cassette vectors for the human TRA and TRB loci, respectively.

A J receiving cassette vector for the TRA locus is described as SEQ0098, whereas those for the TRB locus are described as SEQ0582 and SEQ0583. The TRB locus utilises two Constant gene segments, thus the replica TRB J receiving cassette vectors contain C part sequences specific for each Constant gene segment.

A J receiving cassette vector is combined with a J segment part to create a J donor vector. The J segment part encodes the bulk of the J gene segment.

Said J segment part comprises
a. A first single stranded overhang sequence at the 5′ end that is complementary to the overhang generated in the J receiving cassette vector, when acted on by the enzyme directed by the ninth Type IIS sequence
b. A Joining gene segment part
c. A second single stranded overhang sequence at the 3′ end that is complementary to the overhang generated in the J receiving cassette vector, when acted on by the enzyme directed by the tenth Type IIS sequence
   wherein, a) and c) direct directional cloning of the J segment part into the J receiving cassette vector that is digested by the Type IIS enzyme(s) provided in the assembly reaction; b) encodes the joining gene segment that is to be carried by the J donor vector product.

A schematic representation of a J segment part is presented in FIG. 11. Examples 2 and 3 describe J segment parts used to construct J donor vectors for the human TRA and TRB loci, respectively.

Short J segment parts spanning the J gene segment usage of the human TRA locus are described as SEQ0099 to SEQ0210, wherein the J segment part is generated by annealing partially complimentary single stranded oligonucleotides resulting in a DNA duplex with single stranded overhangs at each termini.

A short J segment part can be used to generate J donor vectors with standardised J segment parts of minimal length, as is presented in Examples 2 and 3.

A long J segment part can be used to generate J donor vectors with extended coverage of the J gene segment to minimise the size of the odeCDR3 used in TCR reconstitution reactions. Shortening of the odeCDR3 element economises on synthesis cost for said element, and also minimises the mutational load within these oligonucleotide duplexes.

Long J segment parts spanning the J gene segment usage of the human TRA locus are described as SEQ0211 to SEQ0322, wherein the J segment part is generated by annealing partially complimentary single stranded oligonucleotides resulting in a DNA duplex with single stranded overhangs at each termini.

Short J segment parts spanning the J gene segment usage of the human TRB locus are described as SEQ0584 to SEQ0609, wherein the J segment part is generated by annealing partially complimentary single stranded oligonucleotides resulting in a DNA duplex with single stranded overhangs at each termini.

Long J segment parts spanning the J gene segment usage of the human TRB locus are described as SEQ0610 to SEQ0635, wherein the J segment part is generated by annealing partially complimentary single stranded oligonucleotides resulting in a DNA duplex with single stranded overhangs at each termini.

In the present context, the Type IIS sequences used for assembly of the J donor vector, designated the ninth and tenth Type IIS sequences above, are the same.

Alternatively, two different Type IIS recognition sequences could be used for this procedure.

The Type IIS sequences used for assembly of the J donor vector, designated the ninth, and tenth Type IIS sequences above, must be different from those used for the reconstitution reaction; designated first, second, third and fourth Type IIS sequences above.

The Type IIS sequences used for assembly of the J donor vector, designated the ninth, and tenth Type IIS sequences above, are the same as those used to assemble the V-C entry vector; designated fifth, sixth, seventh and eighth Type IIS sequences above.

These Type IIS sequences used to assemble the V-C entry and J donor vectors need not be the same or different, as they are treated independently.

A J segment part is combined with a J receiving cassette vector that contains a matched C part to generate a J donor vector.

A J donor vector is constructed by combining a J donor vector backbone with a J segment part, wherein the method comprises
a. Combining J receiving cassette vector with the J segment part, along with DNA ligase enzyme and one or more Type IIS restriction enzyme(s) recognising the ninth and tenth Type IIS sequences, and subjecting the combined mix to a thermocycling reaction, a
b. Transformation of the resulting reaction product into competent host organism and positive selection using said second positive selection marker to obtain complete J donor vector
   wherein, the Type IIS enzyme action on the ninth and tenth Type IIS sequences in step a) creates single stranded overhangs within the J receiving cassette vector, through excision of the negative selection marker sequence.

A schematic representation of a resulting J donor vector is presented in FIG. 3. Examples 2 and 3 describe the use of J segment parts and a J receiving cassette vectors to construct J donor vectors for the human TRA and TRB loci, respectively.

J donor vectors containing short J segments for the human TRA locus are described as SEQ0323 to SEQ0378, whereas J donor vectors containing long J segments for the human TRA locus are described as SEQ0379 to SEQ0434.

J donor vectors containing short J segments for the human TRB locus paired with C1 constant gene segment are described as SEQ0636 to SEQ0648, whereas J donor vectors containing long J segments for the human TRA locus are described as SEQ0662 to SEQ0674.

J donor vectors containing short J segments for the human TRB locus paired with C2 constant gene segment are described as SEQ0649 to SEQ0661, whereas J donor vectors containing long J segments for the human TRA locus are described as SEQ0675 to SEQ0687.

In the provided examples, the Type IIS enzyme used to assemble a J donor vector is BbsI, whereas the Type IIS enzyme used within the reconstitution of a TCR is BsaI. The negative selection marker is a NotI restriction enzyme sequence.

The method to construct a J donor vector also entails a negative selection step to eliminate parental J receiving cassette vector prior to transformation and selection.

In the provided example, this negative selection entails a NotI digestion to eliminate parental J receiving cassette vector prior to transformation and selection.

### Generic Features of J Donor Vectors and Construction Elements

As described above and in Example 5 below, several features of a V-C entry and J donor vector library may be generic, inasmuch that TCR gene segment elements are constructed into a generic context to achieve a TORES system.

To achieve a TORES for any given TCR loci, four sequence elements are required specific for a TCR chain encoded by said TCR loci:
X—a variable (V) gene segment fragment
Y—a constant (C) genes segment fragment
Y′—a constant (C) gene segment part
Z—a joining (J) gene segment fragment

According to the above description and examples below, these four forms of TCR sequence element can be assembled into various vector contexts to construct and deploy a TORES for any given V-J-C combination for any given TCR chain. For example, systems for the native human TRG and TRD locus, variant and/or synthetic TCR chain forms, or native TCR chain forms of an organism other than humans.

The C gene segment part and J gene segment fragment are those assembled into J donor vector via a J receiving cassette fragment and a J cloning fragment, respectively. This process is well described in Examples 2 and 3.

A generic J receiving cassette fragment represented by the sequences SEQ0695 and SEQ0696 wherein the third and fourth Type IIS sequences encode recognition for the enzyme BsaI, and the ninth and tenth Type IIS sequences encode recognition for the enzyme BbsI, and the negative selection marker represents a NotI restriction enzyme recognition sequence. Within this sequence, the encoded C-part is denoted as Y′Nₙ, wherein Y′ is the designation for the C-part, N represents any nucleotide, and n represents the number of nucleotides in said sequence.

The J receiving cassette fragments is generated by annealing the single stranded oligonucleotides represented by the pair of sequences with partially complementary sequence.

A J donor vector backbone to construct a J receiving cassette vector with the above generic J receiving cassette fragment is represented by SEQ0097, wherein the restriction enzymes EcoRI and XhoI are used to generate single stranded DNA overhangs required for insertion of the J receiving cassette fragment sequence.

A J receiving cassette vector constructed by the combination of the J receiving cassette fragment generated from the J receiving cassette fragments represented as sequences SEQ0695 and SEQ0696, and a J donor vector backbone represented by sequence SEQ0097, is thus represented by SEQ0697. Within this resulting J receiving cassette vector, the third and fourth Type IIS sequences encode recognition for the enzyme BsaI, and the ninth and tenth Type IIS sequences encode recognition for the enzyme BbsI, and the negative selection marker represents a NotI restriction enzyme recognition sequence. As derived from the J receiving cassette fragment, the encoded C-part is denoted as Y′Nₙ, wherein Y′ is the designation for the C-part, N represents any nucleotide, and n represents the number of nucleotides in said sequence.

The J receiving cassette vector has a J segment part inserted to obtain a J donor vector.

A generic J segment part used to insert into the generic J receiving cassette vector represented by SEQ0697, is thus represented by complementary sequences SEQ0698 and SEQ0699, wherein the J gene segment part is denoted as ZNₙ, wherein Z is the designation for the J gene segment, N represents any nucleotide, and n represents the number of nucleotides in said sequence.

A J segment part is generated by annealing the two single stranded oligonucleotides represented by SEQ0698 and SEQ0699.

A generic J donor vector assembled from the combination of the J receiving cassette vector represented by SEQ0697 and the J segment part represented by complementary sequences SEQ0698 and SEQ0699 is thus represented by sequence SEQ0700. This resulting J donor vector thus contains two annotated sequence inserts of ZNₙ and Y′Nₙ, wherein Z is the designation for the J gene segment, and Y′ is the designation of the C segment part, N represents any nucleotide, and n represents the number of nucleotides in said sequences.

The above description of V-C entry vector, and the components from which they are assembled, is based on the use of Type IIS enzymes BbsI and BsaI for construction and operation, respectively. Based on the guidance given above, one or more alternative Type 115 enzymes may be used for each of these tasks.

### Use of Full-Length TCR ORFs in Defined Vector Contexts for Diagnostics and Therapeutics

A key challenge in harnessing T-cell immunity for treatment of disease is the inter-individual diversity of the HLA and TCR systems, along with the massive intra-individual content of the TCR repertoire. This means that medicaments and therapeutic strategies that rely on the assessment and/or provision of TCRs require robust assessment of TCR function in a bona fide biological context.

To achieve accurate assessment of native TCR chain pairs, a reliable and cost-effective high-throughput method for delivering captured TCR ORFs in defined vector contexts is needed. A TORES for any given chain pair is a means to deliver such TCR ORFs.

A defined vector context for TCR ORFs can be, for example, a transient expression vector, for instance, whereby the TCRs can be rapidly characterised on the surface of human cells as described in Example 8. In this example, TCRs could be sequenced and re-expressed to confirm expected specificity. This permits the use of validated TCR sequences for following TCR clonotype abundance by sequence or amplification- or probed-based assays for diagnostic procedures during immunotherapeutic interventions on a personalised basis. Similarly, rapid capture and validation of TCR chain pairs mean that said TCR pairs could be provided in personalised medicine—such as the delivery of soluble TCR constructs as a medicinal compound, or provision of the TCR in an effector cell as a cellular therapeutic.

In a similar approach, a TORES system is ideally suited for engineering TCR ORFs to change specificity and/or function, as outlined in Examples 9 and 10. This engineering can be used to enhance or reduce signalling strength and/or change or redirect the specificity of TCR chain pairs towards specific disease antigens. Such engineered TCR sequences could be provide in personalised immunotherapeutic strategies in place of native TCR chain pairs.

### The Bidirectional TCR ORF Reconstitution and Engineering System (TORES²)

The above-described TORES system treats each TCR chain within independent two-part vector library systems. Thus, the products of TORES operation are discrete vectors encoding reconstituted TCR ORFs. In a separate aspect, the present invention provides an alternative system to adjoin reciprocal TCR chain pairs into a single product vector using a two-step method. This is achieved in a five-component vector library system, wherein modified V-C entry vectors are combined with the J-donor vectors of the TORES system and odeCDR3 to achieve a reconstitution of a TCR chain pair in discrete reactions. The modified V-C entry vectors then permit the reconstitution TCR ORFs to be adjoined in a second step by addition of a fifth vector component, the Bidirectional Terminator donor vector (BiT donor), to achieve a vector encoding the two TCR OFR pairs in antiparallel coding sense.

This bidirectional TORES (TORES²) represents a combined five-component vector system as modifications to V-C entry vectors encoding elements of the reciprocal chain pairs are non-symmetrical. Meaning that the V-C entry vectors comprise distinct arrangements. That is, the reconstituted ORF of one chain is excised from the product vector of the first step and ligated in an antisense orientation into the reciprocal product vector of the first step. Therefore the pairing of the V-C entry vectors is critical, as one of the original V-C entry vectors of the TORES² system represents the final product backbone, and thus encodes the desired 3′ and 5′ genetic elements for downstream application of the reconstituted and adjoined TCR pair. For clarity, the description and examples presented below fix the TRA chain as the final product backbone (e.g. V-Cα entry vector) and the TRB as the chain integrated to this final product backbone (e.g. V-Cβ entry vector). The reciprocal arrangement is equally as valid, as is any other combination of reciprocal TCR chain pairs.

### V-C Entry Vector Components of TORES²

As mentioned above, the five-component vector library system comprising TORES² differs from the TORES system in the provision of modified V-C entry vector contexts. These modifications incorporate distinct Type IIS sites (labelled as Type IIS #2) and negative selection elements (labelled as −ve selection #2) from those utilised for TCR ORF reconstitution in a first step, to direct the adjoining of those reconstituted ORFs into a single vector in a second step. In effect, the TORES² system incorporates the TORES system within a new V-C entry vector context. The description below treats the TRA chain as the backbone of the final product, and the TRB chain as that ligated into the reconstituted TRA chain backbone in an antisense orientation. The same framework applies for any pair of TCR chains, and there is no particular reasons as to why either of the TCR chains need to be in one of the described vector arrangements, or the other.

The V-Cα entry vector contains,
a. origin of replication,
b. a first positive selection marker,
c. 5′ genetic element, or elements,
d. Kozak Sequence,
e. TCR alpha variable gene segment,
f. a first Type IIS sequence, for site specific recognition and cleavage by a Type IIS restriction enzyme (for Type IIS enzyme #1),
g. a first negative selection marker,
h. a second Type IIS sequence (for Type IIS enzyme #1),
i. TCR constant gene segment,
j. A third Type IIS sequence (for Type IIS restriction enzyme #2),
k. A second negative selection marker,
l. A fourth Type IIS sequence (for Type IIS restriction enzyme #2), and
m. 3′ genetic element, or elements
   wherein, a) and b) are used for propagation and selection of both parental V-C entry vector and the reconstituted TCR-containing vector in a bacterial host; c) and m) are used to define the downstream application of the reconstituted and adjoined full-length TCR ORFs, and may include elements as described above for the TORES; d) ensures efficient initiation of translation in eukaryotic cells, which could alternatively represent a Shine-Dalgarno sequence for transitional regulation in prokaryotes and archaea; e) represents the variable (V) alpha gene segment from the start codon to a motif at the 5′ edge of the CDR3 region conserved across all V segments in a given two-component vector system; f) represents a Type IIS #1 recognition sequence that directs a Type IIS restriction enzyme to cut in the 5′ direction as to create a standardised single stranded overhang at the 3′ end of the V gene segment; g) represents a negative selection marker #1 to eliminate parental V-C entry vector during operation of the system to reconstitute a full-length TCR ORF; h) represents a Type IIS #1 recognition sequence that directs a Type IIS restriction enzyme to cut in the 3′ direction as to create a standardised single stranded overhang at the 5′ end of the C gene segment; i) represents the constant (C) gene segment from a motif at the 5′ end of the C gene segment conserved across all C segments in a given two-component vector system, and which defines the boundary with the J segment (see FIGS. 2 and 4); j) represents a Type IIS #2 recognition sequence that directs a Type IIS restriction enzyme to cut in the 5′ direction as to create a standardised single stranded overhang at the 3′ end after the C gene segment, this Type IIS enzyme is different to the first and second Type IIS #1 enzymes; k) represents a negative selection marker #2 to eliminate parental TRA ORF vector during the operation of creating a bidirectional vector construct, it is distinct from the first negative selection marker; l) represents a Type IIS #2 recognition sequence that directs a Type IIS restriction enzyme to cut in the 3′ direction as to create a standardised single stranded overhang before the 3′ genetic element, this Type IIS enzyme is different to the first and second Type IIS #1 enzymes; m) represents the 3′ genetic element, or elements.

The arrangement of the above-described V-Cα entry vector is depicted in FIG. 20 a.

Generally, the first and second Type IIS enzymes (designated as Type IIS #1) are the same but may also be different. Similarly, the third and fourth Type IIS enzymes (designated as Type IIS #2) are generally the same but may also be different. Critically, the Type IIS enzyme(s) used for the first and second Type IIS (Type IIS #1) sites must be different from the enzyme(s) used for the third and fourth Type IIS (Type IIS #2) sites.

A V-Cα entry vector backbone is represented by SEQ0756, as presented in Example 11.

Example 11 below presents a V-Cα entry vector of the TORES² for the human TRA locus (SEQ0756), wherein some of the V/C sequences have been modified relative to those incorporated into the TORES system as to be devoid of the Type IIS enzyme used in the third and fourth sites (SEQ0757 to SEQ0763)

The V-Cβ entry vector contains,
a. origin of replication,
b. a first positive selection marker,
c. 5′ genetic element, or elements,
d. a first Type IIS sequence, for site specific recognition and cleavage by a Type IIS #2 restriction enzyme
e. Kozak Sequence,
f. TCR beta variable gene segment,
g. a second Type IIS sequence, for site specific recognition and cleavage by a Type IIS restriction enzyme (Type IIS #1),
h. a first negative selection marker,
i. a third Type IIS sequence (Type IIS #1),
j. TCR constant gene segment,
k. a fourth Type IIS sequence (Type IIS #2),
l. a second negative selection marker, and
m. 3′ genetic element, or elements
   wherein, a) and b) are used for propagation and selection of both parental V-C entry vector and the reconstituted TCR-containing vector in a bacterial host; c) and m) optionally define the downstream application of the reconstituted full-length TCR ORF as described above for the TORES system; d) represents a Type IIS #2 recognition sequence that directs a Type IIS restriction enzyme to cut in the 3′ direction as to create a standardised single stranded overhang after the 5′ genetic element and before the Kozak sequence, this Type IIS enzyme is different to the second and third Type IIS #1 enzymes; e) ensures efficient initiation of translation in eukaryotic cells, which could alternatively represent a Shine-Dalgarno sequence for transitional regulation in prokaryotes and archaea; f) represents the variable (V) gene segment from the start codon to a motif at the 5′ edge of the CDR3 region conserved across all V segments in a given two-component vector system; g) represents a Type IIS #1 recognition sequence that directs a Type IIS restriction enzyme to cut in the 5′ direction as to create a standardised single stranded overhang at the 3′ end of the V gene segment; h) represents a negative selection marker to eliminate parental V-C entry vector during operation of the system to reconstitute a full-length TCR ORF; i) represents a Type IIS #1 recognition sequence that directs a Type IIS restriction enzyme to cut in the 3′ direction as to create a standardised single stranded overhang at the 5′ end of the C gene segment; j) represents the constant (C) gene segment from a motif at the 5′ end of the C gene segment conserved across all C segments in a given two-component vector system, and which defines the boundary with the J segment (see FIGS. 2 and 4); k) represents a Type IIS #2 recognition sequence that directs a Type IIS restriction enzyme to cut in the 5′ direction as to create a standardised single stranded overhang after the C segment but before the 3′ genetic element, this Type IIS enzyme is different to the second and third Type IIS #1 enzymes; m) represents a second negative selection marker, which is different to the first −negative selection marker, l) represents 3′ genetic element or elements.

The arrangement of the above-described V-Cβ entry vector is depicted in FIG. 20b.

The 3′ and 5′ genetic elements are optional in the V-Cβ entry vector context, as the ORF reconstituted within this vector is later excised and ligated into the V-Cα entry vector backbone context. That is, the V-Cα entry vector backbone.

Generally, the second and third Type IIS enzymes (designated Type IIS #1) are the same but may also be different. Similarly, the first and fourth Type IIS enzymes (designated Type IIS #2) are generally the same but may also be different. Critically, the Type IIS enzyme(s) used for the second and third Type IIS sites (Type IIS #1) must be different from the enzyme(s) used for the first and fourth Type IIS sites (Type IIS #2).

Generally, the first and second Type IIS sites (Type IIS #1) of the V-Cα entry vector are the same as the second and third of the V-Cβ entry vector, but need not be as these reactions may be operated independently. The reconstitution of TRA chains and TRB chains in the described system could be conducted in a single reaction if the first and second Type IIS sites of the V-Cα entry vector were distinct from those of the second and third Type IIS sites of the V-Cβ entry vectors.

Generally, the third and fourth Type IIS sites (Type IIS #2) of the V-Cα entry vectors are the same as the first and fourth of the V-Cβ entry vectors as to require only the addition of a single Type IIS enzyme (Type IIS #2) to the second reaction adjoining reaction.

Generally, the second negative selection marker (−ve selection #2) of each V—Ca and V-Cβ entry vectors are the same as to facilitate efficient negative selection of parental vectors in the adjoining step of the TORES² operation, but may also be different.

A V-Cβ entry vector backbone is represented by SEQ0764, as presented in Example 11.

Example 11 below presents a V-Cβ entry vector of the TORES² for the human TRB locus (SEQ0764), wherein some of the V/C sequences have been modified relative to those incorporated into the TORES system as to be devoid of the Type IIS enzyme used in the first and fourth sites (SEQ0765 to SEQ0776)

J Donor and odeCDR3 Components of the TORES²

The J donor vector and the oligonucleotide duplex encoding the CDR3 are the same as already described for the conventional TORES above. Both are used within the first step reaction(s) to reconstitute the reciprocal TCR chain pairs within the designated V-C entry vector backbone contexts in a procedure identical to that described above for the TORES.

### Bidirectional Terminator Donor Vector (BiT Donor) of the TORES²

The first four vector library components of the TORES² represent the V-Cα and V-Cβ entry vectors, along with the J donor vectors for each of the TRA and TRB chain in the TRA/TRB focused description. Again, it should be noted that any TCR chains may be incorporated into this system following the described design principles, and the specific TRA and TRB configuration is fixed for clarity.

The fifth component represents Bidirectional Terminator Donor vector (BiT donor), which provides a bidirectional terminator element that is interposed by the antiparallel sense TRA and TRB chains reconstituted in the first step reaction, and adjoined by this bidirectional terminator element in the second step reaction.

The bidirectional terminator is generally provided as a vector and is also surrounded by two Type IIS restriction enzymes (Type IIS #2). It is equally as relevant to provide the bidirectional terminator element as a linear dsDNA construct with these Type IIS sites, or with target single strand DNA overhangs without Type IIS sites.

The vector containing the bidirectional terminator contains,
a. origin of replication,
b. a second positive selection marker,
c. first Type IIS sequence (Type IIS #2),
d. bidirectional terminator, and
e. second Type IIS sequence (Type IIS #2)
   wherein, a) and b) are used for propagation and selection of the vector carrying the bidirectional terminator in a bacterial host, c) represents a Type IIS recognition sequence that directs a Type IIS restriction enzyme (Type IIS #2) to cut in the 3′ direction as to create a standardised single stranded overhang, d) represents the bidirectional terminator which is used to ensure transcriptional termination on both sense and antisense DNA strands during expression of TCRs reconstituted into the TRA backbone, when said vector containing a reconstituted TCR ORF is transfected into mammalian cells; e) represents a Type IIS recognition sequence (Type IIS #2) that directs a Type IIS restriction enzyme to cut in the 5′ direction as to create a standardised single stranded overhang.

The arrangement of the above-described BiT donor vector is depicted in FIG. 21.

Generally, the positive selection marker contained within the BiT donor is distinct from that of positive selection markers carried by the V-Cα and V-Cβ entry vectors, as to minimise carryover of parental BiT donor in the second step adjoining reaction.

Generally, the first and second Type IIS sites (Type IIS #2) in the BiT donor are the same as both the third and fourth Type IIS sites of the V-Cα entry vectors and the first and fourth of the V-Cβ entry vectors, as to require only the addition of a single Type IIS enzyme (Type IIS #2) to the second adjoining reaction.

The bidirectional terminator element sequence is represented by SEQ0777.

### Operation of TORES² to Reconstitute and Adjoin Full Length TCR ORF Pairs

The operation of TORES² is a two-step process, incorporating a first TCR ORF reconstitution step, and second TCR ORF adjoining step. The method for reconstitution of TCR ORFs is identical to that described above for the TORES (see FIGS. 1 and 4). The products of the first step are therefore analogous to the products of TORES, comprising vectors encoding full-length TCR ORFs containing the V-CDR3-J-C element arrangement. However, the TORES² system provides these TCR ORFs in a distinct backbone context from the TORES system, wherein the TORES² backbone contains additional cloning sites to facilitate the second adjoining step reaction. This second step restriction enzyme and ligase cycle reaction entails the adjoining of the reconstituted TCR ORFs from the first step, in an antiparallel sense and interposing a bidirectional terminator element provided by the BiT donor. The overall process is depicted in FIG. 22, which depicts the above-described TRA/TRB TORES² arrangement.

The reconstituted TRA encoding vector (FIG. 22a), TRB encoding vector (FIG. 22b) and the BiT donor (FIG. 22c) are reacted in a single tube with Type IIS enzyme (Type IIS #2) and ligase and subjected to a restriction enzyme and ligase cycle reaction. The Type IIS restriction enzyme digests the three provided vectors into three reaction by-products, and three reaction intermediates with designed overhangs for directional ligation into the reaction product.

The three reaction by-products are represented by the excised −ve selection element #2 and Type IIS #2 sites from the TRA vector (FIG. 22d), the opened TRB encoding vector backbone from which the reconstituted TRB ORF has been excised (FIG. 22e) and the opened BiT donor backbone from which the bidirectional terminator element has been excised (FIG. 22f).

The first of the reaction intermediates is the open TRA vector, encoding the reconstituted TRA ORF in the V-Cα entry vector backbone context (FIG. 22g). The third Type IIS #2 site in the original V-Cα entry vector as described above directs the enzyme to cut in the 5′ direction to create a standardised single stranded overhang at the 3′ end after the C gene segment (FIG. 22 g, overhang j 1-5′), while the fourth Type IIS #2 site directs enzyme cleavage in the 3′ direction to create a standardised single stranded overhang before the 3′ genetic element (FIG. 22 e overhang j 3-3′).

The second reaction intermediate is the TRB ORF encoding fragment excised from the V-Cβ entry vector context. The first Type IIS #2 site in the V-Cβ entry vector described above directs the enzyme to cut in the 3′ direction to create an overhang prior to the Kozak sequence (FIG. 22h overhang j 3-5′). The fourth Type IIS #2 site directs the enzyme to cut in 5′ direction, creating an overhang after the C-segment (FIG. 22h overhang 2-3′). It should be noted that FIG. 22h depicts this intermediate in the antisense orientation in which it will be ligated into the product vector context.

The third reaction intermediate is the bidirectional terminator element excised from the BiT donor, where the first Type IIS #2 site directs the enzyme to cut in the 5′ direction to yield a standardised single stranded overhang (FIG. 22i overhang ‡ 1-3′). The second Type IIS #2 site directs the enzyme to cut in the 3′ direction to create a standardised single stranded overhang (FIG. 22i overhang ‡ 2-5′).

Within the restriction enzyme and ligase cycle reaction, the single stranded overhangs drive ligase-mediated directional ligation into the product vector, adjoining both TRA and TRB chains within the original V-Cα entry vector backbone context (FIG. 22j).

A TORES² may be constructed for any collection of TCR chains. For clarity, the TRA and TRB loci are used as a case study for description. The construction of such a TORES² is equally applicable in the context of the TRD and TRG loci, encoding the TCR delta and gamma chain pair, respectively, or indeed for any TRA/TRB, TRD/TRG or variant TCR chain pair system found in jawed vertebrates.

In the examples below, a TORES² system is provided for the human TRA/TRB loci, and applied to the reconstitution of a model human TCR ORF, and subsequently integrated into a matched engineered cell line harbouring RMCE sites matched with the 5′ and 3′ genetic elements of the originating V-Cα entry vector backbone context. This demonstrates that TORES² is readily used in combination with matched engineered cell lines optimised for integration and presentation of reconstituted TCR ORFs in a two-part device configuration similar to that described in application WO 2018/083318 A1.

In the following is given a table showing the sequences mentioned herein.

| | | Reference | |
|---|---|---|---|
| SEQ ID | Name | example | Description |
| 0001-0046 | TRA V cloning | Example 1 | Full DNA sequences of |
| | fragments | | the TRA V fragment |
| 0047 | TRA C constant | Example 1 | Full DNA sequence of |
| | cloning fragment | | the TRA C fragment |
| 0048 | V-C entry vector | Example 1 | DNA sequence of the |
| | backbone transient | | vector backbone from |
| | | | the 5′ genetic element |
| | | | encoding the CMV |
| | | | constitutive promoter to |
| | | | the 3′ genetic element |
| | | | encoding the SV40pA |
| | | | polyadenylation signal |
| 0049-0094 | TRA V-C entry vector | Example 1 | DNA sequences of the |
| | library sequence | | cloned V-C fragments |
| | | | that make up the TRA |
| | | | V-C entry vector library |
| 0095-0096 | TRA J receiving | Example 2 | Full DNA sequence of |
| | cassette fragments | | the TRA J receiving |
| | | | cassette fragment |
| | | | oligonucleotides |
| 0097 | J donor backbone | Example 2 | J donor vector back- |
| | | | bone is used to insert |
| | | | the TRA J receiving |
| | | | cassette fragment to |
| | | | create the TRA J |
| | | | receiving cassette vector |
| 0098 | TRA J receiving | Example 2 | See above |
| | cassette vector | | |
| 0099-0210 | TRA J Short segment | Example 2 | Encodes all amino |
| | part | | acids from the start of |
| | | | the CDR3-J border |
| | | | Phe codon |
| 0211-0322 | TRA J Long segment | Example 2 | Encodes more amino |
| | part | | acids N-terminal of the |
| | | | CDR3 border amino |
| | | | acids |
| 0323-0378 | TRA J Short donor | Example 2 | TRA short J donor |
| | vector | | library |
| 0379-0434 | TRA J Long donor | Example 2 | TRA long J donor |
| | vector | | library |
| 0435-0481 | TRB V cloning | Example 3 | Full DNA sequences |
| | fragment | | for the TRB V cloning |
| | | | fragments |
| 0482-0483 | TRB C constant | Example 3 | Full DNA sequences of |
| | cloning fragments | | the TRB C cloning |
| | | | fragments |
| 0484-0577 | TRB V-C entry vector | Example 3 | Sequences of the |
| | library sequence | | cloned V-C fragments |
| | | | that make up the TRA |
| | | | V-C entry vector library |
| 0578-0581 | TRB J receiving | Example 3 | TRB J receiving |
| | cassette fragments | | cassette fragments are |
| | | | constructed and inserted |
| | | | into a J donor vector |
| | | | backbone to create a |
| | | | TRB J receiving |
| | | | cassette vector |
| 0582-0583 | TRB J receiving | Example 3 | See above |
| | cassette vectors | | |
| 0584-0609 | TRB J Short segment | Example 3 | DNA sequences of the |
| | part | | short TRB J segment |
| | | | parts |
| 0610-0635 | TRB J Long segment | Example 3 | DNA sequences of the |
| | part | | long TRB J segment |
| | | | parts |
| 0636-0648 | TRB Cl J Short donor | Example 3 | TRB Cl short J donor |
| | vector | | library |
| 0649-0661 | TRB C2 J Short donor | Example 3 | TRB C2 short J donor |
| | vector | | library |
| 0662-0674 | TRB Cl J Long donor | Example 3 | TRB Cl long J donor |
| | vector | | library |
| 0675-0687 | TRB C2 J Long donor | Example 3 | TRB C2 long J donor |
| | vector | | library |
| 0688 | V-C entry vector | Example 4 | F14/F15 V-C entry |
| | backbone F14-F15 | | vector backbone |
| | | | sequence used to |
| | | | construct TRA V-C |
| | | | entry library |
| 0689 | V-C entry vector | Example 4 | FRT/F3 V-C entry |
| | backbone FRT-F3 | | vector backbone |
| | | | sequence used to |
| | | | construct TRB V-C |
| | | | entry library |
| 0690 | Generic V cloning | Example 5 | Contain the required |
| | fragment XNₙ (left | | V gene segment |
| | part) | | fragment in the context |
| | | | of appropriate cloning |
| | | | sites for final V-C |
| | | | entry vector assembly |
| | | | (full sequence: |
| | | | 0690-XNₙ-0745) |
| 0691 | Generic C cloning | Example 5 | full sequence: |
| | fragment YNₙ (left | | 0691-YNₙ-0746 |
| | part) | | |
| 0692 | Generic Transient V-C | Example 5 | full sequence: |
| | Entry vector XNₙ-YNₙ | | 0692-XNₙ-0747- |
| | (left part) | | YNₙ-0748 |
| 0693 | Generic F14-F15 V-C | Example 5 | full sequence: 0693- |
| | Entry vector XNₙ-YNₙ | | XNⁿ-0749-YNₙ-0750 |
| | (left part) | | |
| 0694 | Generic FRT-F3 V-C | Example 5 | full sequence: 0694- |
| | Entry vector XNₙ-YNₙ | | XNₙ-0751-YNₙ-0752 |
| | (left part) | | |
| 0695-0696 | Generic J receiving | Example 5 | full sequence of 0695: |
| | cassette oligonucleo- | | 0695-Y′Nₙ- |
| | tides Y′Nₙ (left part) | | TGAGACCC; full |
| | | | sequence of 0696: |
| | | | 0696-Y′Nₙ-0753 |
| 0697 | Generic J receiving | Example 5 | full sequence: 0697- |
| | vector Y′Nₙ (left part) | | Y′Nₙ-0754 |
| 0698-0699 | Generic J segment | Example 5 | full sequence of 0698: |
| | oligonucleotides ZNₙ | | ctcgZNₙ; full sequence |
| | | | of 0699: |
| | | | Y′N(4)ZNₙ; for both |
| | | | 0698 and 0699, the |
| | | | dummy sequence |
| | | | ″aaaaaaaaaa″ was |
| | | | included in the sequence |
| | | | listing due to the 10 |
| | | | nucleotide minimum |
| | | | requirement of the |
| | | | ST.25 |
| 0700 | Generic J Donor | Example 5 | full sequence: 0700- |
| | vector ZNₙ-Y′Nₙ | | ZNₙ-Y′Nₙ-0755 |
| | (left part) | | |
| 0701-0702 | JG9 TRA and TRB | Example 6 | DNA sequences of the |
| | full sequences copy | | TRA and TRB chains |
| 0703-0706 | JG9 odeCDR3 | Example 6 | odeCDR3 synthesised |
| | sequences | | for the TRA and TRB |
| | | | chains |
| 0707-0718 | Sequence results from | Example 8 | |
| | Example 8 | | |
| 0719-0742 | odeCDR3 collection | Example 8 | |
| | for reconstitution | | |
| | Example 8 | | |
| 0743-0744 | degenerate TRA | Example 9 | odeCDR3 oligos |
| | odeCDR3s Example 9 | | |
| 0745 | Generic V cloning | Example 5 | Contain the required V |
| | fragment XNₙ (right | | gene segment |
| | part) | | fragment in the context |
| | | | of appropriate cloning |
| | | | sites for final V-C entry |
| | | | vector assembly (full |
| | | | sequence: 0690-XNₙ- |
| | | | 0745) |
| 0746 | Generic C cloning | Example 5 | full sequence: 0691- |
| | fragment YNₙ (right | | YNₙ-0746 |
| | part) | | |
| 0747 | Generic Transient V-C | Example 5 | full sequence: 0692- |
| | Entry vector XNₙ-YNₙ | | XNₙ-0747-YNₙ-0748 |
| | (middle part) | | |
| 0748 | Generic Transient V-C | Example 5 | full sequence: 0692- |
| | Entry vector XNₙ-YNₙ | | XNₙ-0747-YNₙ-0748 |
| | (right part) | | |
| 0749 | Generic F14-F15 V-C | Example 5 | full sequence: 0693- |
| | Entry vector XNₙ-YNₙ | | XNₙ-0749-YNₙ-0750 |
| | (middle part) | | |
| 0750 | Generic F14-F15 V-C | Example 5 | full sequence: 0693- |
| | Entry vector XNₙ-YNₙ | | XNₙ-0749-YNₙ-0750 |
| | (right part) | | |
| 0751 | Generic FRT-F3 V-C | Example 5 | full sequence: 0694- |
| | Entry vector XNₙ-YNₙ | | XNₙ-0751-YNₙ-0752 |
| | (middle part) | | |
| 0752 | Generic FRT-F3 V-C | Example 5 | ull sequence: 0694- |
| | Entry vector XNₙ-YNₙ | | XNₙ-0751-YNₙ-0752 |
| | (right part) | | |
| 0753 | Generic J receiving | Example 5 | full sequence: 0696- |
| | cassette oligonucleo- | | Y′Nₙ-0753 |
| | tides Y′Nₙ (right part) | | |
| 0754 | Generic J receiving | Example 5 | full sequence: 0697- |
| | vector Y′Nₙ (right part) | | Y′Nₙ-0754 |
| 0755 | Generic J Donor | Example 5 | full sequence: 0700- |
| | vector ZNₙ-Y′Nₙ | | ZNₙ-Y′Nₙ-0755 |
| | (right part) | | |
| 0756 | V-Cα entry vector | Example 11 | V-Cα entry vector |
| | backbone FRT/F3 | | backbone for TORES2 |
| 0757-0763 | Modified TRA V-C | Example 11 | Full DNA sequences of |
| | sequences | | the TRA V fragment, |
| | | | modified for TORES2 |
| 0764 | V-Cβ entry vector | Example 11 | V-Cβ entry vector |
| | backbone FRT/F3 | | backbone for TORES2 |
| 0765-0776 | Modified TRB V-C | Example 11 | Full DNA sequences of |
| | sequences | | the TRB V fragment, |
| | | | modified for TORES2 |
| 0777 | Bidirectional | Example 11 | Bidirectional terminator |
| | terminator element | | element (Esp3I to |
| | | | Esp3I site) |
| 0778-0779 | Model TRA/TRB pair | Example 11 | known specificity for a |
| | | | HLA-A*02:01-restricted |
| | | | antigen. |

### List of Abbreviations

APC Antigen-presenting cell
BiT Bidirectional Terminator donor

### CAR-T CAR T-cell

CAR Chimeric antigen receptor
CDR Complementarity-determining regions
C segment Constant segment (also C region)

### CMV Cytomegalovirus

DAMPS Danger associated molecular patterns
DC Dendritic cells
DNA Deoxyribonucleic acid
dsDNA Double stranded Deoxyribonucleic acid molecule
D segment Diversity segment (also D region)
eAPC Engineered antigen-presenting cell
FACS Fluorescence-activated cell sorting
FRT Flippase recognition target
GEM T-cells Germ line-encoded mycolyl-reactive T-cells
GFP Green fluorescent protein
HLAI HLA class I
HLAII HLA class II
HDR Homology directed recombination
HLA Human leukocyte antigen
IgSF Immunoglobulin superfamily
IRES Internal ribosome entry site
ITAM Immunoreceptor tyrosine-based activation motif
iNK T-cells Invariant natural killer T-cells
J segment Joining segment (also J region)
MACS Magnetic-activated cell sorting
MAGE Melanoma associated antigen
MAIT Mucosal-associated invariant T
odeCDR3 Oligonucleotide duplex encoding CDR3
ORF Open reading frame
PAMPS Pathogen-associated molecular patterns
PCR Polymerase chain reaction
RMCE Recombinase mediated cassette exchange
RFP Red fluorescent protein

### RT Reverse Transcription

DNA Ribonucleic acid
SH2 Src homology 2
T-cells T lymphocytes

### TCR T-cell Receptor

TRA TCR alpha
TRB TCR beta
TRD TCR delta
TRG TCR gamma
TAA Tumour-associated-antigens

### TORES TCR ORF Reconstitution and Engineering System

### TORES2 Bidirectional TCR ORF Reconstitution and Engineering System

V segment Variable segment (also V region)
β2M β2-microglobulin
ZAP-70 ζ-chain-associated protein of 70 kDa

### List of Definitions

A pair of complementary TCR chains: two TCR chains wherein the translated proteins are capable of forming a TCRsp on the surface of a TCR presenting cell
Affinity: Kinetic or equilibrium parameter of an interaction between two or more molecules or proteins
Allele: Variant form of a given gene
Amplicon: a piece of DNA or RNA that is the source and/or product of artificial amplification using various methods including PCR.
Analyte: an entity that is of interest to be identified and/or measured and/or queried
Antibody: Affinity molecule that is expressed by specialized cells of the immune system called B-cells and that contains of two chains. B-cells express a very large and very diverse repertoire of antibodies that do generally not bind self proteins but can bind and neutralize pathogens or toxins that would threaten the host. Natural or artificially engineered antibodies are often used as affinity reagents.
APC: Antigen-presenting cell. A cell capable of presenting antigen on its cell surface, generally in the context of an HLA.
C (-segment): Constant segment. Also Constant region. One of the gene segments that is used to assemble the T-cell receptor. The c-region is a distinct segment that rather than driving diversity of the TCR, defines its general function in the immune system.
C cloning fragment: Constant Cloning fragment. Also referred to as a C gene segment cloning fragment. A construct carrying a portion of a C gene segment used to construct a V-C entry vector.
C-part: Constant part. A small portion of Constant gene segment sequence carried by a J receiving cassette fragment, J receiving cassette and J donor vector to standardise overhang sequences for operation of the TORES to reconstitute TCR ORFs.
CDR: complementarity-determining regions. Short sequences on the antigen-facing end of TCRs and antibodies that perform most of the target binding function. Each antibody and TCR contains six CDRs and they are generally the most variable part of the molecules allowing detection of a large number of diverse target molecules.
Cis-acting element: regions of non-coding DNA that regulate the transcription of nearby ORFs.
D (-segment): Diversity segment. Also D region. One of the gene segments that is used to assemble the T-cell receptor. Each individual has a large number of different variations of these regions making it possible for each individual to arm T-cells with a very large variety of different TCR.
Diversification: A process where a sequence is diversified.
DNA: Deoxyribonucleic acid. Chemical name of the molecule that forms genetic material encoding genes and proteins.
Endogenous: Substance that originated from within a cell
Eukaryotic conditional regulatory element: A DNA sequence that can influence the activity of a promoter, which may be induced or repressed under defined conditions
Eukaryotic Promoter: A DNA sequence that encodes an RNA polymerase binding site and response elements. The sequence of the promoter region controls the binding of the RNA polymerase and transcription factors, therefore promoters play a large role in determining where and when your gene of interest will be expressed.
Eukaryotic terminator/Signal terminator: A DNA sequence that are recognized by protein factors that are associated with the RNA polymerase II and which trigger the termination process of transcription. It also encodes the poly-A signal
FACS/Flow Cytometry: Fluorescence-activated cell sorting. Flow cytometry is a technique by which individual cells can be analyzed en masse for the expression of specific cell surface and intracellular markers. A variation of that technique, cell sorting, allows cells that carry a defined set of markers to be retrieved for further analysis.
Flippase: A recombinase (Flippase, Flp) derived from the 2 μm plasmid of baker's yeast Saccharomyces cerevisiae.
Heterospecific recombinase sites: A DNA sequence that is recognized by a recombinase enzyme to promote the crossover of two DNA molecules
HLA I: Human Leukocyte Antigen class I. A gene that is expressed in humans in all nucleated cells and exported to the cell surface where it presents as cargo short fragments, peptides, of internal proteins to T-cell receptors. As such it presents fragments of potential ongoing infections along with intrinsic proteins. The HLA I can additionally present as cargo peptides that are added to the culture medium, generated from proteins expressed form introduced genetic elements or generated from proteins that are taken up by the cell. HLA class I genes are polymorphic meaning that different individuals are likely to have variation in the same gene leading to a variation in presentation. Related to HLA class II.
HLA II: Human Leukocyte Antigen Class II. A gene that is expressed in humans in specific cells that are coordinating and helping the adaptive immune response for example dendritic cells. Related to HLA class I. HLA class II proteins are exported to the cell surface where they present as cargo short fragments, peptides, of external proteins to T-cell receptors. As such it presents fragments of potential ongoing infections along with intrinsic proteins. The HLA II can additionally present as cargo peptides that are added to the culture medium, generated from proteins expressed form introduced genetic elements or generated from proteins that are taken up by the cell. HLA class II genes are polymorphic meaning that different individuals are likely to have variation in the same gene leading to a variation in presentation.
Homologous arms: A stretch of DNA that has near identical sequence identity to a complement homologous arm and therefore promote the exchange of two DNA molecules by the cellular process, homology directed repair.
Immune surveillance: Process in which the immune system detects and becomes activated by infections, malignancies or other potentially pathogenic alterations.
Insulator: A DNA sequence that prevents a gene from being influenced by the activation or repression of nearby genes. Insulators also prevent the spread of heterochromatin from a silenced gene to an actively transcribed gene.
Integration: The physical ligation of a DNA sequence into a chromosome of a cell
Internal ribosome entry site (IRES): A DNA sequence that once transcribed encodes a RNA element that allows the initiation of translation in a cap-independent manner
J (-segment): Joining segment. Also J region. One of the gene segments that is used to assemble the T-cell receptor. Each individual has a large number of different variations of these regions making it possible for each individual to arm T-cells with a very large variety of different TCR.
J-C entry vector: The vector of the two-component vector system that carries the J and C TCR segments, and which receives sequences from the V donor vectors and odeCDR3 during reconstitution of a full-length TCR ORF.
J donor backbone: Joining donor backbone. The vector backbone into which a J receiving cassette fragment is inserted to create a J receiving cassette vector.
J donor vector: The vector of the two-component vector system that carries the J TCR segment, and donates this segment to the V-C entry vector during reconstitution of a full-length TCR ORF.
J receiving cassette fragment: Joining receiving cassette fragment. A cloning fragment that carries a C-part used to construct a J receiving cassette vector.
J receiving cassette vector: Joining receiving cassette vector. The vector, carrying a C-part, into which a J segment part is inserted to create a J donor vector.
J segment part: Joining segment part. A DNA construct carrying a portion of a J gene segment that is inserted into a J receiving cassette vector to generate a J donor vector.
K is a nucleotide code indicating Keto (K=G or T) Kozak Sequence: Short sequence required for the efficient initiation of translation
M is a nucleotide code indicating aMino (M=A or C)
Major HLA class I: a Family of APX that comprise of the genes HLA-A, HLA-B and HLA-C
Matched: When two components encode genetic elements that direct and restrict the interaction between the complemented components
Meganuclease recognition site: A DNA sequence that is recognized by a endodeoxyribonuclease, commonly referred to as a meganuclease
Mobile genetic element: A DNA sequence that can permit the integration of DNA with the activity of transposase enzymes
N is a nucleotide code indicatin4g0 aNy nucleotide (N=A, T, C or G)
Native: an entity that is naturally occurring to the cell
Negative Selection Marker: A selectable marker that confers negative selection of a vector and/or of host organism carrying said marker-bearing vector
odeCDR3: oligonucleotide duplex encoding complementarity-determining regions. A synthetic construct carrying CDR3 genetic sequence with terminal overhangs, used in conjunction with the two-component vector system to reconstitute a full-length TCR ORF.
Origin of replication: a particular sequence in a vector, plasmid or genome at which replication is initiated.
ORF: Open reading frame. Stretch of genetic material that encodes a translation frame for synthesis of a protein (polypeptide) by the ribosome
Overhang: A single stranded sequence at the terminus of a double stranded nucleic acid molecule. Often referred to as sticky or cohesive ends.
PCR: Polymerase chain reaction in which a specific target DNA molecule is exponentially amplified
Peptide: short string of amino acids between 6-30 amino acids in length
Phenotypic analysis: Analysis of the observable characteristics of a cell.
Plasmid: A genetic construct can replicate independently of the chromosomes, typically a small circular DNA strand in the cytoplasm of a bacterium or a protozoan.
Polymorphic: Present in different forms in individuals of the same species through the presence of different alleles of the same gene.
Polypeptide: Protein consisting of a stretch of peptides, forming a three-dimensional structure.
Positive Selection Marker: A selectable marker that confers positive selection of a vector and/or host organism carrying said marker-bearing vector.
Primer: Short DNA sequence that allows specific recognition of a target DNA sequence for example during a PCR.
Promoter: Regulatory DNA element for the controlled initiation of gene expression.
Recombinase: Enzymes that mediate genetic recombination.
Reconstitution: In the present context, reconstitution describes the operation of the TORES and TORES² to assemble a TCR ORF. A ‘reconstitution reaction’ is the reaction mix and thermocycling reaction that this operation entails.
Reporter Element: A genetic element that mediates a reported signal in the organism or vector bearing said element. May be used as a positive or negative selection maker.
Restriction Enzyme Cleavage Sequence: The genetic sequence cleaved by a restriction enzyme, which can be intrinsic or intrinsic to the recognition sequence of said restriction enzyme.
Restriction Enzyme Recognition Sequence: The genetic sequence recognised and engaged by a restriction enzyme.
Selectable marker: A DNA sequence that confers a trait suitable for artificial selection methods.
Slice acceptor site: A DNA sequence at the 3′ end of the intron AM, APX CM or affinity reagent for interaction with cells with TCRsp on the surface, or TCRsp based reagents.
Slice donor site: A DNA sequence at the 5′ end of the intron.
Suicide gene: A gene that will mediate cell death within the host organism carrying said gene. May be used as a positive or negative selection marker.
Synthetic: an entity that is artificially generated.
T-cell: T lymphocyte. White blood cell that expresses a T-cell receptor on its surface. Selected by the immune system to not react with the own body but have the potential to recognize infections and malignancies as well as reject grafts from most members of the same species.
TCR: T-cell Receptor. Affinity molecule expressed by a subgroup of lymphocytes called T-lymphocytes. In humans the TCR recognizes cargo presented by APX CM or APX AM, including fragments from virus or bacterial infections or cancerous cells. Therefore, the TCR recognition is an integral part of the adaptive immune system. The TCR consists of two chains that are paired on the cell surface. The TCR expressed on the surface of each cells is assembled at random from a large pool of varied genes (the v,d,j and c segments) and thus each individual has a pool of T-cells expressing a very large and diverse repertoire of different TCRs.
TRA: TCR alpha encoding locus. One of the four different locus encoding genes that can form a VDJ recombined TCR chain. Translated TCR alpha chain proteins typically pair with translated TCR beta chain proteins to form alpha/beta TCRsp.
TRB: TCR beta encoding locus. One of the four different locus encoding genes that can form a VDJ recombined TCR chain. Translated TCR beta chain proteins typically pair with TCR alpha chain proteins to form alpha/beta TCRsp.
TRD: TCR delta encoding locus. One of the four different locus encoding genes that can form a VDJ recombined TCR chain. Translated TCR delta chain proteins typically pair with translated TCR gamma chain proteins to form gamma/delta TCRsp.
TRG: TCR gamma encoding locus. One of the four different locus encoding genes that can form a VDJ recombined TCR chain. Translated TCR gamma chain proteins typically pair with translate TCR delta chain proteins to form gamma/delta TCRsp.
Type IIS Restriction Enzyme: restriction enzymes that recognize asymmetric DNA sequences and cleave outside of their recognition sequence.
V (-segment): Variable region. Also V region. One of the gene segments that is used to assemble the T-cell receptor. Each individual has a large number of different variations of these regions making it possible for each individual to arm T-cells with a very large variety of different TCR.
V-C entry vector: The vector of the two-component vector system that carries the V and C TCR segments, and which receives sequences from the J donor vectors and odeCDR3 during reconstitution of a full-length TCR ORF.
V cloning fragment: Variable Cloning fragment. Also referred to as a V gene segment cloning fragment. A construct carrying a portion of a V gene segment used to construct a V-C entry vector.
V donor vector: The vector of the two-component vector system that carries the V TCR segment and donates this segment to the J-C entry vector during reconstitution of a full-length TCR ORF.
Vector: A vector is a genetic construct that carries genetic information. In the present context vector usually describes plasmid DNA vectors. A vector can represent any such construct that can be propagated and selected in a host organism.
W is a nucleotide code indicating Weak (W=A or T).

### LEGENDS TO FIGURES

FIG. 1. Two-component vector system for rapid full-length TCR ORF reconstitution.

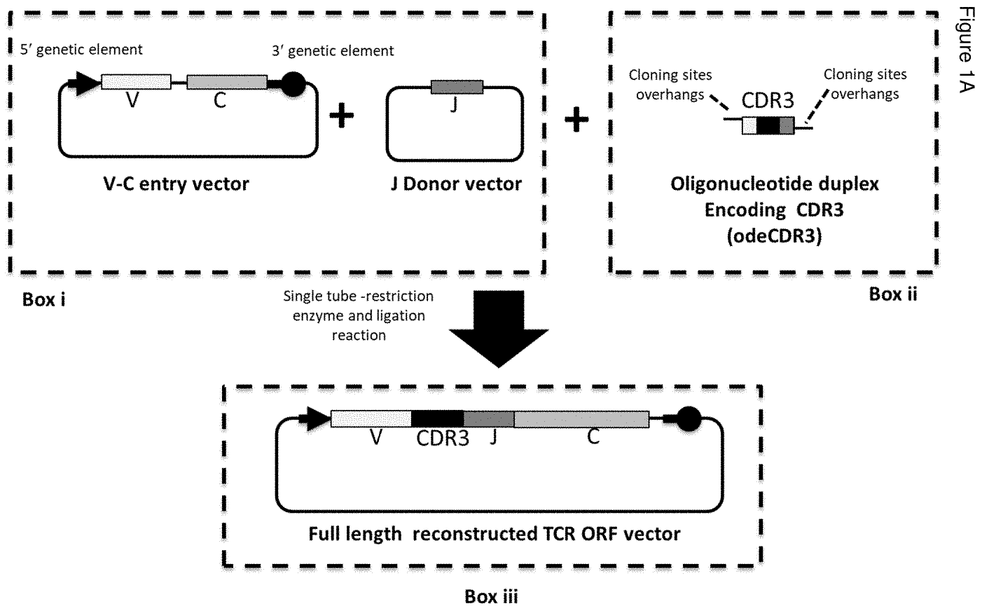

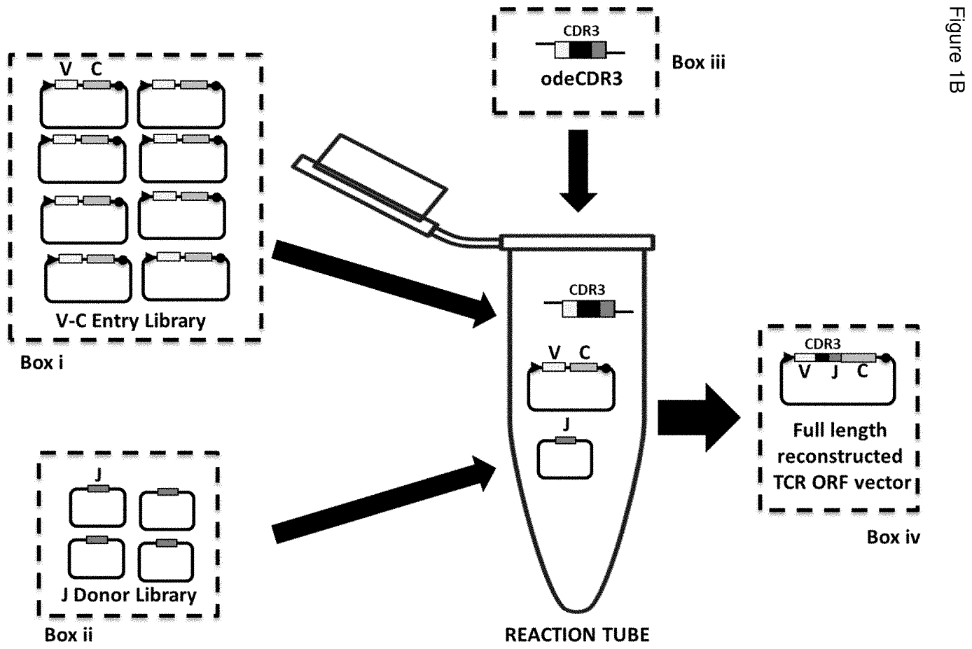

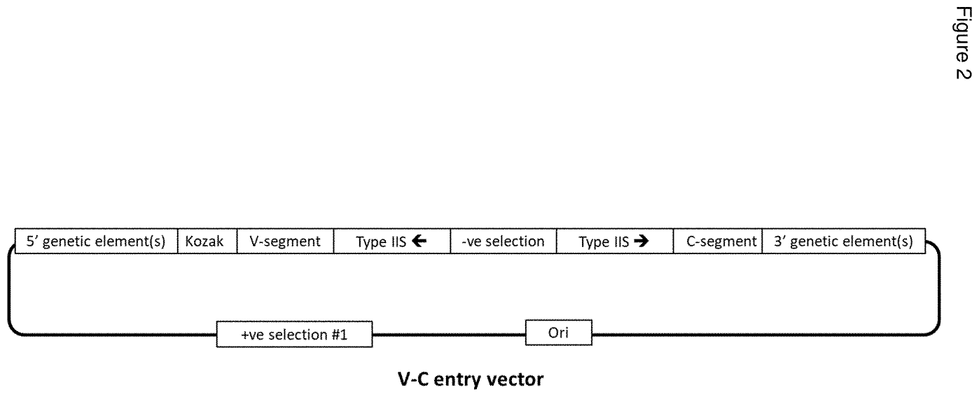

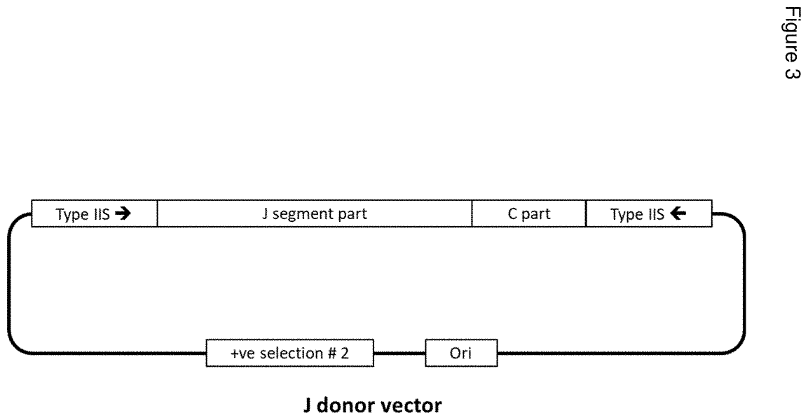

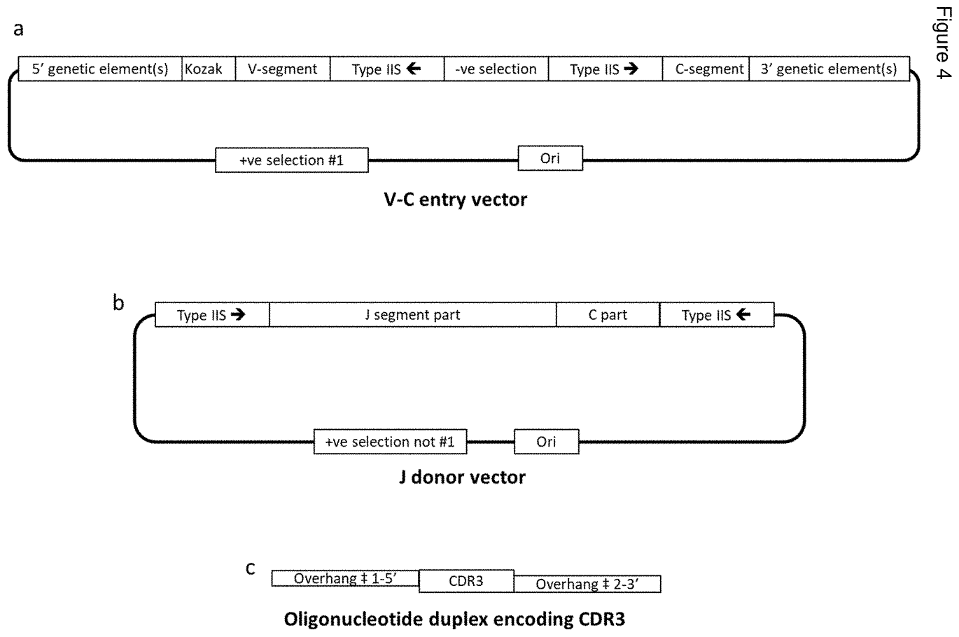

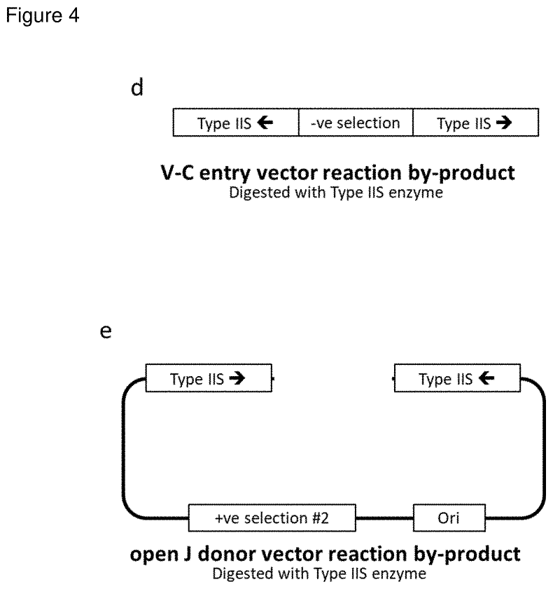

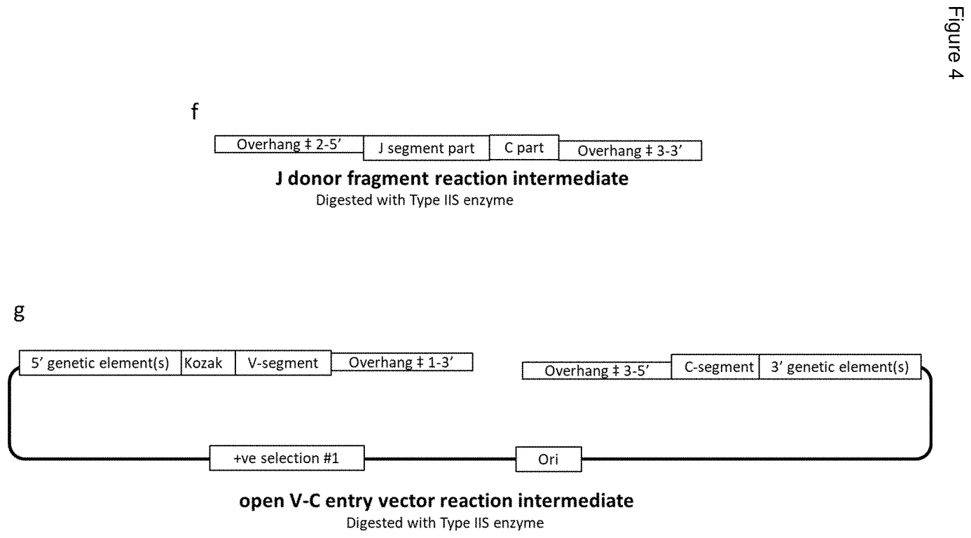

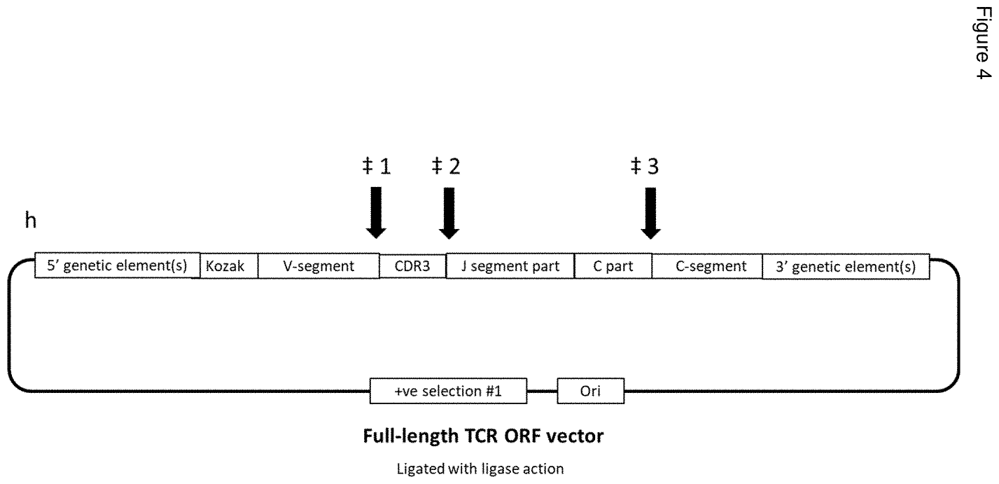

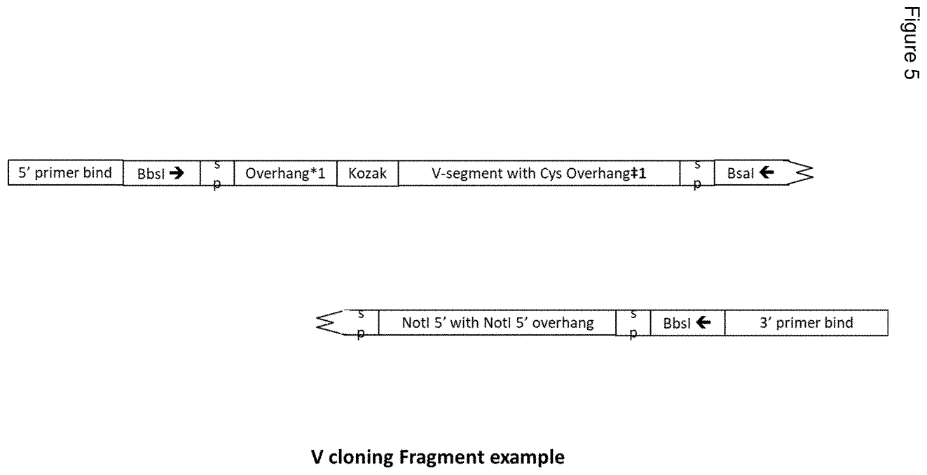

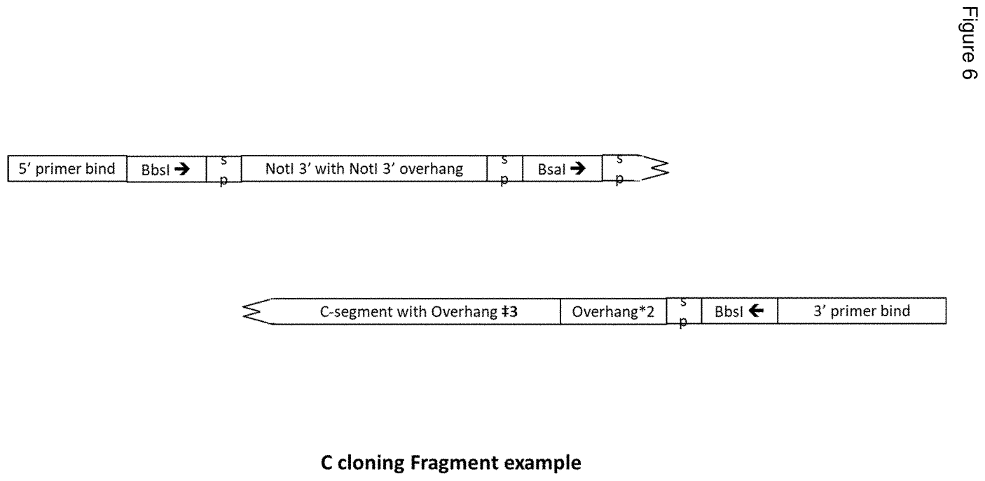

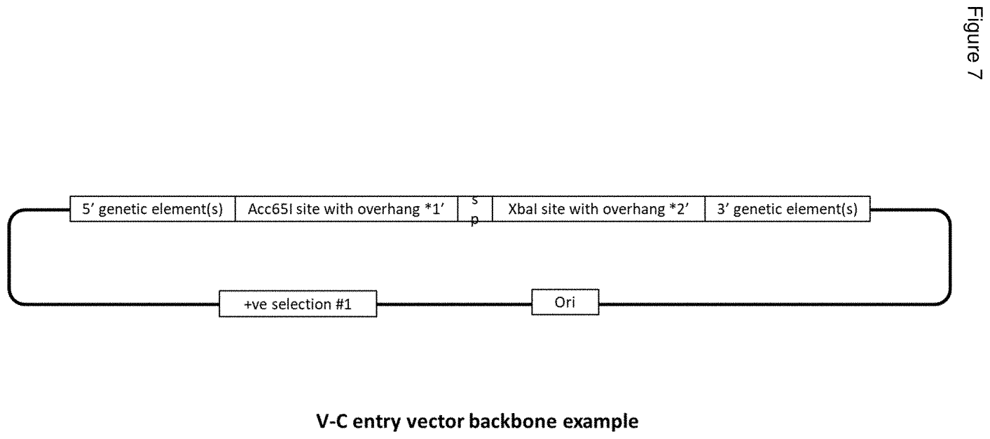

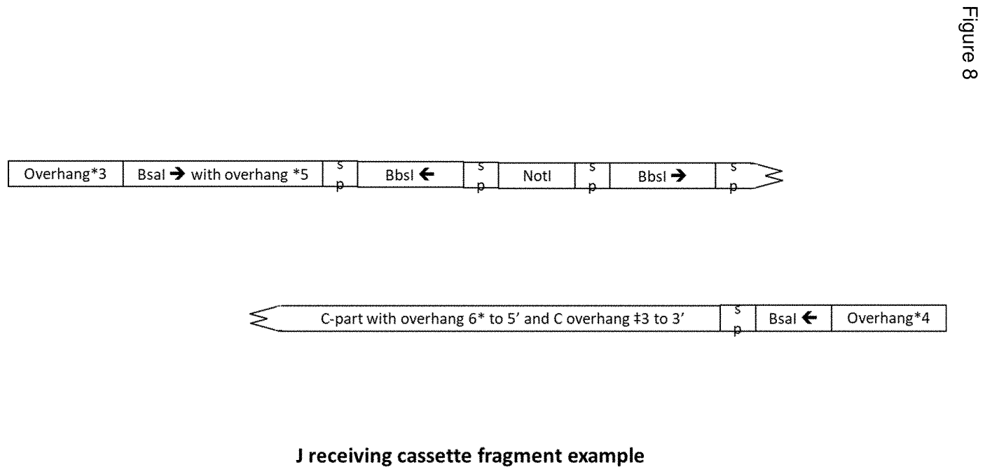

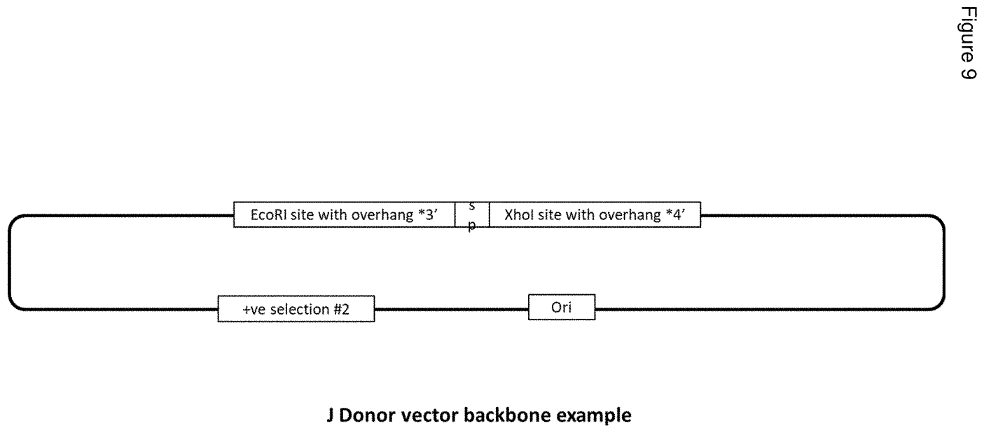

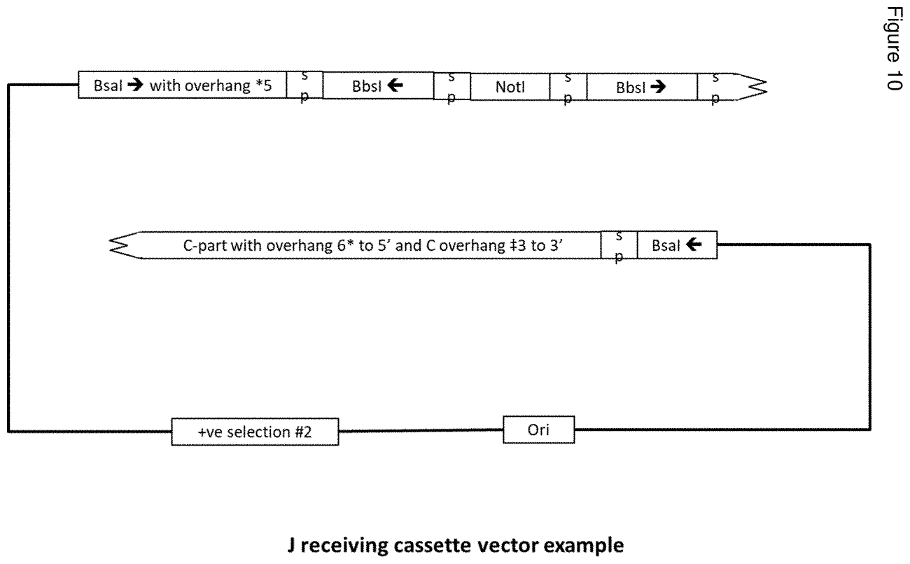

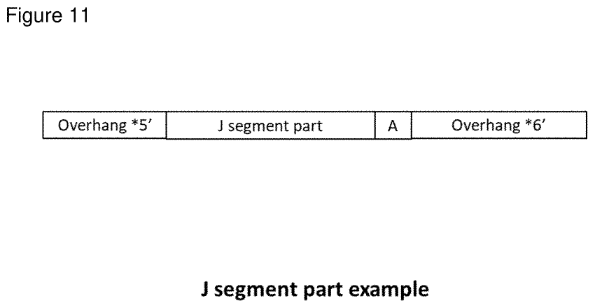

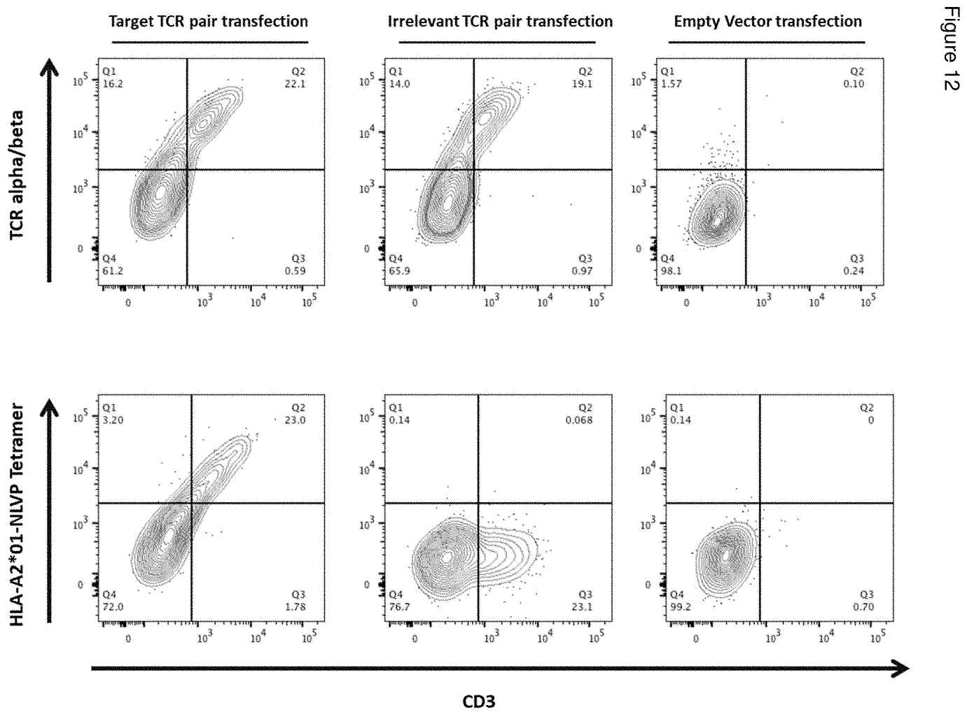

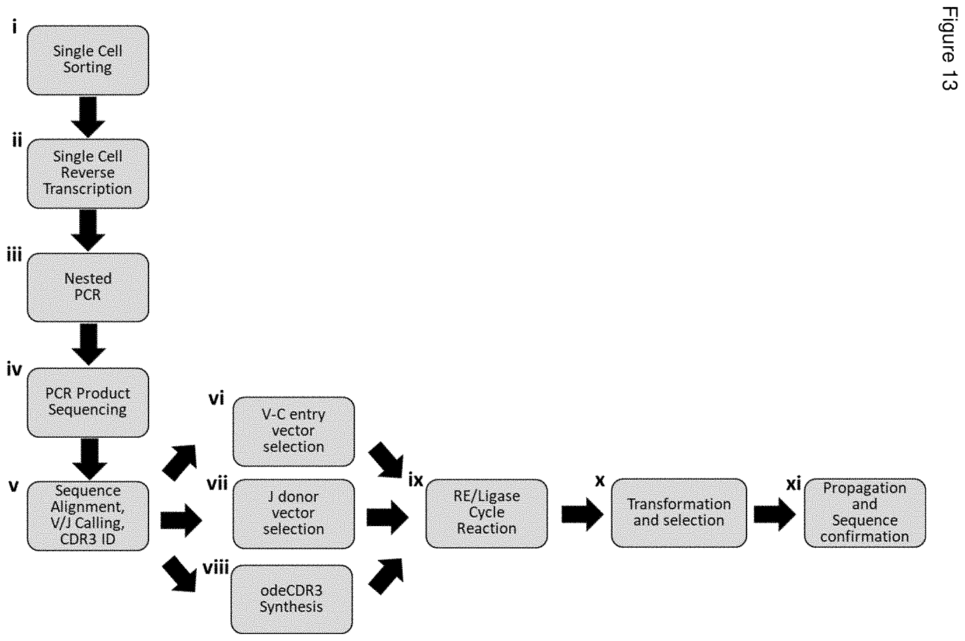

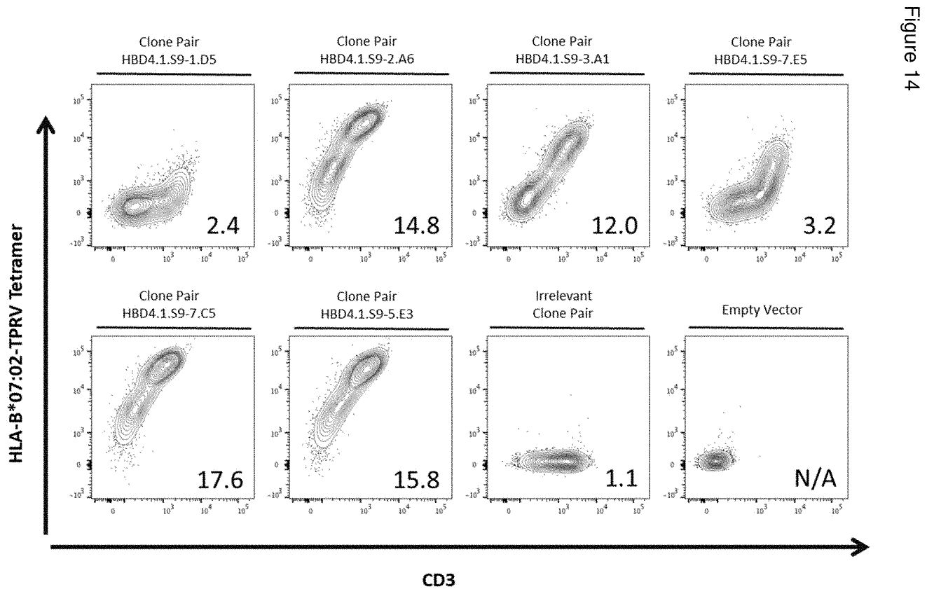

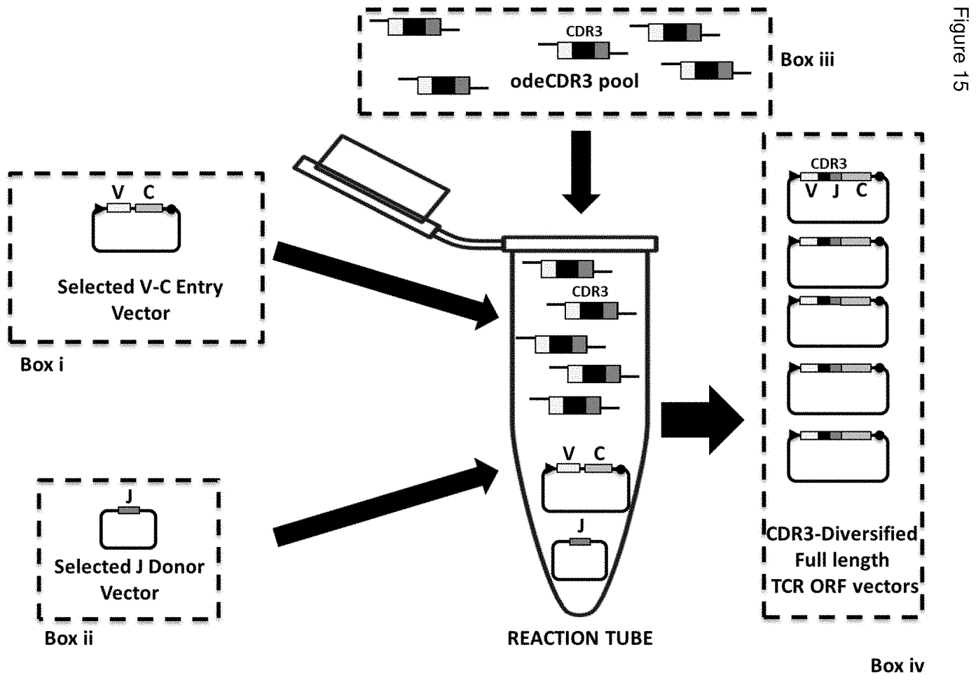

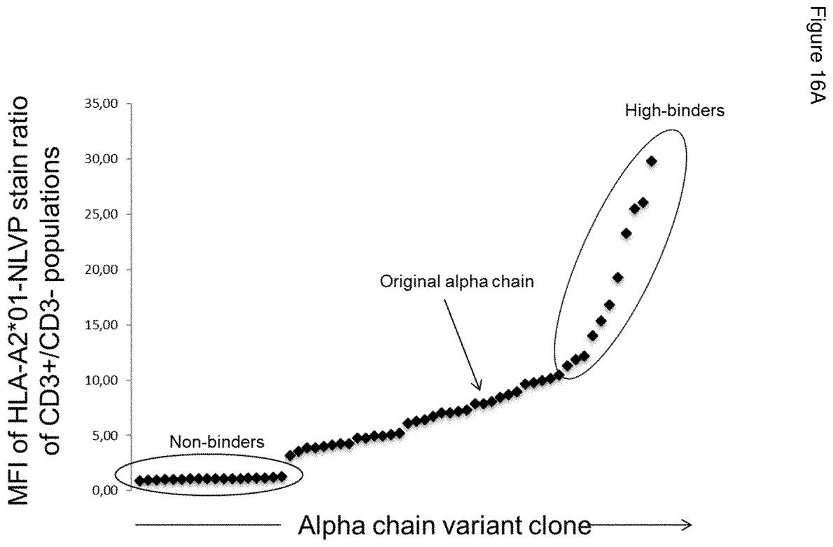

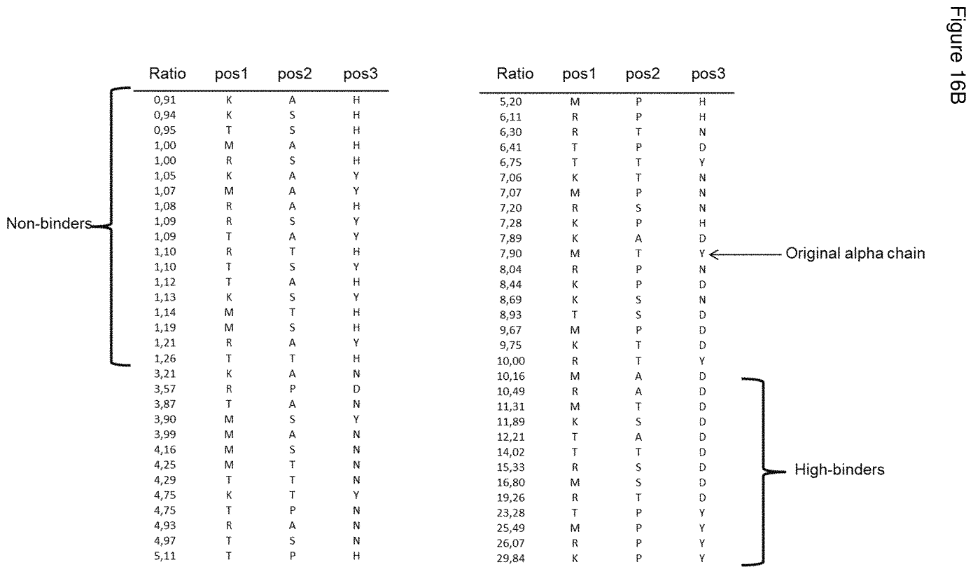

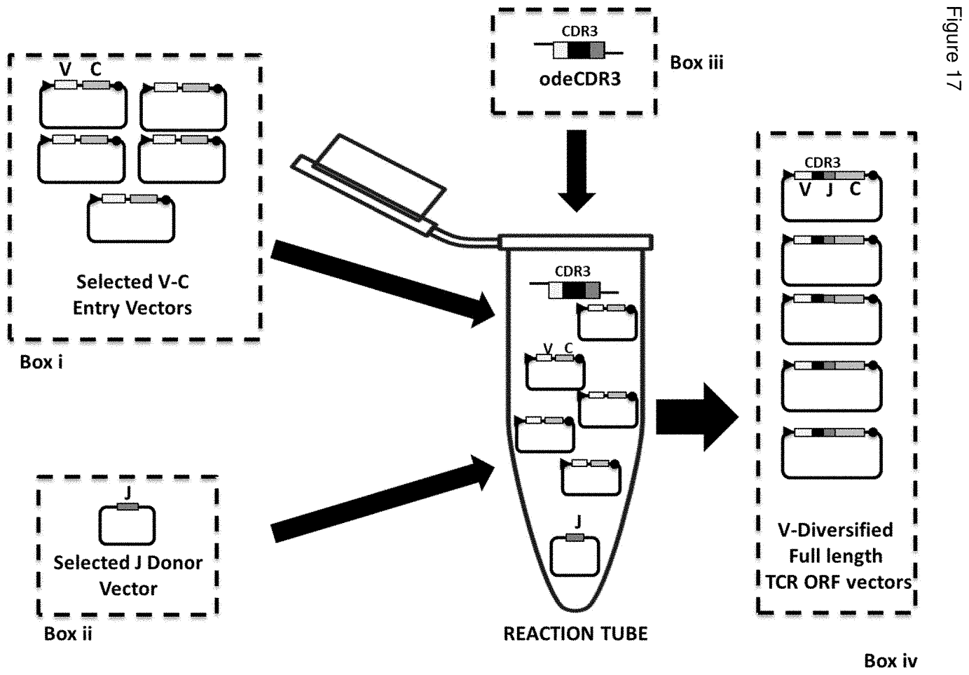

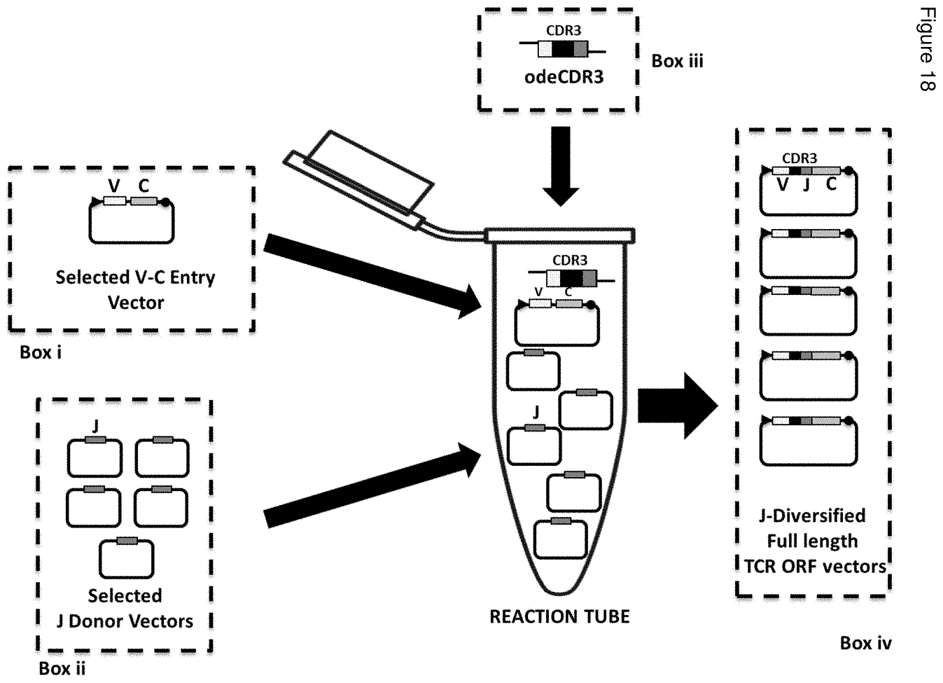

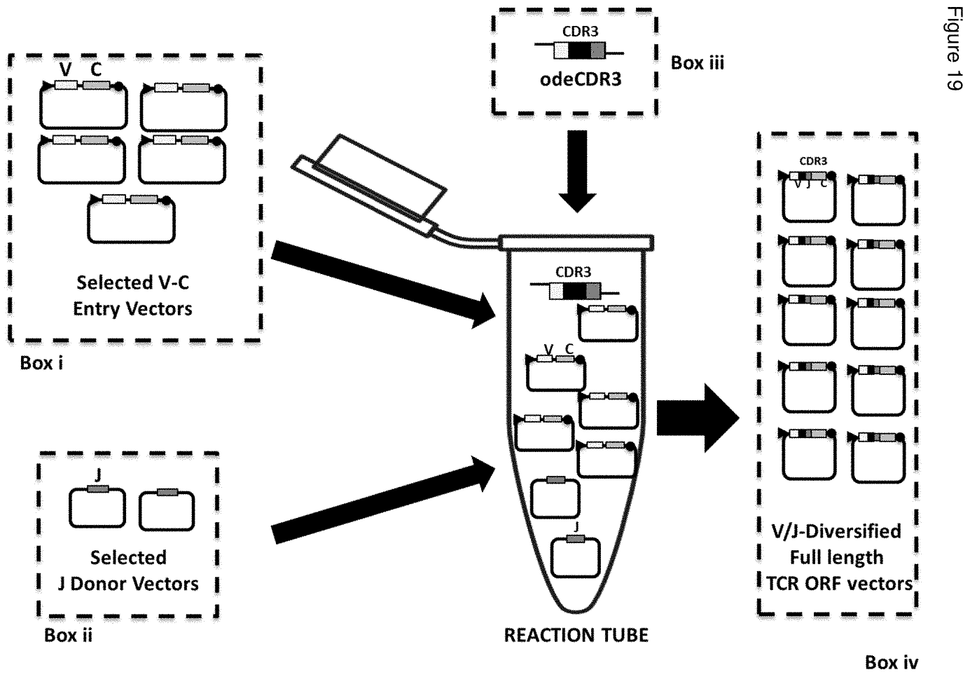

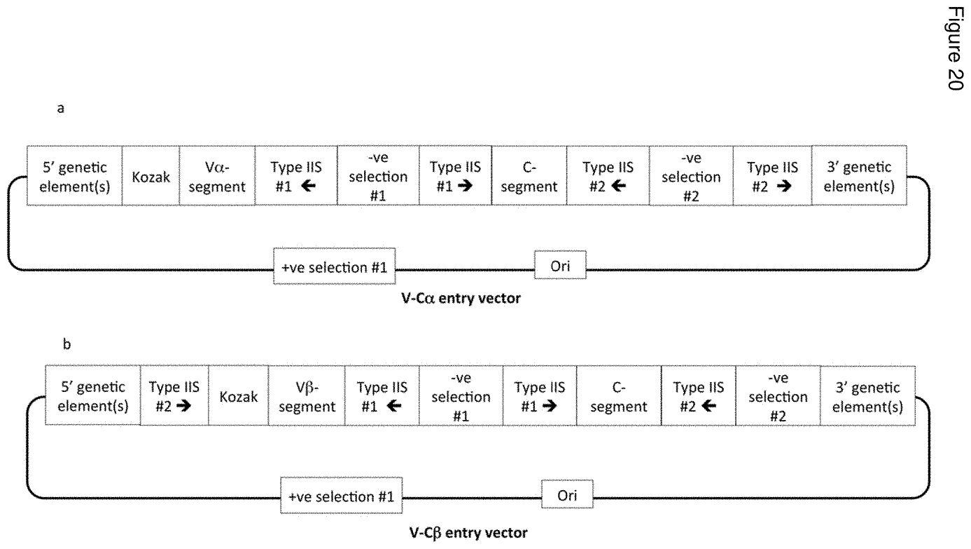

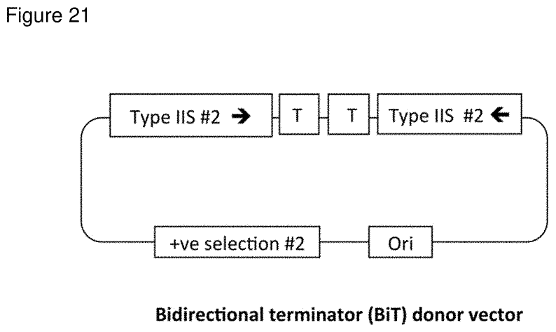

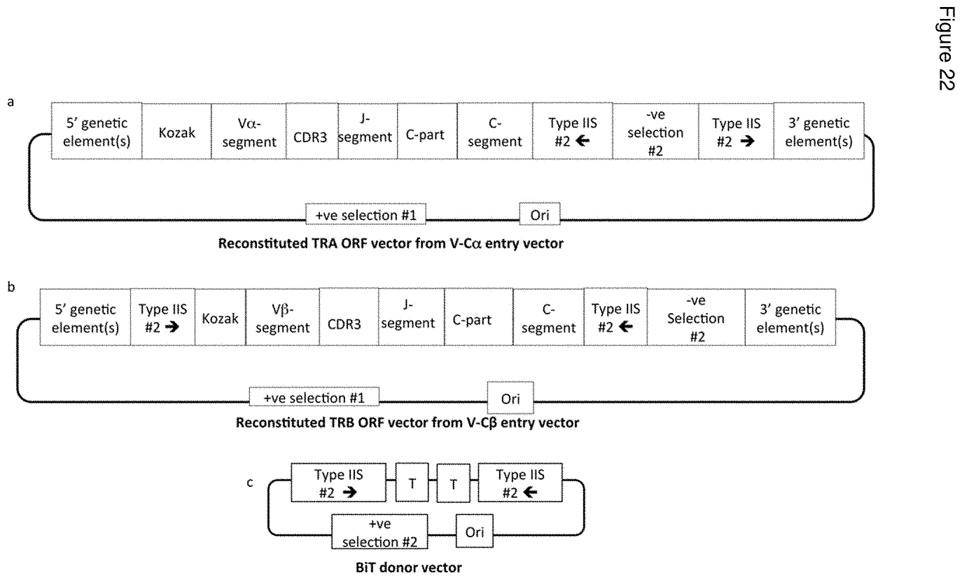

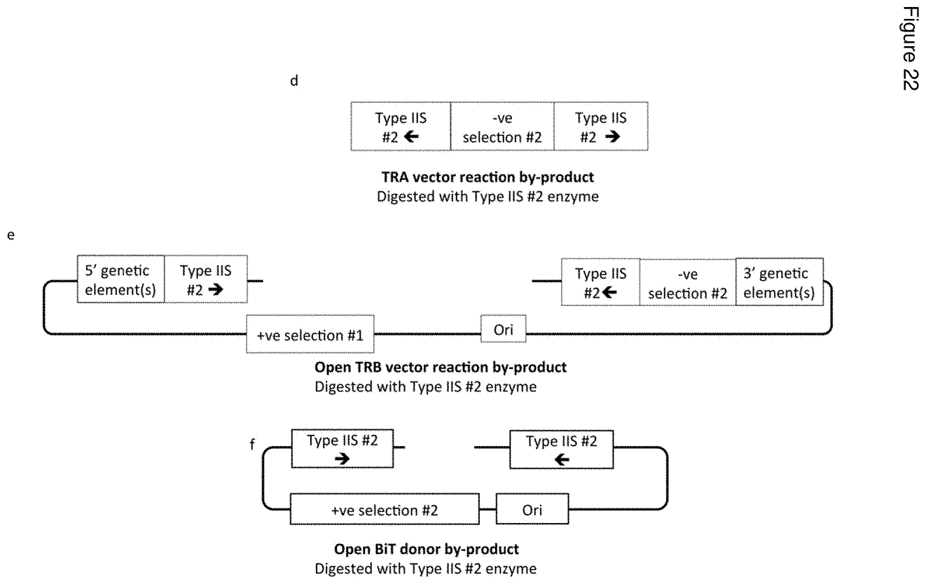

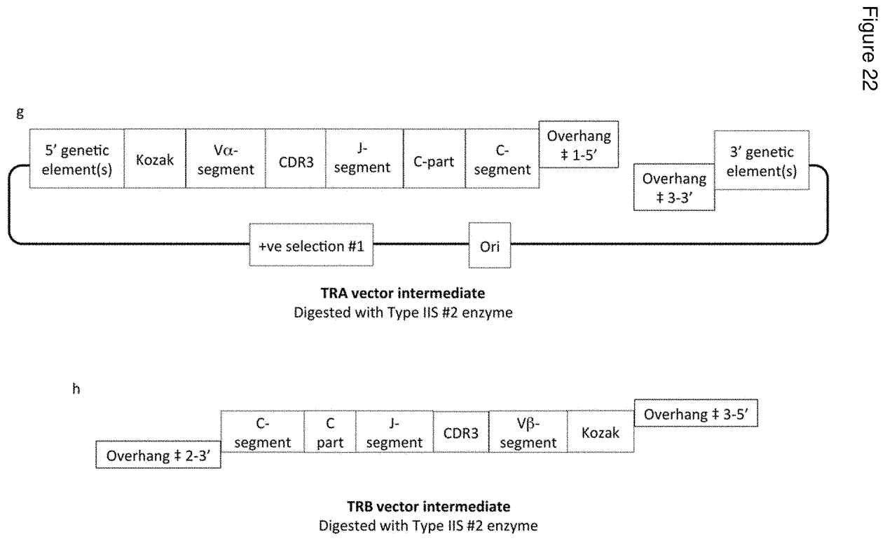

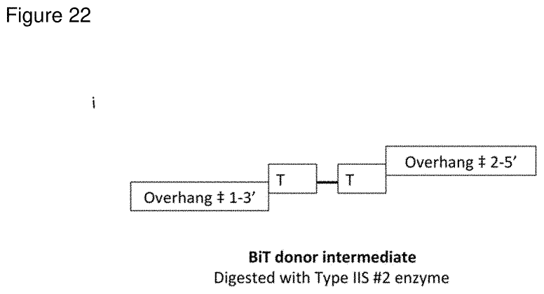

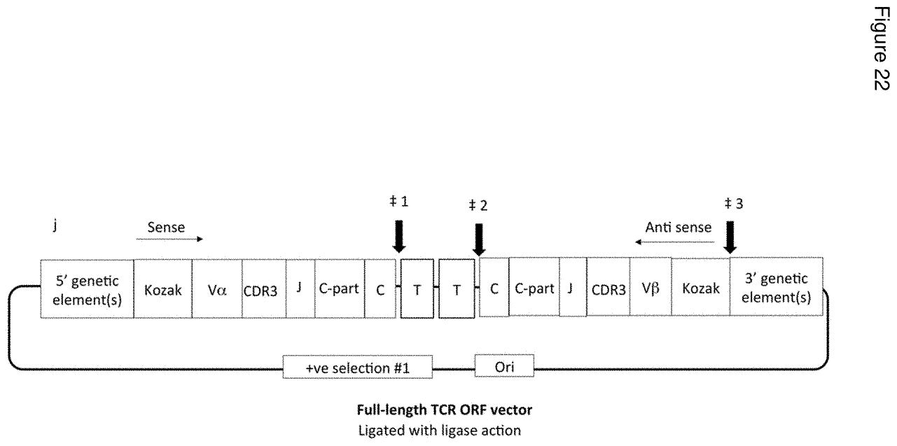

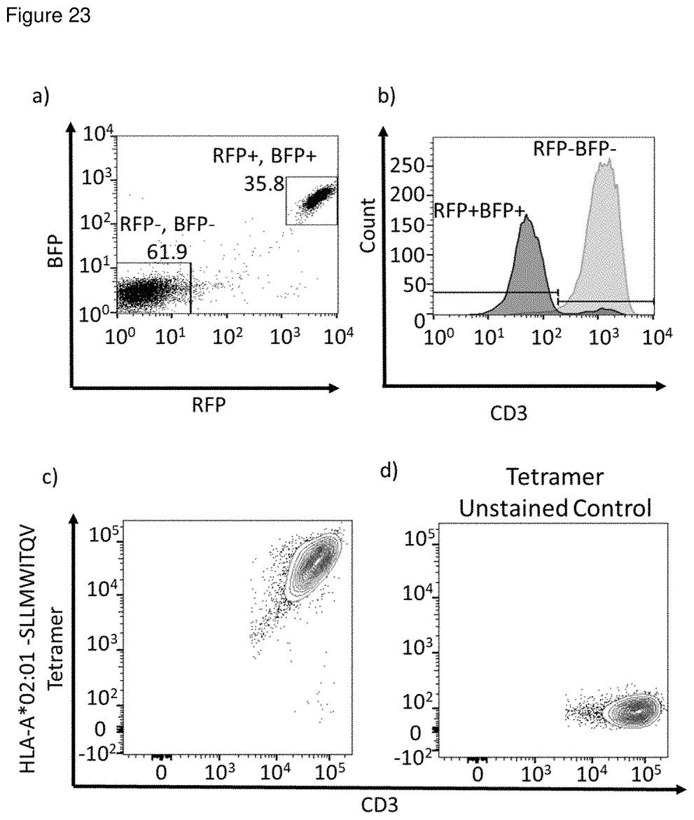

A)

Depicted are the core features of the two-component vector system (Box i), the required oligo duplex input (Box ii) and the resulting full-length TCR ORF vector (Box iii). The first component represents a V-C entry vector, incorporating a selected TCR V (variable) gene segment and a TCR C (constant) gene segment. This entry vector represents the backbone in which the final full-length TCR ORF will be carried. Thus, any desirable features of this vector backbone are tailored to the intended downstream application. Usually, this will be represented by at least a 5′ and 3′ genetic element. The required V gene segment and C gene segment of the target full-length TCR ORF is selected from a library of V-C entry vectors to construct a TCR ORF of any desired V/C combination (Box i). The second component represents a J donor vector, containing a selected J (joining) gene segment. This donor vector acts to donate the J gene segment, and thus the backbone of this vector is a reaction by-product. The desired J gene segment of the target TCR ORF is selected from a library J donor vectors. To complete the sequence required for the full-length TCR ORF, a short oligo duplex is synthesized corresponding largely to the CDR3 region of the mature TCR ORF (Box ii). This oligo duplex is synthesized with single strand overhangs compatible with unique overhangs generated by restriction enzyme digestion of V-C entry and J donor vectors. When combined into a single-tube reaction along with appropriate restriction and ligase enzymes, the full-length TCR ORF is reconstituted from the sequences provided by the V-C entry and J donor vectors selected from a pre-existing library, and the synthesized oligo duplex (Box iii).

B)

Function of the overall library feature of the TCR ORF reconstitution system is illustrated. A single V-C entry vector is selected from a library of V-C entry vectors with varying V-C combinations (Box i). This selection is based on the required V-C combination sequences for a selected TCR chain. A single J entry vector is selected from a library of J donor vectors with varying J gene segments encoded (Box ii). This selection is based on the required J combination sequences for the same selected TCR chain as that of the V-C entry vector. Finally, an oligomeric duplex encoding CDR3 (odeCDR3) is selected as to complete the full-length ORF of the target TCR chain (Box iii). These three components (Box i, ii and iii) are combined into a single reaction along with appropriate restriction and ligase enzymes. The reaction cycle produces a reconstituted full-length TCR ORF in a single step in the V-C entry vector backbone context (Box iv).

FIG. 2. Plasmid schematic of the genetic features of a generic V-C entry vector. Depicted is a circularized plasmid schematic of a V-C entry vector with minimally required genetic elements depicted as labelled boxes. Kozak, refers to consensus sequence that plays a role in the efficient initiation of translation. V-segment, refers to a selected sequence encoding a proportion of a TCR variable germline ORF, or mutant/synthetic ORF. Type IIS [symbol] , refers to a Type IIS restriction enzyme binding site orientated such the enzyme cleaves in the 5′ direction. Type IIS [symbol] , refers to a Type IIS restriction enzyme binding site orientated such the enzyme cleaves in the 3′ direction. −ve selection, refers to a negative selection element designed to be detrimental to a plasmid harbouring the sequence during the full-length TCR reconstruction reaction, or subsequent selection steps. C-segment, refers to a selected sequence encoding a proportion of a TCR constant germline ORF, or mutant/synthetic ORF. +ve selection #1, refers to a the first positive selection marker of the two-component system used to convey a selective advantage to the organism harbouring the vector, and which is different to the positive selection marker of the second vector component (see FIG. 3). Ori, refers to an origin of replication used for the propagation of plasmid within a compatible host. 5′ genetic element, refers to any desired genetic element that provides attributes required for downstream application of the reconstructed full-length TCR, and should be situated 5′ of the reconstructed full-length TCR, for example, a cis-acting element. 3′ genetic element, refers to a to any desired genetic element that provides attributes required for downstream application of the reconstructed full-length TCR, and should be situated 3′ of the full-length TCR ORF, for example, a transcriptional terminator element.

FIG. 3. Plasmid schematic of the genetic features of a generic J donor vector. Depicted is a circularized plasmid schematic of a J donor vector with minimally required genetic features depicted as labelled boxes. J segment part, refers to a DNA sequence encoding a proportion of a TCR joining germline ORF, or mutant/synthetic J gene segment. C part, refers to a small 5′ portion of the TCR Constant gene segment. Type IIS [symbol] , refers to a Type IIS restriction enzyme binding site orientated such the enzyme cleaves in the 5′ direction. Type IIS [symbol] , refers to a Type IIS restriction enzyme binding site orientated such the enzyme cleaves in the 3′ direction. +ve selection #2, refers to the first positive selection marker of the two-component system used to convey a selective advantage to the organism harbouring the vector, and which is different to the positive selection marker of the first vector component (see FIG. 2). Ori, refers to an origin of replication used for the propagation of plasmid within a compatible host.

FIG. 4. Generic description of genetic input, by-products, intermediates and product of the two-component vector system to reconstruct a full-length TCR ORF.

Depicted are the two components of the vector system (a and b), the oligonucleotide duplex (c), when these three components are combined into a single reaction with Type IIS restriction enzyme and ligase, two reaction by-products (d and e), two reaction intermediates (f and g) and one reaction product (h) is generated. Input vectors and product of the two-component system are depicted as circularized plasmid schematics with genetic elements depicted as labelled boxes; open plasmid vectors that represent by-product or intermediate are non-circularized plasmid schematics with genetic elements depicted as labelled boxes; and linear DNA are depicted as series of labelled boxes describing genetic elements.

a) A generic V-C entry plasmid as depicted in FIG. 2.
b) A generic J donor plasmid as depicted in FIG. 3.
c) A DNA oligo duplex containing CDR3 sequence flanked by two single stranded DNA overhangs, overhang [symbol] 1-5′ and overhang [symbol] 1-3′. Overhang [symbol] 1-5′ is compatible with the overhang [symbol] 1-3′ in the open V-C entry vector intermediate (g). Overhang [symbol] 2-3′ is compatible with the overhang [symbol] 2-5′ in the donor fragment intermediate (f).
d) Digestion of the V-C entry vector (a) by the Type IIS restriction enzyme results in a linear DNA V-C entry vector reaction by-product containing the −ve selection element and the Type IIS [symbol] and Type IIS [symbol] elements.
e) Digestion of the J donor vector (b) by the Type IIS restriction enzyme results in a non-circularised plasmid by-product containing all genetic elements of the parental plasmid except those carried in the excised J donor fragment intermediate (f).
f) Digestion of the J donor vector (b) by the Type IIS restriction enzyme results in a linear DNA fragment containing the J segment part and C part flanked by single strand DNA overhangs, overhang [symbol] 2-5′ and overhang [symbol] 3-3′. Overhang [symbol] 2-5′ is compatible with the overhang [symbol] 2-3′ in CDR3 DNA oligonucleotide duplex (c). Overhang [symbol] 3-3′ is compatible with the overhang [symbol] 3-5′ in the open V-C entry vector intermediate (g).
g) Digestion of the V-C entry vector (a) by the Type IIS restriction enzyme results in a non-circularized plasmid intermediate containing all genetic elements of the parental plasmid except those carried in the excised linear DNA V-C entry vector reaction by-product (d). Digestion has additionally created two single stranded DNA overhangs, overhang [symbol] 1-3′ and overhang [symbol] 3-5′. Overhang [symbol] 1-3′ is compatible with the overhang [symbol] 1-5′ in the CDR3 DNA oligonucleotide duplex (c). Overhang [symbol] 3-5′ is compatible with the overhang [symbol] 3-3′ in the J donor fragment intermediate (f).
h) Ligation of all three compatible single stranded DNA overhangs results in the full-length TCR ORF vector as circularized plasmid. This plasmid contains all genetic elements of the parental V-C entry vector (a) with the exception of the excised V-C entry vector reaction by-product (d). In addition, the full-length TCR ORF vector incorporates the CDR3 sequence from the CDR3 DNA oligonucleotide duplex (c) and J segment part and C part from the J donor fragment reaction intermediate. Arrows indicate the approximate points of ligation between compatible single stranded DNA overhangs [symbol] 1, [symbol] 2 and [symbol] 3. Ligation point [symbol] 1 is comprised of the [symbol] 1-3′ and [symbol] 1-5′ elements donated by the V-C entry vector reaction intermediate (g) and CDR3 DNA oligonucleotide duplex (c), respectively. Ligation point [symbol] 2 is comprised [symbol] 2-3′ and [symbol] 2-5′ elements donated by the CDR3 DNA oligonucleotide duplex (c) and the J donor fragment reaction intermediate (f), respectively. Ligation point [symbol] 3 is comprised [symbol] 3-3′ and [symbol] 3-5′ elements donated by the J donor fragment reaction intermediate (f) and the V-C entry vector reaction intermediate (g), respectively.

FIG. 5 Arrangement of V cloning fragments for the construction of V-C entry vectors

Depicted is a representation of the V cloning fragments used to assemble V-C entry vectors of a TORES for human TRA and TRB TCR chains as described in Examples 1 to 4.

The V cloning fragment is flanked by unique primer bind sequences at 5′ and 3′ end to facilitate PCR-mediated amplification of the cloning fragments. BbsI sites represent a specific Type IIS restriction enzyme binding sites used in the assembly of the V-C entry vector, where [symbol] indicates that the recognition site is orientated to cut in the 3′ direction of the site, and [symbol] indicates that the site is orientated to cut in the 5′ direction. The BbsI [symbol] site cuts 5′ of the encoded Kozak sequence to create overhang*1. The BsaI [symbol] site cuts to create the 5′ NotI overhang within the NotI 5′ fragment. Overhang*1 and the 5′ NotI overhang ultimately ligate with overhang*1′ of digested V-C entry vector backbone, and the 3′ NotI overhang of the digested C cloning fragment, respectively, in assembly of the V-C entry vector. The NotI 5′ fragment represents a 6 nucleotide 5′ fragment of the NotI recognition sequence, wherein NotI acts as the negative selection marker to eliminate parental V-C entry vector in operation of the TORES. The complete NotI recognition site is reconstituted with the 3′ NotI fragment, provided by the C cloning fragment. The V-segment represents the TCR V gene segment that is to be encoded by the final V-C entry vector and encodes from the ATG start codon of the give V segment to the last Cys codon of the V segment that defines the border of the CDR3 region. The BsaI [symbol] site is the Type IIS restriction enzyme recognition sequence used during operation of the TORES system to reconstitute a full-length TCR ORF. Action of the BsaI enzyme, wherein the site is orientated to cut in the 5′ direction, results in the creation of overhang [symbol] 1 at the 3′ end of the V segment that encompasses the three nucleotides of the last Cys codon of each V segment, and the third nucleotide of the codon preceding that Cys codon. This overhang is standardized among all V segments in a given TORES set. Ultimately, the overhang [symbol] 1 at the 3′ of the V segment ligates with overhang [symbol] 1 at the 5′ odeCDR3 in operation of the TORES system to reconstitution of a full-length TCR ORF. All sp denote the addition of one or more nucleotides to create the correct spacing between the Type IIS recognition sequences and the target overhang sequences, or to space the NotI recognition and cut site for efficient action.

FIG. 6 Arrangement of C cloning fragments for the construction of V-C entry vectors

Depicted is a representation of the C cloning fragments used to assemble V-C entry vectors of a TORES for human TRA and TRB TCR chains as described in Examples 1 to 4.

The C cloning fragment is flanked by unique primer bind sequences at 5′ and 3′ end to facilitate PCR-mediated amplification of the cloning fragments. BbsI sites represent a specific Type IIS restriction enzyme binding sites used in the assembly of the V-C entry vector, where [symbol] indicates that the recognition site is orientated to cut in the 3′ direction of the site, and [symbol] indicates that the site is orientated to cut in the 5′ direction. The BbsI [symbol] site cuts to create the 3′ NotI overhang within the NotI 3′ fragment. The BsaI [symbol] site cuts 3′ of the stop codon of the C segment to create overhang*2 at the 3′ end of the C segment. Overhang*2 and the 3′ NotI overhang ultimately ligate with Overhang*2′ of the digested V-C entry vector backbone, and the 5′ NotI overhang of the digested V cloning fragment, respectively, in assembly of the V-C entry vector. The NotI 3′ fragment represents a 6 nucleotide 3′ fragment of the NotI recognition sequence, wherein NotI acts as the negative selection marker to eliminate parental V-C entry vector in operation of the TORES. The complete NotI recognition site is reconstituted with the 5′ NotI fragment, provided by the V cloning fragment.

The C-segment represents the TCR C gene segment that is to be encoded by the final V-C entry vector and encodes from the cytosine residue 5′ of the first Glu codon of the C gene segment to the stop codon. The BsaI [symbol] site is the Type IIS restriction enzyme recognition sequence used during operation of the TORES system to reconstitute a full-length TCR ORF. Action of the BsaI enzyme, wherein the site is orientated to cut in the 3′ direction, results in the creation of overhang [symbol] 3 at the 5′ end of the C segment. This overhang is standardized among all C segments in a given TORES set. Ultimately, the overhang [symbol] 3 at the 5′ of the C segment ligates with overhang [symbol] 3 at the 3′ C part of the J donor vector in operation of the TORES system to reconstitution of a full-length TCR ORF. All sp denote the addition of one or more nucleotides to create the correct spacing between the Type IIS recognition sequences and the target overhang sequences, or to space the NotI recognition and cut site for efficient action.

FIG. 7 Arrangement of V-C entry vector backbone for the construction of V-C entry vectors

Depicted is a representation of the V-C entry vector backbone used to assemble V-C entry vectors of a TORES for human TRA and TRB TCR chains as described in Examples 1 to 4.

The circular plasmid DNA contains an origin of replication (Ori) and a positive selection marker #1. This selection marker is used for selection of transformed hosts when isolating clones of V-C entry vector backbone and V-C entry vectors during the assembly, and also for the selection of vectors containing full-length TCR ORFs during operation of the TORES. 5′ and 3′ genetic elements encode the target elements that flank the final TCR ORF after generation of full-length TCR ORF after its generation by TORES operation. A 5′ genetic element might represent a mammalian promoter element to drive the expression of TCR transcripts, and a 3′ genetic element might represent a transcriptional terminator sequence. The ACC65I site represents a restriction enzyme recognition sequence, wherein action of the Acc65I enzyme results in the creation of Overhang*1′. This Overhang*1′ ligates with Overhang*1 in the digested V cloning fragment during assembly of the V-C entry vector. The XbaI site represents a restriction enzyme recognition sequence, wherein action of the XbaI enzyme results in the creation of Overhang*2′. This Overhang*2′ ligates with Overhang*2 in the digested C cloning fragment during assembly of the V-C entry vector. Sp denotes the addition of nucleotides to space the Acc65I and XbaI recognition sites for efficient action of both enzymes.

FIG. 8 Arrangement of the J receiving cassette fragment

Depicted is a representation of a J receiving cassette fragment used in the assembly of J donor vectors of a TORES for human TRA and TRB TCR chains as described in Examples 2 and 3. A J receiving cassette fragment is inserted into a J donor backbone to generate a J receiving cassette vector.

A J receiving cassette fragment is generated by annealing two complimentary oligonucleotides to create a linear double stranded DNA construct with 4-nucleotide single stranded overhangs at the 5′ and 3′ ends that are used for insertion of the fragment to the J donor vector backbone. Overhang*3 at the 5′ end of the J receiving cassette fragment ligates with Overhang*3′ of the digested J donor vector backbone, whereas Overhang*4 at the 3′ end ligates with Overhang*4′ of the digested J donor vector backbone.

The BsaI sites represent the Type IIS restriction recognition sites used in the operation of the TORES to assemble a full-length TCR ORF. BsaI [symbol] site is orientated to cut in the 5′ direction, and acts upon the C part sequence to generate Overhang [symbol] 3 at the 3′ C part. BsaI [symbol] site ultimate acts on the J segment part of the J donor vector to create Overhang [symbol] 2 at the 5′ end of the J segment part. BsaI [symbol] element also contains Overhang*5, which is generated by action of the BbsI on the BbsI [symbol] site during assembly of the J donor vector.

The BbsI sites represent the Type IIS restriction recognition sites used to assemble the J donor vector. The BbsI [symbol] site cuts the BsaI [symbol] element to generate Overhang*5, whereas the BbsI [symbol] site cuts the 5′ end of the C part to generate Overhang*6. Overhang*5 and Overhang*6 ultimately ligate with Overhang*5′ and Overhang*6′ of the J segment part, respectively.

The C part represents a small portion of the target C gene segment to permit standardized generation of non-palindromic overhangs during operation of the TORES. This C part is ultimate carried at the 3′ end of the J segment part, and forms part of the sequence that ligates with the C segment carried by the digested V-C entry vector in operation of the TORES to generate a full-length TCR ORF.

The NotI site represents a negative selection marker used to eliminate the parental J receiving cassette vector during generation of the J donor vector.

All sp denote the addition of one or more nucleotides to create the correct spacing between the Type IIS recognition sequences and the target overhang sequences, or to space the NotI recognition and cut site for efficient action.

FIG. 9 Arrangement of the J donor backbone

Depicted is a representation of J a donor vector backbone used in the assembly of J donor vectors of a TORES for human TRA and TRB TCR chains as described in Examples 2 and 3. A J receiving cassette fragment is inserted into a J donor backbone to generate a J receiving cassette vector.

The circular plasmid DNA contains an origin of replication (Ori) and a positive selection marker #2. This selection marker is used for selection of transformed hosts when isolating clones of J donor vector backbone and J donor vectors during the assembly. Importantly, this positive selection marker is distinct from positive selection marker #1 within the V-C entry vectors, such that parental J donor vectors are eliminated under positive selection on #1 during operation of the TORES to generate full-length TCR ORFs in the context of the V-C entry vector backbone.

The EcoRI site represents a restriction enzyme recognition sequence, wherein action of the EcoRI enzyme results in the creation of Overhang*3′. This Overhang*3′ ligates with Overhang*3 in the annealed J receiving cassette fragment during assembly of the J receiving cassette vector. The XboI site represents a restriction enzyme recognition sequence, wherein action of the XboI enzyme results in the creation of Overhang*4′. This Overhang*4′ ligates with Overhang*4 in the annealed J receiving cassette fragment during assembly of the J receiving cassette vector. Sp denotes the addition of nucleotides to space the Acc65I and XbaI recognition sites for efficient action of both enzymes.

FIG. 10 Arrangement of the J receiving cassette vector

Depicted is a representation of a J donor vector backbone used in the assembly of J donor vectors of a TORES for human TRA and TRB TCR chains as described in Examples 2 and 3. A J receiving cassette vector is created by insertion of a J receiving cassette fragment into a J donor backbone.

The circular plasmid DNA contains an origin of replication (Ori) and a positive selection marker #2. This selection marker is used for selection of transformed hosts when isolating clones of J donor vector backbone and J donor vectors during the assembly. Importantly, this positive selection marker is distinct from positive selection marker #1 within the V-C entry vectors, such that parental J donor vectors are eliminated under positive selection on #1 during operation of the TORES to generate full-length TCR ORFs in the context of the V-C entry vector backbone.

The BsaI sites represent the Type IIS restriction recognition sites used in the operation of the TORES to assemble a full-length TCR ORF. BsaI [symbol] site is orientated to cut in the 5′ direction, and acts upon the C part sequence to generate Overhang [symbol] 3 at the 3′ C part. BsaI [symbol] site ultimate acts on the J segment part of the J donor vector to create Overhang [symbol] 2 at the 5′ end of the J segment part. BsaI [symbol] element also contains Overhang*5, which is generated by action of the BbsI on the BbsI [symbol] site during assembly of the J donor vector.

The BbsI sites represent the Type IIS restriction recognition sites used to assemble the J donor vector. The BbsI [symbol] site cuts the BsaI [symbol] element to generate Overhang*5, whereas the BbsI [symbol] site cuts the 5′ end of the C part to generate Overhang*6. Overhang*5 and Overhang*6 ultimately ligate with Overhang*5′ and Overhang*6′ of the J segment part, respectively.

The C part represents a small portion of the target C gene segment to permit standardized generation of non-palindromic overhangs during operation of the TORES. This C part is ultimate carried at the 3′ end of the J segment part, and forms part of the sequence that ligates with the C segment carried by the digested V-C entry vector in operation of the TORES to generate a full-length TCR ORF.

The NotI site represents a negative selection marker used to eliminate the parental J receiving cassette vector during generation of the J donor vector.

All sp denote the addition of one or more nucleotides to create the correct spacing between the Type IIS recognition sequences and the target overhang sequences, or to space the NotI recognition and cut site for efficient action.

FIG. 11 Arrangement of a J segment part

Depicted is a representation of a J segment part that is used in the assembly of J donor vectors of a TORES for human TRA and TRB TCR chains as described in Examples 2 and 3. A J segment part is inserted into a J receiving cassette vector to create a J donor vector.

Annealing complimentary single stranded oligonucleotides to form a linear double stranded DNA construct with single stranded overhangs at either terminus generates a J segment part. Overhang*5′ at the 5′ terminus anneals with Overhang*5 generated within the J receiving cassette vector digested with BbsI. Overhang*6′ at the 3′ terminus anneals with Overhang*6 generated within the J receiving cassette vector digested with BbsI.

The J segment part represents the target J gene segment sequence. Depending on the style of the J donor vector being constructed (i.e. short or long) the 5′ border of the J segment part is defined differently. For short J donor vectors, the 5′ border of the J segment part is defined as the Phe-Ala/Gly or Trp-Gly motifs that are used to define the canonical border between the J and CDR3 portions of a full-length TCR ORF. For long J donor vectors, the 5′ border of the J segment part is extended ten to twelve nucleotides 5′ of the Phe-Ala/Gly or Trp-Gly motif. This extends the portion of the overall TCR ORF encoded by the J donor vector, and conversely shortens the length of the odeCDR3 required to construct a full-length TCR ORF in operation of the TORES. At the 3′ end of the J segment part is encoded a single Adenine residue (A), which represent the first nucleotide of the C fragment. This adenine is excluded from the J receiving cassette vector.

FIG. 12 Validation of specificity of reconstituted model TCR TRA/TRB pair

A model TRA/TRB TCR chain pair with specificity for a HLA-A2*01-restricted HCMV pp65 derived antigen was reconstituted with the TORES system as described in example 7. The two TRA and TRB plasmid products were transiently transfected into a human cell line constitutively expressing CD3 components, but lacking endogenous expression of TCR TRA and TRB chains. 48 hours after the transfection, cells were with antibodies against CD3, TCRalpha/beta and specific HLA-A2*01-NLVPMVATV tetramer and analysed by flow cytometry. The model TCR pair presented on the cell surface as indicated by positive staining with CD3 and TCRalpha/beta antibodies (left, top panel). The model TCR pair also displayed positive staining for the HLA-A2*01-NLVPMVATV tetramer reagent (left, bottom panel), indicating expected specificity of binding. An irrelevant pair of TRA and TRB plasmid constructs was also transfected in parallel (centre panels), as was empty vector (rightmost panels). The irrelevant TCR was presented on the cell surface as indicated by positive staining for CD3 and TCRalpha/beta antibodies (centre, top panel), but displayed no binding for the HLA-A2*01 NLVPMVATV tetramer reagent (centre, bottom panel). Empty vector transfected cells displayed no staining for either CD3 or TCRalpha/beta antibodies, or for the HLA-A2*01-NLVPMVATV tetramer reagent.

FIG. 13 Workflow for the identification and reconstitution of a set of TCR TRA/TRB chain pairs from human peripheral blood

To demonstrate the use of the TORES system for the reconstitution of native TCR TRA/TRB chain pairs captured from a human specimen, a set of TCRs specific for the HLA-B*07:02 restricted HCMV pp65 antigen, TPRVTGGGAM, was generated via capture of sequence information from single-cell sorting of tetramer-positive cells from a human PBMC fraction. This process is described in Example 8.

i) A PBMC fraction from a donor with HLA-B*07:02 allele is stained with a HLAB*07:02-TPRV tetramer reagent. Single CD8⁺ T-cells showing positive staining for tetramer a deposited into single PCR tubes by way of FACS.
ii) Single cell containing tubes were subjected to a reverse transcription reaction with primer sets anchored in all expressed variable and constant gene segments for both TRA and TRB chains.
iii) Nested PCR reactions are performed on the reverse transcription product with sets of internal primers, still anchored in all expressed V and C gene segments, independently for the TRA and TRB chains.
iv) The nested PCR product is subjected to Sanger sequencing
v) PCR product sequences are aligned against a library of human germline TCR gene segments to determine the V and J (and C) gene segment usage for each amplified TCR chain. The CDR3 sequence from the 5′ Cys codon to the 3′ Phe-Ala/Gly or Trp-Gly motifs are determined.
vi) A V-C entry vector is selected from the TORES library corresponding to the determined V and C gene segment usage of each TRA and TRB chain to be reconstituted.
vii) A J donor vector is selected from the TORES library corresponding to the determined J gene segment usage of each TRA and TRB chain to be reconstituted.
viii) An odeCDR3 is synthesized corresponding the CDR3 region determined from each TRA and TRB chain to be reconstituted.
ix) An independent restriction enzyme (RE)/Ligase cycle reaction is assembled for each TRA and TRB TCR to be reconstituted, containing the selected V-C entry vector, J donor vector and synthesized odeCDR3.
x) The RE/Ligase cycle reaction product is transformed into bacteria and placed under antibiotic selection for the first positive selection marker (i.e. V-C entry vector backbone).
xi) Clones are propagated and confirmed by Sanger sequencing of the final full-length TCR ORF.

FIG. 14 Validation of specificity for a set of TCR TRA/TRB chain pairs captured from human peripheral blood and reconstituted with TORES

A set of six TCR TRA/TRB chain pairs were captured from human peripheral blood and reconstituted via the workflow outlined in FIG. 13 and described in Example 8. The six TCR pairs had expected specificity for the B*07:02-TPRV tetramer reagent used to isolate single CD8⁺ T-cells. Each of the six captured pairs, an irrelevant TCR chain pair, and an empty vector control, were transfected into human cells expressing CD3 components, but lacking endogenous expression of TCR TRA and TRB chains. 48 hours after the transfection, flow cytometric analysis was used to stain the cells with antibodies against CD3, TCR alpha and beta chains and the specificity of the reconstituted and expressed TCR was confirmed by specific HLA-B*07:02-TPRV tetramer reagent. The data are presented as contour plots. Each panel displays CD3 signal on the X-axis and HLA-B*07:02-TPRV tetramer on the Y-axis. Inset at the bottom right of each panel is the ratio of HLA-B*07:02-TPRV tetramer signal of the CD3 positive cells over the CD3 negative cells as an arbitrary unit of the degree of tetramer staining. All six TCR chain pairs presented display some degree of specific tetramer staining compared to the irrelevant TCR pair control.

FIG. 15 Operation of the TORES to generate CDR3-diversified TCR chains

Depicted is a schematic representation of the TORES when used to generate full-length TCR chains with diversified CDR3 inserts. A parental TCR is defined with V-J-C usage, and defined CDR3 region sequence. The corresponding single V-C entry vector (box i) and single J donor vector (box ii) are placed in the reaction tube. A pool of odeCDR3 with defined positional nucleotide degeneracy and/or point mutagenesis that changes the coded amino acid sequence is synthesized (Box iii). Such a CDR3 pool could include completely randomized CDR3 sequences within the bounds of the defined odeCDR3 framework, as to create a ‘synthetic’ full-length TCR ORFs CDR3 with germline V-J-C usage. These three components (Box i, ii and iii) are combined into a single reaction along with appropriate restriction and ligase enzymes. The reaction cycle produces a number of variant reconstituted full-length TCR ORFs, proportional to the number of variant odeCDR3 included, in a single step in the V-C entry vector backbone context (Box iv).

FIG. 16 A Single round of TCR chain diversification using limited CDR3 degeneracy generates a TCR set with a large spectrum of target affinities

The TRA chain of a model TRA/TRB TCR chain pair with specificity for a HCMV pp65 derived antigen as described in example 7, was subject to a CDR3-diversification cycle according to the scheme presented in FIG. 15 and as described in Example 9. The TRA chain odeCDR3 was synthesized with 4-fold nucleotide degeneracy at 3 separate positions, altering the codon usage at those three positions. The resulting reaction product contained 64 full-length TRA chain variants, including the parental sequence. Each of the 64 TRA chains was cloned and sequence confirmed.

A) Each of the 64 TRA chain plasmids was transfected along with the parental TRB plasmid into a human cell line constitutively expressing CD3 components, but lacking endogenous expression of TCR TRA and TRB chains. 48 hours after transfection, the cells were stained with antibodies against CD3 and with a HLA-A2*01-NLVPMVATV tetramer reagent and analysed by flow cytometry. The ratio of the mean fluorescence intensity (MFI) of HLA-A2*01-NLVPMVATV tetramer signal for the CD3+ population over the CD3− population was plotted, as an indication of the binding strength of each TCR chain pair variant. The arrow indicates the data point corresponding to the parental TRA. Ovals indicate high-binder and non-binder variants at either end of the spectrum.

B) Each TRA chain variant is presented in a table with the MFI ratio of HLA-A2*01-NLVPMVATV tetramer signal for the CD3+ population over the CD3− population, and the amino acid present at each of the three diversified positions (Pos1, 2 and 3), in one letter code. The arrow indicates the data point corresponding to the parental TRA. Brackets indicate high-binder and non-binder variants at either end of the spectrum.

FIG. 17 Operation of the TORES to generate V-segment diversified TCR chains

Depicted is a schematic representation of the TORES when used to generate full-length TCR chains with diversified V-segment usage. A parental TCR is defined with VJ-C usage, and defined CDR3 region sequence. The corresponding single J donor vector (box ii) is placed in the reaction tube, as is the single odeCDR3 synthesized to correspond with parental CDR3 region sequence (Box iii). A selection of V-C entry vectors is also added to the reaction tube, corresponding to the V- and C-segments desired in the product V-segment diversified full length TCR ORF product (Box i). These three components (Box i, ii and iii) are combined into a single reaction along with appropriate restriction and ligase enzymes. The reaction cycle produces a number of variant reconstituted full-length TCR ORFs, proportional to the number of variant V-C entry vectors included, in a single step in the V-C entry vector backbone context (Box iv).

FIG. 18 Operation of the TORES to generate J-segment diversified TCR chains

Depicted is a schematic representation of the TORES when used to generate full-length TCR chains with diversified J-segment usage. A parental TCR is defined with V-J-C usage, and defined CDR3 region sequence. The corresponding single V-C entry vector (box i) is placed in the reaction tube, as is the single odeCDR3 synthesized to correspond with parental CDR3 region sequence (Box iii). A selection of J donor is also added to the reaction tube, corresponding to the J segments desired in the product J-segment diversified full length TCR ORF product (Box ii). These three components (Box i, ii and iii) are combined into a single reaction along with appropriate restriction and ligase enzymes. The reaction cycle produces a number of variant reconstituted full-length TCR ORFs, proportional to the number of variant J donor vectors included, in a single step in the V-C entry vector backbone context (Box iv).

FIG. 19 Operation of the TORES to generate V/J-segment diversified TCR chains

Depicted is a schematic representation of the TORES when used to generate full-length TCR chains with diversified V- and J-segment usage. A parental TCR is defined with V-J-C usage, and defined CDR3 region sequence. The corresponding single odeCDR3 synthesized to correspond with parental CDR3 region sequence (Box iii). A selection of V-C entry vectors and J donor vectors are added to the reaction tube, corresponding to the combination of V- (C-) and J-segments desired in the product V/J-segment diversified full length TCR ORF product (Box ii). These three components (Box i, ii and iii) are combined into a single reaction along with appropriate restriction and ligase enzymes. The reaction cycle produces a number of variant reconstituted full-length TCR ORFs, proportional to the number of V-C and J donor vector combinations possible from those included, in a single step in the V-C entry vector backbone context (Box iv).

FIG. 20. Schematic of the genetic features of TORES² V-C□ and V-C□ entry vectors.

Depicted is a circularized plasmid schematic of a V-C□ (a) and V-C□ (b) entry vectors with minimally required genetic elements depicted as labelled boxes. Kozak, refers to consensus sequence that plays a role in the efficient initiation of translation. V-segment, refers to a selected sequence encoding a proportion of a TCR variable germline ORF, or mutant/synthetic ORF. Type IIS #1 [symbol] , refers to a first Type IIS restriction enzyme binding site orientated such the enzyme cleaves in the 5′ direction. Type IIS #1 [symbol] , refers to a first Type IIS restriction enzyme binding site orientated such the enzyme cleaves in the 3′ direction. −ve selection #1, refers to a negative selection element designed to be detrimental to a plasmid harbouring the sequence during the full-length TCR reconstruction reaction, or subsequent selection steps. C-segment, refers to a selected sequence encoding a proportion of a TCR constant germline ORF, or mutant/synthetic ORF. Type IIS #2 [symbol] , refers to a second Type IIS restriction enzyme binding site orientated such the enzyme cleaves in the 5′ direction. The enzyme is different than the first Type IIS. Type IIS #2 [symbol] , refers to a second Type IIS restriction enzyme binding site orientated such the enzyme cleaves in the 3′ direction. The enzyme is different than the first Type IIS. −ve selection #2, refers to a negative selection element designed to be detrimental to a plasmid harbouring the sequence during assembly of the full-length bidirectional TCR ORF vector. −ve selection #2 is different to the first −ve selection. +ve selection #1, refers to the first positive selection marker of the two-component system used to convey a selective advantage to the organism harbouring the vector, and which is different to the positive selection marker a second (J donor) vector component (see FIG. 3). Ori, refers to an origin of replication used for the propagation of plasmid within a compatible host. 5′ genetic element, refers to any desired genetic element that provides attributes required for downstream application of the reconstituted full-length TCR, and should be situated 5′ of the reconstituted full-length TCR, for example, an RMCE element. 3′ genetic element, refers to any desired genetic element that provides attributes required for downstream application of the reconstructed full-length TCR, and should be situated 3′ of the full-length TCR ORF, for example, a second RMCE element.

FIG. 21. Plasmid schematic of the genetic features of the bidirectional terminator donor (BiT donor) vector.

Depicted is a circularized plasmid schematic of a bidirectional terminator donor vector with minimally required genetic features depicted as labelled boxes. The bidirectional terminator element (T-T), refers to a DNA sequence encoding a bidirectional terminator. Type IIS #2 [symbol] , refers to a second Type IIS restriction enzyme binding site orientated such the enzyme cleaves in the 3′ direction. Type IIS #2 [symbol] , refers to a second Type IIS restriction enzyme binding site orientated such the enzyme cleaves in the 5′ direction. +ve selection #2, refers to the first positive selection marker of the two-component system used to convey a selective advantage to the organism harbouring the vector, and which is different to the positive selection marker of the first vector component (see FIG. 20). Ori, refers to an origin of replication used for the propagation of plasmid within a compatible host.

FIG. 22. Generic description of genetic input, by-products, intermediates and product of the bidirectional two-component vector system to reconstruct a paired TRA and TRB vector.

Depicted are the TRA (a), TRB (b) and the bidirectional terminator donor vector (c), when these three components are combined into a single reaction with second Type IIS restriction enzyme and ligase, three reaction by-products (d, e and f), three reaction intermediates (g, h and i) and one reaction product (j) are generated. Input vectors and product of the two-component system are depicted as circularized plasmid schematics with genetic elements depicted as labelled boxes; open plasmid vectors that represent by-product or intermediate are non-circularized plasmid schematics with genetic elements depicted as labelled boxes; and linear DNA are depicted as series of labelled boxes describing genetic elements.

a) A reconstituted TRA ORF in V-C□ entry vector context as depicted in FIG. 20a.
b) A reconstituted TRA ORF in V-C□ entry vector context as depicted in FIG. 20b
c) A BiT donor vector as depicted in FIG. 21
d) Digestion of the TRA vector (a) with Type IIS #2 restriction enzyme results in a linear DNA TRA vector reaction by-product containing the −ve selection element #2 and the Type IIS #2 [symbol] and Type IIS #2 [symbol] binding site.
e) Digestion of the TRB vector (b) by the Type IIS #2 restriction enzyme results in a linear DNA TRB vector reaction by-product containing the 5′ and 3′ genetic elements, the Type IIS #2 [symbol] and Type IIS #2 [symbol] elements, the −ve selection element #2, the +ve selection element #1 and origin of replication element.
f) Digestion of the bidirectional terminator donor vector (c) by the Type IIS #2 restriction enzyme results in a non-circularised plasmid by-product containing all vector elements of the parental plasmid except the excised bidirectional terminator fragment intermediate (i).
g) Digestion of the TRA vector (a) by the Type IIS #2 restriction enzyme results in a non-circularized plasmid intermediate containing all genetic elements of the parental plasmid except those carried in the excised linear DNA TRA vector reaction by-product (d). Digestion has additionally created two single stranded DNA overhangs, overhang [symbol] 1-5′ and overhang [symbol] 3-3′. Overhang [symbol] 1-5′ is compatible with the overhang [symbol] 1-3′ in the bidirectional terminator intermediate (i). Overhang [symbol] 3-3′ is compatible with the overhang [symbol] 3-5′ in the TRB fragment intermediate (h).
h) Digestion of the TRB vector (b) by the Type IIS #2 restriction enzyme results in a linear DNA fragment containing the C segment, C part, J segment, CDR3, V□ segment and Kozak sequence flanked by single strand DNA overhangs, overhang [symbol] 2-3′ and overhang [symbol] 3-5′. Overhang [symbol] 2-3′ is compatible with the overhang [symbol] 2-5′ in the bidirectional terminator intermediate (i). Overhang [symbol] 3-5′ is compatible with the overhang [symbol] 3-3′ in the open TRA vector intermediate (g).
i) Digestion of the bidirectional terminator donor vector (c) by the Type IIS #2 restriction enzyme results in a linear DNA fragment intermediate containing the bidirectional terminator. Digestion has additionally created two single stranded DNA overhangs, overhang [symbol] 1-3′ and overhang [symbol] 2-5′. Overhang [symbol] 1-3′ is compatible with the overhang [symbol] 1-5′ in the open TRA vector intermediate (g). Overhang [symbol] 2-5′ is compatible with the overhang [symbol] 2-3′ in the TRB fragment intermediate (h).
j) Ligation of all three compatible single stranded DNA overhangs results in the bidirectional paired TRA and TRB vector as circularized plasmid. This plasmid contains all genetic elements of the parental TRA vector (a) with the exception of the excised TRA vector reaction by-product (d). In addition, the full-length TCR ORF vector incorporates the TRB fragment intermediate (h) and the bidirectional terminator intermediate (i). Arrows indicate the approximate points of ligation between compatible single stranded DNA overhangs [symbol] 1, [symbol] 2 and [symbol] 3. Ligation point [symbol] 1 is comprised of the [symbol] 1-5′ and [symbol] 1-3′ elements donated by the TRA vector reaction intermediate (a) and the bidirectional terminator intermediate (i), respectively. Ligation point [symbol] 2 is comprised [symbol] 2-3′ and [symbol] 2-5′ elements donated by the TRB fragment intermediate (h) and the bidirectional terminator fragment (i), respectively. Ligation point [symbol] 3 is comprised [symbol] 3-3′ and [symbol] 3-5′ elements donated by the TRA vector reaction intermediate (a) and the TRB fragment reaction intermediate (h), respectively.

FIG. 23 Validation of specificity of a model TCR TRA/TRB pair reconstituted using TORES²

A model TRA/TRB TCR chain pair with specificity for a HLA-A*02:01-restricted antigen was reconstituted with the TORES² system as described in example 11. The bidirectional TRA/TRB donor vector was used to stably integrate the TCR ORFs into a human cell line constitutively expressing CD3 components but lacking endogenous expression of TRA and TRB chains, and further encoding a genomic receiver site compatible with the RMCE sites contained within the product donor vector that have been contributed by the original V-Cα entry vector. The functionality of the reconstituted full length paired TRA and TRB donor vector generated by the TORES² was determined by surface staining of the CD3 complex, and expected specificity of the reconstituted and expressed TCR was confirmed by specific HLA-A*02:01-SLLMWITQV tetramer staining.

a) The model TCR pair was integrated into the genome of the engineered TCR presenting cell line containing genomic receiver sites matched to the donor vector. This genomic receiver site contains promoter elements driving two separate fluorescent markers (red fluorescent protein, RFP, and blue fluorescent protein, BFP) in an antiparallel arrangement, interposed by a bidirectional terminator element similar to the final TRA/TRB donor vector product of the TRA/TRB TORES² described in Example 11. Upon transfection and outgrowth of the receiver cells, flow cytometric analysis was conducted to observe the persistence of these florescent reporters. The absence of the genomic receiver sites (RFP−BFP−) therefore indicated that the cells have successfully integrated the TRA/TRB donor vector. While the cells that did not integrate the TRA/TRB donor vector demonstrated presence of the genomic receiver sites (RFP+BFP+). Majority of the cells have successfully integrated the TRA/TRB donor vector sequences into genomic receiver site.
b) Each of the RFP−BFP− and RFP+BFP+ populations of a) were gated and redisplayed in a second overlay histogram plot. The cells that integrated the bidirectional construct also showed positive staining with CD3 antibody, demonstrating that TCR was expressed on the cell surface (light histogram). The cells that did not integrate the bidirectional construct failed to stain with the CD3 antibody (dark histogram).
c) and d) The RFP−BFP− cells as analysed in a) and b) were gated and redisplayed as contour plots. Parallel samples of the cells had been stained with HLA-A*02:01-SLLMWITQV tetramer reagent c), or not d), wherein tetramer/CD3 double positivity in c) indicates the expected specificity of the reconstituted and integrated TCR ORF pair.

### MATERIALS AND METHODS

### DNA Sequencing

All sequencing referred to within the presented examples was conducted by the Sanger method, and conducted by GATC Biotec AB, Sweden.

### DNA Synthesis

All DNA synthesis referred to within the presented examples was conducted by Integrated DNA technologies BVBA, Belgium.

DNA Fragments >125 bp were synthesised as linear double stranded DNA molecules as a gBlock Gene Fragments' product.

DNA Fragments 15-60 nt were synthesised as single stranded DNA molecules as a ‘Custom Oligonucleotide Fragment’ product.

DNA Fragments 61-124 nt were synthesised as single stranded DNA molecules as a ‘Ultramer DNA oligonucleotide Fragment’ product.

### Vector Library Assembly and Cloning

The construction of vectors described in the examples comprises a variety of methods well known to those skilled in the art, and specific reaction compositions are outlined in detail in Examples 1 to 3. The following key materials were used in the described procedures:

| | | Supplier |
|---|---|---|
| Product | Supplier | Number |
| Acc651 | New England BioLabs | R0599L |
| Bbsl HF | New England BioLabs | R3539L |
| DH5alpha competent cells | Thermo Fisher Scientific | 18265017 |
| DNA clean and concentrator kit | Zymo Research | D4030 |
| EcoR1 | New England BioLabs | R3101S |
| Notl | New England BioLabs | R3189L |
| QIAamp DNA Mini kit | Qiagen | 51306 |
| QIAquick Gel Extraction kit | Qiagen | 28704 |
| Qiagen Plasmid Plus Midi kit | Qiagen | 12945 |
| T4 ligase | New England BioLabs | M0202L |
| T4 ligase buffer 10x | New England BioLabs | B0202S |
| Xbal | New England BioLabs | R0145S |
| Xhol | New England BioLabs | R0146S |

Oligonucleotide Duplex Encoding CDR3 (odeCDR3) Assembly

odeCDR3 were routinely assembled by annealing partially complementary single stranded oligonucleotides. A detailed description of reaction composition and conditions is provide in Example 6. The following key materials were used in the described procedures:

| | | Supplier |
|---|---|---|
| Product | Supplier | Number |
| T4 ligase buffer 10 x | New England BioLabs | B0202S |
| T4 PNK | New England BioLabs | M0201L |

### TCR Reconstitution

Operation of a TORES to reconstitute full-length TCR ORFs is described in detail in Examples 7 to 9. The following key materials were used in the described procedures:

| | | Supplier |
|---|---|---|
| Product | Supplier | Number |
| Bsal-HF | New England BioLabs | R3535L |
| CutSmart buffer 10 x | New England BioLabs | B7204S |
| DH5alpha competent cells | Thermo Fisher Scientific | 18265017 |
| Notl-HF | New England BioLabs | R3189L |
| QIAamp DNA Mini kit | Qiagen | 51306 |
| T4 Ligase | New England BioLabs | M0202L |
| T4 Ligase buffer 10 x | New England BioLabs | B0202S |

### Fluorescence Activated Cell Sorting (FACS)

Single HLA-multimer positive CD8⁺ T-cells were sorted by FACS for amplification and sequencing of TCR chains. This was achieved through standard cell sorting methodologies using a BDInflux instrument. Briefly, cells were stained with HLA-multimer reagent on ice for 10 mins, then with CD3 and CD8 antibodies as markers of CD8⁺ T-cells. Cells with CD3+CD8+Multimer+ signal were sorted to PCR plate pre-loaded with 5 μL of nuclease-free water. Specimens were snap-frozen until subsequent processing. The following key materials were used in the described procedures:

| | | Supplier |
|---|---|---|
| Product | Supplier | Number |
| CMV-B.07:02-TPRVT-pp65 (PE) | Immudex | WH2136 |
| Anti-CD8 (clone RPA-T8) (BV510) | BD | 563256 |
| Anti-CD3 (clone SK7) (APC-H7) | BD | 560176 |

Sequencing of TCR Alpha and Beta Chains from Single T-Cells

Singly FACS-sorted T-cells were subjected to a two-step amplification process that entails a V-region specific primer collection for each TRA and TRB, followed by paired nested PCR reactions that create TRA and TRB amplicons for sequence analysis as described in Example 8. This procedure is described previously (Han et. al. Nat Biotechnol. 2014 32(7): 684-692). The following materials were used in the described procedures:

| | | Supplier |
|---|---|---|
| Product | Supplier | Number |
| 2x Reaction Mix | Thermo Scientific | 12574035 |
| 5X Phusion HF Buffer | Thermo Fisher Scientific | F-549S |
| dNTPs | Thermo Fisher Scientific | 10297018 |
| Nuclease free water | Qiagen | 129114 |
| Phusion Hot Start II DNA | Thermo Fisher Scientific | F-549S |
| Polymerase | | |
| SuperScript ® III One-Step | Thermo Scientific | 12574035 |
| RT-PCR System with Platinum ® | | |
| Taq High Fidelity DNA Polymerase | | |

### Example 1

### Design and Assembly of TRA V-C Entry Vector Library for Native Human TRA Repertoire

A TORES consists of a V-C entry vector library and J donor vector library for a given TCR chain. When combined with a target odeCDR3 sequence to be inserted into a selected V-J-C context, a full-length TCR ORF can be reconstituted. Through varying odeCDR3 sequence features and/or V/J/C selection, this reconstitution step may also represent a sequence diversification step in TCR ORF engineering workflows. In the present example, the design and assembly of a TRA V-C entry vector library that contains the native human TRA V-C sequence repertoire. This is an example of the generic V-C entry vector type depicted in FIG. 2.

The DNA components required for a TRA V-C vector library are:
I. A TRA V cloning fragment for each functional TRA V gene segment encoded in the human genome
II. Single TRA C cloning fragment
III. A V-C entry vector backbone

In the present example, the TRA V and TRA C cloning fragments were synthesized and used to assemble into a target V-C entry vector backbone in a single restriction enzyme and ligase reaction. In the present example, the target V-C entry backbone was designed to permit transient expression of reconstituted TRA ORFs within mammalian cells.

In the present example, the TRA V-C entry vector library is constructed using Type IIS restriction enzyme BbsI. The Type IIS restriction enzyme used in functioning of the complete TORES to reconstitute full-length TRA ORFs is BsaI.

### Design of Synthetic TRA V Cloning Fragments

The arrangement of genetic elements of the TRA V cloning fragments in the present example is depicted in FIG. 5.

Each end of the TRA V cloning fragment encodes a standardized 5′ and 3′ primer bind DNA sequence of 20 nucleotides for propagation of the overall fragment by PCR.

Proximal to the 5′ primer bind a BbsI Type IIS restriction enzyme binding site is encoded, wherein the direction of the BbsI binding site guides the BbsI enzyme to cut the DNA 3′ to its recognition sequence. Overhangs generated by BbsI enzymatic activity are encoded by Overhang *1. This overhang is designed to permit directed ligase-dependent cloning with an arm of the V-C entry vector backbone.

A consensus kozak sequence is encoded 5′ of the ATG start codon within the TRA V gene segment for efficient initiation of translation of the final reconstituted and expressed TRA mRNA. In the present example, each TRA V segment encodes all amino acids from the start methionine residue until its last cysteine (Cys) of the TRA V segment. This Cys residue is generally recognised as a border of the TRA variable gene segments, the deletion of which is rare in naturally occurring recombined and functional TRA chains. Where necessary, native human TRA V consensus sequences have been edited to remove recognition sequences for any restriction enzymes used within assembly or reconstitution operations with the TORES, and also any enzymes used in downstream applications.

To the 3′ end of the TRA V segment a BsaI Type IIS restriction enzyme binding site is encoded, BsaI [symbol] . The direction of the BsaI binding site guides the BsaI enzyme to cut the DNA 5′ to its recognition sequence. The resulting overhang sequence is designed to encompass the last cysteine codon of the V segment element and the 3^{rd} nucleotide for amino acid codon preceding the cysteine. Thus the action of BsaI on the designed sequence creates a TRA V Cys-overhang [symbol] 1 at the 3′ end of the TRA V segment. In the present example, this Cys-overhang [symbol] 1 is standardized among all included TRA V segments to simplify and unify the cloning strategy. Where necessary the nucleotides encoding the TRA V genetic element were changed to encode this standardised overhang but not change the translated amino acid sequence. This BsaI [symbol] site is utilized during the full length TRA reconstitution reaction.

In this present example, the V-C entry vector negative selection marker is a NotI restriction enzyme binding site. To construct a NotI binding site, two halves of the site are combined when the TRA V cloning fragment and TRA C cloning fragment are ligated together. The TRA V cloning fragment encodes the NotI 5′ segment of six nucleotides.

To the 5′ end of the 3′ primer bind sequence encodes a second BbsI restriction site, that directs BbsI enzyme to cut the DNA 5′ to its recognition sequence, BbsI [symbol] . The action of BbsI on the designed sequence thus creates an overhang of 4 nucleotides, NotI 5′ overhang, which is designed to be complementary to the overhang generated on the TRA C DNA fragment and reconstitute a NotI binding site upon ligation.

Sp denote nucleotide additions to specific points of the TRA V cloning fragment to achieve the correct spacing of Type IIS restriction enzyme binding site and the cut site, when adjacent to such sites. Sp blocks flanking the NotI restriction enzyme binding site sequence have been used to space the NotI binding and cut site appropriately for efficient action. The selection of nucleotides considered the potential impact of DAM methylation of the BsaI binding site.

Full DNA sequences for the TRA V cloning fragments in the present example of native human TRA chains are provided as SEQ0001 to SEQ0046. These sequences includes the 5′ primer bind and 3′ primer bind sequences.

### Design of Synthetic TRA C Cloning Fragment

The arrangement of genetic elements of the TRA C cloning fragments in the present example is depicted in FIG. 6.

Each end of the TRA C cloning fragment encodes a standardized 5′ and 3′ primer bind DNA sequence of 20 nucleotides for propagation of the overall fragment by PCR.

Proximal to the 5′ primer bind sequence a BbsI restriction enzyme recognition site is encoded, such that BbsI enzyme will cut the DNA 3′ to its recognition sequence, BbsI [symbol] .

The TRA C cloning fragment encodes the NotI 3′ segment of six nucleotides, which completes a NotI recognition site that will make up the V-C entry vector negative selection marker. The adjacent BbsI [symbol] restriction site acts upon the NotI 3′ element to create the NotI 3′ overhang of four necleotides. This overhang is designed to be complementary to the NotI 5′ overhang generated on the TRA V DNA fragment and reconstitute a full NotI binding site upon assembly of V-C entry vectors.

To the 3′ end of the NotI 3′ element, the TRA C cloning fragment encodes a BsaI restriction enzyme binding site, BsaI [symbol] . The direction of the BsaI binding site guides the BsaI enzyme to cut the DNA 5′ to its recognition sequence. The resulting overhang sequence is designed to start from the first cytosine of the TRA C genetic fragment, TRA C overhang [symbol] 3. This BsaI [symbol] site is utilized during the full length TRA reconstitution reaction. The BsaI [symbol] enzyme acts upon the TRA C segment encoded in the V-C entry vector to create the necessary TRA C overhang [symbol] 3 during reconstitution reactions. A consensus TRA C sequence from the cytosine residue 5′ of the first glutamine codon until the stop codon is included in the TRA C cloning fragment in the present example

To the 5′ of the 3′ primer bind encodes a BbsI restriction enzyme recognition sequence, BbsI [symbol] . The direction of the BbsI binding site guides the BbsI enzyme to cut the DNA 5′ to its recognition sequence. Overhangs generated by BbsI enzymatic activity are encoded by Overhang *2. The design of this overhang permits directed ligase-dependent cloning with an arm of the V-C entry vector backbone during assembly.

Sp denote nucleotide additions to specific points of the TRA C cloning fragment to achieve the correct spacing of Type IIS restriction enzyme binding site and the cut site, when adjacent to such sites. Sp blocks flanking the NotI restriction enzyme binding site sequence have been used to space the NotI binding and cut site appropriately for efficient action. The selection of nucleotides considered the potential impact of DAM methylation of the BsaI binding site

The full DNA sequence for the TRA C cloning fragment in the present example of native human TRA chains are presented as, SEQ0047. This sequence includes the 5′ primer bind and 3′ primer bind sequences.

### Design of V-C Entry Vector Backbone for Transient Expression of Reconstituted TRA ORF in Mammalian Cells

In the present example, the V-C entry vector backbone is derived from the pMA plasmid. It encodes a Col E1 origin of replication, ori, along with antibiotic resistance beta-lactamase gene, positive selection #1. Beta-lactamase confers resistance to the penicillin group of beta-lactam antibiotics such as ampicillin and carbenicillin.

The vector backbone, as depicted in FIG. 7, encodes the required genetic elements that confer the appropriate functionality for downstream applications of the fully reconstituted TRA ORF. In this present example, the 5′ genetic element encodes the CMV constitutive mammalian promoter and the 3′ genetic element encodes the SV40 pA polyadenylation signal to permit transient expression of the fully reconstituted TRA ORF in a mammalian cell.

In the present example, the vector backbone encodes Acc65I and XbaI restriction enzyme binding sites that generate overhang *1′ and overhang *2′, respectively. Overhang *1′ is complementary to overhang *1 within the TRA V cloning fragment (FIG. 5). Overhang *2′ is complementary to overhang *2 within the TRA C cloning fragment (FIG. 6). These complementary overhangs permit directed cloning of the TRA V and TRA C cloning fragments into the V-C entry vector backbone.

Sp feature denotes nucleotides added between the Acc65I and XbaI restriction enzyme recognition sites required for distancing the two sites for efficient action.

The sequence of the vector backbone from the 5′ genetic element encoding the CMV constitutive promoter, to the 3′ genetic element encoding the SV40 pA polyadenylation signal is presented as SEQ0048.

### Method to Assemble TRA V-C Entry Vector Library

This method utilizes standard molecular biology techniques to assemble selected TRA V cloning fragment (FIG. 5) and TRA C cloning fragment (FIG. 6) into a given V-C entry vector backbone (FIG. 7) to create a TRA V-C entry vector (FIG. 2). In this present example, the method performs the restriction enzyme digestion and ligation reaction in a single reaction.

### RE Digestion and Ligation Reaction

100 ng of linear vector backbone (linearised by ACC65I and XbaI digestion)

10 ng of TRA V genetic fragment

20 ng of TRA C genetic fragment

2 μl 10× NEB ligase buffer

0.5 μl of BbsI

1 μl of T4 DNA ligase

Up to 20 μl of H₂O

### Reaction Conditions

Step 1; 2 min at 37° C.

Step 2; 3 min at 16° C.

Repeat step 1 and 2, 20 times

5 min at 50° C.

5 min at 80° C.

Return to room temperature

Resulting product is transformed into competent E. coli cells that are selected for carbenicillin-resistant colonies. Plasmids isolated from selected colonies are sequenced to determine correctly assembled constructs. The procedure is repeated for each independent V segment cloning fragment. The resulting constructs make up the TRA V-C entry vector library for use in reconstitution of full-length TRA ORFs for later use in transient expression of said reconstituted TRA in mammalian cells. The sequence of the cloned V-C fragments that make up the TRA V-C entry vector library is presented as SEQ0049 to SEQ0094. The presented sequences include all the Kozac sequence preceding the start codon of the variable segment, to the stop codon of the C segment.

### Example 2

### Design and Assembly of TRA J Donor Vector Library for Native Human TRA Repertoire

A TORES consists of a V-C entry vector library and J donor vector library for a given TCR chain. When combined with a target odeCDR3 sequence to be inserted into a selected V-J-C context, a full-length TCR ORF can be reconstituted. Through varying odeCDR3 sequence features and/or V/J/C selection, this reconstitution step may also represent a sequence diversification step in TCR ORF engineering workflows

In the present example, the design and assembly of a TRA J Donor vector library that contains the native human TRA J sequence repertoire. This is an example of the generic J Donor vector type depicted in FIG. 3.

In the present example, a TRA J receiving cassette fragments are constructed and inserted to a J donor vector backbone to create a J receiving cassette vector. Subsequently, a synthetic TRA J segment parts may be assembled into a TRA J receiving cassette vector to create the J Donor vector library. This flexible multistep assembly method allows rapid and cost effective engineering of J donor segment features, such as variations in J segment length.

The DNA components required for a TRA J donor vector library are:
I. TRA J receiving cassette fragment
II. J donor vector backbone
III. TRA J receiving cassette vector
IV. TRA J segment part

### Design of Synthetic TRA J Receiving Cassette Fragment

The annealing of two single stranded DNA oligonucleotides is used to generate the receiving site cassette fragment that by design contains 4-nucleotide single-strand overhangs at each end of the DNA fragment; Overhang *3 and Overhang *4. The 4-nucleotide overhangs to permit directed ligase-dependent cloning into a J donor vector backbone to create the TRA J receiving cassette vector, depicted in FIG. 8.

The pair of Type IIS restriction sites, BsaI [symbol] and BsaI [symbol] are positioned at the 5′ and 3′ end of the receiving site cassette DNA fragment. The direction of the BsaI recognition site is to guide BsaI enzyme to cut the DNA towards the centre of the construct. These sites are used during TRA ORF reconstitution protocol by generating overhang [symbol] 2-5′ and overhang [symbol] 3-3′. Overhang [symbol] 3 is a component of the TRA C part encoded in the receiving cassette fragment, while overhang [symbol] 2 is defined after the TRA J segment part is cloned (infra vide).

The BbsI pair of Type IIS recognition sites BbsI [symbol] and BbsI [symbol] are encoded near the middle of the cassette and used for assembly of the TRA J donor vector, in creating complementary overhangs included in synthesized TRA J segment parts (infra vide). The 5′ BbsI site, BbsI [symbol] , cuts into the BsaI site to create overhang *5 at the 3′ end of this feature. The 3 BbsI site, BbsI [symbol] , cuts into the TRA C part element, to create overhang *6 at the 5′ end of this element. These overhangs are encoded within the BsaI and TRA C part features of this construct as to avoid addition of non-native nucleotides that would be incorporated into the final reconstituted TRA ORF.

The region between BbsI [symbol] enzyme generated overhang and the BsaI [symbol] enzyme generated overhang encodes a proportion of the TRA C region starting from the second nucleotide of the TRA C genetic fragment, TRA C part. The motivation for starting from the second nucleotide of the TRA C genetic fragment is because in the present example of a human TRA locus TORES, the resulting overhang is TATC and not a palindromic overhang, which would be the case if the beginning of the TRA C genetic fragment were including (resulting overhang ATAT). A palindromic overhang should be avoided, as it would permit two vector ends joining without the required TRA J segment part insert. The orientation of the BbsI [symbol] site permits the in-frame ligase dependent cloning of all TRA J fragments 3′ end to the 5′ beginning of the TRA C region in the receiving site cassette. The orientation of the BsaI [symbol] site permits the in-frame ligase-dependent cloning of the beginning of the TRA C region with the remaining TRA C fragments in the final step of the TRA full length ORF reconstitution protocol using a complete TORES.

Between the two BbsI binding sites is an 8 nucleotide recognition sequence for the enzyme NotI. This restriction site is utilized as a negative selection marker to reduce the background of the parental plasmid colonies. This is achieved when NotI enzyme is added after the TRA J gene fragment insertion has been performed. Therefore plasmids correctly cloning a TRA J gene fragment would remain circular in the presence of NotI enzyme but parental plasmids that did not exchange its NotI site for a TRA J gene fragment will be linearized, in turn biasing the bacterial transformation to propagate a complete circular TRA J fragment-containing plasmid.

Sp denote nucleotide additions to specific points of the TRA J receiving cassette fragment to achieve the correct spacing of Type IIS restriction enzyme binding site and the cut site, when adjacent to such sites. Sp blocks flanking the NotI restriction enzyme binding site sequence have been used to space the NotI binding and cut site appropriately for efficient action. Additional nucleotides have been included to maintain correct reading frame within the final reconstituted full-length TRA. The selection of nucleotides considered the potential impact of DAM methylation of the BsaI binding site.

The full DNA sequence for the TRA J receiving cassette fragment oligonucleotides in the present example of native human TRA chains are presented as, SEQ0095 and SEQ0096. Both forward (F1) and reverse (R1) oligonucleotide sequences are listed.

### Design of the J Donor Vector Backbone

The J donor vector backbone is used to insert the TRA J receiving cassette fragment to create the TRA J receiving cassette vector. The backbone is thus carried through to the J Donor vector library. In the final reaction to create TRA full-length ORFs, this backbone is a reaction byproduct (FIG. 4e), and thus carries minimal features as depicted in FIG. 9.

In the present example, the J donor vector backbone encodes a Col E1 origin of replication, ori. The antibiotic resistance is the aminoglycoside 3′-phosphotransferase gene, positive selection selection #2. Aminoglycoside 3′-phosphotransferase confers resistance to antibiotic substrates such as kanamycin, streptomycin, neomycin, and gentamicin. This alternate positive selection is used to ensure J donor vectors are not selected for after full-length TCR ORF reconstitution, which are selected on positive selection #1.

In the present example the vector EcoRI and XhoI restriction enzyme binding sites that generates complementary overhang, overhang *3′ and overhang *4′, respectively. Overhang *3′ is complementary with Overhang *3 contained within the TRA J receiving cassette fragment. Overhang *4′ is complementary with Overhang *4 contained within the TRA J receiving cassette fragment. These overhangs permits directed cloning of the TRA J receiving cassette fragment.

Sp block denotes nucleotides added between the EcoRI and XhoI restriction enzyme binding sites for distancing the two sites to ensure efficient action.

In the present example, the J donor backbone is presented as SEQ0097.

### Method to Assemble the TRA J Receiving Cassette Vector

This method utilizes standard molecular biology techniques to assemble the given TRA J receiving cassette fragments (FIG. 8) into a given J donor vector backbone (FIG. 9) to create a TRA J receiving cassette vector (FIG. 10). The resulting TRA J receiving cassette vector is used to insert TRA J segment parts (FIG. 11) to construct TRA J Donor vectors (FIG. 3).

First, the two oligonucleotides to form the TRA J receiving cassette DNA fragment must be phosphorylated and annealed.

### Reaction Mix

| | |
|---|---|
| Oligonucleotide (sense strand) (100 μM) | 1 μl |
| Oligonucleotide (anti-sense strand) (100 μM) | 1 μl |
| T4 ligase buffer 10x | 1 μl |
| T4 PNK | 1 μl |
| H₂O | 6 μl |

### Reaction Conditions

Incubate for 37° C. for 1 hour

Denature at 95° C. for 5 min

Anneal sense and anti-sense oligonucleotides by slowly cooling the reaction down to 25° C. at 3° C. per min

### Assembly Ligation of TRA J Receiving Cassette Fragments and J Donor Vector Backbone.

### Reaction Mix

| | | |
|---|---|---|
| Linear vector backbone | 100 | ng |
| Receiving site cassette DNA fragment (0.5 μM) | 2 | μl |
| T4 ligase buffer 10x | 2 | μl |
| T4 ligase | 0.5 | μl |
| H₂O | up to 20 | μl |

### Reaction Conditions

Incubate for 1 hour at 25° C.

Heat inactivate at 65° C. for 10 min

Resulting product is transformed into competent E. coli cells and selected for Kanamycin resistant colonies. Resistant colonies are selected to determine correctly assembled constructs. The resulting plasmid is the TRA J receiving cassette vector. In the present example, the TRA J receiving cassette vector is presented as SEQ0098 and depicted in FIG. 10.

### Design of Synthetic TRA J Segment Parts

Having generated the TRA J receiving cassette vector synthetic TRA J segment parts must be generated to insert into this vector. Each TRA J sequence is inserted into an independent TRA J receiving cassette vector context to generate the TRA J donor vector library as part of the human TRA TORES.

The TRA J donor vector library comes in two different forms, comprised of a long or short J segment part. The short TRA J segment part encodes all amino acids from the start of the CDR3 border codon. However, considering that the majority of TRA J segments are trimmed back by less than 10 nucleotides during TCR rearrangement, a TRA J donor library containing a longer TRA J germline segment is designed, long TRA J segment part. The motivation for a longer TRA J gene fragment library is that a shorter oligonucleotide duplex encoding CDR3 (odeCDR3) would be required for the full length TRA reconstitution, than if the short TRA J fragment would be used. Since highly variable sequences are provided as short oligonucleotide duplexes, odeCDR3, a shorter CDR3 oligonucleotide synthesis is less likely to contain truncated or mutated oligonucleotide contaminants and therefore reduce the likelihood of oligonucleotide duplex with sequence errors being cloned during full length TRA reconstruction. Furthermore, shorter odeCDR3 syntheses are cost-saving.

The TRA J segment parts are constructed by annealing two single-stranded DNA oligonucleotides designed to contain 4-nucleotide single-strand overhangs at each end of the DNA fragment. The resulting TRA J segment part is depicted in FIG. 11.

The 5′ overhang designated Overhang *5′ is complementary to the Overhang *5 generated within J donor receiving cassette vector by BbsI action. The 3′ overhang designated Overhang *6′ is complementary to the Overhang *6 generated within J donor receiving cassette vector by BbsI action. This pair of complementary overhangs permits directional cloning of the TRA J segment parts into the TRA J receiving cassette vector.

The Short TRA J segment part encodes all amino acids from the start of the CDR3-J border Phe codon. The CDR3 is defined as the sequence flanked by the C-terminal-conserved Cys of the V region, and Phe of the J region which is part of the Phe-Gly/Ala conserved motif. This conserved Phe-Gly/Ala motif is utilized to standardize the 5′ overhangs of the TRA J fragments to TTTG for downstream TRA reconstitution. The exceptions to this standardization in the present example are human TRAJ33 and TRAJ38 that border the CDR3 region with Trp and Gly. The 5′ overhangs are TGGG for both TRAJ33 and TRAJ38 in the present example.

The long TRA J segment part is designed to encode more amino acids N-terminal of the CDR3 border amino acids. The start point of each long gene fragment is at the first nucleotide of an amino acid codon positioned 10-12 nt from the 5′ end of the germline encoded TCR joining element. The 5′ end of each long TRA J segment part remains identical to that of the short TRA J segment part.

To both short and long TRA J segment parts an adenine, represented as the A block in FIG. 11, is added to the 3′ end of each TRA J segment part. This adenine represents the first nucleotide of the TRA C fragment that is excluded from the TRA J receiving cassette.

The sequences of the short TRA J segment parts of the present example of native human J segments are presented as SEQ0099 to SEQ0210 and the long TRA J segment parts SEQ0211 to SEQ0322. In both cases, both forward (F1) and reverse (R1) oligonucleotide sequences are listed.

### Method to Assemble the Short or Long J-Donor Vector Library

This method utilizes standard molecular biology techniques to clone the Short TRA J segment or Long TRA J segment part part (FIG. 11) into the TRA J receiving cassette vector (FIG. 10) to create TRA J donor vectors (FIG. 3) containing the short or long TRA J segments. In this present example, the method performs the restriction enzyme digestion and ligation reaction in a single reaction.

The DNA components required for a J donor vector library is as follows:
I. Short TRA J segment part or Long TRA J segment part
II. J donor receiving cassette vector

### Phosphorylation and Annealing Two Oligonucleotides to Form the TRA J Segment Part DNA Fragment

### Reaction Mix

| | |
|---|---|
| Oligonucleotide (sense strand) (100 μM) | 1 μl |
| Oligonucleotide (anti-sense strand) (100 μM) | 1 μl |
| T4 ligase buffer 10x | 1 μl |
| T4 PNK | 1 μl |
| H₂O | 6 μl |

### Reaction Conditions

Incubate for 37° C. for 1 hour

Denature at 95° C. for 5 min

Anneal sense and anti-sense oligonucleotides by slowly cooling the reaction down to 25° C. at 3° C. per min

### RE Digestion and Ligation Reaction

| | | |
|---|---|---|
| TRA J receiving cassette backbone | 100 | ng |
| TRA J DNA fragment (0.5 μM) | 2 | μl |
| 10x NEB T4 ligase buffer | 2 | μl |
| Bbsl | 0.5 | μl |
| T4 DNA ligase | 0.5 | μl |
| H₂O | up to 20 | μl |

### Reaction Conditions

Step 1; 2 min at 37° C.

Step 2; 3 min at 16° C.

Repeat step 1 and 2, 20 times

5 min at 50° C.

5 min at 80° C.

Return to room temperature

Add 0.5 μl of NotI enzyme and incubate for 30 min at 37° C. to linearize parental vector.

Reaction product is transformed into competent E. coli cells and selected for Kanamycin resistance. Selected resistant colonies are sequenced to determine correctly assembled constructs. The resulting constructs make up the TRA J donor vector library, encoding either a long or a short TRA J gene fragment.

The sequence of the resulting libraries, excluding backbone sequence outside of the BsaI recognition sites, are presented as SEQ0323 to SEQ0378 for the TRA short J donor library and SEQ0379 to SEQ0434 for the TRA long J donor library.

### Example 3

### Design and Assembly of TRB V-C Entry Vector and TRB J Donor Vector Libraries for Native Human TRB Repertoire

In the above examples, the design and assembly of V-C entry vector and J donor vector libraries for the native human TRA repertoire was described in detail. The overall design and assembly of such vector libraries encoding sequences of the TRB repertoire is essentially the same. In the present example, the design and assembly of the TRB V-C entry vector and TRB J Donor vector libraries will be briefly outlined in order to construct a TORE or the native human TRB TCR locus.

### Design and Assembly of TRB V-C Entry Vector Library for Native Human TRB Repertoire

In the present example, the design and assembly of a TRB V-C entry vector library that contains the native human TRB V-C sequence repertoire. This is an example of the generic V-C entry vector type depicted in FIG. 2.

The DNA components required for a TRB V-C vector library are:
I. A TRB V cloning fragment for each functional TRB V gene segment encoded in the human genome
II. TRB C1 or TRB C2 cloning fragment
III. A V-C entry vector backbone

In contrast to the human TRA locus, the human TRB locus encodes two distinct constant segments, TRB C1 and C2. Thus, to capture both constant regions, two V-C entry vector sets are constructed to pair each of the V segments with each C1 and C2 segments.

In the present example, the TRB V and TRB C cloning fragments were synthesized and used to assemble into a target V-C entry vector backbone in a single restriction enzyme and ligase reaction. In the present example, the target V-C entry backbone was designed to permit transient expression of reconstituted TRB ORFs within mammalian cells.

In the present example, the TRB V-C entry vector library is constructed using Type IIS restriction enzyme BbsI. The Type IIS restriction enzyme used in functioning of the library to reconstitute full-length TRB ORFs is BsaI.

### Design of Synthetic TRB V Cloning Fragments

The arrangement of genetic elements of the TRB V cloning fragments is identical to those of the TRA V cloning fragments described in Example 1, as depicted in FIG. 5.

Full DNA sequences for the TRB V cloning fragments in the present example of native human TRB chains are presented as SEQ0435 to SEQ481.

### Design of Synthetic TRB C Cloning Fragment

The arrangement of genetic elements of the TRB C cloning fragments is identical to those of the TRA C cloning fragments described in Example 1, as depicted in FIG. 6.

The TRB locus encodes two distinct C segments, and both are included in the design of the TRB V-C entry vector library.

The full DNA sequence for the TRB C cloning fragments in the present example of native human TRB chains are presented as SEQ0482 and SEQ0483.

### Method to Assemble TRB V-C Entry Vector Library

The method to assemble the given TRB V and TRB C cloning fragments into a given V-C entry vector backbone to create a TRB V-C entry vector is identical to that described in Example 1. The V-C entry vector backbone used, designed for transient expression of full-length TRA or TRB ORFs in mammalian cells is used in the present example, and in example 1 (SEQ0048).

The sequence of the cloned V-C fragments that make up the TRA V-C entry vector library is presented as SEQ0484 to SEQ0577.

### Design and Assembly of TRB J Donor Vector Library for Native Human TRB Repertoire

In the present example, the design and assembly of a TRB J Donor vector library that contains the native human TRB J sequence repertoire. This is an example of the generic J Donor vector type depicted in FIG. 3.

In the present example, a TRB J receiving cassette fragments are constructed and inserted to a J donor vector backbone to create a TRB J receiving cassette vector. Subsequently, a synthetic TRB J segment part may be assembled into a TRB J receiving cassette vector to create the TRB J Donor vector library. This flexible multistep assembly method allows rapid and cost effective engineering of J donor segment features, such as variations in J segment length.

This procedure follows the same pattern as the TRA J donor vector assembly described in Example 2. However, it should be noted that since the J receiving cassette fragments contain parts of the C segment, the TRA J and TRB J receiving cassette fragments differ with regard to the C part sequence, that must correspond to the respective C gene segments. Moreover, in contrast to TRA J scenario that only requires a single J receiving cassette fragments, the TRB J requires two distinct J receiving cassette fragments to account for the use of alternate C1 and C2 segments.

The DNA components required for a TRB J donor vector library are:
I. TRB J C1 or TRB J C2 receiving cassette fragment
II. J donor vector backbone
III. TRB J C1 or TRB J C2 receiving cassette vector
IV. TRB J segment part

### Design of Synthetic TRA J Receiving Cassette Fragment

The annealing of two single stranded DNA oligonucleotides is used to generate the receiving cassette fragments, which contain 4-nucleotide single-strand overhangs at each end of the DNA fragment, depicted in FIG. 8. The 4-nucleotide overhangs permit directed ligase-dependent cloning into a J donor vector backbone to create the TRB J receiving cassette vector,

The two receiving cassette fragments required for alternate use of C1 and C2 segments are presented as SEQ0578 and SEQ0581. For each fragment, the forward (F1) and reverse (R1) oligonucleotide sequences are provided.

### Method to Assemble the TRB J Receiving Cassette Vectors

The method for assembly of the TRB J receiving cassette vectors is identical to that of the method for assembly of TRA J receiving cassette vectors described in Example 2. The same J donor vector backbone (SEQ0097) is used to generate two TRB J receiving cassette vectors, each containing one C1 or C2 part corresponding to the alternate C segments for the TRB locus.

The resulting two TRB J receiving cassette vector is used to insert TRB J segment parts to construct TRB J Donor vectors.

The resulting TRB J receiving cassette vectors are presented as SEQ0582 and SEQ0583.

### Design of Synthetic TRB J Segment Parts

The TRB J segment parts are constructed by annealing two single-stranded DNA oligonucleotides designed to contains 4-nucleotide single-strand overhangs at each end of the DNA fragment. The arrangement of this part and method of assembly are identical to that of the TRA J segment parts, and depicted in FIG. 11.

In the case of the Short TRB J segment part encodes all amino acids from the start of the CDR3-J border Phe codon. The CDR3 is defined as the sequence flanked by the C-terminal-conserved Cys of the V region, and Phe of the J region, which is part of the Phe-Gly motif conserved across all human TRB J segments. This conserved Phe-Gly motif is utilized to standardize the 5′ overhangs of the TRA J fragments to TTTG for downstream TRB reconstitution. Unlike the TRA J segments, there are no exceptions to this standardized overhang in the TRA J segment parts in the present example.

To both short and long TRB J segment parts an adenine, represented as the A block in FIG. 11, is added to the 3′ end of each TRB J segment part. This adenine represents the first nucleotide of the TRB C fragment that is excluded from the TRB J receiving cassettes.

The sequences of the short TRB J segment parts of the present example of native human J segments are presented as SEQ0584 to SEQ0609, and the long TRB J segment parts SEQ0610 to SEQ0635. In both cases, both forward (F1) and reverse (R1) oligonucleotide sequences are listed.

### Method to Assemble TRB Short or Long J Donor Vector Library

The procedure to assemble the TRB J donor libraries is identical to that of the TRA libraries described in Example 2. However, in the case of the TRB libraries, there are four libraries to generate, in contrast to the short and long libraries for the TRA locus segments.

In the case of TRB libraries, each short and long libraries can be constructed to carry each of the alternate C1 and C2 C segments, resulting in four subsets within the TRB J donor library.

The DNA components required for a J donor vector library is as follows:
I. Short TRB J segment part or Long TRB J segment part
II. TRB J C1 or TRB J C2 receiving cassette vector

Following the same procedure as described in example 2, the four resulting subsets within the TRB J donor library are generated. The sequence of the resulting libraries, excluding backbone sequence outside of the BsaI recognition sites is presented.

TRB C1 short J donor library presented as SEQ0636 to SEQ0648

TRB C2 short J donor library presented as SEQ0649 to SEQ0661

TRB C1 long J donor library presented as SEQ0662 to SEQ0674

TRB C2 long J donor library presented as SEQ0675 to SEQ0687

### Example 4

### Design of TRA and TRB V-C Entry Vectors for Recombinase Mediated Cassette Exchange Application

The design of the TRA and TRB V-C entry vectors in examples 1 and 3, respectively, are such that the full-length TCRs reconstituted with the use of these TORES for each TCR chain may be directly applicable to transient expression in mammalian cells by transfection. The overall design of the TORES allows the V-C entry vector backbone to be exchanged for any backbone suitable for downstream applications, and the necessary V-C entry vector libraries to be rapidly assembled from V and C cloning fragments.

In the present example, a pair of heterospecific FRT V-C entry vector backbones are used to assemble TRA and TRB V-C entry vector libraries. Each TRA and TRB V-C entry vector libraries are constructed with vector backbones containing distinct flippase recognition target (FRT) sequences. One downstream application of such vectors, is the submission of final reconstituted TRA and TRB pairs to rapid genomic integration into mammalian cells harboring relevant FRT sites.

In the present example, TRA V-C entry vector of the design depicted in FIG. 7, contains F14 and F15 FRT sequences as the 5′ and 3′ genetic elements, respectively. This F14/F15 V-C entry vector backbone sequence is presented as SEQ0688.

This backbone was used to construct TRA V-C entry library of the design outlined in FIG. 2, with the use of TRA V cloning fragments (SEQ0001 to SEQ0046) and TRA C cloning fragment (SEQ0047) using methods outlined in Example 1. This construction yielded a TRA V-C entry library with sequences identical to that of the transient expression TRA V-C entry vector library (SEQ0049 to SEQ0094), but flanked by the FRT sequences in the context of the F14/F15 V-C entry vector backbone.

In the present example, TRB V-C entry vector of the design depicted in FIG. 7, contains FRT and F3 FRT sequences as the 5′ and 3′ genetic elements, respectively. This FRT/F3 V-C entry vector backbone sequence is presented as SEQ0689.

This backbone was used to construct TRB V-C entry library of the design outlined in FIG. 2, with the use of TRA V cloning fragments (SEQ0435 to SEQ0481) and TRB C cloning fragments (SEQ0482 and SEQ0483) using methods outlined in Example 3. This construction yielded a TRB V-C entry library with sequences identical to that of the transient expression TRB V-C entry vector library (SEQ0484 to SEQ0577), but flanked by the FRT sequences in the context of the FRT/F3 V-C entry vector backbone. Overall, the TRA and TRB V-C entry vectors in the present example may be used interchangeably in with V-C entry vectors in above examples. In each case, the J donor vector remains unchanged, as does design and provision of odeCDR3 (see Example 6). In effect, different V-C entry vector backbones provide full-length TCR ORFs in desired vector contexts for downstream applications as a direct product of the TCR reconstitution reaction.

### Example 5

### Design of V-C Entry Vectors and J Donor Vectors Suitable for Provision of any VJ-C Combinations

In the above examples of V-C entry vectors and J donor vectors, native human TRA and TRC TCR chains are used as a practical demonstration of the TORES. However, it is clear that any native or non-native TCR chain can be employed in the TORES format upon insertion of required TCR chain sequences into the specified vector formats.

To achieve a TORES for any given TCR chain, four sequence elements are required specific for said TCR chain:
X—a variable (V) gene segment fragment
Y—a constant (C) genes segment fragment
Y′—a constant (C) gene segment part
Z—a joining (J) gene segment fragment

According to the above examples, these four forms of sequence element can be assembled into various vector contexts to construct and deploy a TORES for any given V-J-C combination for any given TCR chain. For example, systems for the native human TRG and TRD locus, variant synthetic human TCR chain forms, or native TCR chain forms of an organism other than humans.

### V-C Entry Vector Assembly and Final Construct

In order to achieve assemble of the V-C entry vector, two intermediates can be constructed that contain the relevant V and C gene segment fragments.

A V cloning fragment, according to FIG. 5, is designed to contain required V gene segment fragment in the context of appropriate cloning sites for final V-C entry vector assembly. In the present example, as is also presented in examples 1, 3 and 4, the assembly of V-C entry vectors was achieved through use of BbsI restriction enzyme sites create target overhangs at the 5′ and 3′ end as to match complementary overhangs in the V-C entry vector backbone and C cloning fragment, respectively. The exemplified strategy also incorporates a NotI restriction enzyme site as a negative selection marker.

The generic sequence of such a cloning approach is presented as SEQ-0690, wherein the V gene segment fragment is bounded by BbsI sites at the 5′ and 3′ ends, and the V gene segment fragment is denoted as XNₙ, wherein X is the designation for the V gene segment, N represents any nucleotide, and n represents the number of nucleotides in said sequence. The V gene segment fragment is bounded by Kozac and spacer nucleotides at the 5′ and 3′ ends, respectively, and include the translation start codon.

The second intermediate that can constructed to assemble a V-C entry vector libraries for specific TCR chain sequences is the C cloning fragment as depicted in FIG. 6. In the present example, as is also presented in examples 1, 3 and 4, the assembly of V-C entry vectors was achieved through use of BbsI restriction enzyme sites create target overhangs at the 5′ and 3′ end as to match complementary overhangs in the V cloning fragment and backbone the V-C entry vector, respectively. The exemplified strategy also incorporates a NotI restriction enzyme site as a negative selection marker.

The generic sequence of such a cloning approach is presented as SEQ-0691, wherein the C gene segment fragment is bounded by BbsI sites at the 5′ and 3′ ends, and the C gene segment fragment is denoted as YNₙ, wherein Y is the designation for the C gene segment, N represents any nucleotide, and n represents the number of nucleotides in said sequence. The C gene segment fragment is bounded by the cytosine 5′ of the first Glu codon of the C gene segment, or equivalent conserved site, and spacer nucleotide(s) at the 5′ and 3′ ends, respectively, and would include the translation stop codon.

To assemble a V-C entry vector using the above defined V and C cloning fragments, these fragments are combined with a V-C entry vector backbone as depicted in FIG. 7 and described in examples 1 and 3. In the present and above examples, this backbone is defined with the Acc65I and XbaI restriction enzyme binding sites that generate overhang *1′ and overhang *2′, respectively. Overhang *1′ is complementary to overhang *1 within the V cloning fragment (FIG. 5). Overhang *2′ is complementary to overhang *2 within the C cloning fragment (FIG. 6). These complementary overhangs permit directed cloning of the V and C cloning fragments into the V-C entry vector backbone.

Sp feature denotes nucleotides added between the Acc65I and XbaI restriction enzyme recognition sites required for distancing the two sites for efficient action.

As outlined in example 4, a V-C entry vector backbone (FIG. 7), by way of providing the 5′ and 3′ genetic elements to the V-C entry vector (FIG. 2), ultimately provides the vector context into which a full length TCR ORF is reconstituted (FIG. 4h). Thus, a range of selections can be made for the V-C entry vector backbone/V-C entry vector to satisfy a range of downstream applications for the reconstituted TCR ORF.

In the present example, as in examples 1, 3 and 4 above, three different V-C entry vector backbones are provided.

The sequence of the V-C entry vector backbone with the 5′ genetic element encoding the CMV constitutive promoter, and the 3′ genetic element encoding the SV40 pA polyadenylation signal is presented as SEQ0048. This vector context permits transient expression of reconstituted TCR ORFs in mammalian cells.

When this transient expression V-C entry vector backbone SEQ0048 is combined with the generic V cloning fragment (SEQ0690) and C cloning fragment (SEQ0691) in the present example, a generalized V-C entry vector can be defined by the sequence SEQ0692.

The sequence of the V-C entry vector backbone with the 5′ genetic element encoding the FTR site, F14, sequence, and the 3′ genetic element encoding the FRT site, F15, sequence is presented as SEQ0688. This vector context permits flippase mediate cassette exchange of reconstituted TCR ORFs into genetic contexts bounded by F14 and F15 heterospecific FRT sites. For example, such an approach can be employed for stable integration of TCR ORFs into mammalian cell lines harboring such heterospecific FRT receiver sites.

When this F14/F15 RCME V-C entry vector backbone SEQ0688 is combined with the generic V cloning fragment (SEQ0690) and C cloning fragment (SEQ0691) in the present example, a generalized V-C entry vector can be defined by the sequence SEQ0693.

The sequence of a second V-C entry vector backbone with the 5′ genetic element encoding the FRT site, FRT, sequence, and the 3′ genetic element encoding the FRT site, F3, sequence is presented as SEQ0689. This vector context permits flippase mediate cassette exchange of reconstituted TCR ORFs into genetic contexts bounded by FRT and F3 heterospecific FRT sites. For example, such an approach can be employed for stable integration of TCR ORFs into mammalian cell lines harboring such heterospecific FRT receiver sites.

When this FRT/F3 RCME V-C entry vector backbone SEQ0689 is combined with the generic V cloning fragment (SEQ0691) and C cloning fragment (SEQ0692) in the present example, a generalized V-C entry vector can be defined by the sequence SEQ0694.

In the present example, one TCR chain can be reconstituted in one of the two RCME contexts, and a second chain in the other RCME context. For example, a TRA ORF can be reconstituted in the F14/F15 context, and a TRB ORF can be reconstituted in the FRT/F3 context. Thus, paired TRA/TRB constructs can be integrated into a mammalian cell line harboring F14/F15 and FRT/F3 sites by RCME for stable expression of the TRA/TRB pair in said cell line.

### J Donor Vector Assembly and Final Construct

Described above are generic constructs that may be used to assemble various V and C

TCR gene segment combinations into V-C entry vectors as a component of a TORES.

In order to complete a TORES, appropriate J donor vectors are required. As described above in example 2, a J donor vector may be assembled in a multistep process to achieve a J donor system with flexibility to design features of J segments. One important aspect to note is that the J donor vector contributes a C part corresponding to the C segment of a matched V-C entry vector library.

To assemble a J donor vector library, four different constructs are required;
I. J receiving cassette fragment
II. J donor vector backbone
III. J receiving cassette vector
IV. J segment part

In the present example, as in example 2, the J donor vector is assembled using BbsI restriction sites, and the overall TORES operates with the use if BsaI restriction sites carried in both the resulting J donor vector and matched V-C entry vector.

The J receiving cassette fragment as depicted in FIG. 8 importantly contains a C part corresponding to the C segment carried by the matched V-C donor vector library. The generic sequence of such a cloning approach is presented as SEQ0695 to SEQ0696, wherein the C part encoding a 5′ portion on the C segment is bounded by a BbsI recognition site to the 5′ and a BsaI site to the 3′. The two presented sequences represent the sense and antisense oligonucleotides annealed to generate the J receiving cassette fragment, wherein the Y′ sequences provided are complementary. The C part in the presented sequence is designated Y′Nₙ, wherein Y′ is the designation for the C part matching the Y C segment described above, N represents any nucleotide, and n represents the number of nucleotides in said sequence. The BbsI site is spaced and orientated to cut in the 3′ direction, to create a cloning overhang to match a J segment part to be inserted. The BsaI site is spaced and cuts in the 5′ direction to create the C overhang in the final assembly reaction for generating full-length TCR ORFs in operation of the TORES. A second BsaI site at the 5′ terminus of the J receiving cassette fragment, cutting in the 3′ direction, is also used for the final operation of the TORES. The internal BbsI site cutting in the 5′ direction cerates the second cloning overhang for assembly of the J-donor vector. In the present example, a spaced NotI restriction site is encoded between the BbsI recognition sites to act as a negative selection marker to eliminate parental fragments from the J donor vector assembly process. The J receiving fragment is assembled by annealing two complimentary oligonucleotides designed to produce two 4-nucleodtide single-stranded overhangs, designated overhang*3 at the 5′ terminus, and overhang*4 at the 3′ terminus as described in Example 2.

To assemble the intermediate J receiving cassette vector with the above described J receiving cassette fragment, a J donor backbone must be created, as depicted in FIG. 9. In the present example, the cloning features of this vector backbone are EcoRI and XhoI restriction sites spaced to allow efficient restriction enzyme action. Digestion of this backbone with EcoRI and XhoI generate overhang*3′ and overhang*4′ complementary with overhang*3 and overhang*4 in the assembled J receiving cassette fragment. This permits directional cloning of the J receiving cassette fragment into the J donor backbone to create the J receiving cassette vector as described in Example 2. An example of the J donor vector backbone with a positive selection marker different from that of the V-C entry vector described above is presented as SEQ0097.

The J receiving cassette vector obtained above is depicted in FIG. 10, and essentially represents the J receiving cassette fragment in the context of the J donor vector backbone, and is presented as SEQ0697, wherein Y′ is the designation for the C part matching the C segment that is designated Y and described above, N represents any nucleotide, and n represents the number of nucleotides in said sequence.

It is into the J receiving cassette vector that an array of J segments are inserted to construct a J donor vector library. The design of the J gene segment is depicted in FIG. 11, wherein a J gene segment is bounded by overhang*5′ and overhang*6′ to the 5′ and 3′ ends, respectively. The J segment part is spaced from the 3′ overhang by a single adenine nucleotide in the present example, as to maintain enzyme recognition sequences and correct reading frame when inserting the J segment part into the J receiving cassette vector to create a J donor vector, as descried in Example 2. In the present example, a generic sequence for the J segment part is presented as SEQ0698 to SEQ0699 wherein the J gene segment part is denoted as ZNₙ, wherein Z is the designation for the J gene segment, N represents any nucleotide, and n represents the number of nucleotides in said sequence. The two sequences presented represent sense and antisense oligonucleotides annealed to assemble the J segment part, wherein the Z sequences are complementary.

The generic final obtained J donor vector in the present example is presented as SEQ0700, a representation of which is presented in FIG. 3. This J donor vector in the present example represents an inserted J segment part and the C part from the J receiving cassette vector. The sequence is presented with two annotated sequence inserts of ZNₙ and Y′Nₙ, wherein Z is the designation for the J gene segment, and Y′ is the designation of the C segment part, N represents any nucleotide, and n represents the number of nucleotides in said sequences.

Within all provided sequences, the selection of nucleotides considered the potential impact of DAM methylation of the BsaI binding sites. All additional recognition sequences for assembly and selection enzymes within the vectors were removed. Any such recognition sequences should also be removed from germline or synthetic TCR V, J and C elements inserted into the described fragment and vector contexts.

### Example 6

Design and Generation of Oligonucleotide Duplex Encoding CDR3 (odeCDR3)

In the above example, various formats of TORES are described in the design and generation of matched V-C entry vector and J donor vector libraries for various applications. The utilization of these V-C entry vector and J donor vector libraries for one-step reconstitution of full-length TCR open reading frames requires an oligonucleotide duplex encoding CDR3 (odeCDR3) construct to be provided in order to complete the target full-length TCR chain sequence (FIG. 4c). Once V-C entry vector and J donor vector libraries are generated, these vectors represent stock items that may be drawn upon indefinitely to select desired V-J-C combinations of target full-length TCR chains sequences. In contrast, the odeCDR3 represents a short unique sequence specific to the target full-length TCR ORF.

The present example describes the design and generation of odeCDR3 for use in the native human TRA and TRB vector platforms.

Design of the TRA odeCDR3

The annealing of two single stranded DNA oligonucleotides generates an odeCDR3 that contains 4-nucleotide single-strand overhangs at each end of the DNA fragment, as depicted in FIG. 4c. The 4-nucleotide overhangs are designed to permit directed ligase dependent cloning to the 3′ end of the TRA V segment encoded in the entry vector, (Overhang [symbol] 1-5′) and the 5′ end of the TRA J fragment during TRA reconstitution (Overhang [symbol] 2-3′). Overhang [symbol] 1-5′ is standardised to CTGC, complementary to the standardized Overhang [symbol] 1-5 encoded in the V segment of the TRA V-C entry vector. In the case of Overhang [symbol] 2-3′, there are two sequence forms that this can take, which is determined by sequence divergence among J segments from the human TRA locus. For native human TRA J segments TRAJ33 and TRAJ38, the Overhang [symbol] 2-3′ is standardized to TGGG, complementary to the Overhang [symbol] 2-3 encoded in the J donor vector of these two J segments. For all other human TRA J segments Overhang [symbol] 2-3′ is standardized to TTTG, complementary to the Overhang [symbol] 2-3 encoded in the J donor vector of these J segments (see Example 2).

Design of the TRB odeCDR3

As for the TRB odeCDR3, the annealing of two single stranded DNA oligonucleotides generates an odeCDR3 that contains 4-nucleotide single-strand overhangs at each end of the DNA fragment, as depicted in FIG. 4c. The 4-nucleotide overhangs are designed to permit directed ligase dependent cloning to the 3′ end of the TRB V segment encoded in the entry vector, Overhang [symbol] 1-5′, and the 5′ end of the TRB J fragment during TRB reconstitution, Overhang [symbol] 2-3′. The Overhang [symbol] 1-5′ is standardised to TTGC, complementary to the standardized Overhang [symbol] 1-5 encoded in the V segment of the TRB V-C entry vectors. In contrast to the TRA odeCDR3 where two alternative Overhang [symbol] 2-3 forms are required, for the TRB odeCDR3 Overhang [symbol] 2-3 is standardized to TTTG, complementary to the Overhang [symbol] 2-3 encoded in the J donor vector of all TRB J segments (see Example 3).

General odeCDR3 Design

In general, an odeCDR3 design must be matched to the overhangs the 4-nucleotide overhangs are designed to permit directed ligase dependent cloning to the 3′ end of the V segment encoded in the entry vector (Overhang [symbol] 1-5′) and the 5′ end of the J fragment during reconstitution, (Overhang [symbol] 2-3′).

### Method to Generate Phosphorylated CDR3 DNA Oligonucleotide Duplex

Phosphorylation and Annealing Two Oligonucleotides to Form the odeCDR3

### Reaction Mix

| | |
|---|---|
| Oligonucleotide (sense strand) (100 μM) | 1 μl |
| Oligonucleotide (anti-sense strand) (100 μM) | 1 μl |
| T4 ligase buffer 10x | 1 μl |
| T4 PNK | 1 μl |
| H₂O | 6 μl |

### Reaction Conditions

Incubate for 37° C. for 1 hour

Denature at 95° C. for 5 min

Anneal sense and anti-sense oligonucleotides by slowly cooling the reaction down to 25° C. at 3° C. per min

### Example 7

Reconstitution of TRA and TRB Full-Length ORFs from Respective V-C Entry and J Donor Vector Libraries
JG9a/b example.

This example describes the steps used for defining the vector library components and odeCDR3 required to reconstitute TRA and TRB full length TCR ORFs given sequence information of the target TCRs. The present example also demonstrates the assembly process of full-length a model TRA and TRB TCR chain pair, and confirms its specificity via transient expression in a human cell model, and staining of surface-presented TCR with specific HLA-multimer reagent.

Selection of V-C Entry Vector, J Donor Vector and odeCDR3

The sequences of all possible germline fragments that are represented in the cloning library are aligned to a TRA or TRB sequences of interest. The genetic fragments with the highest identity to the TRA or TRB sequence determines which V, J and C genetic element will constitute the desired TRA or TRB clonotype sequences. For TRA, the appropriate V-C entry vector is selected based on the determination of the V usage of the desired TRA. For TRB, when sequence coverage is sufficient to determine the V and C usage, the appropriate V-C entry vector will be selected that corresponds to the V usage of the desired TRB clonotype, in addition to whether said clonotype uses TRBC1 or TRBC2.

In the case when both the short and long version of the specific TRAJ or TRB J genetic element align to the TRA and TRB sequence, respectively, the corresponding plasmids encoding the longer genetic elements will be used for the TRA reconstruction.

The odeCDR3 sequence required for the TRA to be synthesised is determined as the region between the 3′ end of the TRA V aligned genetic fragment and the 5′ end of the aligned TRAJ genetic fragment. The oligonucleotide sense strand requires the additional 5′ 4-nucleotide overhang, Overhang [symbol] 1-5′, CTGC that is universal to the overhang generated on the TRA V entry vector when digested with BsaI, Overhang [symbol] 1-3′. The complementary oligonucleotide anti-senses strand requires the additional 5′ 4-nucleotide overhang, Overhang [symbol] 2-3′, that is unique to the overhang specifically for the TRAJ vector added to the TRA reconstruction reaction, Overhang [symbol] 2-5′.

The CDR3 sequence required for the TRB to be synthesised is determined as the region between the 3′ end of the TRB V aligned genetic fragment and the 5′ end of the aligned TRB J genetic fragment. The oligonucleotide sense strand requires the additional 5′ 4-nucleotide overhang, Overhang [symbol] 1-5′, TTGC that is universal to the overhang generated on the TRB V entry vector when digested with BsaI, Overhang [symbol] 1-3′. The complementary oligonucleotide anti-senses strand requires the additional 5′ 4-nucleotide overhang, Overhang [symbol] 2-3′, that is unique to the overhang specifically for the TRB J vector added to the TCR reconstruction reaction Overhang [symbol] 2-5′.

In the present example, a model TCR TRA/TRB pair is used with a known specificity for a HLA-A2*01-restricted antigen. The sequences of the TRA and TRB chains are presented as SEQ701 and SEQ702, respectively.

Based on this full-length sequence it was straightforward to select the appropriate V-C entry and J donor vectors from the TRA and TRB libraries. In the present example, V-C entry vector of the transient expression type were used, that is, with the backbone presented as SEQ0048.

In the present example the TRA V-C entry vector SEQ0088 (from list 0049 to 0094) and J donor vector SEQ0371 (from list 0323 to 0378) were selected.

In the present example the TRB V-C entry vector SEQ0563 (from list 0484 to 0577) and J donor vector SEQ0637 (from list 0636 to 0687) were selected.

The odeCDR3 synthesised for the TRA chain is presented in SEQ703 and SEQ704 as sense and antisense, respectively.

The odeCDR3 synthesised for the TRB chain is presented in SEQ705 and SEQ706 as sense and antisense, respectively.

### Method for Full-Length Reconstitution

For each of the TRA and TRB components selected above, restriction enzyme/ligase cycle reactions were performed as described below.

### RE Digestion and Ligation Reaction

| | | |
|---|---|---|
| V-C entry vector | 100 | ng |
| J donor vector | 60 | ng |
| odeCDR3 oligonucleotide duplex (0.5 μM) | 2 | μl |
| 10x T4 ligase buffer | 2 | μl |
| Bsal | 0.5 | μl |
| T4 DNA ligase | 0.5 | μl |
| H₂O | up to 20 | μl |

### Reaction Conditions

Step 1; 2 min at 37° C.

Step 2; 3 min at 16° C.

Repeat step 1 and 2, 20 times

5 min at 50° C.

5 min at 80° C.

Return to room temperature

Add 0.5 μl of NotI enzyme and incubate for 30 min at 37° C.

The resulting reaction product was transformed into competent E. coli cells and plated on carbenicillin containing plates.

Screening and sequencing carbenicillin resistant colonies was conducted to determine correctly assembled constructs. Screening of colonies was performed by restriction enzyme diagnostic digest of isolated plasmid DNA, and the expected DNA fragment sizes were observed by DNA electrophoresis. The resulting constructs encode the full length TCR alpha and beta clone sequences.

### Validation of Reconstituted TRA and TRB Vectors.

To verify the specificity of the reconstituted TCR TRA/TRB pair above, both constructs were transiently transfected into a cell line that expresses all CD3 components, but lacks TRA and TRB expression. This analysis is presented in FIG. 12, wherein the reconstituted TCR is transfected along side an irrelevant TCR, and an empty vector control. The empty vector shows no surface staining with antibodies for either TCRalpha/beta or CD3, nor do these control cells stain for HLA-A2*01-NLVPMVATV tetramer reagent (FIG. 12, rightmost panels). TCR expression is required for surface presentation of the CD3 complex. The irrelevant TCR stains positively for TCRalpha/beta and CD3, but shows no staining for the HLA-A2*01-NLVPMVATV tetramer reagent (FIG. 12, centre panels). This indicates that a TCR is expressed on the cell surface, but is not specific for the target antigen loaded in the HLA-A2*01 tetrameric reagent. The target TCR stains positively for TCRalpha/beta and CD3, and also shows positive staining for the HLA-A2*01-NLVPMVATV tetramer reagent (leftmost panels). This indicates that the reconstituted TCR both presents on the cell surface, and is also specific for the target antigen loaded in the HLA-A2*01 tetrameric reagent, as expected.

### Example 8

Reconstitution of a Set of Antigen-Specific TCR TRA/TRB Chain Pairs Identified from Human Peripheral Blood

One of the key requirements of upcoming strategies in personalised TCR-based diagnostics and therapeutics is the ability to rapidly capture and characterise antigen-specific TCR chain pairs from individuals. In the present example, a workflow is conducted wherein TCR TRA/TRB chain pairs are amplified and sequenced from single T-cells isolated from peripheral human blood, followed by reconstitution and validation of these pairs via transient expression in mammalian cells.

### Sequencing and Reconstitution of TRA and TRB Chain Pairs

FIG. 13 presents the overall workflow. Briefly, a fresh PBMC specimen was collected from a HLA-B*07:02 positive donor, and stained with an HLA-B*07:02-TPRV HLA-multimer reagent. This HLA-multimer is the HLA-B*07:02 allele loaded with a known immunodominant antigen from the HCMV pp65 gene, the full peptide sequence of which is; TPRVTGGGAM. Single HLA-B*07:02-TPRV multimer-positive cells were FACS sorted into 0.2 mL PCR tubes containing 5 μL of water. Samples were snap-frozen and stored at −80° C. until further processing (FIG. 13 step i).

Single-cell isolates were subjected to a two-step amplification process that entails a V-region specific primer collection for each TRA and TRB (FIG. 13 step ii), followed by paired nested PCR reactions that create TRA and TRB amplicons (FIG. 13 step iii) for sequence analysis (FIG. 13 step iv). This procedure is described previously (Han et. al. Nat Biotechnol. 2014 32(7): 684-692).

In the present example, 6 paired clones are presented as an example of downstream processing. The obtained sequences are presented as SEQ0707 to SEQ0718.

These sequences were then aligned against the a library of V, C and J gene segment for their corresponding TRA and TRB chains to determine the V, C and J segment usage of each amplified chain. This sequence analysis step also permits the definition of the CDR3 coding sequence, and thus the definition of odeCDR3 sequence (FIG. 13 step v). This sequence analysis permits the selection of V-C entry vectors (FIG. 13 step vi) and J donor vectors (FIG. 13 step vii) for TCR chain reconstitution. The analysis also permits the synthesis of odeCDR3 for each chain reconstitution reaction (FIG. 13 step viii). The selections and sequences are summarised in Table 1.

The table below presents as summary of sequence IDs for identified TCR TRA/TRB pairs in de novo sequencing and reconstitution example

| | | V | V-C Entry | J Seg- | J Donor | odCDR3 | odCDR3 |
|---|---|---|---|---|---|---|---|
| | | Segment | Vector | ment | Vector | Forward | Reverse |
| Chain | SEQ ID | Usage | SEQ | Usage | SEQ | SEQ | SEQ |
| TRA | SEQ0707 | TRAV26-1 | SEQ0082 | TRAJ49 | SEQ0370 | SEQ0719 | SEQ0720 |
| TRB | SEQ0708 | TRBV27 | SEQ0574 | TRBJ2-7 | SEQ0661 | SEQ0721 | SEQ0722 |
| TRA | SEQ0709 | TRAV14DV4 | SEQ0071 | TRAJ12 | SEQ0334 | SEQ0723 | SEQ0724 |
| TRB | SEQ0710 | TRBV7-9 | SEQ0554 | TRBJ1-1 | SEQ0636 | SEQ0725 | SEQ0726 |
| TRA | SEQ0711 | TRAV27 | SEQ0084 | TRAJ9 | SEQ0331 | SEQ0727 | SEQ0728 |
| TRB | SEQ0712 | TRBV27 | SEQ0574 | TRBJ1-2 | SEQ0637 | SEQ0729 | SEQ0730 |
| TRA | SEQ0713 | TRAV14DV4 | SEQ0071 | TRAJ9 | SEQ0331 | SEQ0731 | SEQ0732 |
| TRB | SEQ0714 | TRBV27 | SEQ0574 | TRBJ1-2 | SEQ0637 | SEQ0733 | SEQ0734 |
| TRA | SEQ0715 | TRAV3 | SEQ0052 | TRAJ12 | SEQ0334 | SEQ0735 | SEQ0736 |
| TRB | SEQ0716 | TRBV7-9 | SEQ0554 | TRBJ2-1 | SEQ0655 | SEQ0737 | SEQ0738 |
| TRA | SEQ0717 | TRAV17 | SEQ0073 | TRAJ12 | SEQ0334 | SEQ0739 | SEQ0740 |
| TRB | SEQ0718 | TRBV7-9 | SEQ0554 | TRBJ2-5 | SEQ0659 | SEQ0741 | SEQ0742 |

Selection of these components allows the assembly of independent restriction enzyme/ligase cycle reactions for each TRA and TRB chain to perform reconstitution of the full-length TCR ORFs (FIG. 13 step ix). Each reaction is performed as described in Example 7 above, which includes transformation and antibiotic selection and (FIG. 13 step x), then propagation and sequencing confirmation of the resulting reconstituted TCR ORFs (FIG. 13 step xi).

### Validation of TRA and TRB Chain Pairs

To verify the specificity of the set of reconstituted TCR TRA/TRB pairs above, paired constructs were transiently transfected into a cell line that expresses all CD3 components, but lacks TRA and TRB expression, as presented in example 7. This analysis is presented in FIG. 14. Cells were transfected with each of the 6 TCR ORF construct pairs, in parallel with an irrelevant TRA/TRB clone pair, and an empty vector control. Cells were analysed by flow cytometry after staining with an antibody against CD3 and the HLA-B*07:02-TPRV tetramer reagent initially used to isolate the single T-cells from the PBMC specimen. The paired constructs in the present example all displayed binding to the target HLA-B*07:02-TPRV tetramer reagent, and quantitative differences are observed. The number inset into each box represents the MFI ratio of CD3 negative over CD3 positive cell events.

### Example 9

Rapid TCR Chain Diversification Via odeCDR3 Degeneracy

The diversification and selection of TCR ORFs is desirable to engineer TCRs chain pairs with altered specificities, affinities and/or signalling capacity. The TORES system is suited to the rapid generation of collections of TCR chains that are systematically altered from the original target sequence. In the present example, an approach of diversifying a model TCR chain pair by including an odeCDR3 to a reconstitution reaction with a defined and limited nucleotide degeneracy at selected codon positions is presented. This approach was used to diversify the TRA chain of the model TCR TRA/TRB pair presented in Example 7. This single-reaction diversification is shown to produce a TCR set with a wide range of affinities to a specific HLA-multimer reagent when presented on the surface of mammalian cells with its natural TRB chain pair. This approach is ideally suited for rapid TCR-engineering using full-length TCR ORFs that may be presented and selected in a functional context of viable mammalian cells.

### Generation of Diverse TRA Chain Collection

FIG. 15 presents the overall strategy for generating a sequence-diversified collection of TCR chains in a single reaction by use of an odeCDR3 pool. A single C-V entry vector and J donor vector are selected to represent the target V,J and C gene segments in the final full-length TCR product (FIG. 15, box i and box ii). An odeCDR3 pool is generated with selected diversity, such that there are a number of different CDR3 sequences represented in the odeCDR3 pool (FIG. 15, Box iii). When all components are combined into a restriction enzyme/ligase cycle reaction, the resulting product are a collection of constructs containing full-length TCR chains of defined V,J C gene segment usage, and a defined diversity in the CDR3 region (FIG. 15, Box iv). The number of diversified full-length TCR chains in the final product is directly proportional to the number of odeCDR3 variants in the initial odeCDR3 pool added to the reaction.

In the present example, a model TCR TRA/TRB pair is used with a known specificity for a Human cytomegalovirus (HCMV) antigen presented in HLA-A2*01 (The same pair as Example 7). This antigenic peptide is derived from the HCMV pp65 protein, and the full amino acid sequence of the peptide antigen that is presented in HLA-A2*01 is NLVPMVATV. The sequences of the TRA and TRB chains are presented as SEQ701 and SEQ702, respectively.

In the present example, the TRA chain was the target of sequence diversification, and this was achieved through synthesis of odeCDR3 sense and antisense oligos with nucleotide degeneracy at 3 distinct positions, each altering a separate codon to result in the possibility of 4 different amino acids at each of the three codons. The codons were selected for degeneracy were spaced across the CDR3 loop. The odeCDR3 oligos are presented as SEQ0743 and SEQ0744, wherein degenerate codons are denoted N.

The odeCDR3 oligos were annealed by the method outlined in Example 6, with the 4-fold amino acid degeneracy at 3 separate codon positions resulting in an odeCDR3 product pool with 64 unique sequences, including the original coding sequence (i.e. SEQ0701).

The odeCDR3 was used to assemble the full-length TRA ORFs by the method outlined in Example 7 to create 64 unique TRA ORFs with 4-fold amino acid degeneracy at 3 distinct codon positions. In the present example, the odeCDR3 was synthesised with degenerated nucleotide usage at the indicated positions, and thus reconstitution was performed in a single tube to generate all 64 chain variants. The approach is equally valid whereby a unique odeCDR3 is provided to discrete reactions in parallel. All of the expected clones were prepared and sequence confirmed from a single reaction. Each TRA chain was prepared as a separate plasmid stock for subsequent characterisation.

Characterisation of Diversified TRA Chains with TRB Chain Pair

To characterise the specificity each of the 64 TCR TRA chains derived above were cotransfected with the TRB chain (SEQ0702) into a cell line that expresses all CD3 components, but lacks TRA and TRB expression. These cells were then stained with an antibody against CD3, and a HLA-A2*01-NLVPMVATV multimer reagent and analysed by flow cytometry. This analysis is presented in FIG. 16.

Each transfectant population is expressed as the ratio of the mean fluorescence intensity (MFI) of HLA-A2*01-NLVPMVATV multimer signal of CD3 positive over the CD3 negative populations, and plotted as a scatter plot ranked on ascending ratio (FIG. 16a). It is clear that the diversified TRA chains create a range of affinities to the HLA-A2*01-NLVPMVATV multimer. The original alpha chain is indicated with an arrow. The majority of diversified clones showed poorer binding than the original. A significant number of non-binders are also observed. Strikingly, a number of clones showed significant increase in HLA-A2*01-NLVPMVATV multimer staining (high-binders), including clones that improve the relative signal by over 3-fold. The clones are also presented as a table of ascending rank of MFI ratio, with the amino acids presented and diversified positions 1, 2 and 3 listed (FIG. 16b). It can be observed that a Pro at position 2 is highly stabilising of HLA-A2*01-NLVPMVATV multimer binding, and to a lesser degree an Asp at position 3. Conversely, a His at position 3 completely abolishes binding, except in the presence of a Pro at position 2. This positional bias of amino acid substitutions towards increased and decreased binding of the HLA-A2*01-NLVPMVATV multimer strongly suggests a bona fide alteration in binding affinity upon targeted amino acid degeneracy created in the TRA chain using the TORES.

Overall, this example demonstrates that minimal diversity in CDR3 loop introduced in a single step by odeCDR3 degeneracy within the TORES can create a collection of TCRs with diverse binding characteristics towards analyte HLA-antigen complex. This can be directly incorporated into TCR maturation workflows to generate synthetic TCRs with altered characteristics within various TCR engineering scenarios, while still maintaining the full-length native context of the TCR chains along with native V,J and C gene segment usage.

### Example 10

### Rapid TCR Chain Diversification Via V and J Segment Shuffling

The diversification and selection of TCR ORFs is desirable to engineer TCRs chain pairs with altered specificities, affinities and/or signalling capacity. The TORES system is suited to the rapid generation of collections of TCR chains that are systematically altered from the original target sequence. In the present example, a TCR chain diversification approach is outlined, wherein a single odeCDR3 is provided to a full-length TCR chain reaction with multiple V-C entry vectors, J donor vectors or multiples of both V-C entry vectors and J donor vectors. Such an approach can be used to diversify a given TCR chain using native germline V, J and C segments, while maintaining the possibility of carrying through core CDR3 contacts with cognate HLA-antigen complex in de novo TCR chains. This approach is ideally suited for rapid TCR-engineering using full-length TCR ORFs that may be presented and selected in a functional context of viable mammalian cells.

An example of shuffling V gene segment usage is presented in FIG. 17. This schematic example outlines an approach of maintaining a single odeCDR3 and J donor vector usage, with sequence matching the parental TCR chain, while providing a selection of V gene segments via provision of a number of V-C entry vectors to the reconstitution reaction. In the case of TCR chains that have the opportunity of multiple C gene segment usage (e.g. human TRB), this can also be factored into the diversification workflow. The product of a reaction containing single odeCRD3 and J donor vector, and a selection of V-C entry vectors, will be a number of full-length TCR chains containing parental CDR3/J sequence, and each of the provided V (and/or C) gene segments. Such an approach is equally valid to achieve in discrete reactions with a number of selected V-C entry vectors, rather than the pooled reaction depicted in FIG. 17.

An example of shuffling J gene segment usage is presented in FIG. 18. This schematic example outlines an approach of maintaining a single odeCDR3 and V-C entry vector usage, with sequence matching the parental TCR chain, while providing a selection of J gene segments via provision of a selection of J donor vectors to the reconstitution reaction. The product of a reaction containing single odeCRD3 and V-C entry vector, and a selection of J donor vectors, will be a number of full-length TCR chains containing parental CDR3/V-C sequence, and each of the provided J donor gene segments. Such an approach is equally valid to achieve in discrete reactions with a number of selected J donor vectors, rather than the pooled reaction depicted in FIG. 18.

A final example of shuffling both V and J gene segment usage is presented in FIG. 19. This schematic example outlines an approach of maintaining a single odeCDR3, with sequence matching the parental TCR chain, while providing a selection of both V(-C) and J gene segments via provision of a selection of both V-C and J donor vectors to the reconstitution reaction. The product of a reaction containing single odeCRD3, and a selection of both V-C entry and J donor vectors, will be a number of full-length TCR chains containing parental CDR3 sequence, and each combination of V(-C) and J donor gene segments provided to the reaction. Such an approach is equally valid to achieve in discrete reactions with a number of selected V-C entry and J donor vectors, rather than the pooled reaction depicted in FIG. 19.

### Example 11

Reconstitution of Full Length TCR ORFs from TRA and TRB Using TORES²

This example describes a TORES² for the human TRA/TRB loci with RMCE sites as 5′ and 3′ genetic elements, and demonstrates the reconstitution of a model human TCR pair, and subsequent integration of the reconstituted ORFs into a engineered cell line harbouring RMCE sites matched with the 5′ and 3′ genetic elements of the originating V-Cα entry vector backbone context. The model TCR TRA/TRB pair has known specificity for a HLA-A*02:01-restricted antigen. The model TRA and TRB sequences are represented by SEQ0778 and SEQ0779, respectively. The antigen peptide has the amino acid sequence SLLMWITQV.

V-C Entry Vectors, J Donor Vector, odeCDR3 and Bidirectional Terminator for TORES²

The V-Cα, V-Cβ, J donor vector and the oligonucleotide duplex encoding the odeCDR3 are selected using the same selection criteria as outlined in example 7. As described above, the TORES² requires adaptation of the V-Cα and V-Cβ to incorporate distinct Type IIS sites and negative selection elements. The V-Cα and V-Cβ entry vector backbone sequences are represented by SEQ7056 and SEQ0764, respectively. Due to the addition of new restriction sites to these backbone sequences, some of the V-segment sequences require modification to underlying nucleotide sequences compared to those presented for the TORES system above, as to eliminate these new restriction sites from the TCR coding sequence. Modified TRA V-C sequences are represented by SEQ0757 to SEQ0763, and modified TRB V-C sequences by SEQ0765 to SEQ0776. All other TRA and TRB sequences used were as described for the TORES system above.

### Bidirectional Terminator Donor Component

The Bidirectional Terminator Donor vector (BiT donor), provides the bidirectional terminator element introduced in the second step reaction, and which ultimately adjoins the antiparallel TRA and TRB chains reconstituted in the first step reaction. In the present example, the bidirectional terminator element is represented by SEQ0777, and is provided in a suitable vector backbone context.

### Reconstitution Reactions

The first restriction enzyme/ligase cycle reaction reaction to reconstitute the target TRA and TRB ORFs is conducted as is described in Example 7. This results in reconstituted TRA and TRB, each within their respective V-C entry backbone contexts. In the second reaction, these two product vectors are combined with the BiT donor vector in a new enzyme ligase cycle reaction to generate the final product vector, encoding the reconstituted TRA and TRB ORFs in an antiparallel sense, and adjoined by the introduced bidirectional terminator element.

### RE Digestion and Ligation Reaction for TORES²

| | | |
|---|---|---|
| Reconstituted TRA ORF vector | 25 | ng |
| Reconstituted TRB ORF vector | 25 | ng |
| BiT donor vector | 25 | ng |
| 10x T4 ligase buffer | 2 | μl |
| Esp3I | 0.5 | μl |
| T4 DNA ligase | 0.5 | μl |
| H₂O | up to 20 | μl |

### Reaction Conditions

Step 1; 2 min at 37° C.

Step 2; 3 min at 16° C.

Repeat step 1 and 2, 20 times

5 min at 50° C.

5 min at 80° C.

Return to room temperature

Add 0.5 μl of SalI and MluI enzymes and incubate for 30 min at 37° C.

The resulting reaction product was transformed into competent E. coli cells and plated on carbenicillin containing plates.

Screening and sequencing carbenicillin resistant colonies was conducted to determine correctly assembled constructs. Screening of colonies was performed by restriction enzyme diagnostic digest of isolated plasmid DNA, and the expected DNA fragment sizes were observed by DNA electrophoresis. The resulting constructs encode the full length TCR alpha and beta clone sequences.

### Validation of the Reconstituted Full Length TCR ORF

To verify the functionality of the reconstituted bi-directional TRA/TRB donor vector generated by the TORES² system, the construct was delivered into a cell line that expresses all CD3 components but lacks TRA and TRB expression, and further encodes genomic receiver sites compatible with the RMCE sites contained within the product donor vector and which are contributed by the original V-Cα entry vector, and suitable antiparallel promoter elements at the 5′ and 3′ ends. The analysis is presented in FIG. 23, wherein the reconstituted donor vector is transfected into cells. Cells that have lost the genomic receiver site markers (FIG. 23 a, RFP−BFP−) are the cells that express the bidirectional TCR ORF, resulting in CD3 surface expression (FIG. 23 b, light grey histogram). While the cells that did not lose the genomic receiver site markers are cells that failed to integrate the bidirectional TCR construct (FIG. 23 a) RFP+BFP+) and do not stain for CD3 surface expression (FIG. 23 b, dark grey histogram). TCR expression is required for surface presentation of the CD3 complex. All the RFP−BFP− cells that have lost the genomic receiver markers are positive for CD3 surface expression (FIG. 23 b, light grey histogram) All the RFP+BFP+ cells that have not lost the genomic receiver markers are negative for CD3 surface expression (FIG. 23 b) dark grey histogram). These CD3+ cells expressing the reconstituted TCR also show positive staining for the HLA-A*02:01-SLLMWITQV tetramer reagent (FIG. 23 c) compared to the cells that have not been stained with the said tetramer (FIG. 23 d). Together the results indicate that the reconstituted TCR both presents on the cell surface and is specific for the target antigen loaded in the HLA-A*02:01-SLLMWITQV tetramer reagent.

### SPECIFIC EMBODIMENTS

In the following is given more details regarding the invention and various aspects thereof.
1. A combined system comprising two separate components, wherein the first component is a vector carrying variable and constant (V-C) T-cell receptor (TCR) gene segments, and the second component is a vector carrying joining (J) TCR gene segments.
2. A combined system according to item 1 wherein the first component is a V-C entry vector containing
   a. origin of replication,
   b. a first positive selection marker
   c. 5′ genetic element, or elements,
   d. Kozak Sequence,
   e. TCR variable gene segment,
   f. a first Type IIS sequence, for site specific recognition and cleavage by a Type IIS restriction enzyme,
   g. a negative selection marker,
   h. a second Type IIS sequence
   i. TCR constant gene segment, and
   j. 3′ genetic element, or elements.
3. A combined system according to item 1 or 2 wherein the second component is a J donor vector containing
   a. origin of replication,
   b. a second positive selection marker,
   c. a third Type IIS sequence,
   d. TCR Joining gene segment,
   e. A C part, corresponding to a small 5′ portion of a constant gene segment, and
   f. a fourth Type IIS sequence.
4. A combined system according to item 2 or 3, wherein said first, second, third and fourth Type 115 sequences are the same or different.
5. A combined system according to item 2 or 3, wherein said first, second, third and fourth Type 115 sequences are the same.
6. A combined system according to any of items 2-5, wherein the first and second positive selection markers are different and are selected from an antibiotic resistance gene or auxotroph complementing gene.
7. A combined system according to any of items 2-6, wherein the 5′ genetic element comprises one or more elements selected from
   a. gene cis/acting element,
   b. heterospecific recognition site for recombinase enzymes,
   c. a 5′ homologous recombination arm for a genomic site of interest′
   d. a mRNA splice acceptor site,
   e. an internal ribosomal entry site, and
   f. epigenetic insulator sequence.
8. A combined system according to any of items 2-7, wherein the TCR variable gene segment does not contain a type IIS sequence contained in the first and second component.
9. A combined system according to any of items 2-8, wherein the first Type IIS sequence is orientated to cleave 5′ of said recognition sequence and within the TCR variable gene segment.
10. A combined system according any of items 2-9, wherein the negative selection marker is selected from
   a. a restriction enzyme recognition site not contained elsewhere in the first component or within the TCR joining gene segment,
   b. a bacterial suicide gene, and
   c. a reporter element.
11. A combined system according items 2-10 wherein the second Type IIS sequence is orientated to cleave 3′ of said recognition sequence and within the TCR constant gene segment.
12. A combined system according to any of items 2-11, wherein the TCR constant gene segment does not contain any of the type IIS sequences contained in the first and second component.
13. A combined system according to any of items 2-12, wherein the 3′ genetic element comprises one or more elements selected from
   a. a terminator element,
   b. heterospecific recognition site for recombinase enzymes,
   c. a 3′ homologous recombination arm for a genomic site of interest,
   d. a mRNA splice donor site,
   e. an internal ribosomal entry site, and
   f. epigenetic insulator sequence.
14. A combined system according to any of items 3-13, wherein the third Type IIS sequence is orientated to cleave 3′ of said recognition sequence and within the TCR joining gene segment.
15. A combined system according to any of items 3-14, wherein the TCR joining gene segments do not contain any of the Type IIS sequences contained in the first and second component.
16. A combined system according to any of items 3-15, wherein the fourth Type IIS sequence is orientated to cleave 5′ of said recognition sequence and within the TCR C part portion or the construct.
17. A combined system according to any of items 3-16, wherein the TCR C part does not contain any of the type IIS sequences contained in the first and second component.
18. A combined system according to any of the preceding items, wherein all contained sequences are devoid of any negative selection element apart from the negative selection marker as defined in item 10.
19. A combined system according to any of the preceding items further comprising a third component comprising an oligonucleotide duplex encoding CDR3 (odeCDR3).
20. A combined system according to item 19, wherein the odeCDR3 has
   a. a first single strand overhang sequence complimentary to first Type IIS restriction enzyme recognition and cleavage site orientated to cleave 5′ of the recognition sequence and within the TCR variable gene segment,
   b. a double strand segment encoding a TCR CDR3 region and devoid of negative selection element, which negative selection element is as defined in item 10, and also devoid of any Type IIS restriction sequences of the first or second part, and
   c. a second single strand overhang sequence complimentary to the third Type IIS restriction enzyme recognition and cleavage site orientated to cleave 3′ of the recognition sequence and within the TCR joining gene segment.
21. A library of V-C entry vectors as defined in any of items 1-18, wherein the library is a collection of one or more vectors representing all germline TCR variable and constant gene segments of an organism having such TCRs.
22. A library of J donor vectors as defined in any of items 1-18, wherein the library is a collection of one or more vectors representing germline TCR joining gene segments of an organism having such TCRs.
23. A library of V-C entry vectors as defined in any of items 1-18, wherein the library is a collection of one or more vectors representing a collection of variant germline TCR variable and constant gene segments of an organism having such TCRs, such that translated amino acid sequence of the encoded protein is unmodified in relation to the protein sequence encoded by the germline gene segment.
24. A library of J donor vectors as defined in any of items 118, wherein the library is a collection of one or more vectors representing variant germline TCR joining gene segments of an organism having such TCRs, such that translated amino acid sequence of the encoded protein is unmodified in relation to the protein sequence encoded by the germline gene segment.
25. A library of V-C entry vectors as defined in any of items 1-18, wherein the library is a collection of one or more vectors representing a collection of variant germline TCR variable and constant gene segments of an organism having such TCRs, such that translated amino acid sequence of the encoded protein is modified in relation to the protein sequence encoded by the germline gene segment.
26. A library of J donor vectors as defined in any of items 1, 3-18, wherein the library is a collection of one or more vectors representing variant germline TCR joining gene segments of an organism having such TCRs, such that translated amino acid sequence of the encoded protein is modified in relation to the protein sequence encoded by the germline gene segment.
27. A library comprising a combination of V-C entry vectors and J donor vectors as defined in any of items 1-26.
28. A kit comprising a combination of
   a. one or more V-C entry vectors encoding combinations of Variable and Constant gene segments, and
   b. one or more J donor vectors encoding J gene segments, and optionally
   c. one or more standardised odeCDR3 with single stranded overhangs matched to V-C entry vector and J donor vector single strand overhangs as positive control odeCDR3, and optionally
   d. A pre-assembled full-length TCR ORF as a reference
29. A method for in vitro reconstitution of a full length TCR open reading frame (ORF), said method comprising
   a. selecting a V-C entry vector,
   b. selecting a J donor vector,
   c. selecting an odeCDR3,
   d. combining a, b and c to react with i) Type IIS restriction enzyme(s) to cleave all Type IIS restriction enzyme recognition and cleavage sites present in the V-C entry vector and in the J donor vector and ii) DNA ligase enzyme and subjecting the combined mix to a thermocycling reaction,
   e. transforming the reaction product obtained from step d. to a selectable host organism competent for DNA vector propagation, and
   f. performing a selection of host organism to obtain full length reconstituted TCR open reading frame in the V-C entry vector backbone.
30. A method according to item 29, wherein the V-C entry vector is as defined in item 2, the J donor vector is as defined in item 3, and step f of item 29 is performed by using the first positive selection marker as defined in item 6 on the transformed host cells.
31. A method according to items 29 or 30, wherein a selected TCR open reading frame is reconstituted from a selected TCR sequence, said method comprising
   a. Obtaining a TCR open reading frame sequence wherein said sequence information is sufficient to identify i) variable gene segment usage ii) constant gene segment usage iii) joining gene segment usage iv) a full CDR3 sequence spanning the variable gene segment border to the joining gene segment border, and
   b. selecting a V-C entry vector corresponding to the variable and constant gene segments identified in step a. i) and a. ii), respectively, and
   c. selecting a J donor vector corresponding to the joining gene segment identified in step a. iii), and
   d. generating an odeCDR3 corresponding to CDR3 sequence identified in step a. iv, and
   e. combining b, c and d to react with i) Type IIS restriction enzyme(s) to cleave all Type IIS restriction enzyme recognition and cleavage sites present in the V-C entry vector and in the J donor vector and ii) DNA ligase enzyme and subjecting the combined mix to a thermocycling reaction, and
   f. transforming the reaction product obtained from step d. to a selectable host organism competent for vector replication, and
   g. performing a selection of host organism to obtain full length reconstituted TCR open reading frame in the V-C entry vector backbone.
32. A method according to any of items 29-31, wherein step d. i) is performed using a single Type IIS restriction enzyme.
33. A method according to any of items 29-31, wherein step d. i) is performed using at least two Type IIS restriction enzymes.
34. A method according to any of items 29-33 further comprising at least one step of negative selection comprising
   a. performing restriction enzyme digest of reaction product to eliminate parental V-C entry vector prior to host transformation prior to step e. of item 29 or step f. of item 31, and/or
   b. performing a suicide gene selection to eliminate competent hosts transformed with parental V-C entry vector, and/or
   c. performing selection of host cells transformed with parental V-C entry vector by way of reporter identification.
35. A method according to any of items 29-34, wherein selection of host organisms comprises selecting a host organism having resistance to antibiotic present in the culture system or competition of auxotrophic factors not present in the culture system, and said antibiotic resistance or auxotrophic complementation being conferred by a gene product encoded in the V-C entry vector.
36. A method according to any of items 29-34, wherein one or more steps are performed in a culture system suitable for culture and selection of the host organism.
37. A method according to any of items 29 to 36 used to create a pool of two or more TCR ORFs with diversified CDR3 region, wherein two or more odeCDR3 forms are selected according to item 29 c, and combined to a single restriction enzyme/ligase cycle reaction according to item 29 d.
38. A method according to any of items 29 to 36 used to create a pool of two or more TCR ORFs with diversified V and/or C gene segment usage, wherein two or more V-C entry vectors are selected according to item 29 a, and combined to a single restriction enzyme/ligase cycle reaction according to item 29 d.
39. A method according to any of items 29 to 36 used to create a pool of two or more TCR ORFs with diversified J gene segment usage, wherein two or more J donor vectors are selected according to item 29 b, and combined to a single restriction enzyme/ligase cycle reaction according to item 29 d.
40. A method according to any of items 29 to 36 used to create a pool of two or more TCR ORFs with diversified V and/or C and J gene segment usage, wherein two or more V-C entry and J donor vectors are selected according to item 29 a and b, and combined to a single restriction enzyme/ligase cycle reaction according to item 29 d.
41. A method according to any of items 29 to 36 used to create a pool of two or more TCR ORFs with diversified CDR3 in addition to diversified V and/or C and J gene segment usage, wherein two or more odeCDR3 are selected according to item 29 c, and two or more V-C entry and J donor vectors are selected according to item 29 a and b, and combined to a single restriction enzyme/ligase cycle reaction according to item 29 d.
42. A method to construct a V-C entry vector according to items 1 and 2, wherein the method comprises combining three DNA components selected from
   a. a Variable gene segment cloning fragment
   b. a Constant gene segment cloning fragment
   c. a V-C entry vector backbone
43. A method according to item 42 wherein said Variable gene segment cloning fragment comprises
   a. a 5′ primer bind sequence for polymerase chain reaction dependent propagation of the fragment
   b. a fifth Type IIS sequence orientated to cut in the 3′ direction
   c. a first overhang sequence that encodes a defined single stranded overhang upon Type IIs enzyme action on the fifth Type IIs sequence in b
   d. a Kozak sequence
   e. a TCR variable gene segment
   f. a first Type IIS sequence
   g. a 5′ sequence segment of a negative selection marker
   h. a sixth Type IIS sequence orientated to cut in the 5′ direction such that a single stranded overhang is generated within the 5′ sequence segment of the negative selection marker in g
   i. a 3′ primer bind sequence for polymerase chain reaction dependent propagation of the fragment
44. A method according to item 42 or 43, wherein said Constant gene segment cloning fragment comprises
   a. a 5′ primer bind sequence for polymerase chain reaction dependent propagation of the fragment
   b. a seventh Type IIS sequence orientated to cut in the 3′ direction such that a single stranded overhang is generated within the 3′ sequence segment of the negative selection marker in c
   c. a 3′ sequence segment of a negative selection marker
   d. a second type IIS sequence
   e. a TCR constant gene segment
   f. a second overhang sequence that encodes a defined single stranded overhang upon Type IIs enzyme action on the eighth Type IIs sequence in g
   g. an eighth Type IIS sequence orientated to cut in the 5′ direction such that a single stranded overhang is generated in overhang sequence of f
   h. a 3′ primer bind sequence for polymerase chain reaction dependent propagation of the fragment
45. A method according to any of items 42-44 wherein said V-C entry vector backbone comprises
   a. an origin or replication
   b. a first positive selection marker
   c. a 5′ genetic element
   d. a first restriction enzyme recognition sequence permitting digestion of the backbone to create a single stranded overhang complimentary to said first overhang mentioned in item 43 c
   e. a second restriction enzyme recognition sequence permitting digestion of the backbone to create a single stranded overhang complimentary to said second overhang mentioned in item 44 f
   f. a 3′ genetic element.
46. A method according to any of items 42 to 45, wherein the fifth, sixth, seventh and eighth Type IIS sequences are the same or different.
47. A method according to any of items 42 to 45, wherein the fifth, sixth, seventh and eighth Type IIS sequences are the same.
48. A method according to any of items 42 to 45, wherein the fifth, sixth, seventh and eighth Type IIS sequences are different from the first, second third and fourth Type IIS sequences as defined in items 2 to 5.
49. A method according to any of items 42 to 48 comprising
   a. Digestion of the V-C entry vector backbone with said first restriction enzyme and said second restriction enzyme to create said first and second overhang, respectively
   b. Combining the digested V-C entry vector backbone with the V cloning fragment and the C cloning fragment, along with DNA ligase enzyme and one or more Type IIS restriction enzyme(s) recognising the fifth, sixth, seventh and eighth Type IIS sequences
   c. Transformation of the resulting reaction product into competent host organism and positive selection using said first positive selection marker to obtain complete V-C entry vector
50. A Variable gene segment cloning fragment represented by the sequence SEQ0690.
51. A Constant gene segment cloning fragment represented by the sequence SEQ0691.
52. A V-C entry vector represented by sequence SEQ0048, to construct V-C entry vectors suitable for transient expression or reconstituted full-length TCR open reading frames in a mammalian cells.
53. A V-C entry vector represented by sequence SEQ0688, to construct V-C entry vectors suitable for recombinase mediated cassette exchange with matched genetic targets with suitable heterospecific recombinase sequences.
54. A V-C entry vector backbone represented by sequence SEQ0689, to construct V-C entry vectors suitable for recombinase mediated cassette exchange with genetic targets with matched heterospecific recombinase sequences.

V-C Entry Vector Generics
55. A V-C entry vector represented by sequence SEQ0692, suitable for transient expression or reconstituted full-length TCR open reading frames in a mammalian host cell.
56. A V-C entry represented by sequence SEQ0693 suitable for recombinase mediated cassette exchange with genetic targets with matched heterospecific recombinase sequences.
57. A V-C entry vector represented by sequence SEQ0694, suitable for recombinase mediated cassette exchange with genetic targets with matched heterospecific recombinase sequences.
58. A method to construct J donor vector according to items 1 or 3, wherein the method comprises combining four DNA components selected from
   a. J receiving cassette fragment
   b. J donor vector backbone
   c. J receiving cassette vector
   d. J segment part
59. A method according to item 58 wherein said J receiving cassette fragment comprises
   a. a first single stranded overhang at the 5′ end commentary to overhang sequence mentioned in 60 c
   b. a third Type IIS sequence orientated to cut in the 3′ direction, joined with a sequence that forms a single stranded overhang when acted upon by the enzyme directed by a ninth Type IIS sequence mentioned in c
   c. a ninth Type IIs sequence orientated to cut in the 5′ direction, and to create a single stranded overhang mentioned in b
   d. a negative selection marker
   e. a tenth Type IIS sequence orientated to cut in the 3′ direction and create a single stranded overhang at the 5′ of the sequence described in f
   f. a C-part representing a 5′ portion of the Constant gene fragment, with an overhang sequence at the 5′ end generated by enzyme action directed by the tenth Type IIS sequence, and an overhang sequence at the 3′ end generated by enzyme action directed by the fourth Type IIS sequence mentioned in g
   g. a fourth Type IIS sequence orientated to cut in the 5′ direction such that a single stranded overhang is generated within the 5′ sequence containing the C-part mentioned in f
   h. a second single stranded overhang at the 3′ end commentary to overhang sequence mentioned in 60 d
60. A method according to item 58 or 59 wherein said J donor vector backbone comprises
   a. an origin or replication
   b. a second positive selection marker
   c. a first restriction enzyme recognition sequence permitting digestion of the backbone to create a single stranded overhang complimentary to said first overhang mentioned in item 59 a
   d. a second restriction enzyme recognition sequence permitting digestion of the backbone to create a single stranded overhang complimentary to said second overhang mentioned in item 59 h
61. A method according to any of items 58 to 60 wherein said J receiving cassette vector in constructed by combining the TRAJ receiving cassette fragment described in item 53 and the TRAJ J donor vector backbone described in item 54, wherein the method comprises
   a. Digestion of the J donor vector backbone with said first restriction enzyme and said second restriction enzyme to create said first and second overhang, respectively
   b. Combining the digested J donor vector backbone with the J receiving cassette fragment
   c. Transformation of the resulting reaction product into competent host organism and positive selection using said the second positive selection marker to obtain complete J receiving cassette vector
62. A method according to any of items 58 to 61 wherein said J segment part comprises
   a. A first single stranded overhang sequence at the 5′ end that is complementary to the overhang generated in the J receiving cassette fragment mentioned in item 59 b, when acted on by the enzyme directed by the ninth Type IIS sequence mentioned in item 59 b
   b. A Joining gene segment part
   c. A second single stranded overhang sequence at the 3′ end that is complementary to the overhang generated in the J receiving cassette fragment mentioned in item 59 f, when acted on by the enzyme directed by the ninth Type IIS sequence mentioned in item 59 e
63. A method according to any of items 58 to 62, wherein the ninth and tenth Type 115 sequences are the same or different.
64. A method according to any of items 58 to 62, wherein the ninth and tenth Type IIS sequences are the same.
65. A method according to any of items 42 to 62, wherein the ninth and tenth Type IIS sequences are different from the first, second third and fourth Type IIS sequences as defined in items 2 to 5.
66. A method according to any of items 42 to 62, wherein the ninth and tenth Type IIS sequences are the same or different from the fifth, sixth, seventh and eighth Type IIS sequences as defined in items 2 to 5.
67. A method according to any of items 42 to 62, wherein the ninth and tenth Type IIS sequences are the same as the fifth, sixth, seventh and eighth Type IIS sequences as defined in items 2 to 5.
68. A method according to any of items 42 to 62 wherein said J donor vector is constructed be combining a J donor vector backbone with a J segment part, wherein the method comprises
   a. Combining J receiving cassette vector with the J segment part, along with DNA ligase enzyme and one or more Type IIS restriction enzyme(s) recognising the ninth and tenth Type IIS sequences and subjecting the combined mix to a thermocycling reaction
   b. Transformation of the resulting reaction product into competent host organism and positive selection using said second positive selection marker to obtain complete J donor vector
69. A J receiving cassette fragment represented by the sequences SEQ0695 and SEQ0696.
70. A J donor vector backbone represented by sequence SEQ0097.
71. A J receiving cassette vector represented by SEQ0697, constructed by the combination of the J receiving cassette fragment mentioned in item 69 and the J donor vector backbone mentioned in item 70.
72. A J segment part represented by sequences SEQ0698 and SEQ0699, to construct a J donor vector by insertion of the J segment part into a J receiving cassette vector mentioned in item 71.
73.
A J donor vector represented by the sequence SEQ0700, assembled from J receiving cassette vector mentioned in item 71 and the J segment part mentioned in item 72.
74. A library of V-C entry vectors as defined in any items 2-19, containing Variable and Constant gene segments to recapitulate the gene segment usage of the human TRA locus, represented by sequences SEQ0049 to SEQ0094, wherein the vectors are suited for transient expression of a reconstituted full-length TCR open reading frame.
75. A library of J donor vectors as defined in any of items 3-19, containing Joining gene segments to recapitulate the gene segment usage of the human TRA locus, represented by sequences SEQ0323 to SEQ0378, wherein the vectors are used in conjunction with an odeCDR3 spanning the entire CDR3 region.
76. A library of J donor vectors as defined in any of items 3-, containing Joining gene segments to recapitulate the gene segment usage of the human TRA locus, represented by sequences SEQ0379 to SEQ0434, wherein the vectors are used in conjunction with an odeCDR3 reduced in length by three to four codons.
77. A library of V-C entry vectors as defined in any of items 2-19 containing Variable and Constant gene segments to recapitulate the gene segment usage of the human TRB locus, represented by sequences SEQ0484 to SEQ0577, wherein the vectors are suited for transient expression of a reconstituted full-length TCR open reading frame.
78. A library of J donor vectors as defined in any of items 3-19 containing Joining gene segments to recapitulate the gene segment usage of the human TRB locus, represented by sequences SEQ0636 to SEQ0661, wherein the vectors are used in conjunction with an odeCDR3 spanning the entire CDR3 region.
79. A library of J donor vectors as defined in any of items 3-19 containing Joining gene segments to recapitulate the gene segment usage of the human TRB locus, represented by sequences SEQ0662 to SEQ0687, wherein the vectors are used in conjunction with an odeCDR3 reduced in length by three to four codons.
80. A combined system according to items 1 to 20, for use in providing native germline and/or diversified synthetic TCR open reading frames in defined vector contexts.
81. TCR open reading frames in defined vector contexts for use in diagnostic or therapeutic procedures.

1.-31. (canceled)

32. A vector system comprising:
(I) a variable-constant (VC) entry vector comprising a VC oligonucleotide sequence comprising a T cell receptor (TCR) variable gene segment and a TCR constant gene segment; and
(II) a joining donor (J donor) vector comprising a J donor oligonucleotide sequence comprising a TCR joining gene segment,
wherein one or more of:
(A) the VC oligonucleotide sequence further comprises:
a. an origin of replication,
b. a first positive selection marker
c. a 5′ genetic element,
d. a Kozak Sequence,
e. a first Type IIS sequence,
f. a negative selection marker,
g. a second Type IIS sequence, and
h. a 3′ genetic element,
wherein the TCR variable gene segment is flanked by the Kozak Sequence on one end and the first Type IIS sequence on the other end, and wherein the TCR constant gene segment is downstream of the second Type IIS sequence and upstream of the 3′ genetic element; and
(B) the J donor vector oligonucleotide sequence further comprises:
a. an origin of replication,
b. a second positive selection marker,
c. a third Type IIS sequence,
d. a 5′ portion of a constant gene segment, and
e. a fourth Type IIS sequence,
wherein the TCR joining gene segment is flanked by the third Type IIS sequence on one end and the 5′ portion of the constant gene segment on the other end.

33. The vector system of claim 32, wherein the 5′ genetic element comprises one or more elements selected from:
a. a gene cis/acting element,
b. a heterospecific recognition site for a recombinase enzyme,
c. a 5′ homologous recombination arm for a genomic site of interest,
d. an mRNA splice acceptor site,
e. an internal ribosomal entry site, and
f. an epigenetic insulator sequence.

34. The vector system of claim 32, wherein the negative selection marker is one or more selected from:
a. a restriction enzyme recognition site not contained elsewhere in the first component and not within the TCR joining gene segment,
b. a bacterial suicide gene, and
c. a reporter element.

35. The vector system of claim 32, wherein the 3′ genetic element comprises one or more elements selected from:
a. a terminator element,
b. a heterospecific recognition site for a recombinase enzyme,
c. a 3′ homologous recombination arm for a genomic site of interest,
d. an mRNA splice donor site,
e. an internal ribosomal entry site, and
f. an epigenetic insulator sequence.

36. The vector system of claim 32, further comprising (III) an oligonucleotide duplex encoding CDR3 (odeCDR3), wherein the odeCDR3 comprises an oligonucleotide sequence encoding a TCR CDR3.

37. The vector system of claim 36, wherein the oligonucleotide sequence encoding the TCR CDR3 is flanked by two Type IIS sequences.

38. The vector system of claim 32, wherein the V-C entry vector comprises an oligonucleotide sequence having the sequence of any one of SEQ ID NOs: 692-694.

39. The vector system of claim 32, wherein the J donor vector comprises an oligonucleotide having the sequence of SEQ ID NO: 700.

40. The vector system of claim 32, wherein the TCR variable gene segment is a TCR alpha (TCRα) variable gene segment.

41. The vector system of claim 40, wherein the VC oligonucleotide sequence further comprises:
i. a fifth Type IIS sequence,
j. a second negative selection marker, and
k. a sixth Type IIS sequence.

42. The vector system of claim 40, wherein the VC entry vector comprises an oligonucleotide sequence having the sequence of SEQ ID NO: 756.

43. The vector system of claim 40, further comprising a VC-β entry vector, wherein the VC-β entry vector comprises:
a. an origin of replication,
b. a third positive selection marker,
c. a 5′ genetic element,
d. a seventh Type IIS sequence,
e. a Kozak Sequence,
f. a TCR beta variable gene segment,
g. a eighth Type IIS sequence,
h. a third negative selection marker,
i. a ninth Type IIS sequence,
j. a TCR constant gene segment,
k. a tenth Type IIS sequence,
l. a fourth negative selection marker, and
m. a 3′ genetic element.

44. The vector system of claim 43, wherein the VC-β entry vector comprises an oligonucleotide sequence having the sequence of SEQ ID NO: 764.

45. The vector system of claim 43, further comprising a bidirectional terminator donor (BiT donor) vector, wherein the BiT donor vector comprises an oligonucleotide sequence comprising:
a. an origin of replication,
b. a fourth positive selection marker,
c. an eleventh Type IIS sequence,
d. a bidirectional terminator, and
e. a twelfth Type IIS sequence.

46. The vector system of claim 45, wherein the BiT donor vector comprises an oligonucleotide sequence having the sequence of SEQ ID NO: 777.

47. A vector system library comprising two or more vector systems of claim 32.

48. A vector system library comprising two or more vector systems of claim 43.

49. A vector library comprising one or more of:
a. two or more variable constant alpha (VC-α) entry donor vectors comprising two or more oligonucleotide sequences having sequences selected from SEQ ID NOs: 49-94;
b. two or more VC-β entry vectors comprising two or more oligonucleotide sequences having sequences selected from SEQ ID NOs: 484-577; and
c. two or more joining donor (J donor) vectors comprising two or more oligonucleotide sequences having sequences selected from SEQ ID NOs: 323-434 and 636-687.

50. A method for preparing a TCR open reading frame (ORF), comprising:
a. combining the vector system of claim 37 with (i) one or more Type IIS restriction enzymes, to cleave the Type IIS sequences in the VC entry vector, J donor vector, and odeCDR3; and (ii) a DNA ligase enzyme, to produce a mixture; and
b. subjecting the mixture to a thermocycling reaction to produce a reaction product;
c. transforming the reaction product into a host organism; and
d. propagating the host organism to produce a TCR ORF comprising the TCR variable gene segment, TCR constant gene segment, and TCR joining gene segment.

51. A method for preparing a TCR open reading frame (ORF) from the vector system of claim 45, comprising:
a. combining the VC entry vector, J donor vector, and odeCDR3 of the vector system of claim 45 with (i) one or more Type IIS restriction enzymes, to cleave the Type IIS sequences in the VC entry vector, J donor vector, and odeCDR3; and (ii) a DNA ligase enzyme, to produce a first mixture;
b. combining the VC-β entry vector, J donor vector and odeCDR3 of the vector system of claim 45 with (i) one or more Type IIS restriction enzymes, to cleave the Type IIS restriction sequences in the VC-β entry vector, J donor vector, and odeCDR3; and (ii) a DNA ligase enzyme, to produce a second mixture;
c. subjecting the first and second mixtures to a thermocycling reaction to produce a first and second reaction product;
d. combining the first and second reaction products with (i) the BiT donor vector of the vector system of claim 45, (ii) one or more Type IIS restriction enzymes, to cleave the Type IIS restriction sequences in the first and second reaction products and the BiT donor vector, and (iii) a DNA ligase enzyme, to produce a third mixture;
e. subjecting the third mixture to a thermocycling reaction to produce a third reaction product;
f. transforming the third reaction product into a host organism; and
g. propagating the host organism to produce a TCR ORF comprising the TCR variable gene segment, TCR constant gene segment, and the TCR joining gene segment.


SEQUENCE LISTING (779 sequences)

SEQ ID NO: 1 - DNA, 396 bp, Homo sapiens
  TRAV1-1_BB_1
gtcagatactccatgagcacgaagacttgtacgccaccatgtggggagctttccttctctatgtttccatgaagatgggaggcactgcaggacaaagccttgagcagccctctgaagtgacagctgtggaaggagccattgtccagataaactgcacgtaccagacatctgggttttatgggctgtcctggtaccagcaacatgatggcggagcacccacatttctttcttacaatgctctggatggtttggaggagacaggtcgtttttcttcattccttagtcgctctgatagttatggttacctccttctacaggagctccagatgaaagactctgcctcttacttctgcagagaccttgcggccgtgtcttcgactagtagctcacctacga

SEQ ID NO: 2 - DNA, 396 bp, Homo sapiens
  TRAV1-2_BB_1
gtcagatactccatgagcacgaagacttgtacgccaccatgtggggagttttccttctttatgtttccatgaagatgggaggcactacaggacaaaacattgaccagcccactgagatgacagctacggaaggtgccattgtccagatcaactgcacgtaccagacatctgggttcaacgggctgttctggtaccagcaacatgctggcgaagcacccacatttctgtcttacaatgttctggatggtttggaggagaaaggtcgtttttcttcattccttagtcggtctaaagggtacagttacctccttttgaaggagctccagatgaaagactctgcctcttacctctgcagagaccttgcggccgtgtcttcgactagtagctcacctacga

SEQ ID NO: 3 - DNA, 408 bp, Homo sapiens
  TRAV2_BB_1
gtcagatactccatgagcacgaagacttgtacgccaccatggctttgcagagcactctgggggcggtgtggctagggcttctcctcaactctctctggaaggttgcagaaagcaaggaccaagtgtttcagccttccacagtggcatcttcagagggagctgtggtggaaatcttctgtaatcactctgtgtccaatgcttacaacttcttctggtaccttcacttcccgggatgtgcaccaagactccttgttaaaggctcaaagccttctcagcagggacgatacaacatgacctatgaacggttctcttcatcgctgctcatcctccaggtgcgggaggcagatgctgctgtttactactgcagagaccttgcggccgtgtcttcgactagtagctcacctacga

SEQ ID NO: 4 - DNA, 411 bp, Homo sapiens
  TRAV3_BB_1
gtcagatactccatgagcacgaagacttgtacgccaccatggcctctgcacccatctcgatgcttgcgatgctcttcacattgagtgggctgagagctcagtcagtggctcagccggaagatcaggtcaacgttgctgaagggaatcctctgactgtgaaatgcacctattcagtctctggaaacccttatcttttttggtatgttcaataccccaaccgaggcctccagttccttctgaaatacatcacaggggataacctggttaaaggcagctatggctttgaagctgaatttaacaagagccaaacctccttccacctgaagaaaccatctgcccttgtgagcgactccgctttgtacttctgcagagaccttgcggccgtgtcttcgactagtagctcacctacga

SEQ ID NO: 5 - DNA, 396 bp, Homo sapiens
  TRAV4_BB_1
gtcagatactccatgagcacgaagacttgtacgccaccatgaggcaagtggcgagagtgatcgtgttcctgaccctgagtactttgagccttgctaagaccacccagcccatctccatggactcatatgaaggacaagaagtgaacataacctgtagccacaacaacattgctacaaatgattatatcacgtggtaccaacagtttcccagccaaggaccacgatttattattcaaggatacaagacaaaagttacaaacgaagtggcctccctgtttatccctgccgacagaaagtccagcactctgagcctgccccgggtttccctgagcgacactgctgtgtactactgcagagaccttgcggccgtgtcttcgactagtagctcacctacga

SEQ ID NO: 6 - DNA, 411 bp, Homo sapiens
  TRAV5_BB_1
gtcagatactccatgagcacgaagacttgtacgccaccatgaaaacatttgctggattttcgttcctgtttttgtggctgcagctggactgtatgagtagaggagaggatgtggagcagagtcttttcctgagtgtccgagagggagacagctccgttataaactgcacttacacagacagctcctccacctacttatactggtataagcaagaacctggagcaggtctacagttgctgacgtatattttttcaaatatggacatgaaacaagaccaaagactcactgttctattgaataaaaaggataaacatctgtctctgcgcattgcagacacccagactggggactcagctatctacttctgcagagaccttgcggccgtgtcttcgactagtagctcacctacga

SEQ ID NO: 7 - DNA, 411 bp, Homo sapiens
  TRAV6_BB_1
gtcagatactccatgagcacgaagacttgtacgccaccatggagtcattcctgggaggtgttttgctgattttgtggcttcaagtggactgggtgaagagccaaaagatagaacagaattccgaggccctgaacattcaggagggtaaaacggccaccctgacctgcaactatacaaactattccccagcatacttacagtggtaccgacaagatccaggaagaggccctgttttcttgctactcatacgtgaaaatgagaaagaaaaaaggaaagaaagactgaaggtcacctttgataccacccttaaacagagtttgtttcatatcacagcctcccagcctgcagactcagctacctacctctgcagagaccttgcggccgtgtcttcgactagtagctcacctacga

SEQ ID NO: 8 - DNA, 408 bp, Homo sapiens
  TRAV7_BB_1
gtcagatactccatgagcacgaagacttgtacgccaccatggagaagatgcgtagacctgtcctaattatattttgtctatgtcttggctgggcaaatggagaaaaccaggtggagcacagccctcattttctgggaccccagcagggagacgttgcctccatgagctgcacgtactctgtcagtcgttttaacaatttgcagtggtacaggcaaaatacagggatgggtcccaaacacctattatccatgtattcagctggatatgagaagcagaaaggaaggctaaatgctacattactgaagaatggaagcagcttgtacattacagccgtgcagcctgaagattcagccacctatttctgcagagaccttgcggccgtgtcttcgactagtagctcacctacga

SEQ ID NO: 9 - DNA, 411 bp, Homo sapiens
  TRAV8-1_BB_1
gtcagatactccatgagcacgaagacttgtacgccaccatgctcctgttgctcataccagtgctggggatgatttttgccctgagagatgccagagcccagtctgtgagccagcataaccaccacgtaattctctctgaagcagcctcactggagttgggatgcaactattcctatggtggaactgttaatctcttctggtatgtccagtaccctggtcaacaccttcagcttctcctcaagtacttttcaggggatccactggttaaaggcatcaagggctttgaggctgaatttataaagagtaaattctcctttaatctgaggaaaccctctgtgcagtggagtgacacagctgagtacttctgcagagaccttgcggccgtgtcttcgactagtagctcacctacga

SEQ ID NO: 10 - DNA, 411 bp, Homo sapiens
  TRAV8-2_BB_1
gtcagatactccatgagcacgaagacttgtacgccaccatgctcctgctgctcgtcccagtgctcgaggtgatttttactctgggaggaaccagagcccagtcggtgacccagcttgacagccacgtctctgtctctgaaggaaccccggtgctgctgaggtgcaactactcatcttcttattcaccatctctcttctggtatgtgcaacaccccaacaaaggactccagcttctcctgaagtacacatcagcggccaccctggttaaaggcatcaacggttttgaggctgaatttaagaagagtgaaacctccttccacctgacgaaaccctcagcccatatgagcgacgcggctgagtacttctgcagagaccttgcggccgtgtcttcgactagtagctcacctacga

SEQ ID NO: 11 - DNA, 411 bp, Homo sapiens
  TRAV8-3_BB_1
gtcagatactccatgagcacgaagacttgtacgccaccatgctcctggagcttatcccactgctggggatacattttgtcctgagaactgccagagcccagtcagtgacccagcctgacatccacatcactgtctctgaaggagcctcactggagttgagatgtaactattcctatggggcaacaccttatctcttctggtatgtccagtcccccggccaaggcctccagctgctcctgaagtacttttcaggagacactctggttcaaggcattaaaggctttgaggctgaatttaagaggagtcaatcttccttcaatctgaggaaaccctctgtgcattggagtgatgctgctgagtacttctgcagagaccttgcggccgtgtcttcgactagtagctcacctacga

SEQ ID NO: 12 - DNA, 411 bp, Homo sapiens
  TRAV8-4_BB_1
gtcagatactccatgagcacgaagacttgtacgccaccatgctcctgctgctcgtcccagtgctcgaggtgatttttaccctgggaggaaccagagcccagtcggtgacccagcttggcagccacgtctctgtctctgaaggagccctggttctgctgaggtgcaactactcatcgtctgttccaccatatctcttctggtatgtgcaataccccaaccaaggactccagcttctcctgaagtacacatcagcggccaccctggttaaaggcatcaacggttttgaggctgaatttaagaagagtgaaacctccttccacctgacgaaaccctcagcccatatgagcgacgcggctgagtacttctgcagagaccttgcggccgtgtcttcgactagtagctcacctacga

SEQ ID NO: 13 - DNA, 411 bp, Homo sapiens
  TRAV8-6_BB_1
gtcagatactccatgagcacgaagacttgtacgccaccatgctcctgctgctcgtcccagcgttccaggtgatttttaccctgggaggaaccagagcccagtctgtgacccagcttgacagccaagtccctgtctttgaagaagcccctgtggagctgaggtgcaactactcatcgtctgtttcagtgtatctcttctggtatgtgcaataccccaaccaaggactccagcttctcctgaagtatttatcaggatccaccctggttaaaggcatcaacggttttgaggctgaatttaacaagagtcaaacttccttccacttgaggaaaccctcagtccatataagcgacacggctgagtacttctgcagagaccttgcggccgtgtcttcgactagtagctcacctacga

SEQ ID NO: 14 - DNA, 408 bp, Homo sapiens
  TRAV8-7_BB_1
gtcagatactccatgagcacgaagacttgtacgccaccatgctcttagtggtcattctgctgcttggaatgttcttcacactgagaaccagaacccagtcggtgacccagcttgatggccacatcactgtctctgaagaagcccctctggaactgaagtgcaactattcctatagtggagttccttctctcttctggtatgtccaatactctagccaaagcctccagcttctcctcaaagacctaacagaggccacccaggttaaaggcatcagaggttttgaggctgaatttaagaagagcgaaacctccttctacctgaggaaaccatcaacccatgtgagtgatgctgctgagtacttctgcagagaccttgcggccgtgtcttcgactagtagctcacctacga

SEQ ID NO: 15 - DNA, 408 bp, Homo sapiens
  TRAV9-1_BB_1
gtcagatactccatgagcacgaagacttgtacgccaccatgaattcttctccaggaccagcgattgcactattcttaatgtttgggggaatcaatggagattcagtggtccagacagaaggccaagtgctcccctctgaaggggattccctgattgtgaactgctcctatgaaaccacacagtacccttcccttttttggtatgtccaatatcctggagaaggtccacagctccacctgaaagccatgaaggccaatgacaagggaaggaacaaaggttttgaagccatgtaccgtaaagaaaccacttctttccacttggagaaagactcagttcaagagtcagactccgctgtgtacttctgcagagaccttgcggccgtgtcttcgactagtagctcacctacga

SEQ ID NO: 16 - DNA, 408 bp, Homo sapiens
  TRAV9-2_BB_1
gtcagatactccatgagcacgaagacttgtacgccaccatgaactattctccaggcttagtatctctgatactcttactgcttggaagaacccgtggaaattcagtgacccagatggaagggccagtgactctctcagaagaggccttcctgactataaactgcacgtacacagccacaggatacccttcccttttctggtatgtccaatatcctggagaaggtctacagctcctcctgaaagccacgaaggctgatgacaagggaagcaacaaaggttttgaagccacataccgtaaagaaaccacttctttccacttggagaaaggctcagttcaagtgtcagactcagcggtgtacttctgcagagaccttgcggccgtgtcttcgactagtagctcacctacga

SEQ ID NO: 17 - DNA, 414 bp, Homo sapiens
  TRAV10_BB_1
gtcagatactccatgagcacgaagacttgtacgccaccatgaaaaagcatctgacgaccttcttggtgattttgtggctttatttttatagggggaatggcaaaaaccaagtggagcagagtcctcagtccctgatcatcctggagggaaagaactgcactcttcaatgcaattatacagtgagccccttcagcaacttaaggtggtataagcaagatactgggagaggtcctgtttccctgacaatcatgactttcagtgagaacacaaagtcgaacggaagatatacagcaactctggatgcagacacaaagcaaagctctctgcacatcacagcctcccagctcagcgattcagcctcctacatctgcagagaccttgcggccgtgtcttcgactagtagctcacctacga

SEQ ID NO: 18 - DNA, 408 bp, Homo sapiens
  TRAV12-1_BB_1
gtcagatactccatgagcacgaagacttgtacgccaccatgatatccttgagagttttactggtgatcctgtggcttcagttaagctgggtttggagccaacggaaggaggtggagcaggatcctggacccttcaatgttccagagggagccactgtcgctttcaactgtacttacagcaacagtgcttctcagtctttcttctggtacagacaggattgcaggaaagaacctaagttgctgatgtccgtatactccagtggtaatgaagatggaaggtttacagcacagctcaatagagccagccagtatatttccctgctcatcagagactccaagctcagtgattcagccacctacctctgcagagaccttgcggccgtgtcttcgactagtagctcacctacga

SEQ ID NO: 19 - DNA, 414 bp, Homo sapiens
  TRAV12-2_BB_1
gtcagatactccatgagcacgaagacttgtacgccaccatgatgaaatccttgagagttttactagtgatcctgtggcttcagttgagctgggtttggagccaacagaaggaggtggagcagaattctggacccctcagtgttccagagggagccattgcctctctcaactgcacttacagtgaccgaggttcccagtccttcttctggtacagacaatattctgggaaaagccctgagttgataatgttcatatactccaatggtgacaaagaagatggaaggtttacagcacagctcaataaagccagccagtatgtttctctgctcatcagagactcccagcccagtgattcagccacctacctctgcagagaccttgcggccgtgtcttcgactagtagctcacctacga

SEQ ID NO: 20 - DNA, 414 bp, Homo sapiens
  TRAV12-3_BB_1
gtcagatactccatgagcacgaagacttgtacgccaccatgatgaaatccttgagagttttactggtgatcctgtggcttcagttaagctgggtttggagccaacagaaggaggtggagcaggatcctggaccactcagtgttccagagggagccattgtttctctcaactgcacttacagcaacagtgcttttcaatacttcatgtggtacagacagtattccagaaaaggccctgagttgctgatgtacacatactccagtggtaacaaagaagatggaaggtttacagcacaggtcgataaatccagcaagtatatctccttgttcatcagagactcacagcccagtgattcagccacctacctctgcagagaccttgcggccgtgtcttcgactagtagctcacctacga

SEQ ID NO: 21 - DNA, 408 bp, Homo sapiens
  TRAV13-1_BB_1
gtcagatactccatgagcacgaagacttgtacgccaccatgacatccattcgagctgtatttatattcctgtggctgcagctggacttggtgaatggagagaatgtggagcagcatccttcaaccctgagtgtccaggagggagacagcgctgttatcaagtgtacttattcagacagtgcctcaaactacttcccttggtataagcaagaacttggaaaaggacctcagcttattatagacattcgttcaaatgtgggcgaaaagaaagaccaacgaattgctgttacattgaacaagacagccaaacatttctccctgcacatcacagaaacccaacctgaggactcggctgtctacttctgcagagaccttgcggccgtgtcttcgactagtagctcacctacga

SEQ ID NO: 22 - DNA, 411 bp, Homo sapiens
  TRAV13-2_BB_1
gtcagatactccatgagcacgaagacttgtacgccaccatggcaggcattcgagctttatttatgtacttgtggctgcagctggactgggtgagcagaggagagagtgtggggctgcatcttcctaccctgagtgtccaggagggtgacaactctattatcaactgtgcttattcaaacagcgcctcagactacttcatttggtacaagcaagaatctggaaaaggtcctcaattcattatagacattcgttcaaatatggacaaaaggcaaggccaaagagtcaccgttttattgaataagacagtgaaacatctctctctgcaaattgcagctactcaacctggagactcagctgtctacttctgcagagaccttgcggccgtgtcttcgactagtagctcacctacga

SEQ ID NO: 23 - DNA, 417 bp, Homo sapiens
  TRAV14DV4_BB_1
gtcagatactccatgagcacgaagacttgtacgccaccatgtcactttctagcctgctgaaggtggtcacagcttcactgtggctaggacctggcattgcccagaagataactcaaacccaaccaggaatgttcgtgcaggaaaaggaggctgtgactctggactgcacatatgacaccagtgatcaaagttatggtctattctggtacaagcagcccagcagtggggaaatgatttttcttatttatcaggggtcttatgacgagcaaaatgcaacagaaggtcgctactcattgaatttccagaaggcaagaaaatccgccaaccttgtcatctccgcttcacaactgggggactcagcaatgtatttctgcagagaccttgcggccgtgtcttcgactagtagctcacctacga

SEQ ID NO: 24 - DNA, 399 bp, Homo sapiens
  TRAV16_BB_1
gtcagatactccatgagcacgaagacttgtacgccaccatgaagcccaccctcatctcagtgcttgtgataatatttatactcagaggaacaagagcccagagagtgactcagcccgagaagctcctctctgtctttaaaggggccccagtggagctgaagtgcaactattcctattctgggagtcctgaactcttctggtatgtccagtactccagacaacgcctccagttactcttgagacacatctctagagagagcatcaaaggcttcactgctgaccttaacaaaggcgagacatctttccacctgaagaaaccatttgctcaagaggaagattcagccatgtattactgcagagaccttgcggccgtgtcttcgactagtagctcacctacga

SEQ ID NO: 25 - DNA, 408 bp, Homo sapiens
  TRAV17_BB_1
gtcagatactccatgagcacgaagacttgtacgccaccatggaaactctcctgggagtgtctttggtgattctatggcttcaactggctagggtgaacagtcaacagggagaagaggatcctcaggccttgagcatccaggagggtgaaaatgccaccatgaactgcagttacaaaactagtataaacaatttacagtggtatagacaaaattcaggtagaggccttgtccacctaattttaatacgttcaaatgaaagagagaaacacagtggaagattaagagtcacgcttgacacttccaagaaaagcagttccttgttgatcacggcttcccgggcagcagacactgcttcttacttctgcagagaccttgcggccgtgtcttcgactagtagctcacctacga

SEQ ID NO: 26 - DNA, 405 bp, Homo sapiens
  TRAV18_BB_1
gtcagatactccatgagcacgaagacttgtacgccaccatgctgtctgcttcctgctcaggacttgtgatcttgttgatattcagaaggaccagtggagactcggttacccagacagaaggcccagttaccctccctgagagggcagctctgacattaaactgcacttatcagtccagctattcaacttttctattctggtatgtccagtatctaaacaaagagcctgagctcctcctgaaaagttcagaaaaccaggagacggacagcagaggttttcaggccagtcctatcaagagtgacagttccttccacctggagaagccctcggtgcagctgtcggactctgccgtgtactactgcagagaccttgcggccgtgtcttcgactagtagctcacctacga

SEQ ID NO: 27 - DNA, 417 bp, Homo sapiens
  TRAV19_BB_1
gtcagatactccatgagcacgaagacttgtacgccaccatgctgactgccagcctgttgagggcagtcatagcctccatctgtgttgtatccagcatggctcagaaggtaactcaagcgcagactgaaatttctgtggtggagaaggaggatgtgaccttggactgtgtgtatgaaacccgtgatactacttattacttattctggtacaagcaaccaccaagtggagaattggttttccttattcgtcggaactcttttgatgagcaaaatgaaataagtggtcggtattcttggaacttccagaaatccaccagttccttcaacttcaccatcacagcctcacaagtcgtggactcagcagtatacttctgcagagaccttgcggccgtgtcttcgactagtagctcacctacga

SEQ ID NO: 28 - DNA, 408 bp, Homo sapiens
  TRAV20_BB_1
gtcagatactccatgagcacgaagacttgtacgccaccatggagaaaatgttggagtgtgcattcatagtcttgtggcttcagcttggctggttgagtggagaggaccaggtgacgcagagtcccgaggccctgagactccaggagggagagagtagcagtcttaactgcagttacacagtcagcggtttaagagggctgttctggtataggcaagatcctgggaaaggccctgaattcctcttcaccctgtattcagctggggaagaaaaggagaaagaaaggctaaaagccacattaacaaagaaggaaagctttctgcacatcacagcccctaaacctgaggactcagccacttatctctgcagagaccttgcggccgtgtcttcgactagtagctcacctacga

SEQ ID NO: 29 - DNA, 408 bp, Homo sapiens
  TRAV21_BB_1
gtcagatactccatgagcacgaagacttgtacgccaccatggagaccctcttgggcctgcttatcctttggctgcagctgcaatgggtgagcagcaaacaggaggtgacgcagattcctgcagctctgagtgtcccagaaggagaaaacttggttctcaactgcagtttcactgatagcgctatttacaacctccagtggtttaggcaggaccctgggaaaggactcacatctctgttgcttattcagtcaagtcagagagagcaaacaagtggacgccttaatgcctcgctggataaatcatcaggacgtagtactttatacattgcagcttctcagcctggtgactcagccacctacctctgcagagaccttgcggccgtgtcttcgactagtagctcacctacga

SEQ ID NO: 30 - DNA, 402 bp, Homo sapiens
  TRAV22_BB_1
gtcagatactccatgagcacgaagacttgtacgccaccatgaagaggatattgggagctctgctggggctcttgagtgcccaggtttgctgtgtgagaggaatacaagtggagcagagtcctccagacctgattctccaggagggagccaattccacgctgcggtgcaatttttctgactctgtgaacaatttgcagtggtttcatcaaaacccttggggacagctcatcaacctgttttacattccctcagggacaaaacagaatggaagattaagcgccacgactgtcgctacggaacgctacagcttattgtacatttcctcttcccagaccacagactcaggcgtttatttctgcagagaccttgcggccgtgtcttcgactagtagctcacctacga

SEQ ID NO: 31 - DNA, 435 bp, Homo sapiens
  TRAV23DV6_BB_1
gtcagatactccatgagcacgaagacttgtacgccaccatggacaagatcttaggagcatcatttttagttctgtggcttcaactatgctgggtgagtggccaacagaaggagaaaagtgaccagcagcaggtgaaacaaagtcctcaatctttgatagtccagaaaggagggatttcaattataaactgtgcttatgagaacactgcgtttgactactttccatggtaccaacaattccctgggaaaggccctgcattattgatagccatacgtccagatgtgagtgaaaagaaagaaggaagattcacaatctccttcaataaaagtgccaagcagttctcattgcatatcatggattcccagcctggagactcagccacctacttctgcagagaccttgcggccgtgtcttcgactagtagctcacctacga

SEQ ID NO: 32 - DNA, 417 bp, Homo sapiens
  TRAV24_BB_1
gtcagatactccatgagcacgaagacttgtacgccaccatggagaagaatcctttggcagccccattactaatcctctggtttcatcttgactgcgtgagcagcatactgaacgtggaacaaagtcctcagtcactgcatgttcaggagggagacagcaccaatttcacctgcagcttcccttccagcaatttttatgccttacactggtacagatgggaaactgcaaaaagccccgaggccttgtttgtaatgactttaaatggggatgaaaagaagaaaggacgaataagtgccactcttaataccaaggagggttacagctatttgtacatcaaaggatcccagcctgaagattcagccacatacctctgcagagaccttgcggccgtgtcttcgactagtagctcacctacga

SEQ ID NO: 33 - DNA, 402 bp, Homo sapiens
  TRAV25_BB_1
gtcagatactccatgagcacgaagacttgtacgccaccatgctactcatcacatcaatgttggtcttatggatgcaattgtcacaggtgaatggacaacaggtaatgcaaattcctcagtaccagcatgtacaagaaggagaggacttcaccacgtactgcaattcctcaactactttaagcaatatacagtggtataagcaaaggcctggtggacatcccgtattcttgatacagttagtgaagagtggagaagtgaagaagcagaaaagactgacatttcagtttggagaagcaaaaaagaacagctccctgcacatcacagccacccagactacagatgtaggaacctacttctgcagagaccttgcggccgtgtcttcgactagtagctcacctacga

SEQ ID NO: 34 - DNA, 396 bp, Homo sapiens
  TRAV26-1_BB_1
gtcagatactccatgagcacgaagacttgtacgccaccatgaggctggtggcaagagtaactgtgtttctgacctttggaactataattgatgctaagaccacccagcccccctccatggattgcgctgaaggaagagctgcaaacctgccttgtaatcactctaccatcagtggaaatgagtatgtgtattggtatcgacagattcactcccaggggccacagtatatcattcatggtctaaaaaacaatgaaaccaatgaaatggcctctctgatcatcacagaagatagaaagtccagcaccttgatcctgccccacgctacgctgagagacactgctgtgtactactgcagagaccttgcggccgtgtcttcgactagtagctcacctacga

SEQ ID NO: 35 - DNA, 396 bp, Homo sapiens
  TRAV26-2_BB_1
gtcagatactccatgagcacgaagacttgtacgccaccatgaagttggtgacaagcattactgtactcctatctttgggtattatgggtgatgctaagaccacacagccaaattcaatggagagtaacgaagaagagcctgttcacttgccttgtaaccactccacaatcagtggaactgattacatacattggtatcgacagcttccctcccagggtccagagtacgtgattcatggtcttacaagcaatgtgaacaacagaatggcctctctggcaatcgctgaggacagaaagtccagtaccttgatcctgcaccgtgctaccttgagagatgctgctgtgtactactgcagagaccttgcggccgtgtcttcgactagtagctcacctacga

SEQ ID NO: 36 - DNA, 402 bp, Homo sapiens
  TRAV27_BB_1
gtcagatactccatgagcacgaagacttgtacgccaccatggtcctgaaattctccgtgtccattctttggattcagttggcatgggtgagcacccagctgctggagcagagccctcagtttctaagcatccaagagggagaaaatctcactgtgtactgcaactcctcaagtgttttttccagcttacaatggtacagacaggagcctggggaaggtcctgtcctcctggtgacagtagttacgggtggagaagtgaagaagctgaagagactaacctttcagtttggtgatgcaagaaaggacagttctctccacatcactgcagcccagcctggtgatacaggcctctacctctgcagagaccttgcggccgtgtcttcgactagtagctcacctacga

SEQ ID NO: 37 - DNA, 429 bp, Homo sapiens
  TRAV29DV5_BB_1
gtcagatactccatgagcacgaagacttgtacgccaccatggccatgctcctgggggcatcagtgctgattctgtggcttcagccagactgggtaaacagtcaacagaagaatgatgaccagcaagttaagcaaaattcaccatccctgagcgtccaggaaggaagaatttctattctgaactgtgactatactaacagcatgtttgattatttcctatggtacaaaaaataccctgctgaaggtcctacattcctgatatctataagttccattaaggataaaaatgaagatggaagattcactgttttcttaaacaaaagtgccaagcacctctctctgcacattgtgccctcccagcctggagactctgcagtgtacttctgcagagaccttgcggccgtgtcttcgactagtagctcacctacga

SEQ ID NO: 38 - DNA, 408 bp, Homo sapiens
  TRAV30_BB_1
gtcagatactccatgagcacgaagacttgtacgccaccatggagactctcctgaaagtgctttcaggcaccttgttgtggcagttgacctgggtgagaagccaacaaccagtgcagagtcctcaagccgtgatcctccgagaaggggaagatgctgtcatcaactgcagttcctccaaggctttatattctgtacactggtacaggcagaagcatggtgaagcacccgttttcctgatgatattactgaagggtggagaacagaagggtcatgaaaaaatatctgcttcatttaatgaaaaaaagcagcaaagctccctgtaccttacggcctcccagctcagttactcaggaacctacttctgcagagaccttgcggccgtgtcttcgactagtagctcacctacga

SEQ ID NO: 39 - DNA, 408 bp, Homo sapiens
  TRAV34_BB_1
gtcagatactccatgagcacgaagacttgtacgccaccatggagactgttctgcaagtactcctagggatattggggttccaagcagcctgggtcagtagccaagaactggagcagagtcctcagtccttgatcgtccaagagggaaagaatctcaccataaactgcacgtcatcaaagacgttatatggcttatactggtataagcaaaagtatggtgaaggtcttatcttcttgatgatgctacagaaaggtggggaagagaaaagtcatgaaaagataactgccaagttggatgagaaaaagcagcaaagttccctgcatatcacagcctcccagcccagccatgcaggcatctacctctgcagagaccttgcggccgtgtcttcgactagtagctcacctacga

SEQ ID NO: 40 - DNA, 402 bp, Homo sapiens
  TRAV35_BB_1
gtcagatactccatgagcacgaagacttgtacgccaccatgctccttgaacatttattaataatcttgtggatgcagctgacatgggtcagtggtcaacagctgaatcagagtcctcaatctatgtttatccaggaaggagaagatgtctccatgaactgcacttcttcaagcatatttaacacctggctatggtacaagcaggaacctggggaaggtcctgtcctcttgatagccttatataaggctggtgaattgacctcaaatggaaggctgactgctcagtttggtataaccagaaaggacagcttcctgaatatctcagcatccatacctagtgatgtaggcatctacttctgcagagaccttgcggccgtgtcttcgactagtagctcacctacga

SEQ ID NO: 41 - DNA, 411 bp, Homo sapiens
  TRAV36DV7_BB_1
gtcagatactccatgagcacgaagacttgtacgccaccatgatgaagtgtccacaggctttactagctatcttttggcttctactgagctgggtgagcagtgaagataaggtggtacaaagccctctatctctggttgtccacgagggagacaccgtaactctcaattgcagttatgaagtgactaactttcgaagcctactatggtacaagcaggaaaagaaagctcccacatttctatttatgctaacttcaagtggaattgaaaagaagtcaggtagactaagtagcatattagataagaaagaactttccagcatcctgaacatcacagccacccagaccggagactcggccatctacctctgcagagaccttgcggccgtgtcttcgactagtagctcacctacga

SEQ ID NO: 42 - DNA, 417 bp, Homo sapiens
  TRAV38-1_BB_1
gtcagatactccatgagcacgaagacttgtacgccaccatgacacgagttagcttgctgtgggcagtcgtggtcagtacctgtcttgaatccggcatggcccagacagtcactcagtctcaaccagagatgtctgtgcaggaggcagagactgtgaccctgagttgcacatatgacaccagtgagaataattattatttgttctggtacaagcagcctcccagcaggcagatgattctcgttattcgccaagaagcttataagcaacagaatgcaacggagaatcgtttctctgtgaacttccagaaagcagccaaatccttcagtctcaagatctcagactcacagctgggggacactgcgatgtatttctgcagagaccttgcggccgtgtcttcgactagtagctcacctacga

SEQ ID NO: 43 - DNA, 417 bp, Homo sapiens
  TRAV38-2DV8_BB_1
gtcagatactccatgagcacgaagacttgtacgccaccatggcatgccctggcttcctgtgggcacttgtgatctccacctgtcttgaatttagcatggctcagacagtcactcagtctcaaccagagatgtctgtgcaggaggcagagacggtgaccctgagctgcacatatgacaccagtgagagtgattattatttattctggtacaagcagcctcccagcaggcagatgattctcgttattcgccaagaagcttataagcaacagaatgcaacagagaatcgtttctctgtgaacttccagaaagcagccaaatccttcagtctcaagatctcagactcacagctgggggatgccgcgatgtatttctgcagagaccttgcggccgtgtcttcgactagtagctcacctacga

SEQ ID NO: 44 - DNA, 402 bp, Homo sapiens
  TRAV39_BB_1
gtcagatactccatgagcacgaagacttgtacgccaccatgaagaagctactagcaatgattctgtggcttcaactagaccggttaagtggagagctgaaagtggaacaaaaccctctgttcctgagcatgcaggagggaaaaaactataccatctactgcaattattcaaccacttcagacagactgtattggtacaggcaggatcctgggaaaagtctggaatctctgtttgtgttgctatcaaatggagcagtgaagcaggagggacgattaatggcctcacttgataccaaagcccgtctcagcaccctccacatcacagctgccgtgcatgacctctctgccacctacttctgcagagaccttgcggccgtgtcttcgactagtagctcacctacga

SEQ ID NO: 45 - DNA, 387 bp, Homo sapiens
  TRAV40_BB_1
gtcagatactccatgagcacgaagacttgtacgccaccatgaactcctctctggactttctaattctgatcttaatgtttggaggaaccagcagcaattcagtcaagcagacgggccaaataaccgtctcggagggagcatctgtgactatgaactgcacatacacatccacggggtaccctacccttttctggtatgtggaataccccagcaaacctctgcagcttcttcagagagagacaatggaaaacagcaaaaacttcggaggcggaaatattaaagacaaaaactcccccattgtgaaatattcagtccaggtatcagactcagccgtgtactactgcagagaccttgcggccgtgtcttcgactagtagctcacctacga

SEQ ID NO: 46 - DNA, 408 bp, Homo sapiens
  TRAV41_BB_1
gtcagatactccatgagcacgaagacttgtacgccaccatggtgaagatccggcaatttttgttggctattttgtggcttcagctaagctgtgtaagtgccgccaaaaatgaagtggagcagagtcctcagaacctgactgcccaggaaggagaatttatcacaatcaactgcagttactcggtaggaataagtgccttacactggctgcaacagcatccaggaggaggcattgtttccttgtttatgctgagctcagggaagaagaagcatggaagattaattgccacaataaacatacaggaaaagcacagctccctgcacatcacagcctcccatcccagagactctgccgtctacatctgcagagaccttgcggccgtgtcttcgactagtagctcacctacga

SEQ ID NO: 47 - DNA, 497 bp, Homo sapiens
  TRAC1 cloning fragment
gtgcactcctatgactaacggaagactaggccgcataggtctcaccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaggacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctgactaggtgtcttccctatgctgaatcgatggtc

SEQ ID NO: 48 - DNA, 3210 bp, Artificial Sequence
  V-C entry backbone transient
tctagacctgatcataatcaagccatatcacatctgtagaggtttacttgctttaaaaaacctccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgcggatccaatctcgagctgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcatgaattcgctaccggtatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcaggtacc

SEQ ID NO: 49 - DNA, 769 bp, Homo sapiens
  V-C entry TRAV1-1_TRAC
gccaccatgtggggagctttccttctctatgtttccatgaagatgggaggcactgcaggacaaagccttgagcagccctctgaagtgacagctgtggaaggagccattgtccagataaactgcacgtaccagacatctgggttttatgggctgtcctggtaccagcaacatgatggcggagcacccacatttctttcttacaatgctctggatggtttggaggagacaggtcgtttttcttcattccttagtcgctctgatagttatggttacctccttctacaggagctccagatgaaagactctgcctcttacttctgcagagaccttgcggccgcataggtctcaccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaggacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 50 - DNA, 769 bp, Homo sapiens
  V-C entry TRAV1-2_TRAC
gccaccatgtggggagttttccttctttatgtttccatgaagatgggaggcactacaggacaaaacattgaccagcccactgagatgacagctacggaaggtgccattgtccagatcaactgcacgtaccagacatctgggttcaacgggctgttctggtaccagcaacatgctggcgaagcacccacatttctgtcttacaatgttctggatggtttggaggagaaaggtcgtttttcttcattccttagtcggtctaaagggtacagttacctccttttgaaggagctccagatgaaagactctgcctcttacctctgcagagaccttgcggccgcataggtctcaccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaggacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 51 - DNA, 781 bp, Homo sapiens
  V-C entry TRAV2_TRAC
gccaccatggctttgcagagcactctgggggcggtgtggctagggcttctcctcaactctctctggaaggttgcagaaagcaaggaccaagtgtttcagccttccacagtggcatcttcagagggagctgtggtggaaatcttctgtaatcactctgtgtccaatgcttacaacttcttctggtaccttcacttcccgggatgtgcaccaagactccttgttaaaggctcaaagccttctcagcagggacgatacaacatgacctatgaacggttctcttcatcgctgctcatcctccaggtgcgggaggcagatgctgctgtttactactgcagagaccttgcggccgcataggtctcaccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaggacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 52 - DNA, 784 bp, Homo sapiens
  V-C entry TRAV3_TRAC
gccaccatggcctctgcacccatctcgatgcttgcgatgctcttcacattgagtgggctgagagctcagtcagtggctcagccggaagatcaggtcaacgttgctgaagggaatcctctgactgtgaaatgcacctattcagtctctggaaacccttatcttttttggtatgttcaataccccaaccgaggcctccagttccttctgaaatacatcacaggggataacctggttaaaggcagctatggctttgaagctgaatttaacaagagccaaacctccttccacctgaagaaaccatctgcccttgtgagcgactccgctttgtacttctgcagagaccttgcggccgcataggtctcaccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaggacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 53 - DNA, 769 bp, Homo sapiens
  V-C entry TRAV4_TRAC
gccaccatgaggcaagtggcgagagtgatcgtgttcctgaccctgagtactttgagccttgctaagaccacccagcccatctccatggactcatatgaaggacaagaagtgaacataacctgtagccacaacaacattgctacaaatgattatatcacgtggtaccaacagtttcccagccaaggaccacgatttattattcaaggatacaagacaaaagttacaaacgaagtggcctccctgtttatccctgccgacagaaagtccagcactctgagcctgccccgggtttccctgagcgacactgctgtgtactactgcagagaccttgcggccgcataggtctcaccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaggacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 54 - DNA, 784 bp, Homo sapiens
  V-C entry TRAV5_TRAC
gccaccatgaaaacatttgctggattttcgttcctgtttttgtggctgcagctggactgtatgagtagaggagaggatgtggagcagagtcttttcctgagtgtccgagagggagacagctccgttataaactgcacttacacagacagctcctccacctacttatactggtataagcaagaacctggagcaggtctacagttgctgacgtatattttttcaaatatggacatgaaacaagaccaaagactcactgttctattgaataaaaaggataaacatctgtctctgcgcattgcagacacccagactggggactcagctatctacttctgcagagaccttgcggccgcataggtctcaccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaggacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 55 - DNA, 784 bp, Homo sapiens
  V-C entry TRAV6_TRAC
gccaccatggagtcattcctgggaggtgttttgctgattttgtggcttcaagtggactgggtgaagagccaaaagatagaacagaattccgaggccctgaacattcaggagggtaaaacggccaccctgacctgcaactatacaaactattccccagcatacttacagtggtaccgacaagatccaggaagaggccctgttttcttgctactcatacgtgaaaatgagaaagaaaaaaggaaagaaagactgaaggtcacctttgataccacccttaaacagagtttgtttcatatcacagcctcccagcctgcagactcagctacctacctctgcagagaccttgcggccgcataggtctcaccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaggacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 56 - DNA, 781 bp, Homo sapiens
  V-C entry TRAV7_TRAC
gccaccatggagaagatgcgtagacctgtcctaattatattttgtctatgtcttggctgggcaaatggagaaaaccaggtggagcacagccctcattttctgggaccccagcagggagacgttgcctccatgagctgcacgtactctgtcagtcgttttaacaatttgcagtggtacaggcaaaatacagggatgggtcccaaacacctattatccatgtattcagctggatatgagaagcagaaaggaaggctaaatgctacattactgaagaatggaagcagcttgtacattacagccgtgcagcctgaagattcagccacctatttctgcagagaccttgcggccgcataggtctcaccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaggacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 57 - DNA, 784 bp, Homo sapiens
  V-C entry TRAV8-1_TRAC
gccaccatgctcctgttgctcataccagtgctggggatgatttttgccctgagagatgccagagcccagtctgtgagccagcataaccaccacgtaattctctctgaagcagcctcactggagttgggatgcaactattcctatggtggaactgttaatctcttctggtatgtccagtaccctggtcaacaccttcagcttctcctcaagtacttttcaggggatccactggttaaaggcatcaagggctttgaggctgaatttataaagagtaaattctcctttaatctgaggaaaccctctgtgcagtggagtgacacagctgagtacttctgcagagaccttgcggccgcataggtctcaccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaggacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 58 - DNA, 784 bp, Homo sapiens
  V-C entry TRAV8-2_TRAC
gccaccatgctcctgctgctcgtcccagtgctcgaggtgatttttactctgggaggaaccagagcccagtcggtgacccagcttgacagccacgtctctgtctctgaaggaaccccggtgctgctgaggtgcaactactcatcttcttattcaccatctctcttctggtatgtgcaacaccccaacaaaggactccagcttctcctgaagtacacatcagcggccaccctggttaaaggcatcaacggttttgaggctgaatttaagaagagtgaaacctccttccacctgacgaaaccctcagcccatatgagcgacgcggctgagtacttctgcagagaccttgcggccgcataggtctcaccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaggacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 59 - DNA, 784 bp, Homo sapiens
  V-C entry TRAV8-3_TRAC
gccaccatgctcctggagcttatcccactgctggggatacattttgtcctgagaactgccagagcccagtcagtgacccagcctgacatccacatcactgtctctgaaggagcctcactggagttgagatgtaactattcctatggggcaacaccttatctcttctggtatgtccagtcccccggccaaggcctccagctgctcctgaagtacttttcaggagacactctggttcaaggcattaaaggctttgaggctgaatttaagaggagtcaatcttccttcaatctgaggaaaccctctgtgcattggagtgatgctgctgagtacttctgcagagaccttgcggccgcataggtctcaccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaggacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 60 - DNA, 784 bp, Homo sapiens
  V-C entry TRAV8-4_TRAC
gccaccatgctcctgctgctcgtcccagtgctcgaggtgatttttaccctgggaggaaccagagcccagtcggtgacccagcttggcagccacgtctctgtctctgaaggagccctggttctgctgaggtgcaactactcatcgtctgttccaccatatctcttctggtatgtgcaataccccaaccaaggactccagcttctcctgaagtacacatcagcggccaccctggttaaaggcatcaacggttttgaggctgaatttaagaagagtgaaacctccttccacctgacgaaaccctcagcccatatgagcgacgcggctgagtacttctgcagagaccttgcggccgcataggtctcaccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaggacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 61 - DNA, 784 bp, Homo sapiens
  V-C entry TRAV8-6_TRAC
gccaccatgctcctgctgctcgtcccagcgttccaggtgatttttaccctgggaggaaccagagcccagtctgtgacccagcttgacagccaagtccctgtctttgaagaagcccctgtggagctgaggtgcaactactcatcgtctgtttcagtgtatctcttctggtatgtgcaataccccaaccaaggactccagcttctcctgaagtatttatcaggatccaccctggttaaaggcatcaacggttttgaggctgaatttaacaagagtcaaacttccttccacttgaggaaaccctcagtccatataagcgacacggctgagtacttctgcagagaccttgcggccgcataggtctcaccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaggacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 62 - DNA, 781 bp, Homo sapiens
  V-C entry TRAV8-7_TRAC
gccaccatgctcttagtggtcattctgctgcttggaatgttcttcacactgagaaccagaacccagtcggtgacccagcttgatggccacatcactgtctctgaagaagcccctctggaactgaagtgcaactattcctatagtggagttccttctctcttctggtatgtccaatactctagccaaagcctccagcttctcctcaaagacctaacagaggccacccaggttaaaggcatcagaggttttgaggctgaatttaagaagagcgaaacctccttctacctgaggaaaccatcaacccatgtgagtgatgctgctgagtacttctgcagagaccttgcggccgcataggtctcaccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaggacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 63 - DNA, 781 bp, Homo sapiens
  V-C entry TRAV9-1_TRAC
gccaccatgaattcttctccaggaccagcgattgcactattcttaatgtttgggggaatcaatggagattcagtggtccagacagaaggccaagtgctcccctctgaaggggattccctgattgtgaactgctcctatgaaaccacacagtacccttcccttttttggtatgtccaatatcctggagaaggtccacagctccacctgaaagccatgaaggccaatgacaagggaaggaacaaaggttttgaagccatgtaccgtaaagaaaccacttctttccacttggagaaagactcagttcaagagtcagactccgctgtgtacttctgcagagaccttgcggccgcataggtctcaccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaggacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 64 - DNA, 781 bp, Homo sapiens
  V-C entry TRAV9-2_TRAC
gccaccatgaactattctccaggcttagtatctctgatactcttactgcttggaagaacccgtggaaattcagtgacccagatggaagggccagtgactctctcagaagaggccttcctgactataaactgcacgtacacagccacaggatacccttcccttttctggtatgtccaatatcctggagaaggtctacagctcctcctgaaagccacgaaggctgatgacaagggaagcaacaaaggttttgaagccacataccgtaaagaaaccacttctttccacttggagaaaggctcagttcaagtgtcagactcagcggtgtacttctgcagagaccttgcggccgcataggtctcaccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaggacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 65 - DNA, 787 bp, Homo sapiens
  V-C entry TRAV10_TRAC
gccaccatgaaaaagcatctgacgaccttcttggtgattttgtggctttatttttatagggggaatggcaaaaaccaagtggagcagagtcctcagtccctgatcatcctggagggaaagaactgcactcttcaatgcaattatacagtgagccccttcagcaacttaaggtggtataagcaagatactgggagaggtcctgtttccctgacaatcatgactttcagtgagaacacaaagtcgaacggaagatatacagcaactctggatgcagacacaaagcaaagctctctgcacatcacagcctcccagctcagcgattcagcctcctacatctgcagagaccttgcggccgcataggtctcaccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaggacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 66 - DNA, 781 bp, Homo sapiens
  V-C entry TRAV12-1_TRAC
gccaccatgatatccttgagagttttactggtgatcctgtggcttcagttaagctgggtttggagccaacggaaggaggtggagcaggatcctggacccttcaatgttccagagggagccactgtcgctttcaactgtacttacagcaacagtgcttctcagtctttcttctggtacagacaggattgcaggaaagaacctaagttgctgatgtccgtatactccagtggtaatgaagatggaaggtttacagcacagctcaatagagccagccagtatatttccctgctcatcagagactccaagctcagtgattcagccacctacctctgcagagaccttgcggccgcataggtctcaccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaggacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 67 - DNA, 787 bp, Homo sapiens
  V-C entry TRAV12-2_TRAC
gccaccatgatgaaatccttgagagttttactagtgatcctgtggcttcagttgagctgggtttggagccaacagaaggaggtggagcagaattctggacccctcagtgttccagagggagccattgcctctctcaactgcacttacagtgaccgaggttcccagtccttcttctggtacagacaatattctgggaaaagccctgagttgataatgttcatatactccaatggtgacaaagaagatggaaggtttacagcacagctcaataaagccagccagtatgtttctctgctcatcagagactcccagcccagtgattcagccacctacctctgcagagaccttgcggccgcataggtctcaccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaggacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 68 - DNA, 787 bp, Homo sapiens
  V-C entry TRAV12-3_TRAC
gccaccatgatgaaatccttgagagttttactggtgatcctgtggcttcagttaagctgggtttggagccaacagaaggaggtggagcaggatcctggaccactcagtgttccagagggagccattgtttctctcaactgcacttacagcaacagtgcttttcaatacttcatgtggtacagacagtattccagaaaaggccctgagttgctgatgtacacatactccagtggtaacaaagaagatggaaggtttacagcacaggtcgataaatccagcaagtatatctccttgttcatcagagactcacagcccagtgattcagccacctacctctgcagagaccttgcggccgcataggtctcaccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaggacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 69 - DNA, 781 bp, Homo sapiens
  V-C entry TRAV13-1_TRAC
gccaccatgacatccattcgagctgtatttatattcctgtggctgcagctggacttggtgaatggagagaatgtggagcagcatccttcaaccctgagtgtccaggagggagacagcgctgttatcaagtgtacttattcagacagtgcctcaaactacttcccttggtataagcaagaacttggaaaaggacctcagcttattatagacattcgttcaaatgtgggcgaaaagaaagaccaacgaattgctgttacattgaacaagacagccaaacatttctccctgcacatcacagaaacccaacctgaggactcggctgtctacttctgcagagaccttgcggccgcataggtctcaccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaggacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 70 - DNA, 784 bp, Homo sapiens
  V-C entry TRAV13-2_TRAC
gccaccatggcaggcattcgagctttatttatgtacttgtggctgcagctggactgggtgagcagaggagagagtgtggggctgcatcttcctaccctgagtgtccaggagggtgacaactctattatcaactgtgcttattcaaacagcgcctcagactacttcatttggtacaagcaagaatctggaaaaggtcctcaattcattatagacattcgttcaaatatggacaaaaggcaaggccaaagagtcaccgttttattgaataagacagtgaaacatctctctctgcaaattgcagctactcaacctggagactcagctgtctacttctgcagagaccttgcggccgcataggtctcaccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaggacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 71 - DNA, 790 bp, Homo sapiens
  V-C entry TRAV14DV4_TRAC
gccaccatgtcactttctagcctgctgaaggtggtcacagcttcactgtggctaggacctggcattgcccagaagataactcaaacccaaccaggaatgttcgtgcaggaaaaggaggctgtgactctggactgcacatatgacaccagtgatcaaagttatggtctattctggtacaagcagcccagcagtggggaaatgatttttcttatttatcaggggtcttatgacgagcaaaatgcaacagaaggtcgctactcattgaatttccagaaggcaagaaaatccgccaaccttgtcatctccgcttcacaactgggggactcagcaatgtatttctgcagagaccttgcggccgcataggtctcaccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaggacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 72 - DNA, 772 bp, Homo sapiens
  V-C entry TRAV16_TRAC
gccaccatgaagcccaccctcatctcagtgcttgtgataatatttatactcagaggaacaagagcccagagagtgactcagcccgagaagctcctctctgtctttaaaggggccccagtggagctgaagtgcaactattcctattctgggagtcctgaactcttctggtatgtccagtactccagacaacgcctccagttactcttgagacacatctctagagagagcatcaaaggcttcactgctgaccttaacaaaggcgagacatctttccacctgaagaaaccatttgctcaagaggaagattcagccatgtattactgcagagaccttgcggccgcataggtctcaccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaggacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 73 - DNA, 781 bp, Homo sapiens
  V-C entry TRAV17_TRAC
gccaccatggaaactctcctgggagtgtctttggtgattctatggcttcaactggctagggtgaacagtcaacagggagaagaggatcctcaggccttgagcatccaggagggtgaaaatgccaccatgaactgcagttacaaaactagtataaacaatttacagtggtatagacaaaattcaggtagaggccttgtccacctaattttaatacgttcaaatgaaagagagaaacacagtggaagattaagagtcacgcttgacacttccaagaaaagcagttccttgttgatcacggcttcccgggcagcagacactgcttcttacttctgcagagaccttgcggccgcataggtctcaccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaggacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 74 - DNA, 778 bp, Homo sapiens
  V-C entry TRAV18_TRAC
gccaccatgctgtctgcttcctgctcaggacttgtgatcttgttgatattcagaaggaccagtggagactcggttacccagacagaaggcccagttaccctccctgagagggcagctctgacattaaactgcacttatcagtccagctattcaacttttctattctggtatgtccagtatctaaacaaagagcctgagctcctcctgaaaagttcagaaaaccaggagacggacagcagaggttttcaggccagtcctatcaagagtgacagttccttccacctggagaagccctcggtgcagctgtcggactctgccgtgtactactgcagagaccttgcggccgcataggtctcaccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaggacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 75 - DNA, 790 bp, Homo sapiens
  V-C entry TRAV19_TRAC
gccaccatgctgactgccagcctgttgagggcagtcatagcctccatctgtgttgtatccagcatggctcagaaggtaactcaagcgcagactgaaatttctgtggtggagaaggaggatgtgaccttggactgtgtgtatgaaacccgtgatactacttattacttattctggtacaagcaaccaccaagtggagaattggttttccttattcgtcggaactcttttgatgagcaaaatgaaataagtggtcggtattcttggaacttccagaaatccaccagttccttcaacttcaccatcacagcctcacaagtcgtggactcagcagtatacttctgcagagaccttgcggccgcataggtctcaccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaggacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 76 - DNA, 781 bp, Homo sapiens
  V-C entry TRAV20_TRAC
gccaccatggagaaaatgttggagtgtgcattcatagtcttgtggcttcagcttggctggttgagtggagaggaccaggtgacgcagagtcccgaggccctgagactccaggagggagagagtagcagtcttaactgcagttacacagtcagcggtttaagagggctgttctggtataggcaagatcctgggaaaggccctgaattcctcttcaccctgtattcagctggggaagaaaaggagaaagaaaggctaaaagccacattaacaaagaaggaaagctttctgcacatcacagcccctaaacctgaggactcagccacttatctctgcagagaccttgcggccgcataggtctcaccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaggacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 77 - DNA, 781 bp, Homo sapiens
  V-C entry TRAV21_TRAC
gccaccatggagaccctcttgggcctgcttatcctttggctgcagctgcaatgggtgagcagcaaacaggaggtgacgcagattcctgcagctctgagtgtcccagaaggagaaaacttggttctcaactgcagtttcactgatagcgctatttacaacctccagtggtttaggcaggaccctgggaaaggactcacatctctgttgcttattcagtcaagtcagagagagcaaacaagtggacgccttaatgcctcgctggataaatcatcaggacgtagtactttatacattgcagcttctcagcctggtgactcagccacctacctctgcagagaccttgcggccgcataggtctcaccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaggacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 78 - DNA, 775 bp, Homo sapiens
  V-C entry TRAV22_TRAC
gccaccatgaagaggatattgggagctctgctggggctcttgagtgcccaggtttgctgtgtgagaggaatacaagtggagcagagtcctccagacctgattctccaggagggagccaattccacgctgcggtgcaatttttctgactctgtgaacaatttgcagtggtttcatcaaaacccttggggacagctcatcaacctgttttacattccctcagggacaaaacagaatggaagattaagcgccacgactgtcgctacggaacgctacagcttattgtacatttcctcttcccagaccacagactcaggcgtttatttctgcagagaccttgcggccgcataggtctcaccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaggacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 79 - DNA, 808 bp, Homo sapiens
  V-C entry TRAV23DV6_TRAC
gccaccatggacaagatcttaggagcatcatttttagttctgtggcttcaactatgctgggtgagtggccaacagaaggagaaaagtgaccagcagcaggtgaaacaaagtcctcaatctttgatagtccagaaaggagggatttcaattataaactgtgcttatgagaacactgcgtttgactactttccatggtaccaacaattccctgggaaaggccctgcattattgatagccatacgtccagatgtgagtgaaaagaaagaaggaagattcacaatctccttcaataaaagtgccaagcagttctcattgcatatcatggattcccagcctggagactcagccacctacttctgcagagaccttgcggccgcataggtctcaccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaggacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 80 - DNA, 790 bp, Homo sapiens
  V-C entry TRAV24_TRAC
gccaccatggagaagaatcctttggcagccccattactaatcctctggtttcatcttgactgcgtgagcagcatactgaacgtggaacaaagtcctcagtcactgcatgttcaggagggagacagcaccaatttcacctgcagcttcccttccagcaatttttatgccttacactggtacagatgggaaactgcaaaaagccccgaggccttgtttgtaatgactttaaatggggatgaaaagaagaaaggacgaataagtgccactcttaataccaaggagggttacagctatttgtacatcaaaggatcccagcctgaagattcagccacatacctctgcagagaccttgcggccgcataggtctcaccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaggacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 81 - DNA, 775 bp, Homo sapiens
  V-C entry TRAV25_TRAC
gccaccatgctactcatcacatcaatgttggtcttatggatgcaattgtcacaggtgaatggacaacaggtaatgcaaattcctcagtaccagcatgtacaagaaggagaggacttcaccacgtactgcaattcctcaactactttaagcaatatacagtggtataagcaaaggcctggtggacatcccgtattcttgatacagttagtgaagagtggagaagtgaagaagcagaaaagactgacatttcagtttggagaagcaaaaaagaacagctccctgcacatcacagccacccagactacagatgtaggaacctacttctgcagagaccttgcggccgcataggtctcaccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaggacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 82 - DNA, 769 bp, Homo sapiens
  V-C entry TRAV26-1_TRAC
gccaccatgaggctggtggcaagagtaactgtgtttctgacctttggaactataattgatgctaagaccacccagcccccctccatggattgcgctgaaggaagagctgcaaacctgccttgtaatcactctaccatcagtggaaatgagtatgtgtattggtatcgacagattcactcccaggggccacagtatatcattcatggtctaaaaaacaatgaaaccaatgaaatggcctctctgatcatcacagaagatagaaagtccagcaccttgatcctgccccacgctacgctgagagacactgctgtgtactactgcagagaccttgcggccgcataggtctcaccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaggacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 83 - DNA, 769 bp, Homo sapiens
  V-C entry TRAV26-2_TRAC
gccaccatgaagttggtgacaagcattactgtactcctatctttgggtattatgggtgatgctaagaccacacagccaaattcaatggagagtaacgaagaagagcctgttcacttgccttgtaaccactccacaatcagtggaactgattacatacattggtatcgacagcttccctcccagggtccagagtacgtgattcatggtcttacaagcaatgtgaacaacagaatggcctctctggcaatcgctgaggacagaaagtccagtaccttgatcctgcaccgtgctaccttgagagatgctgctgtgtactactgcagagaccttgcggccgcataggtctcaccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaggacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 84 - DNA, 775 bp, Homo sapiens
  V-C entry TRAV27_TRAC
gccaccatggtcctgaaattctccgtgtccattctttggattcagttggcatgggtgagcacccagctgctggagcagagccctcagtttctaagcatccaagagggagaaaatctcactgtgtactgcaactcctcaagtgttttttccagcttacaatggtacagacaggagcctggggaaggtcctgtcctcctggtgacagtagttacgggtggagaagtgaagaagctgaagagactaacctttcagtttggtgatgcaagaaaggacagttctctccacatcactgcagcccagcctggtgatacaggcctctacctctgcagagaccttgcggccgcataggtctcaccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaggacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 85 - DNA, 802 bp, Homo sapiens
  V-C entry TRAV29DV5_TRAC
gccaccatggccatgctcctgggggcatcagtgctgattctgtggcttcagccagactgggtaaacagtcaacagaagaatgatgaccagcaagttaagcaaaattcaccatccctgagcgtccaggaaggaagaatttctattctgaactgtgactatactaacagcatgtttgattatttcctatggtacaaaaaataccctgctgaaggtcctacattcctgatatctataagttccattaaggataaaaatgaagatggaagattcactgttttcttaaacaaaagtgccaagcacctctctctgcacattgtgccctcccagcctggagactctgcagtgtacttctgcagagaccttgcggccgcataggtctcaccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaggacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 86 - DNA, 781 bp, Homo sapiens
  V-C entry TRAV30_TRAC
gccaccatggagactctcctgaaagtgctttcaggcaccttgttgtggcagttgacctgggtgagaagccaacaaccagtgcagagtcctcaagccgtgatcctccgagaaggggaagatgctgtcatcaactgcagttcctccaaggctttatattctgtacactggtacaggcagaagcatggtgaagcacccgttttcctgatgatattactgaagggtggagaacagaagggtcatgaaaaaatatctgcttcatttaatgaaaaaaagcagcaaagctccctgtaccttacggcctcccagctcagttactcaggaacctacttctgcagagaccttgcggccgcataggtctcaccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaggacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 87 - DNA, 781 bp, Homo sapiens
  V-C entry TRAV34_TRAC
gccaccatggagactgttctgcaagtactcctagggatattggggttccaagcagcctgggtcagtagccaagaactggagcagagtcctcagtccttgatcgtccaagagggaaagaatctcaccataaactgcacgtcatcaaagacgttatatggcttatactggtataagcaaaagtatggtgaaggtcttatcttcttgatgatgctacagaaaggtggggaagagaaaagtcatgaaaagataactgccaagttggatgagaaaaagcagcaaagttccctgcatatcacagcctcccagcccagccatgcaggcatctacctctgcagagaccttgcggccgcataggtctcaccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaggacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 88 - DNA, 775 bp, Homo sapiens
  V-C entry TRAV35_TRAC
gccaccatgctccttgaacatttattaataatcttgtggatgcagctgacatgggtcagtggtcaacagctgaatcagagtcctcaatctatgtttatccaggaaggagaagatgtctccatgaactgcacttcttcaagcatatttaacacctggctatggtacaagcaggaacctggggaaggtcctgtcctcttgatagccttatataaggctggtgaattgacctcaaatggaaggctgactgctcagtttggtataaccagaaaggacagcttcctgaatatctcagcatccatacctagtgatgtaggcatctacttctgcagagaccttgcggccgcataggtctcaccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaggacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 89 - DNA, 784 bp, Homo sapiens
  V-C entry TRAV36DV7_TRAC
gccaccatgatgaagtgtccacaggctttactagctatcttttggcttctactgagctgggtgagcagtgaagataaggtggtacaaagccctctatctctggttgtccacgagggagacaccgtaactctcaattgcagttatgaagtgactaactttcgaagcctactatggtacaagcaggaaaagaaagctcccacatttctatttatgctaacttcaagtggaattgaaaagaagtcaggtagactaagtagcatattagataagaaagaactttccagcatcctgaacatcacagccacccagaccggagactcggccatctacctctgcagagaccttgcggccgcataggtctcaccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaggacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 90 - DNA, 790 bp, Homo sapiens
  V-C entry TRAV38-1_TRAC
gccaccatgacacgagttagcttgctgtgggcagtcgtggtcagtacctgtcttgaatccggcatggcccagacagtcactcagtctcaaccagagatgtctgtgcaggaggcagagactgtgaccctgagttgcacatatgacaccagtgagaataattattatttgttctggtacaagcagcctcccagcaggcagatgattctcgttattcgccaagaagcttataagcaacagaatgcaacggagaatcgtttctctgtgaacttccagaaagcagccaaatccttcagtctcaagatctcagactcacagctgggggacactgcgatgtatttctgcagagaccttgcggccgcataggtctcaccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaggacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 91 - DNA, 790 bp, Homo sapiens
  V-C entry TRAV38-2DV8_TRAC
gccaccatggcatgccctggcttcctgtgggcacttgtgatctccacctgtcttgaatttagcatggctcagacagtcactcagtctcaaccagagatgtctgtgcaggaggcagagacggtgaccctgagctgcacatatgacaccagtgagagtgattattatttattctggtacaagcagcctcccagcaggcagatgattctcgttattcgccaagaagcttataagcaacagaatgcaacagagaatcgtttctctgtgaacttccagaaagcagccaaatccttcagtctcaagatctcagactcacagctgggggatgccgcgatgtatttctgcagagaccttgcggccgcataggtctcaccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaggacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 92 - DNA, 775 bp, Homo sapiens
  V-C entry TRAV39_TRAC
gccaccatgaagaagctactagcaatgattctgtggcttcaactagaccggttaagtggagagctgaaagtggaacaaaaccctctgttcctgagcatgcaggagggaaaaaactataccatctactgcaattattcaaccacttcagacagactgtattggtacaggcaggatcctgggaaaagtctggaatctctgtttgtgttgctatcaaatggagcagtgaagcaggagggacgattaatggcctcacttgataccaaagcccgtctcagcaccctccacatcacagctgccgtgcatgacctctctgccacctacttctgcagagaccttgcggccgcataggtctcaccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaggacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 93 - DNA, 760 bp, Homo sapiens
  V-C entry TRAV40_TRAC
gccaccatgaactcctctctggactttctaattctgatcttaatgtttggaggaaccagcagcaattcagtcaagcagacgggccaaataaccgtctcggagggagcatctgtgactatgaactgcacatacacatccacggggtaccctacccttttctggtatgtggaataccccagcaaacctctgcagcttcttcagagagagacaatggaaaacagcaaaaacttcggaggcggaaatattaaagacaaaaactcccccattgtgaaatattcagtccaggtatcagactcagccgtgtactactgcagagaccttgcggccgcataggtctcaccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaggacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 94 - DNA, 781 bp, Homo sapiens
  V-C entry TRAV41_TRAC
gccaccatggtgaagatccggcaatttttgttggctattttgtggcttcagctaagctgtgtaagtgccgccaaaaatgaagtggagcagagtcctcagaacctgactgcccaggaaggagaatttatcacaatcaactgcagttactcggtaggaataagtgccttacactggctgcaacagcatccaggaggaggcattgtttccttgtttatgctgagctcagggaagaagaagcatggaagattaattgccacaataaacatacaggaaaagcacagctccctgcacatcacagcctcccatcccagagactctgccgtctacatctgcagagaccttgcggccgcataggtctcaccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaggacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 95 - DNA, 53 bp, Artificial Sequence
  TRAJ_receiving_F1
aattcggtctcgaagtcttctgcggccgctgaagacactatccagtgagaccc

SEQ ID NO: 96 - DNA, 53 bp, Artificial Sequence
  TRAJ_receiving_R1
tcgagggtctcactggatagtgtcttcagcggccgcagaagacttcgagaccg

SEQ ID NO: 97 - DNA, 2308 bp, Artificial Sequence
  J donor backbone
ctcgagctgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaattcgtccagcatcagatgaaattgcagtttgttcatgtccgggttatcaataccatatttctggaacagacgtttctgcaggctcgggctaaattcacccagacaattccacagaattgccagatcctgataacgatctgcaataccaacacgaccaacatcaatgcagccaatcagtttaccctcatcaaaaatcaggttatccaggctaaaatcaccatgggtaacaacgctatccggactaaacggcagcagtttatgcatttctttccaaacctgttcaacaggccaaccattacgttcatcatcaaaatcgcttgcatcaaccagaccattattcatacggctctgtgcctgtgccagacgaaaaacacgatcgctattaaacggacaattacaaaccggaatgctatgcagacgacgcagaaaaactgccagtgcatcaacaatattttcgcctgaatccggatattcttccagaacctgaaatgcggttttacccggaattgcggtggtcagcagccatgcatcatccggtgtacgaataaaatgtttaatggtcggcagcggcataaattcggtcagccaattcagacgaaccatttcatcggtcacatcatttgcaacgctacctttaccatgtttcagaaacagttccggtgcatccggtttaccatacagacgataaatggttgcaccgctctgaccaacattatcacgtgcccatttatagccatacagatctgcatccatattgctattcagacgcggacggctacagctggtttcacgctgaatatggctcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcatgaattc

SEQ ID NO: 98 - DNA, 2355 bp, Artificial Sequence
  TRA J receiving cassette vector
cgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcatgaattcggtctcgaagtcttctgcggccgctgaagacactatccagtgagaccctcgagctgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaattcgtccagcatcagatgaaattgcagtttgttcatgtccgggttatcaataccatatttctggaacagacgtttctgcaggctcgggctaaattcacccagacaattccacagaattgccagatcctgataacgatctgcaataccaacacgaccaacatcaatgcagccaatcagtttaccctcatcaaaaatcaggttatccaggctaaaatcaccatgggtaacaacgctatccggactaaacggcagcagtttatgcatttctttccaaacctgttcaacaggccaaccattacgttcatcatcaaaatcgcttgcatcaaccagaccattattcatacggctctgtgcctgtgccagacgaaaaacacgatcgctattaaacggacaattacaaaccggaatgctatgcagacgacgcagaaaaactgccagtgcatcaacaatattttcgcctgaatccggatattcttccagaacctgaaatgcggttttacccggaattgcggtggtcagcagccatgcatcatccggtgtacgaataaaatgtttaatggtcggcagcggcataaattcggtcagccaattcagacgaaccatttcatcggtcacatcatttgcaacgctacctttaccatgtttcagaaacagttccggtgcatccggtttaccatacagacgataaatggttgcaccgctctgaccaacattatcacgtgcccatttatagccatacagatctgcatccatattgctattcagacgcggacggctacagctggtttcacgctgaatatggctcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctctt

SEQ ID NO: 99 - DNA, 39 bp, Homo sapiens
  TRAJ1*01_BB-S_F1
ctcgtttggcaaaggaaccagagtttccacttctcccca

SEQ ID NO: 100 - DNA, 39 bp, Homo sapiens
  TRAJ2*01_BB-S_F1
ctcgtttgggaaagggacccatgtattcattatatctga

SEQ ID NO: 101 - DNA, 39 bp, Homo sapiens
  TRAJ3*01_BB-S_F1
ctcgtttggatcagggaccagactcagcatccggccaaa

SEQ ID NO: 102 - DNA, 39 bp, Homo sapiens
  TRAJ4*01_BB-S_F1
ctcgtttggagcagggaccaggctggctgtacacccata

SEQ ID NO: 103 - DNA, 39 bp, Homo sapiens
  TRAJ5*01_BB-S_F1
ctcgtttgggagtggaacaagactccaagtgcaaccaaa

SEQ ID NO: 104 - DNA, 39 bp, Homo sapiens
  TRAJ6*01_BB-S_F1
ctcgtttggaagaggaaccagccttattgttcatccgta

SEQ ID NO: 105 - DNA, 39 bp, Homo sapiens
  TRAJ7*01_BB-S_F1
ctcgtttgggaaggggaaccaagtggtggtcataccaaa

SEQ ID NO: 106 - DNA, 39 bp, Homo sapiens
  TRAJ8*01_BB-S_F1
ctcgtttggaactggcacccgacttctggtcagtccaaa

SEQ ID NO: 107 - DNA, 39 bp, Homo sapiens
  TRAJ9*01_BB-S_F1
ctcgtttggagcaggaacaagactatttgttaaagcaaa

SEQ ID NO: 108 - DNA, 39 bp, Homo sapiens
  TRAJ10*01_BB-S_F1
ctcgtttgggacaggcactcagctaaaagtggaactcaa

SEQ ID NO: 109 - DNA, 39 bp, Homo sapiens
  TRAJ11*01_BB-S_F1
ctcgtttgggaaggggactatgcttctagtctctccaga

SEQ ID NO: 110 - DNA, 39 bp, Homo sapiens
  TRAJ12*01_BB-S_F1
ctcgtttgggagtgggaccagactgctggtcaggcctga

SEQ ID NO: 111 - DNA, 39 bp, Homo sapiens
  TRAJ13*01_BB-S_F1
ctcgtttggaattggaacaaagctccaagtcatcccaaa

SEQ ID NO: 112 - DNA, 39 bp, Homo sapiens
  TRAJ14*01_BB-S_F1
ctcgtttgggagtgggacaagattatcagtaaaacctga

SEQ ID NO: 113 - DNA, 39 bp, Homo sapiens
  TRAJ15*01_BB-S_F1
ctcgtttgggaagggaacccacctatcagtgagttccaa

SEQ ID NO: 114 - DNA, 39 bp, Homo sapiens
  TRAJ16*01_BB-S_F1
ctcgtttgcaaggggaaccatgttaaaggtggatcttaa

SEQ ID NO: 115 - DNA, 39 bp, Homo sapiens
  TRAJ17*01_BB-S_F1
ctcgtttggaggaggaaccagggtgctagttaaaccaaa

SEQ ID NO: 116 - DNA, 39 bp, Homo sapiens
  TRAJ18*01_BB-S_F1
ctcgtttggaagaggaactcagttgactgtctggcctga

SEQ ID NO: 117 - DNA, 39 bp, Homo sapiens
  TRAJ19*01_BB-S_F1
ctcgtttggaaagggatccaaacataatgtcactccaaa

SEQ ID NO: 118 - DNA, 39 bp, Homo sapiens
  TRAJ20*01_BB-S_F1
ctcgtttggagccggaaccacagtaactgtaagagcaaa

SEQ ID NO: 119 - DNA, 39 bp, Homo sapiens
  TRAJ21*01_BB-S_F1
ctcgtttggatctgggaccaaactcaatgtaaaaccaaa

SEQ ID NO: 120 - DNA, 39 bp, Homo sapiens
  TRAJ22*01_BB-S_F1
ctcgtttggatctgggacacaattgactgttttacctga

SEQ ID NO: 121 - DNA, 39 bp, Homo sapiens
  TRAJ23*01_BB-S_F1
ctcgtttggacagggaacggagttatctgtgaaacccaa

SEQ ID NO: 122 - DNA, 39 bp, Homo sapiens
  TRAJ24*01_BB-S_F1
ctcgtttggagcagggacccaggttgtggtcaccccaga

SEQ ID NO: 123 - DNA, 39 bp, Homo sapiens
  TRAJ25*01_BB-S_F1
ctcgtttgggaaggggacaaggctgcttgtcaagccaaa

SEQ ID NO: 124 - DNA, 39 bp, Homo sapiens
  TRAJ26*01_BB-S_F1
ctcgtttggtcccggaaccagattgtccgtgctgcccta

SEQ ID NO: 125 - DNA, 39 bp, Homo sapiens
  TRAJ27*01_BB-S_F1
ctcgtttggggatgggactacgctcactgtgaagccaaa

SEQ ID NO: 126 - DNA, 39 bp, Homo sapiens
  TRAJ28*01_BB-S_F1
ctcgtttgggaaggggaccaaactctcggtcataccaaa

SEQ ID NO: 127 - DNA, 39 bp, Homo sapiens
  TRAJ29*01_BB-S_F1
ctcgtttggaaagggcacaagactttctgtgattgcaaa

SEQ ID NO: 128 - DNA, 39 bp, Homo sapiens
  TRAJ30*01_BB-S_F1
ctcgtttggaaaagggacacgacttcatattctccccaa

SEQ ID NO: 129 - DNA, 39 bp, Homo sapiens
  TRAJ31*01_BB-S_F1
ctcgtttggagatggaactcagctggtggtgaagcccaa

SEQ ID NO: 130 - DNA, 39 bp, Homo sapiens
  TRAJ32*01_BB-S_F1
ctcgtttggaactggcactctgcttgctgtccagccaaa

SEQ ID NO: 131 - DNA, 39 bp, Homo sapiens
  TRAJ33*01_BB-S_F1
ctcgtggggcgctgggaccaagctaattataaagccaga

SEQ ID NO: 132 - DNA, 38 bp, Homo sapiens
  TRAJ34*01_BB-S_F1
ctcgtttgggactgggaccagattacaagtctttccaa

SEQ ID NO: 133 - DNA, 39 bp, Homo sapiens
  TRAJ36*01_BB-S_F1
ctcgtttgggactggaacgagactcaccgttattcccta

SEQ ID NO: 134 - DNA, 39 bp, Homo sapiens
  TRAJ37*01_BB-S_F1
ctcgtttgggcaagggacaactttacaagtaaaaccaga

SEQ ID NO: 135 - DNA, 39 bp, Homo sapiens
  TRAJ38*01_BB-S_F1
ctcgtggggattgggaacaagcctggcagtaaatccgaa

SEQ ID NO: 136 - DNA, 39 bp, Homo sapiens
  TRAJ39*01_BB-S_F1
ctcgtttggaggaggaacaaggttaatggtcaaacccca

SEQ ID NO: 137 - DNA, 39 bp, Homo sapiens
  TRAJ40*01_BB-S_F1
ctcgtttggaacaggcaccaggctgaaggttttagcaaa

SEQ ID NO: 138 - DNA, 39 bp, Homo sapiens
  TRAJ41*01_BB-S_F1
ctcgtttggcaaaggcacctcgctgttggtcacacccca

SEQ ID NO: 139 - DNA, 39 bp, Homo sapiens
  TRAJ42*01_BB-S_F1
ctcgtttggaaaaggcactaaactctctgttaaaccaaa

SEQ ID NO: 140 - DNA, 39 bp, Homo sapiens
  TRAJ43*01_BB-S_F1
ctcgtttggagcagggaccagactgacagtaaaaccaaa

SEQ ID NO: 141 - DNA, 39 bp, Homo sapiens
  TRAJ44*01_BB-S_F1
ctcgtttgggactggaacaagacttcaggtcacgctcga

SEQ ID NO: 142 - DNA, 39 bp, Homo sapiens
  TRAJ45*01_BB-S_F1
ctcgtttggcaaagggactcatctaatcatccagcccta

SEQ ID NO: 143 - DNA, 39 bp, Homo sapiens
  TRAJ46*01_BB-S_F1
ctcgtttgggaccgggactcgtttagcagttaggcccaa

SEQ ID NO: 144 - DNA, 39 bp, Homo sapiens
  TRAJ47*01_BB-S_F1
ctcgtttggcgcaggaaccattctgagagtcaagtccta

SEQ ID NO: 145 - DNA, 39 bp, Homo sapiens
  TRAJ48*01_BB-S_F1
ctcgtttgggactggaacaagactcaccatcatacccaa

SEQ ID NO: 146 - DNA, 39 bp, Homo sapiens
  TRAJ49*01_BB-S_F1
ctcgtttgggacagggacaagtttgacggtcattccaaa

SEQ ID NO: 147 - DNA, 39 bp, Homo sapiens
  TRAJ50*01_BB-S_F1
ctcgtttgggccagggacaagcttatcagtcattccaaa

SEQ ID NO: 148 - DNA, 39 bp, Homo sapiens
  TRAJ52*01_BB-S_F1
ctcgtttggacaagggaccatcttgactgtccatccaaa

SEQ ID NO: 149 - DNA, 39 bp, Homo sapiens
  TRAJ53*01_BB-S_F1
ctcgtttggaaaaggaactctcttaaccgtgaatccaaa

SEQ ID NO: 150 - DNA, 39 bp, Homo sapiens
  TRAJ54*01_BB-S_F1
ctcgtttggccaaggaaccaggctgactatcaacccaaa

SEQ ID NO: 151 - DNA, 39 bp, Homo sapiens
  TRAJ56*01_BB-S_F1
ctcgtttggaaaaggaataactctgagtgttagaccaga

SEQ ID NO: 152 - DNA, 39 bp, Homo sapiens
  TRAJ57*01_BB-S_F1
ctcgtttggaaagggaacgaaactgacagtaaacccata

SEQ ID NO: 153 - DNA, 33 bp, Homo sapiens
  TRAJ59*01_BB-S_F1
ctcgtttggaatggggacgcaagtgagagtgaa

SEQ ID NO: 154 - DNA, 39 bp, Homo sapiens
  TRAJ61*01_BB-S_F1
ctcgtttggagccaacactagaggaatcatgaaactcaa

SEQ ID NO: 155 - DNA, 39 bp, Homo sapiens
  TRAJ1*01_BB-S_R1
gatatggggagaagtggaaactctggttcctttgccaaa

SEQ ID NO: 156 - DNA, 39 bp, Homo sapiens
  TRAJ2*01_BB-S_R1
gatatcagatataatgaatacatgggtccctttcccaaa

SEQ ID NO: 157 - DNA, 39 bp, Homo sapiens
  TRAJ3*01_BB-S_R1
gatatttggccggatgctgagtctggtccctgatccaaa

SEQ ID NO: 158 - DNA, 39 bp, Homo sapiens
  TRAJ4*01_BB-S_R1
gatatatgggtgtacagccagcctggtccctgctccaaa

SEQ ID NO: 159 - DNA, 39 bp, Homo sapiens
  TRAJ5*01_BB-S_R1
gatatttggttgcacttggagtcttgttccactcccaaa

SEQ ID NO: 160 - DNA, 39 bp, Homo sapiens
  TRAJ6*01_BB-S_R1
gatatacggatgaacaataaggctggttcctcttccaaa

SEQ ID NO: 161 - DNA, 39 bp, Homo sapiens
  TRAJ7*01_BB-S_R1
gatatttggtatgaccaccacttggttccccttcccaaa

SEQ ID NO: 162 - DNA, 39 bp, Homo sapiens
  TRAJ8*01_BB-S_R1
gatatttggactgaccagaagtcgggtgccagttccaaa

SEQ ID NO: 163 - DNA, 39 bp, Homo sapiens
  TRAJ9*01_BB-S_R1
gatatttgctttaacaaatagtcttgttcctgctccaaa

SEQ ID NO: 164 - DNA, 39 bp, Homo sapiens
  TRAJ10*01_BB-S_R1
gatattgagttccacttttagctgagtgcctgtcccaaa

SEQ ID NO: 165 - DNA, 39 bp, Homo sapiens
  TRAJ11*01_BB-S_R1
gatatctggagagactagaagcatagtccccttcccaaa

SEQ ID NO: 166 - DNA, 39 bp, Homo sapiens
  TRAJ12*01_BB-S_R1
gatatcaggcctgaccagcagtctggtcccactcccaaa

SEQ ID NO: 167 - DNA, 39 bp, Homo sapiens
  TRAJ13*01_BB-S_R1
gatatttgggatgacttggagctttgttccaattccaaa

SEQ ID NO: 168 - DNA, 39 bp, Homo sapiens
  TRAJ14*01_BB-S_R1
gatatcaggttttactgataatcttgtcccactcccaaa

SEQ ID NO: 169 - DNA, 39 bp, Homo sapiens
  TRAJ15*01_BB-S_R1
gatattggaactcactgataggtgggttcccttcccaaa

SEQ ID NO: 170 - DNA, 39 bp, Homo sapiens
  TRAJ16*01_BB-S_R1
gatattaagatccacctttaacatggttccccttgcaaa

SEQ ID NO: 171 - DNA, 39 bp, Homo sapiens
  TRAJ17*01_BB-S_R1
gatatttggtttaactagcaccctggttcctcctccaaa

SEQ ID NO: 172 - DNA, 39 bp, Homo sapiens
  TRAJ18*01_BB-S_R1
gatatcaggccagacagtcaactgagttcctcttccaaa

SEQ ID NO: 173 - DNA, 39 bp, Homo sapiens
  TRAJ19*01_BB-S_R1
gatatttggagtgacattatgtttggatccctttccaaa

SEQ ID NO: 174 - DNA, 39 bp, Homo sapiens
  TRAJ20*01_BB-S_R1
gatatttgctcttacagttactgtggttccggctccaaa

SEQ ID NO: 175 - DNA, 39 bp, Homo sapiens
  TRAJ21*01_BB-S_R1
gatatttggttttacattgagtttggtcccagatccaaa

SEQ ID NO: 176 - DNA, 39 bp, Homo sapiens
  TRAJ22*01_BB-S_R1
gatatcaggtaaaacagtcaattgtgtcccagatccaaa

SEQ ID NO: 177 - DNA, 39 bp, Homo sapiens
  TRAJ23*01_BB-S_R1
gatattgggtttcacagataactccgttccctgtccaaa

SEQ ID NO: 178 - DNA, 39 bp, Homo sapiens
  TRAJ24*01_BB-S_R1
gatatctggggtgaccacaacctgggtccctgctccaaa

SEQ ID NO: 179 - DNA, 39 bp, Homo sapiens
  TRAJ25*01_BB-S_R1
gatatttggcttgacaagcagccttgtccccttcccaaa

SEQ ID NO: 180 - DNA, 39 bp, Homo sapiens
  TRAJ26*01_BB-S_R1
gatatagggcagcacggacaatctggttccgggaccaaa

SEQ ID NO: 181 - DNA, 39 bp, Homo sapiens
  TRAJ27*01_BB-S_R1
gatatttggcttcacagtgagcgtagtcccatccccaaa

SEQ ID NO: 182 - DNA, 39 bp, Homo sapiens
  TRAJ28*01_BB-S_R1
gatatttggtatgaccgagagtttggtccccttcccaaa

SEQ ID NO: 183 - DNA, 39 bp, Homo sapiens
  TRAJ29*01_BB-S_R1
gatatttgcaatcacagaaagtcttgtgccctttccaaa

SEQ ID NO: 184 - DNA, 39 bp, Homo sapiens
  TRAJ30*01_BB-S_R1
gatattggggagaatatgaagtcgtgtcccttttccaaa

SEQ ID NO: 185 - DNA, 39 bp, Homo sapiens
  TRAJ31*01_BB-S_R1
gatattgggcttcaccaccagctgagttccatctccaaa

SEQ ID NO: 186 - DNA, 39 bp, Homo sapiens
  TRAJ32*01_BB-S_R1
gatatttggctggacagcaagcagagtgccagttccaaa

SEQ ID NO: 187 - DNA, 39 bp, Homo sapiens
  TRAJ33*01_BB-S_R1
gatatctggctttataattagcttggtcccagcgcccca

SEQ ID NO: 188 - DNA, 39 bp, Homo sapiens
  TRAJ34*01_BB-S_R1
gatatttggaaagacttgtaatctggtcccagtcccaaa

SEQ ID NO: 189 - DNA, 39 bp, Homo sapiens
  TRAJ36*01_BB-S_R1
gatatagggaataacggtgagtctcgttccagtcccaaa

SEQ ID NO: 190 - DNA, 39 bp, Homo sapiens
  TRAJ37*01_BB-S_R1
gatatctggttttacttgtaaagttgtcccttgcccaaa

SEQ ID NO: 191 - DNA, 39 bp, Homo sapiens
  TRAJ38*01_BB-S_R1
gatattcggatttactgccaggcttgttcccaatcccca

SEQ ID NO: 192 - DNA, 39 bp, Homo sapiens
  TRAJ39*01_BB-S_R1
gatatggggtttgaccattaaccttgttcctcctccaaa

SEQ ID NO: 193 - DNA, 39 bp, Homo sapiens
  TRAJ40*01_BB-S_R1
gatatttgctaaaaccttcagcctggtgcctgttccaaa

SEQ ID NO: 194 - DNA, 39 bp, Homo sapiens
  TRAJ41*01_BB-S_R1
gatatggggtgtgaccaacagcgaggtgcctttgccaaa

SEQ ID NO: 195 - DNA, 39 bp, Homo sapiens
  TRAJ42*01_BB-S_R1
gatatttggtttaacagagagtttagtgccttttccaaa

SEQ ID NO: 196 - DNA, 39 bp, Homo sapiens
  TRAJ43*01_BB-S_R1
gatatttggttttactgtcagtctggtccctgctccaaa

SEQ ID NO: 197 - DNA, 39 bp, Homo sapiens
  TRAJ44*01_BB-S_R1
gatatcgagcgtgacctgaagtcttgttccagtcccaaa

SEQ ID NO: 198 - DNA, 39 bp, Homo sapiens
  TRAJ45*01_BB-S_R1
gatatagggctggatgattagatgagtccctttgccaaa

SEQ ID NO: 199 - DNA, 39 bp, Homo sapiens
  TRAJ46*01_BB-S_R1
gatattgggcctaactgctaaacgagtcccggtcccaaa

SEQ ID NO: 200 - DNA, 39 bp, Homo sapiens
  TRAJ47*01_BB-S_R1
gatataggacttgactctcagaatggttcctgcgccaaa

SEQ ID NO: 201 - DNA, 39 bp, Homo sapiens
  TRAJ48*01_BB-S_R1
gatattgggtatgatggtgagtcttgttccagtcccaaa

SEQ ID NO: 202 - DNA, 39 bp, Homo sapiens
  TRAJ49*01_BB-S_R1
gatatttggaatgaccgtcaaacttgtccctgtcccaaa

SEQ ID NO: 203 - DNA, 39 bp, Homo sapiens
  TRAJ50*01_BB-S_R1
gatatttggaatgactgataagcttgtccctggcccaaa

SEQ ID NO: 204 - DNA, 39 bp, Homo sapiens
  TRAJ52*01_BB-S_R1
gatatttggatggacagtcaagatggtcccttgtccaaa

SEQ ID NO: 205 - DNA, 39 bp, Homo sapiens
  TRAJ53*01_BB-S_R1
gatatttggattcacggttaagagagttccttttccaaa

SEQ ID NO: 206 - DNA, 39 bp, Homo sapiens
  TRAJ54*01_BB-S_R1
gatatttgggttgatagtcagcctggttccttggccaaa

SEQ ID NO: 207 - DNA, 39 bp, Homo sapiens
  TRAJ56*01_BB-S_R1
gatatctggtctaacactcagagttattccttttccaaa

SEQ ID NO: 208 - DNA, 39 bp, Homo sapiens
  TRAJ57*01_BB-S_R1
gatatatgggtttactgtcagtttcgttccctttccaaa

SEQ ID NO: 209 - DNA, 33 bp, Homo sapiens
  TRAJ59*01_BB-S_R1
gatattcactctcacttgcgtccccattccaaa

SEQ ID NO: 210 - DNA, 39 bp, Homo sapiens
  TRAJ61*01_BB-S_R1
gatattgagtttcatgattcctctagtgttggctccaaa

SEQ ID NO: 211 - DNA, 57 bp, Homo sapiens
  TRAJ1*01_BB-L_F1
ctcgattacctcccagttgcaatttggcaaaggaaccagagtttccacttctcccca

SEQ ID NO: 212 - DNA, 60 bp, Homo sapiens
  TRAJ2*01_BB-L_F1
ctcgggaacaattgataaactcacatttgggaaagggacccatgtattcattatatctga

SEQ ID NO: 213 - DNA, 57 bp, Homo sapiens
  TRAJ3*01_BB-L_F1
ctcgagtgcttccaagataatctttggatcagggaccagactcagcatccggccaaa

SEQ ID NO: 214 - DNA, 57 bp, Homo sapiens
  TRAJ4*01_BB-L_F1
ctcgggctacaataagctgatctttggagcagggaccaggctggctgtacacccata

SEQ ID NO: 215 - DNA, 54 bp, Homo sapiens
  TRAJ5*01_BB-L_F1
ctcgaggagagcacttacttttgggagtggaacaagactccaagtgcaaccaaa

SEQ ID NO: 216 - DNA, 57 bp, Homo sapiens
  TRAJ6*01_BB-L_F1
ctcgggaagctacatacctacatttggaagaggaaccagccttattgttcatccgta

SEQ ID NO: 217 - DNA, 54 bp, Homo sapiens
  TRAJ7*01_BB-L_F1
ctcgaacaacagactcgcttttgggaaggggaaccaagtggtggtcataccaaa

SEQ ID NO: 218 - DNA, 54 bp, Homo sapiens
  TRAJ8*01_BB-L_F1
ctcgtttcagaaacttgtatttggaactggcacccgacttctggtcagtccaaa

SEQ ID NO: 219 - DNA, 54 bp, Homo sapiens
  TRAJ9*01_BB-L_F1
ctcgggcttcaaaactatctttggagcaggaacaagactatttgttaaagcaaa

SEQ ID NO: 220 - DNA, 57 bp, Homo sapiens
  TRAJ10*01_BB-L_F1
ctcgggaggaaacaaactcacctttgggacaggcactcagctaaaagtggaactcaa

SEQ ID NO: 221 - DNA, 54 bp, Homo sapiens
  TRAJ11*01_BB-L_F1
ctcgtacagcaccctcacctttgggaaggggactatgcttctagtctctccaga

SEQ ID NO: 222 - DNA, 54 bp, Homo sapiens
  TRAJ12*01_BB-L_F1
ctcgagttataaattgatctttgggagtgggaccagactgctggtcaggcctga

SEQ ID NO: 223 - DNA, 57 bp, Homo sapiens
  TRAJ13*01_BB-L_F1
ctcgggttaccagaaagttacctttggaattggaacaaagctccaagtcatcccaaa

SEQ ID NO: 224 - DNA, 45 bp, Homo sapiens
  TRAJ14*01_BB-L_F1
ctcgttcatctttgggagtgggacaagattatcagtaaaacctga

SEQ ID NO: 225 - DNA, 54 bp, Homo sapiens
  TRAJ15*01_BB-L_F1
ctcgggaactgctctgatctttgggaagggaacccacctatcagtgagttccaa

SEQ ID NO: 226 - DNA, 54 bp, Homo sapiens
  TRAJ16*01_BB-L_F1
ctcgggacagaagctgctctttgcaaggggaaccatgttaaaggtggatcttaa

SEQ ID NO: 227 - DNA, 57 bp, Homo sapiens
  TRAJ17*01_BB-L_F1
ctcggcaggcaacaagctaacttttggaggaggaaccagggtgctagttaaaccaaa

SEQ ID NO: 228 - DNA, 60 bp, Homo sapiens
  TRAJ18*01_BB-L_F1
ctcgtcaaccctggggaggctatactttggaagaggaactcagttgactgtctggcctga

SEQ ID NO: 229 - DNA, 54 bp, Homo sapiens
  TRAJ19*01_BB-L_F1
ctcgttctacaatttcacctttggaaagggatccaaacataatgtcactccaaa

SEQ ID NO: 230 - DNA, 51 bp, Homo sapiens
  TRAJ20*01_BB-L_F1
ctcgtacaagctcagctttggagccggaaccacagtaactgtaagagcaaa

SEQ ID NO: 231 - DNA, 48 bp, Homo sapiens
  TRAJ21*01_BB-L_F1
ctcgaaattttactttggatctgggaccaaactcaatgtaaaaccaaa

SEQ ID NO: 232 - DNA, 57 bp, Homo sapiens
  TRAJ22*01_BB-L_F1
ctcgtctgcaaggcaactgacctttggatctgggacacaattgactgttttacctga

SEQ ID NO: 233 - DNA, 57 bp, Homo sapiens
  TRAJ23*01_BB-L_F1
ctcgcagggaggaaagcttatctttggacagggaacggagttatctgtgaaacccaa

SEQ ID NO: 234 - DNA, 57 bp, Homo sapiens
  TRAJ24*01_BB-L_F1
ctcgagttggggtaaattgcagtttggagcagggacccaggttgtggtcaccccaga

SEQ ID NO: 235 - DNA, 54 bp, Homo sapiens
  TRAJ25*01_BB-L_F1
ctcgggcttctcctttatctttgggaaggggacaaggctgcttgtcaagccaaa

SEQ ID NO: 236 - DNA, 54 bp, Homo sapiens
  TRAJ26*01_BB-L_F1
ctcgggtcagaattttgtctttggtcccggaaccagattgtccgtgctgcccta

SEQ ID NO: 237 - DNA, 54 bp, Homo sapiens
  TRAJ27*01_BB-L_F1
ctcggcaggcaaatcaacctttggggatgggactacgctcactgtgaagccaaa

SEQ ID NO: 238 - DNA, 60 bp, Homo sapiens
  TRAJ28*01_BB-L_F1
ctcggctgggagttaccaactcacttttgggaaggggaccaaactctcggtcataccaaa

SEQ ID NO: 239 - DNA, 54 bp, Homo sapiens
  TRAJ29*01_BB-L_F1
ctcgaacacacctcttgtctttggaaagggcacaagactttctgtgattgcaaa

SEQ ID NO: 240 - DNA, 51 bp, Homo sapiens
  TRAJ30*01_BB-L_F1
ctcggacaagatcatctttggaaaagggacacgacttcatattctccccaa

SEQ ID NO: 241 - DNA, 51 bp, Homo sapiens
  TRAJ31*01_BB-L_F1
ctcggccagactcatgtttggagatggaactcagctggtggtgaagcccaa

SEQ ID NO: 242 - DNA, 60 bp, Homo sapiens
  TRAJ32*01_BB-L_F1
ctcgggtgctacaaacaagctcatctttggaactggcactctgcttgctgtccagccaaa

SEQ ID NO: 243 - DNA, 51 bp, Homo sapiens
  TRAJ33*01_BB-L_F1
ctcgtatcagttaatctggggcgctgggaccaagctaattataaagccaga

SEQ ID NO: 244 - DNA, 51 bp, Homo sapiens
  TRAJ34*01_BB-L_F1
ctcggacaagctcatctttgggactgggaccagattacaagtctttccaaa

SEQ ID NO: 245 - DNA, 54 bp, Homo sapiens
  TRAJ36*01_BB-L_F1
ctcggcaaacaacctcttctttgggactggaacgagactcaccgttattcccta

SEQ ID NO: 246 - DNA, 57 bp, Homo sapiens
  TRAJ37*01_BB-L_F1
ctcgaacacaggcaaactaatctttgggcaagggacaactttacaagtaaaaccaga

SEQ ID NO: 247 - DNA, 57 bp, Homo sapiens
  TRAJ38*01_BB-L_F1
ctcgaacaaccgtaagctgatttggggattgggaacaagcctggcagtaaatccgaa

SEQ ID NO: 248 - DNA, 57 bp, Homo sapiens
  TRAJ39*01_BB-L_F1
ctcggcaggcaacatgctcacctttggaggaggaacaaggttaatggtcaaacccca

SEQ ID NO: 249 - DNA, 54 bp, Homo sapiens
  TRAJ40*01_BB-L_F1
ctcgacctacaaatacatctttggaacaggcaccaggctgaaggttttagcaaa

SEQ ID NO: 250 - DNA, 57 bp, Homo sapiens
  TRAJ41*01_BB-L_F1
ctcgtccgggtatgcactcaactttggcaaaggcacctcgctgttggtcacacccca

SEQ ID NO: 251 - DNA, 60 bp, Homo sapiens
  TRAJ42*01_BB-L_F1
ctcgggaagccaaggaaatctcatctttggaaaaggcactaaactctctgttaaaccaaa

SEQ ID NO: 252 - DNA, 48 bp, Homo sapiens
  TRAJ43*01_BB-L_F1
ctcggacatgcgctttggagcagggaccagactgacagtaaaaccaaa

SEQ ID NO: 253 - DNA, 57 bp, Homo sapiens
  TRAJ44*01_BB-L_F1
ctcgactgccagtaaactcacctttgggactggaacaagacttcaggtcacgctcga

SEQ ID NO: 254 - DNA, 60 bp, Homo sapiens
  TRAJ45*01_BB-L_F1
ctcgggaggtgctgacggactcacctttggcaaagggactcatctaatcatccagcccta

SEQ ID NO: 255 - DNA, 57 bp, Homo sapiens
  TRAJ46*01_BB-L_F1
ctcgagcggagacaagctgacttttgggaccgggactcgtttagcagttaggcccaa

SEQ ID NO: 256 - DNA, 51 bp, Homo sapiens
  TRAJ47*01_BB-L_F1
ctcgaacaaactggtctttggcgcaggaaccattctgagagtcaagtccta

SEQ ID NO: 257 - DNA, 57 bp, Homo sapiens
  TRAJ48*01_BB-L_F1
ctcgggaaatgagaaattaacctttgggactggaacaagactcaccatcatacccaa

SEQ ID NO: 258 - DNA, 51 bp, Homo sapiens
  TRAJ49*01_BB-L_F1
ctcgaaccagttctattttgggacagggacaagtttgacggtcattccaaa

SEQ ID NO: 259 - DNA, 54 bp, Homo sapiens
  TRAJ50*01_BB-L_F1
ctcgtacgacaaggtgatatttgggccagggacaagcttatcagtcattccaaa

SEQ ID NO: 260 - DNA, 63 bp, Homo sapiens
  TRAJ52*01_BB-L_F1
ctcgggtactagctatggaaagctgacatttggacaagggaccatcttgactgtccatccaaa

SEQ ID NO: 261 - DNA, 60 bp, Homo sapiens
  TRAJ53*01_BB-L_F1
ctcgggtagcaactataaactgacatttggaaaaggaactctcttaaccgtgaatccaaa

SEQ ID NO: 262 - DNA, 54 bp, Homo sapiens
  TRAJ54*01_BB-L_F1
ctcggcccagaagctggtatttggccaaggaaccaggctgactatcaacccaaa

SEQ ID NO: 263 - DNA, 57 bp, Homo sapiens
  TRAJ56*01_BB-L_F1
ctcggccaatagtaagctgacatttggaaaaggaataactctgagtgttagaccaga

SEQ ID NO: 264 - DNA, 57 bp, Homo sapiens
  TRAJ57*01_BB-L_F1
ctcgggatctgaaaagctggtctttggaaagggaacgaaactgacagtaaacccata

SEQ ID NO: 265 - DNA, 48 bp, Homo sapiens
  TRAJ59*01_BB-L_F1
ctcgaacaggaaatttacatttggaatggggacgcaagtgagagtgaa

SEQ ID NO: 266 - DNA, 54 bp, Homo sapiens
  TRAJ61*01_BB-L_F1
ctcgaataggaaactgacatttggagccaacactagaggaatcatgaaactcaa

SEQ ID NO: 267 - DNA, 57 bp, Homo sapiens
  TRAJ1*01_BB-L_R1
gatatggggagaagtggaaactctggttcctttgccaaattgcaactgggaggtaat

SEQ ID NO: 268 - DNA, 60 bp, Homo sapiens
  TRAJ2*01_BB-L_R1
gatatcagatataatgaatacatgggtccctttcccaaatgtgagtttatcaattgttcc

SEQ ID NO: 269 - DNA, 57 bp, Homo sapiens
  TRAJ3*01_BB-L_R1
gatatttggccggatgctgagtctggtccctgatccaaagattatcttggaagcact

SEQ ID NO: 270 - DNA, 57 bp, Homo sapiens
  TRAJ4*01_BB-L_R1
gatatatgggtgtacagccagcctggtccctgctccaaaaatcagcttattgtagcc

SEQ ID NO: 271 - DNA, 54 bp, Homo sapiens
  TRAJ5*01_BB-L_R1
gatatttggttgcacttggagtcttgttccactcccaaaagtaagtgctctcct

SEQ ID NO: 272 - DNA, 57 bp, Homo sapiens
  TRAJ6*01_BB-L_R1
gatatacggatgaacaataaggctggttcctcttccaaatgtaggtatgtagcttcc

SEQ ID NO: 273 - DNA, 54 bp, Homo sapiens
  TRAJ7*01_BB-L_R1
gatatttggtatgaccaccacttggttccccttcccaaaagcgagtctgttgtt

SEQ ID NO: 274 - DNA, 54 bp, Homo sapiens
  TRAJ8*01_BB-L_R1
gatatttggactgaccagaagtcgggtgccagttccaaatacaagtttctgaaa

SEQ ID NO: 275 - DNA, 54 bp, Homo sapiens
  TRAJ9*01_BB-L_R1
gatatttgctttaacaaatagtcttgttcctgctccaaagatagttttgaagcc

SEQ ID NO: 276 - DNA, 57 bp, Homo sapiens
  TRAJ10*01_BB-L_R1
gatattgagttccacttttagctgagtgcctgtcccaaaggtgagtttgtttcctcc

SEQ ID NO: 277 - DNA, 54 bp, Homo sapiens
  TRAJ11*01_BB-L_R1
gatatctggagagactagaagcatagtccccttcccaaaggtgagggtgctgta

SEQ ID NO: 278 - DNA, 54 bp, Homo sapiens
  TRAJ12*01_BB-L_R1
gatatcaggcctgaccagcagtctggtcccactcccaaagatcaatttataact

SEQ ID NO: 279 - DNA, 57 bp, Homo sapiens
  TRAJ13*01_BB-L_R1
gatatttgggatgacttggagctttgttccaattccaaaggtaactttctggtaacc

SEQ ID NO: 280 - DNA, 45 bp, Homo sapiens
  TRAJ14*01_BB-L_R1
gatatcaggttttactgataatcttgtcccactcccaaagatgaa

SEQ ID NO: 281 - DNA, 54 bp, Homo sapiens
  TRAJ15*01_BB-L_R1
gatattggaactcactgataggtgggttcccttcccaaagatcagagcagttcc

SEQ ID NO: 282 - DNA, 54 bp, Homo sapiens
  TRAJ16*01_BB-L_R1
gatattaagatccacctttaacatggttccccttgcaaagagcagcttctgtcc

SEQ ID NO: 283 - DNA, 57 bp, Homo sapiens
  TRAJ17*01_BB-L_R1
gatatttggtttaactagcaccctggttcctcctccaaaagttagcttgttgcctgc

SEQ ID NO: 284 - DNA, 60 bp, Homo sapiens
  TRAJ18*01_BB-L_R1
gatatcaggccagacagtcaactgagttcctcttccaaagtatagcctccccagggttga

SEQ ID NO: 285 - DNA, 54 bp, Homo sapiens
  TRAJ19*01_BB-L_R1
gatatttggagtgacattatgtttggatccctttccaaaggtgaaattgtagaa

SEQ ID NO: 286 - DNA, 51 bp, Homo sapiens
  TRAJ20*01_BB-L_R1
gatatttgctcttacagttactgtggttccggctccaaagctgagcttgta

SEQ ID NO: 287 - DNA, 48 bp, Homo sapiens
  TRAJ21*01_BB-L_R1
gatatttggttttacattgagtttggtcccagatccaaagtaaaattt

SEQ ID NO: 288 - DNA, 57 bp, Homo sapiens
  TRAJ22*01_BB-L_R1
gatatcaggtaaaacagtcaattgtgtcccagatccaaaggtcagttgccttgcaga

SEQ ID NO: 289 - DNA, 57 bp, Homo sapiens
  TRAJ23*01_BB-L_R1
gatattgggtttcacagataactccgttccctgtccaaagataagctttcctccctg

SEQ ID NO: 290 - DNA, 57 bp, Homo sapiens
  TRAJ24*01_BB-L_R1
gatatctggggtgaccacaacctgggtccctgctccaaactgcaatttaccccaact

SEQ ID NO: 291 - DNA, 54 bp, Homo sapiens
  TRAJ25*01_BB-L_R1
gatatttggcttgacaagcagccttgtccccttcccaaagataaaggagaagcc

SEQ ID NO: 292 - DNA, 54 bp, Homo sapiens
  TRAJ26*01_BB-L_R1
gatatagggcagcacggacaatctggttccgggaccaaagacaaaattctgacc

SEQ ID NO: 293 - DNA, 54 bp, Homo sapiens
  TRAJ27*01_BB-L_R1
gatatttggcttcacagtgagcgtagtcccatccccaaaggttgatttgcctgc

SEQ ID NO: 294 - DNA, 60 bp, Homo sapiens
  TRAJ28*01_BB-L_R1
gatatttggtatgaccgagagtttggtccccttcccaaaagtgagttggtaactcccagc

SEQ ID NO: 295 - DNA, 54 bp, Homo sapiens
  TRAJ29*01_BB-L_R1
gatatttgcaatcacagaaagtcttgtgccctttccaaagacaagaggtgtgtt

SEQ ID NO: 296 - DNA, 51 bp, Homo sapiens
  TRAJ30*01_BB-L_R1
gatattggggagaatatgaagtcgtgtcccttttccaaagatgatcttgtc

SEQ ID NO: 297 - DNA, 51 bp, Homo sapiens
  TRAJ31*01_BB-L_R1
gatattgggcttcaccaccagctgagttccatctccaaacatgagtctggc

SEQ ID NO: 298 - DNA, 60 bp, Homo sapiens
  TRAJ32*01_BB-L_R1
gatatttggctggacagcaagcagagtgccagttccaaagatgagcttgtttgtagcacc

SEQ ID NO: 299 - DNA, 51 bp, Homo sapiens
  TRAJ33*01_BB-L_R1
gatatctggctttataattagcttggtcccagcgccccagattaactgata

SEQ ID NO: 300 - DNA, 51 bp, Homo sapiens
  TRAJ34*01_BB-L_R1
gatatttggaaagacttgtaatctggtcccagtcccaaagatgagcttgtc

SEQ ID NO: 301 - DNA, 54 bp, Homo sapiens
  TRAJ36*01_BB-L_R1
gatatagggaataacggtgagtctcgttccagtcccaaagaagaggttgtttgc

SEQ ID NO: 302 - DNA, 57 bp, Homo sapiens
  TRAJ37*01_BB-L_R1
gatatctggttttacttgtaaagttgtcccttgcccaaagattagtttgcctgtgtt

SEQ ID NO: 303 - DNA, 57 bp, Homo sapiens
  TRAJ38*01_BB-L_R1
gatattcggatttactgccaggcttgttcccaatccccaaatcagcttacggttgtt

SEQ ID NO: 304 - DNA, 57 bp, Homo sapiens
  TRAJ39*01_BB-L_R1
gatatggggtttgaccattaaccttgttcctcctccaaaggtgagcatgttgcctgc

SEQ ID NO: 305 - DNA, 54 bp, Homo sapiens
  TRAJ40*01_BB-L_R1
gatatttgctaaaaccttcagcctggtgcctgttccaaagatgtatttgtaggt

SEQ ID NO: 306 - DNA, 57 bp, Homo sapiens
  TRAJ41*01_BB-L_R1
gatatggggtgtgaccaacagcgaggtgcctttgccaaagttgagtgcatacccgga

SEQ ID NO: 307 - DNA, 60 bp, Homo sapiens
  TRAJ42*01_BB-L_R1
gatatttggtttaacagagagtttagtgccttttccaaagatgagatttccttggcttcc

SEQ ID NO: 308 - DNA, 48 bp, Homo sapiens
  TRAJ43*01_BB-L_R1
gatatttggttttactgtcagtctggtccctgctccaaagcgcatgtc

SEQ ID NO: 309 - DNA, 57 bp, Homo sapiens
  TRAJ44*01_BB-L_R1
gatatcgagcgtgacctgaagtcttgttccagtcccaaaggtgagtttactggcagt

SEQ ID NO: 310 - DNA, 60 bp, Homo sapiens
  TRAJ45*01_BB-L_R1
gatatagggctggatgattagatgagtccctttgccaaaggtgagtccgtcagcacctcc

SEQ ID NO: 311 - DNA, 57 bp, Homo sapiens
  TRAJ46*01_BB-L_R1
gatattgggcctaactgctaaacgagtcccggtcccaaaagtcagcttgtctccgct

SEQ ID NO: 312 - DNA, 51 bp, Homo sapiens
  TRAJ47*01_BB-L_R1
gatataggacttgactctcagaatggttcctgcgccaaagaccagtttgtt

SEQ ID NO: 313 - DNA, 57 bp, Homo sapiens
  TRAJ48*01_BB-L_R1
gatattgggtatgatggtgagtcttgttccagtcccaaaggttaatttctcatttcc

SEQ ID NO: 314 - DNA, 51 bp, Homo sapiens
  TRAJ49*01_BB-L_R1
gatatttggaatgaccgtcaaacttgtccctgtcccaaaatagaactggtt

SEQ ID NO: 315 - DNA, 54 bp, Homo sapiens
  TRAJ50*01_BB-L_R1
gatatttggaatgactgataagcttgtccctggcccaaatatcaccttgtcgta

SEQ ID NO: 316 - DNA, 63 bp, Homo sapiens
  TRAJ52*01_BB-L_R1
gatatttggatggacagtcaagatggtcccttgtccaaatgtcagctttccatagctagtacc

SEQ ID NO: 317 - DNA, 60 bp, Homo sapiens
  TRAJ53*01_BB-L_R1
gatatttggattcacggttaagagagttccttttccaaatgtcagtttatagttgctacc

SEQ ID NO: 318 - DNA, 51 bp, Homo sapiens
  TRAJ54*01_BB-L_R1
gatatttgggttgatagtcagcctggttccttggccaaataccagcttctg

SEQ ID NO: 319 - DNA, 57 bp, Homo sapiens
  TRAJ56*01_BB-L_R1
gatatctggtctaacactcagagttattccttttccaaatgtcagcttactattggc

SEQ ID NO: 320 - DNA, 57 bp, Homo sapiens
  TRAJ57*01_BB-L_R1
gatatatgggtttactgtcagtttcgttccctttccaaagaccagcttttcagatcc

SEQ ID NO: 321 - DNA, 48 bp, Homo sapiens
  TRAJ59*01_BB-L_R1
gatattcactctcacttgcgtccccattccaaatgtaaatttcctgtt

SEQ ID NO: 322 - DNA, 54 bp, Homo sapiens
  TRAJ61*01_BB-L_R1
gatattgagtttcatgattcctctagtgttggctccaaatgtcagtttcctatt

SEQ ID NO: 323 - DNA, 56 bp, Homo sapiens
  J Donor_Short_TRAJ1
ggtctcgtttggcaaaggaaccagagtttccacttctccccatatccagtgagacc

SEQ ID NO: 324 - DNA, 56 bp, Homo sapiens
  J Donor_Short_TRAJ2
ggtctcgtttgggaaagggacccatgtattcattatatctgatatccagtgagacc

SEQ ID NO: 325 - DNA, 56 bp, Homo sapiens
  J Donor_Short_TRAJ3
ggtctcgtttggatcagggaccagactcagcatccggccaaatatccagtgagacc

SEQ ID NO: 326 - DNA, 56 bp, Homo sapiens
  J Donor_Short_TRAJ4
ggtctcgtttggagcagggaccaggctggctgtacacccatatatccagtgagacc

SEQ ID NO: 327 - DNA, 56 bp, Homo sapiens
  J Donor_Short_TRAJ5
ggtctcgtttgggagtggaacaagactccaagtgcaaccaaatatccagtgagacc

SEQ ID NO: 328 - DNA, 56 bp, Homo sapiens
  J Donor_Short_TRAJ6
ggtctcgtttggaagaggaaccagccttattgttcatccgtatatccagtgagacc

SEQ ID NO: 329 - DNA, 56 bp, Homo sapiens
  J Donor_Short_TRAJ7
ggtctcgtttgggaaggggaaccaagtggtggtcataccaaatatccagtgagacc

SEQ ID NO: 330 - DNA, 56 bp, Homo sapiens
  J Donor_Short_TRAJ8
ggtctcgtttggaactggcacccgacttctggtcagtccaaatatccagtgagacc

SEQ ID NO: 331 - DNA, 56 bp, Homo sapiens
  J Donor_Short_TRAJ9
ggtctcgtttggagcaggaacaagactatttgttaaagcaaatatccagtgagacc

SEQ ID NO: 332 - DNA, 56 bp, Homo sapiens
  J Donor_Short_TRAJ10
ggtctcgtttgggacaggcactcagctaaaagtggaactcaatatccagtgagacc

SEQ ID NO: 333 - DNA, 56 bp, Homo sapiens
  J Donor_Short_TRAJ11
ggtctcgtttgggaaggggactatgcttctagtctctccagatatccagtgagacc

SEQ ID NO: 334 - DNA, 56 bp, Homo sapiens
  J Donor_Short_TRAJ12
ggtctcgtttgggagtgggaccagactgctggtcaggcctgatatccagtgagacc

SEQ ID NO: 335 - DNA, 56 bp, Homo sapiens
  J Donor_Short_TRAJ13
ggtctcgtttggaattggaacaaagctccaagtcatcccaaatatccagtgagacc

SEQ ID NO: 336 - DNA, 56 bp, Homo sapiens
  J Donor_Short_TRAJ14
ggtctcgtttgggagtgggacaagattatcagtaaaacctgatatccagtgagacc

SEQ ID NO: 337 - DNA, 56 bp, Homo sapiens
  J Donor_Short_TRAJ15
ggtctcgtttgggaagggaacccacctatcagtgagttccaatatccagtgagacc

SEQ ID NO: 338 - DNA, 56 bp, Homo sapiens
  J Donor_Short_TRAJ16
ggtctcgtttgcaaggggaaccatgttaaaggtggatcttaatatccagtgagacc

SEQ ID NO: 339 - DNA, 56 bp, Homo sapiens
  J Donor_Short_TRAJ17
ggtctcgtttggaggaggaaccagggtgctagttaaaccaaatatccagtgagacc

SEQ ID NO: 340 - DNA, 56 bp, Homo sapiens
  J Donor_Short_TRAJ18
ggtctcgtttggaagaggaactcagttgactgtctggcctgatatccagtgagacc

SEQ ID NO: 341 - DNA, 56 bp, Homo sapiens
  J Donor_Short_TRAJ19
ggtctcgtttggaaagggatccaaacataatgtcactccaaatatccagtgagacc

SEQ ID NO: 342 - DNA, 56 bp, Homo sapiens
  J Donor_Short_TRAJ20
ggtctcgtttggagccggaaccacagtaactgtaagagcaaatatccagtgagacc

SEQ ID NO: 343 - DNA, 56 bp, Homo sapiens
  J Donor_Short_TRAJ21
ggtctcgtttggatctgggaccaaactcaatgtaaaaccaaatatccagtgagacc

SEQ ID NO: 344 - DNA, 56 bp, Homo sapiens
  J Donor_Short_TRAJ22
ggtctcgtttggatctgggacacaattgactgttttacctgatatccagtgagacc

SEQ ID NO: 345 - DNA, 56 bp, Homo sapiens
  J Donor_Short_TRAJ23
ggtctcgtttggacagggaacggagttatctgtgaaacccaatatccagtgagacc

SEQ ID NO: 346 - DNA, 56 bp, Homo sapiens
  J Donor_Short_TRAJ24
ggtctcgtttggagcagggacccaggttgtggtcaccccagatatccagtgagacc

SEQ ID NO: 347 - DNA, 56 bp, Homo sapiens
  J Donor_Short_TRAJ25
ggtctcgtttgggaaggggacaaggctgcttgtcaagccaaatatccagtgagacc

SEQ ID NO: 348 - DNA, 56 bp, Homo sapiens
  J Donor_Short_TRAJ26
ggtctcgtttggtcccggaaccagattgtccgtgctgccctatatccagtgagacc

SEQ ID NO: 349 - DNA, 56 bp, Homo sapiens
  J Donor_Short_TRAJ27
ggtctcgtttggggatgggactacgctcactgtgaagccaaatatccagtgagacc

SEQ ID NO: 350 - DNA, 56 bp, Homo sapiens
  J Donor_Short_TRAJ28
ggtctcgtttgggaaggggaccaaactctcggtcataccaaatatccagtgagacc

SEQ ID NO: 351 - DNA, 56 bp, Homo sapiens
  J Donor_Short_TRAJ29
ggtctcgtttggaaagggcacaagactttctgtgattgcaaatatccagtgagacc

SEQ ID NO: 352 - DNA, 56 bp, Homo sapiens
  J Donor_Short_TRAJ30
ggtctcgtttggaaaagggacacgacttcatattctccccaatatccagtgagacc

SEQ ID NO: 353 - DNA, 56 bp, Homo sapiens
  J Donor_Short_TRAJ31
ggtctcgtttggagatggaactcagctggtggtgaagcccaatatccagtgagacc

SEQ ID NO: 354 - DNA, 56 bp, Homo sapiens
  J Donor_Short_TRAJ32
ggtctcgtttggaactggcactctgcttgctgtccagccaaatatccagtgagacc

SEQ ID NO: 355 - DNA, 56 bp, Homo sapiens
  J Donor_Short_TRAJ33
ggtctcgtggggcgctgggaccaagctaattataaagccagatatccagtgagacc

SEQ ID NO: 356 - DNA, 56 bp, Homo sapiens
  J Donor_Short_TRAJ34
ggtctcgtttgggactgggaccagattacaagtctttccaaatatccagtgagacc

SEQ ID NO: 357 - DNA, 56 bp, Homo sapiens
  J Donor_Short_TRAJ36
ggtctcgtttgggactggaacgagactcaccgttattccctatatccagtgagacc

SEQ ID NO: 358 - DNA, 56 bp, Homo sapiens
  J Donor_Short_TRAJ37
ggtctcgtttgggcaagggacaactttacaagtaaaaccagatatccagtgagacc

SEQ ID NO: 359 - DNA, 56 bp, Homo sapiens
  J Donor_Short_TRAJ38
ggtctcgtggggattgggaacaagcctggcagtaaatccgaatatccagtgagacc

SEQ ID NO: 360 - DNA, 56 bp, Homo sapiens
  J Donor_Short_TRAJ39
ggtctcgtttggaggaggaacaaggttaatggtcaaaccccatatccagtgagacc

SEQ ID NO: 361 - DNA, 56 bp, Homo sapiens
  J Donor_Short_TRAJ40
ggtctcgtttggaacaggcaccaggctgaaggttttagcaaatatccagtgagacc

SEQ ID NO: 362 - DNA, 56 bp, Homo sapiens
  J Donor_Short_TRAJ41
ggtctcgtttggcaaaggcacctcgctgttggtcacaccccatatccagtgagacc

SEQ ID NO: 363 - DNA, 56 bp, Homo sapiens
  J Donor_Short_TRAJ42
ggtctcgtttggaaaaggcactaaactctctgttaaaccaaatatccagtgagacc

SEQ ID NO: 364 - DNA, 56 bp, Homo sapiens
  J Donor_Short_TRAJ43
ggtctcgtttggagcagggaccagactgacagtaaaaccaaatatccagtgagacc

SEQ ID NO: 365 - DNA, 56 bp, Homo sapiens
  J Donor_Short_TRAJ44
ggtctcgtttgggactggaacaagacttcaggtcacgctcgatatccagtgagacc

SEQ ID NO: 366 - DNA, 56 bp, Homo sapiens
  J Donor_Short_TRAJ45
ggtctcgtttggcaaagggactcatctaatcatccagccctatatccagtgagacc

SEQ ID NO: 367 - DNA, 56 bp, Homo sapiens
  J Donor_Short_TRAJ46
ggtctcgtttgggaccgggactcgtttagcagttaggcccaatatccagtgagacc

SEQ ID NO: 368 - DNA, 56 bp, Homo sapiens
  J Donor_Short_TRAJ47
ggtctcgtttggcgcaggaaccattctgagagtcaagtcctatatccagtgagacc

SEQ ID NO: 369 - DNA, 56 bp, Homo sapiens
  J Donor_Short_TRAJ48
ggtctcgtttgggactggaacaagactcaccatcatacccaatatccagtgagacc

SEQ ID NO: 370 - DNA, 56 bp, Homo sapiens
  J Donor_Short_TRAJ49
ggtctcgtttgggacagggacaagtttgacggtcattccaaatatccagtgagacc

SEQ ID NO: 371 - DNA, 56 bp, Homo sapiens
  J Donor_Short_TRAJ50
ggtctcgtttgggccagggacaagcttatcagtcattccaaatatccagtgagacc

SEQ ID NO: 372 - DNA, 56 bp, Homo sapiens
  J Donor_Short_TRAJ52
ggtctcgtttggacaagggaccatcttgactgtccatccaaatatccagtgagacc

SEQ ID NO: 373 - DNA, 56 bp, Homo sapiens
  J Donor_Short_TRAJ53
ggtctcgtttggaaaaggaactctcttaaccgtgaatccaaatatccagtgagacc

SEQ ID NO: 374 - DNA, 56 bp, Homo sapiens
  J Donor_Short_TRAJ54
ggtctcgtttggccaaggaaccaggctgactatcaacccaaatatccagtgagacc

SEQ ID NO: 375 - DNA, 56 bp, Homo sapiens
  J Donor_Short_TRAJ56
ggtctcgtttggaaaaggaataactctgagtgttagaccagatatccagtgagacc

SEQ ID NO: 376 - DNA, 56 bp, Homo sapiens
  J Donor_Short_TRAJ57
ggtctcgtttggaaagggaacgaaactgacagtaaacccatatatccagtgagacc

SEQ ID NO: 377 - DNA, 50 bp, Homo sapiens
  J Donor_Short_TRAJ59
ggtctcgtttggaatggggacgcaagtgagagtgaatatccagtgagacc

SEQ ID NO: 378 - DNA, 56 bp, Homo sapiens
  J Donor_Short_TRAJ61
ggtctcgtttggagccaacactagaggaatcatgaaactcaatatccagtgagacc

SEQ ID NO: 379 - DNA, 74 bp, Homo sapiens
  J Donor_Long_TRAJ1
ggtctcgattacctcccagttgcaatttggcaaaggaaccagagtttccacttctccccatatccagtgagacc

SEQ ID NO: 380 - DNA, 77 bp, Homo sapiens
  J Donor_Long_TRAJ2
ggtctcgggaacaattgataaactcacatttgggaaagggacccatgtattcattatatctgatatccagtgagacc

SEQ ID NO: 381 - DNA, 74 bp, Homo sapiens
  J Donor_Long_TRAJ3
ggtctcgagtgcttccaagataatctttggatcagggaccagactcagcatccggccaaatatccagtgagacc

SEQ ID NO: 382 - DNA, 74 bp, Homo sapiens
  J Donor_Long_TRAJ4
ggtctcgggctacaataagctgatctttggagcagggaccaggctggctgtacacccatatatccagtgagacc

SEQ ID NO: 383 - DNA, 71 bp, Homo sapiens
  J Donor_Long_TRAJ5
ggtctcgaggagagcacttacttttgggagtggaacaagactccaagtgcaaccaaatatccagtgagacc

SEQ ID NO: 384 - DNA, 74 bp, Homo sapiens
  J Donor_Long_TRAJ6
ggtctcgggaagctacatacctacatttggaagaggaaccagccttattgttcatccgtatatccagtgagacc

SEQ ID NO: 385 - DNA, 71 bp, Homo sapiens
  J Donor_Long_TRAJ7
ggtctcgaacaacagactcgcttttgggaaggggaaccaagtggtggtcataccaaatatccagtgagacc

SEQ ID NO: 386 - DNA, 71 bp, Homo sapiens
  J Donor_Long_TRAJ8
ggtctcgtttcagaaacttgtatttggaactggcacccgacttctggtcagtccaaatatccagtgagacc

SEQ ID NO: 387 - DNA, 71 bp, Homo sapiens
  J Donor_Long_TRAJ9
ggtctcgggcttcaaaactatctttggagcaggaacaagactatttgttaaagcaaatatccagtgagacc

SEQ ID NO: 388 - DNA, 74 bp, Homo sapiens
  J Donor_Long_TRAJ10
ggtctcgggaggaaacaaactcacctttgggacaggcactcagctaaaagtggaactcaatatccagtgagacc

SEQ ID NO: 389 - DNA, 71 bp, Homo sapiens
  J Donor_Long_TRAJ11
ggtctcgtacagcaccctcacctttgggaaggggactatgcttctagtctctccagatatccagtgagacc

SEQ ID NO: 390 - DNA, 71 bp, Homo sapiens
  J Donor_Long_TRAJ12
ggtctcgagttataaattgatctttgggagtgggaccagactgctggtcaggcctgatatccagtgagacc

SEQ ID NO: 391 - DNA, 74 bp, Homo sapiens
  J Donor_Long_TRAJ13
ggtctcgggttaccagaaagttacctttggaattggaacaaagctccaagtcatcccaaatatccagtgagacc

SEQ ID NO: 392 - DNA, 62 bp, Homo sapiens
  J Donor_Long_TRAJ14
ggtctcgttcatctttgggagtgggacaagattatcagtaaaacctgatatccagtgagacc

SEQ ID NO: 393 - DNA, 71 bp, Homo sapiens
  J Donor_Long_TRAJ15
ggtctcgggaactgctctgatctttgggaagggaacccacctatcagtgagttccaatatccagtgagacc

SEQ ID NO: 394 - DNA, 71 bp, Homo sapiens
  J Donor_Long_TRAJ16
ggtctcgggacagaagctgctctttgcaaggggaaccatgttaaaggtggatcttaatatccagtgagacc

SEQ ID NO: 395 - DNA, 74 bp, Homo sapiens
  J Donor_Long_TRAJ17
ggtctcggcaggcaacaagctaacttttggaggaggaaccagggtgctagttaaaccaaatatccagtgagacc

SEQ ID NO: 396 - DNA, 77 bp, Homo sapiens
  J Donor_Long_TRAJ18
ggtctcgtcaaccctggggaggctatactttggaagaggaactcagttgactgtctggcctgatatccagtgagacc

SEQ ID NO: 397 - DNA, 71 bp, Homo sapiens
  J Donor_Long_TRAJ19
ggtctcgttctacaatttcacctttggaaagggatccaaacataatgtcactccaaatatccagtgagacc

SEQ ID NO: 398 - DNA, 68 bp, Homo sapiens
  J Donor_Long_TRAJ20
ggtctcgtacaagctcagctttggagccggaaccacagtaactgtaagagcaaatatccagtgagacc

SEQ ID NO: 399 - DNA, 65 bp, Homo sapiens
  J Donor_Long_TRAJ21
ggtctcgaaattttactttggatctgggaccaaactcaatgtaaaaccaaatatccagtgagacc

SEQ ID NO: 400 - DNA, 74 bp, Homo sapiens
  J Donor_Long_TRAJ22
ggtctcgtctgcaaggcaactgacctttggatctgggacacaattgactgttttacctgatatccagtgagacc

SEQ ID NO: 401 - DNA, 74 bp, Homo sapiens
  J Donor_Long_TRAJ23
ggtctcgcagggaggaaagcttatctttggacagggaacggagttatctgtgaaacccaatatccagtgagacc

SEQ ID NO: 402 - DNA, 74 bp, Homo sapiens
  J Donor_Long_TRAJ24
ggtctcgagttggggtaaattgcagtttggagcagggacccaggttgtggtcaccccagatatccagtgagacc

SEQ ID NO: 403 - DNA, 71 bp, Homo sapiens
  J Donor_Long_TRAJ25
ggtctcgggcttctcctttatctttgggaaggggacaaggctgcttgtcaagccaaatatccagtgagacc

SEQ ID NO: 404 - DNA, 71 bp, Homo sapiens
  J Donor_Long_TRAJ26
ggtctcgggtcagaattttgtctttggtcccggaaccagattgtccgtgctgccctatatccagtgagacc

SEQ ID NO: 405 - DNA, 71 bp, Homo sapiens
  J Donor_Long_TRAJ27
ggtctcggcaggcaaatcaacctttggggatgggactacgctcactgtgaagccaaatatccagtgagacc

SEQ ID NO: 406 - DNA, 77 bp, Homo sapiens
  J Donor_Long_TRAJ28
ggtctcggctgggagttaccaactcacttttgggaaggggaccaaactctcggtcataccaaatatccagtgagacc

SEQ ID NO: 407 - DNA, 71 bp, Homo sapiens
  J Donor_Long_TRAJ29
ggtctcgaacacacctcttgtctttggaaagggcacaagactttctgtgattgcaaatatccagtgagacc

SEQ ID NO: 408 - DNA, 68 bp, Homo sapiens
  J Donor_Long_TRAJ30
ggtctcggacaagatcatctttggaaaagggacacgacttcatattctccccaatatccagtgagacc

SEQ ID NO: 409 - DNA, 68 bp, Homo sapiens
  J Donor_Long_TRAJ31
ggtctcggccagactcatgtttggagatggaactcagctggtggtgaagcccaatatccagtgagacc

SEQ ID NO: 410 - DNA, 77 bp, Homo sapiens
  J Donor_Long_TRAJ32
ggtctcgggtgctacaaacaagctcatctttggaactggcactctgcttgctgtccagccaaatatccagtgagacc

SEQ ID NO: 411 - DNA, 68 bp, Homo sapiens
  J Donor_Long_TRAJ33
ggtctcgtatcagttaatctggggcgctgggaccaagctaattataaagccagatatccagtgagacc

SEQ ID NO: 412 - DNA, 68 bp, Homo sapiens
  J Donor_Long_TRAJ34
ggtctcggacaagctcatctttgggactgggaccagattacaagtctttccaaatatccagtgagacc

SEQ ID NO: 413 - DNA, 71 bp, Homo sapiens
  J Donor_Long_TRAJ36
ggtctcggcaaacaacctcttctttgggactggaacgagactcaccgttattccctatatccagtgagacc

SEQ ID NO: 414 - DNA, 74 bp, Homo sapiens
  J Donor_Long_TRAJ37
ggtctcgaacacaggcaaactaatctttgggcaagggacaactttacaagtaaaaccagatatccagtgagacc

SEQ ID NO: 415 - DNA, 74 bp, Homo sapiens
  J Donor_Long_TRAJ38
ggtctcgaacaaccgtaagctgatttggggattgggaacaagcctggcagtaaatccgaatatccagtgagacc

SEQ ID NO: 416 - DNA, 74 bp, Homo sapiens
  J Donor_Long_TRAJ39
ggtctcggcaggcaacatgctcacctttggaggaggaacaaggttaatggtcaaaccccatatccagtgagacc

SEQ ID NO: 417 - DNA, 71 bp, Homo sapiens
  J Donor_Long_TRAJ40
ggtctcgacctacaaatacatctttggaacaggcaccaggctgaaggttttagcaaatatccagtgagacc

SEQ ID NO: 418 - DNA, 74 bp, Homo sapiens
  J Donor_Long_TRAJ41
ggtctcgtccgggtatgcactcaactttggcaaaggcacctcgctgttggtcacaccccatatccagtgagacc

SEQ ID NO: 419 - DNA, 77 bp, Homo sapiens
  J Donor_Long_TRAJ42
ggtctcgggaagccaaggaaatctcatctttggaaaaggcactaaactctctgttaaaccaaatatccagtgagacc

SEQ ID NO: 420 - DNA, 65 bp, Homo sapiens
  J Donor_Long_TRAJ43
ggtctcggacatgcgctttggagcagggaccagactgacagtaaaaccaaatatccagtgagacc

SEQ ID NO: 421 - DNA, 74 bp, Homo sapiens
  J Donor_Long_TRAJ44
ggtctcgactgccagtaaactcacctttgggactggaacaagacttcaggtcacgctcgatatccagtgagacc

SEQ ID NO: 422 - DNA, 77 bp, Homo sapiens
  J Donor_Long_TRAJ45
ggtctcgggaggtgctgacggactcacctttggcaaagggactcatctaatcatccagccctatatccagtgagacc

SEQ ID NO: 423 - DNA, 74 bp, Homo sapiens
  J Donor_Long_TRAJ46
ggtctcgagcggagacaagctgacttttgggaccgggactcgtttagcagttaggcccaatatccagtgagacc

SEQ ID NO: 424 - DNA, 68 bp, Homo sapiens
  J Donor_Long_TRAJ47
ggtctcgaacaaactggtctttggcgcaggaaccattctgagagtcaagtcctatatccagtgagacc

SEQ ID NO: 425 - DNA, 74 bp, Homo sapiens
  J Donor_Long_TRAJ48
ggtctcgggaaatgagaaattaacctttgggactggaacaagactcaccatcatacccaatatccagtgagacc

SEQ ID NO: 426 - DNA, 68 bp, Homo sapiens
  J Donor_Long_TRAJ49
ggtctcgaaccagttctattttgggacagggacaagtttgacggtcattccaaatatccagtgagacc

SEQ ID NO: 427 - DNA, 71 bp, Homo sapiens
  J Donor_Long_TRAJ50
ggtctcgtacgacaaggtgatatttgggccagggacaagcttatcagtcattccaaatatccagtgagacc

SEQ ID NO: 428 - DNA, 80 bp, Homo sapiens
  J Donor_Long_TRAJ52
ggtctcgggtactagctatggaaagctgacatttggacaagggaccatcttgactgtccatccaaatatccagtgagacc

SEQ ID NO: 429 - DNA, 77 bp, Homo sapiens
  J Donor_Long_TRAJ53
ggtctcgggtagcaactataaactgacatttggaaaaggaactctcttaaccgtgaatccaaatatccagtgagacc

SEQ ID NO: 430 - DNA, 71 bp, Homo sapiens
  J Donor_Long_TRAJ54
ggtctcggcccagaagctggtatttggccaaggaaccaggctgactatcaacccaaatatccagtgagacc

SEQ ID NO: 431 - DNA, 74 bp, Homo sapiens
  J Donor_Long_TRAJ56
ggtctcggccaatagtaagctgacatttggaaaaggaataactctgagtgttagaccagatatccagtgagacc

SEQ ID NO: 432 - DNA, 74 bp, Homo sapiens
  J Donor_Long_TRAJ57
ggtctcgggatctgaaaagctggtctttggaaagggaacgaaactgacagtaaacccatatatccagtgagacc

SEQ ID NO: 433 - DNA, 65 bp, Homo sapiens
  J Donor_Long_TRAJ59
ggtctcgaacaggaaatttacatttggaatggggacgcaagtgagagtgaatatccagtgagacc

SEQ ID NO: 434 - DNA, 71 bp, Homo sapiens
  J Donor_Long_TRAJ61
ggtctcgaataggaaactgacatttggagccaacactagaggaatcatgaaactcaatatccagtgagacc

SEQ ID NO: 435 - DNA, 414 bp, Homo sapiens
  TRBV2_BB_1
gttcgaagctggcatgacacgaagacttgtacgccaccatggatacctggctcgtatgctgggcaatttttagtctcttgaaagcaggactcacagaacctgaagtcacccagactcccagccatcaggtcacacagatgggacaggaagtgatcttgcgctgtgtccccatctctaatcacttatacttctattggtacagacaaatcttggggcagaaagtcgagtttctggtttccttttataataatgaaatctcagagaagtctgaaatattcgatgatcaattctcagttgaaaggcctgatggatcaaatttcactctgaagatccggtccacaaagctggaggactcagccatgtacttttgcagagaccttgcggccgtgtcttccgacgctgacagtgtagata

SEQ ID NO: 436 - DNA, 411 bp, Homo sapiens
  TRBV3-1_BB_1
gttcgaagctggcatgacacgaagacttgtacgccaccatgggctgcaggctcctctgctgtgtggtcttttgcctcctccaagcaggtcccttggacacagctgtttcccagactccaaaatacctggtcacacagatgggaaacgacaagtccattaaatgtgaacaaaatctgggccatgatactatgtattggtataaacaggactctaagaaatttctgaagataatgtttagctacaataataaggagctcattataaatgaaacagttccaaatcgcttctcacctaaatctccagacaaagctcacttaaatcttcacatcaattccctggagcttggtgactctgctgtgtatttttgcagagaccttgcggccgtgtcttccgacgctgacagtgtagata

SEQ ID NO: 437 - DNA, 411 bp, Homo sapiens
  TRBV4-1_BB_1
gttcgaagctggcatgacacgaagacttgtacgccaccatgggctgcaggctgctctgctgtgcggttctctgtctcctgggagcagttcccatagacactgaagttacccagacaccaaaacacctggtcatgggaatgacaaataagaagtctttgaaatgtgaacaacatatggggcacagggctatgtattggtacaagcagaaagctaagaagccaccggagctcatgtttgtctacagctatgagaaactctctataaatgaaagtgtgccaagtcgcttctcacctgaatgccccaacagctctctcttaaaccttcacctacacgccctgcagccagaagattcagccctgtatctttgcagagaccttgcggccgtgtcttccgacgctgacagtgtagata

SEQ ID NO: 438 - DNA, 411 bp, Homo sapiens
  TRBV4-2_BB_1
gttcgaagctggcatgacacgaagacttgtacgccaccatgggctgcaggctgctctgctgtgcggttctctgtctcctgggagcggtccccatggaaacgggagttacgcagacaccaagacacctggtcatgggaatgacaaataagaagtctttgaaatgtgaacaacatctggggcataacgctatgtattggtacaagcaaagtgctaagaagccactggagctcatgtttgtctacaactttaaagaacagactgaaaacaacagtgtgccaagtcgcttctcacctgaatgccccaacagctctcacttattccttcacctacacaccctgcagccagaagattcggccctgtatctttgcagagaccttgcggccgtgtcttccgacgctgacagtgtagata

SEQ ID NO: 439 - DNA, 411 bp, Homo sapiens
  TRBV4-3_BB_1
gttcgaagctggcatgacacgaagacttgtacgccaccatgggctgcaggctgctctgctgtgcggttctctgtctcctgggagcggtccccatggaaacgggagttacgcagacaccaagacacctggtcatgggaatgacaaataagaagtctttgaaatgtgaacaacatctgggtcataacgctatgtattggtacaagcaaagtgctaagaagccactggagctcatgtttgtctacagtcttgaagaacgggttgaaaacaacagtgtgccaagtcgcttctcacctgaatgccccaacagctctcacttattccttcacctacacaccctgcagccagaagattcggccctgtatctttgcagagaccttgcggccgtgtcttccgacgctgacagtgtagata

SEQ ID NO: 440 - DNA, 411 bp, Homo sapiens
  TRBV5-1_BB_1
gttcgaagctggcatgacacgaagacttgtacgccaccatgggctccaggctgctctgttgggtgctgctttgtctcctgggagcaggcccagtaaaggctggagtcactcaaactccaagatatctgatcaaaacgagaggacagcaagtgacactgagctgctcccctatctctgggcataggagtgtatcctggtaccaacagaccccaggacagggccttcagttcctctttgaatacttcagtgagacacagagaaacaaaggaaacttccctggtcgattctcagggcgccagttctctaactctcgctctgagatgaatgtgagcaccttggagctgggggactcggccctttatctttgcagagaccttgcggccgtgtcttccgacgctgacagtgtagata

SEQ ID NO: 441 - DNA, 411 bp, Homo sapiens
  TRBV5-4_BB_1
gttcgaagctggcatgacacgaagacttgtacgccaccatgggccctgggctcctctgctgggtgctgctttgtctcctgggagcaggctcagtggagactggagtcacccaaagtcccacacacctgatcaaaacgagaggacagcaagtgactctgagatgctcttctcagtctgggcacaacactgtgtcctggtaccaacaggccctgggtcaggggccccagtttatctttcagtattatagggaggaagagaatggcagaggaaacttccctcctagattctcaggactccagttccctaattatagctctgagctgaatgtgaacgccttggagctggacgactcggccctgtatctttgcagagaccttgcggccgtgtcttccgacgctgacagtgtagata

SEQ ID NO: 442 - DNA, 411 bp, Homo sapiens
  TRBV5-5_BB_1
gttcgaagctggcatgacacgaagacttgtacgccaccatgggccctgggctcctctgctgggtgctgctttgtctcctgggagcaggcccagtggacgctggagtcacccaaagtcccacacacctgatcaaaacgagaggacagcaagtgactctgagatgctctcctatctctgggcacaagagtgtgtcctggtaccaacaggtcctgggtcaggggccccagtttatctttcagtattatgagaaagaagagagaggaagaggaaacttccctgatcgattctcagctcgccagttccctaactatagctctgagctgaatgtgaacgccttgttgctgggggactcggccctgtatctttgcagagaccttgcggccgtgtcttccgacgctgacagtgtagata

SEQ ID NO: 443 - DNA, 411 bp, Homo sapiens
  TRBV5-6_BB_1
gttcgaagctggcatgacacgaagacttgtacgccaccatgggccccgggctcctctgctgggcactgctttgtctcctgggagcaggcttagtggacgctggagtcacccaaagtcccacacacctgatcaaaacgagaggacagcaagtgactctgagatgctctcctaagtctgggcatgacactgtgtcctggtaccaacaggccctgggtcaggggccccagtttatctttcagtattatgaggaggaagagagacagagaggcaacttccctgatcgattctcaggtcaccagttccctaactatagctctgagctgaatgtgaacgccttgttgctgggggactcggccctctatctttgcagagaccttgcggccgtgtcttccgacgctgacagtgtagata

SEQ ID NO: 444 - DNA, 411 bp, Homo sapiens
  TRBV5-7_BB_1
gttcgaagctggcatgacacgaagacttgtacgccaccatgggccccgggctcctctgctgggtgctgctttgtcccctaggagaaggcccagtggacgctggagtcacccaaagtcccacacacctgatcaaaacgagaggacagcacgtgactctgagatgctctcctatctctgggcacaccagtgtgtcctcgtaccaacaggccctgggtcaggggccccagtttatctttcagtattatgagaaagaagagagaggaagaggaaacttccctgatcaattctcaggtcaccagttccctaactatagctctgagctgaatgtgaacgccttgttgctaggggactcggccctctatctttgcagagaccttgcggccgtgtcttccgacgctgacagtgtagata

SEQ ID NO: 445 - DNA, 411 bp, Homo sapiens
  TRBV5-8_BB_1
gttcgaagctggcatgacacgaagacttgtacgccaccatgggacccaggctcctcttctgggcactgctttgtctcctcggaacaggcccagtggaggctggagtcacacaaagtcccacacacctgatcaaaacgagaggacagcaagcgactctgagatgctctcctatctctgggcacaccagtgtgtactggtaccaacaggccctgggtctgggcctccagttcctcctttggtatgacgagggtgaagagagaaacagaggaaacttccctcctagattttcaggtcgccagttccctaattatagctctgagctgaatgtgaacgccttggagctggaggactcggccctgtatctttgcagagaccttgcggccgtgtcttccgacgctgacagtgtagata

SEQ ID NO: 446 - DNA, 411 bp, Homo sapiens
  TRBV6-1_BB_1
gttcgaagctggcatgacacgaagacttgtacgccaccatgagcatcgggctcctgtgctgtgtggccttttctctcctgtgggcaagtccagtgaatgctggtgtcactcagaccccaaaattccaggtcctgaaaacaggacagagcatgacactgcagtgtgcccaggatatgaaccataactccatgtactggtatcgacaagacccaggcatgggactgaggctgatttattactcagcttctgagggtaccactgacaaaggagaagtccccaatggctacaatgtctccagattaaacaaacgggagttctcgctcaggctggagtcggctgctccctcccagacatctgtgtacttttgcagagaccttgcggccgtgtcttccgacgctgacagtgtagata

SEQ ID NO: 447 - DNA, 411 bp, Homo sapiens
  TRBV6-3_BB_1
gttcgaagctggcatgacacgaagacttgtacgccaccatgagcctcgggctcctgtgctgtggggtcttttctctcctgtgggcaggtccagtgaatgctggtgtcactcagaccccaaaattccgggtcctgaaaacaggacagagcatgacactgctgtgtgcccaggatatgaaccatgaatacatgtactggtatcgacaagacccaggcatggggctgaggctgattcattactcagttggtgagggtacaactgccaaaggagaggtccctgatggctacaatgtctccagattaaaaaaacagaatttcctgctggggttggagtcggctgctccctcccaaacatctgtgtacttttgcagagaccttgcggccgtgtcttccgacgctgacagtgtagata

SEQ ID NO: 448 - DNA, 411 bp, Homo sapiens
  TRBV6-4_BB_1
gttcgaagctggcatgacacgaagacttgtacgccaccatgagaatcaggctcctgtgctgtgtggccttttctctcctgtgggcaggtccagtgattgctgggatcacccaggcaccaacatctcagatcctggcagcaggacggcgcatgacactgagatgtacccaggatatgagacataatgccatgtactggtatagacaagatctaggactggggctaaggctcatccattattcaaatactgcaggtaccactggcaaaggagaagtccctgatggttatagtgtctccagagcaaacacagatgatttccccctcacgttggcgtctgctgtaccctctcagacatctgtgtacttttgcagagaccttgcggccgtgtcttccgacgctgacagtgtagata

SEQ ID NO: 449 - DNA, 411 bp, Homo sapiens
  TRBV6-5_BB_1
gttcgaagctggcatgacacgaagacttgtacgccaccatgagcatcggcctcctgtgctgtgcagccttgtctctcctgtgggcaggtccagtgaatgctggtgtcactcagaccccaaaattccaggtcctgaaaacaggacagagcatgacactgcagtgtgcccaggatatgaaccatgaatacatgtcctggtatcgacaagacccaggcatggggctgaggctgattcattactcagttggtgctggtatcactgaccaaggagaagtccccaatggctacaatgtctccagatcaaccacagaggatttcccgctcaggctgctgtcggctgctccctcccagacatctgtgtacttttgcagagaccttgcggccgtgtcttccgacgctgacagtgtagata

SEQ ID NO: 450 - DNA, 411 bp, Homo sapiens
  TRBV6-6_BB_1
gttcgaagctggcatgacacgaagacttgtacgccaccatgagcatcagcctcctgtgctgtgcagcctttcctctcctgtgggcaggtccagtgaatgctggtgtcactcagaccccaaaattccgcatcctgaagataggacagagcatgacactgcagtgtacccaggatatgaaccataactacatgtactggtatcgacaagacccaggcatggggctgaagctgatttattattcagttggtgctggtatcactgataaaggagaagtcccgaatggctacaacgtctccagatcaaccacagaggatttcccgctcaggctggagttggctgctccctcccagacatctgtgtacttttgcagagaccttgcggccgtgtcttccgacgctgacagtgtagata

SEQ ID NO: 451 - DNA, 408 bp, Homo sapiens
  TRBV6-8_BB_1
gttcgaagctggcatgacacgaagacttgtacgccaccatgagcctcgggctcctgtgctgtgcggccttttctctcctgtgggcaggtcccgtgaatgctggtgtcactcagaccccaaaattccacatcctgaaaacaggacagagcatgacactgcagtgtgcccaggatatgaaccatggatacatgtcctggtatcgacaagacccaggcatggggctgagactgatttactactcagctgctgctggtactactgacaaagaagtccccaatggctacaatgtctctagattaaacacagaggatttcccactcaggctggtgtcggctgctccctcccagacatctgtgtacctttgcagagaccttgcggccgtgtcttccgacgctgacagtgtagata

SEQ ID NO: 452 - DNA, 411 bp, Homo sapiens
  TRBV6-9_BB_1
gttcgaagctggcatgacacgaagacttgtacgccaccatgagcatcgggctcctgtgctgtgtggccttttctctcctgtgggcaggtccagtgaatgctggtgtcactcagaccccaaaattccacatcctgaaaacaggacagagcatgacactgcagtgtgcccaggatatgaaccatggatacttgtcctggtatcgacaagacccaggcatggggctgaggcgcattcattactcagttgctgctggtatcactgacaaaggagaagtccccgatggctacaatgtatccagatcaaacacagaggatttcccgctcaggctggagtcagctgctccctcccagacatctgtatacttttgcagagaccttgcggccgtgtcttccgacgctgacagtgtagata

SEQ ID NO: 453 - DNA, 414 bp, Homo sapiens
  TRBV7-2_BB_1
gttcgaagctggcatgacacgaagacttgtacgccaccatgggcaccaggctcctcttctgggtggccttctgtctcctgggggcagatcacacaggagctggagtctcccagtcccccagtaacaaggtcacagagaagggaaaggatgtagagctcaggtgtgatccaatttcaggtcatactgccctttactggtaccgacagagcctggggcagggcctggagtttttaatttacttccaaggcaacagtgcaccagacaaatcagggctgcccagtgatcgcttctctgcagagaggactgggggatccgtctccactctgacgatccagcgcacacagcaggaggactcggccgtgtatctttgcagagaccttgcggccgtgtcttccgacgctgacagtgtagata

SEQ ID NO: 454 - DNA, 414 bp, Homo sapiens
  TRBV7-3_BB_1
gttcgaagctggcatgacacgaagacttgtacgccaccatgggcaccaggctcctctgctgggcagccctgtgcctcctgggggcagatcacacaggtgctggagtctcccagacccccagtaacaaggtcacagagaagggaaaatatgtagagctcaggtgtgatccaatttcaggtcatactgccctttactggtaccgacaaagcctggggcagggcccagagtttctaatttacttccaaggcacgggtgcggcagatgactcagggctgcccaacgatcggttctttgcagtcaggcctgagggatccgtctctactctgaagatccagcgcacagagcggggggactcagccgtgtatctttgcagagaccttgcggccgtgtcttccgacgctgacagtgtagata

SEQ ID NO: 455 - DNA, 414 bp, Homo sapiens
  TRBV7-6_BB_1
gttcgaagctggcatgacacgaagacttgtacgccaccatgggcaccagtctcctatgctgggtggtcctgggtttcctagggacagatcacacaggtgctggagtctcccagtctcccaggtacaaagtcacaaagaggggacaggatgtagctctcaggtgtgatccaatttcgggtcatgtatccctttattggtaccgacaggccctggggcagggcccagagtttctgacttacttcaattatgaagcccaacaagacaaatcagggctgcccaatgatcggttctctgcagagaggcctgagggatccatctccactctgacgatccagcgcacagagcagcgggactcggccatgtatcgttgcagagaccttgcggccgtgtcttccgacgctgacagtgtagata

SEQ ID NO: 456 - DNA, 414 bp, Homo sapiens
  TRBV7-7_BB_1
gttcgaagctggcatgacacgaagacttgtacgccaccatgggtaccagtctcctatgctgggtggtcctgggtttcctagggacagatcacacaggtgctggagtctcccagtctcccaggtacaaagtcacaaagaggggacaggatgtaactctcaggtgtgatccaatttcgagtcatgcaaccctttattggtatcaacaggccctggggcagggcccagagtttctgacttacttcaattatgaagctcaaccagacaaatcagggctgcccagtgatcggttctctgcagagaggcctgagggatccatctccactctgacgattcagcgcacagagcagcgggactcagccatgtatcgttgcagagaccttgcggccgtgtcttccgacgctgacagtgtagata

SEQ ID NO: 457 - DNA, 414 bp, Homo sapiens
  TRBV7-8_BB_1
gttcgaagctggcatgacacgaagacttgtacgccaccatgggcaccaggctcctctgctgggtggtcctgggtttcctagggacagatcacacaggtgctggagtctcccagtcccctaggtacaaagtcgcaaagagaggacaggatgtagctctcaggtgtgatccaatttcgggtcatgtatcccttttttggtaccaacaggccctggggcaggggccagagtttctgacttatttccagaatgaagctcaactagacaaatcggggctgcccagtgatcgcttctttgcagaaaggcctgagggatccgtctccactctgaagatccagcgcacacagcaggaggactccgccgtgtatctttgcagagaccttgcggccgtgtcttccgacgctgacagtgtagata

SEQ ID NO: 458 - DNA, 414 bp, Homo sapiens
  TRBV7-9_BB_1
gttcgaagctggcatgacacgaagacttgtacgccaccatgggcaccagcctcctctgctggatggccctgtgtctcctgggggcagatcacgcagatactggagtctcccagaaccccagacacaagatcacaaagaggggacagaatgtaactttcaggtgtgatccaatttctgaacacaaccgcctttattggtaccgacagaccctggggcagggcccagagtttctgacttacttccagaatgaagctcaactagaaaaatcaaggctgctcagtgatcggttctctgcagagaggcctaagggatctttctccaccttggagatccagcgcacagagcagggggactcggccatgtatctttgcagagaccttgcggccgtgtcttccgacgctgacagtgtagata

SEQ ID NO: 459 - DNA, 411 bp, Homo sapiens
  TRBV9_BB_1
gttcgaagctggcatgacacgaagacttgtacgccaccatgggcttcaggctcctctgctgtgtggccttttgtctcctgggagcaggcccagtggattctggagtcacacaaaccccaaagcacctgatcacagcaactggacagcgagtgacgctgagatgctcccctaggtctggtgacctctctgtgtactggtaccaacagagcctggaccagggcctccagttcctcattcagtattataatggagaagagagagcaaaaggaaacattcttgaacgattctccgcacaacagttccctgacttgcactctgaactaaacctgagctctctggagctgggggactcagctttgtacttttgcagagaccttgcggccgtgtcttccgacgctgacagtgtagata

SEQ ID NO: 460 - DNA, 411 bp, Homo sapiens
  TRBV10-1_BB_1
gttcgaagctggcatgacacgaagacttgtacgccaccatgggcacgaggctcttcttctatgtggccctttgtctgctgtgggcaggacacagggatgctgaaatcacccagagcccaagacacaagatcacagagacaggaaggcaggtgaccttggcgtgtcaccagacttggaaccacaacaatatgttctggtatcgacaagacctgggacatgggctgaggctgatccattactcatatggtgttcaagacactaacaaaggagaagtctcagatggctacagtgtctctagatcaaacacagaggacctccccctcactctggagtctgctgcctcctcccagacatctgtatacttttgcagagaccttgcggccgtgtcttccgacgctgacagtgtagata

SEQ ID NO: 461 - DNA, 411 bp, Homo sapiens
  TRBV10-2_BB_1
gttcgaagctggcatgacacgaagacttgtacgccaccatgggcaccaggctcttcttctatgtggccctttgtctgctgtgggcaggacacagggatgctggaatcacccagagcccaagatacaagatcacagagacaggaaggcaggtgaccttgatgtgtcaccagacttggagccacagctatatgttctggtatcgacaagacctgggacatgggctgaggctgatctattactcagcagctgctgatattacagataaaggagaagtccccgatggctatgttgtctccagatccaagacagagaatttccccctcactctggagtcagctacccgctcccagacatctgtgtacttttgcagagaccttgcggccgtgtcttccgacgctgacagtgtagata

SEQ ID NO: 462 - DNA, 411 bp, Homo sapiens
  TRBV10-3_BB_1
gttcgaagctggcatgacacgaagacttgtacgccaccatgggcacaaggttgttcttctatgtggccctttgtctcctgtggacaggacacatggatgctggaatcacccagagcccaagacacaaggtcacagagacaggaacaccagtgactctgagatgtcaccagactgagaaccaccgctatatgtactggtatcgacaagacccggggcatgggctgaggctgatccattactcatatggtgttaaagatactgacaaaggagaagtctcagatggctatagtgtctctagatcaaagacagaggatttcctcctcactctggagtccgctaccagctcccagacatctgtgtacttttgcagagaccttgcggccgtgtcttccgacgctgacagtgtagata

SEQ ID NO: 463 - DNA, 414 bp, Homo sapiens
  TRBV11-1_BB_1
gttcgaagctggcatgacacgaagacttgtacgccaccatgagcaccaggcttctctgctggatggccctctgtctcctgggggcagaactctcagaagctgaagttgcccagtcccccagatataagattacagagaaaagccaggctgtggctttttggtgtgatcctatttctggccatgctaccctttactggtaccggcagatcctgggacagggcccggagcttctggttcaatttcaggatgagagtgtagtagatgattcacagttgcctaaggatcgattttctgcagagaggctcaaaggagtagactccactctcaagatccagcctgcagagcttggggactcggccatgtatctttgcagagaccttgcggccgtgtcttccgacgctgacagtgtagata

SEQ ID NO: 464 - DNA, 414 bp, Homo sapiens
  TRBV11-2_BB_1
gttcgaagctggcatgacacgaagacttgtacgccaccatgggcaccaggctcctctgctgggcggccctctgtctcctgggagcagaactcacagaagctggagttgcccagtctcccagatataagattatagagaaaaggcagagtgtggctttttggtgcaatcctatatctggccatgctaccctttactggtaccagcagatcctgggacagggcccaaagcttctgattcagtttcagaataacggtgtagtggatgattcacagttgcctaaggatcgattttctgcagagaggctcaaaggagtagactccactctcaagatccagcctgcaaagcttgaggactcggccgtgtatctttgcagagaccttgcggccgtgtcttccgacgctgacagtgtagata

SEQ ID NO: 465 - DNA, 414 bp, Homo sapiens
  TRBV11-3_BB_1
gttcgaagctggcatgacacgaagacttgtacgccaccatgggtaccaggctcctctgctgggtggccttctgtctcctggtggaagaactcatagaagctggagtggttcagtctcccagatataagattatagagaaaaaacagcctgtggctttttggtgcaatcctatttctggccacaataccctttactggtacctgcagaacttgggacagggcccggagcttctgattcgatatgagaatgaggaagcagtagacgattcacagttgcctaaggatcgattttctgcagagaggctcaaaggagtagactccactctcaagatccagcctgcagagcttggggactcggccgtgtatctttgcagagaccttgcggccgtgtcttccgacgctgacagtgtagata

SEQ ID NO: 466 - DNA, 414 bp, Homo sapiens
  TRBV12-3_BB_1
gttcgaagctggcatgacacgaagacttgtacgccaccatggactcctggaccttctgctgtgtgtccctttgcatcctggtagcgaagcatacagatgctggagttatccagtcaccccgccatgaggtgacagagatgggacaagaagtgactctgagatgtaaaccaatttcaggccacaactcccttttctggtacagacagaccatgatgcggggactggagttgctcatttactttaacaacaacgttccgatagatgattcagggatgcccgaggatcgattctcagctaagatgcctaatgcatcattctccactctgaagatccagccctcagaacccagggactcagctgtgtacttttgcagagaccttgcggccgtgtcttccgacgctgacagtgtagata

SEQ ID NO: 467 - DNA, 414 bp, Homo sapiens
  TRBV12-4_BB_1
gttcgaagctggcatgacacgaagacttgtacgccaccatggactcctggaccctctgctgtgtgtccctttgcatcctggtagcaaagcacacagatgctggagttatccagtcaccccggcacgaggtgacagagatgggacaagaagtgactctgagatgtaaaccaatttcaggacacgactaccttttctggtacagacagaccatgatgcggggactggagttgctcatttactttaacaacaacgttccgatagatgattcagggatgcccgaggatcgattctcagctaagatgcctaatgcatcattctccactctgaagatccagccctcagaacccagggactcagctgtgtacttttgcagagaccttgcggccgtgtcttccgacgctgacagtgtagata

SEQ ID NO: 468 - DNA, 414 bp, Homo sapiens
  TRBV12-5_BB_1
gttcgaagctggcatgacacgaagacttgtacgccaccatggccaccaggctcctctgctgtgtggttctttgtctcctgggagaagagcttatagatgctagagtcacccagacaccaaggcacaaggtgacagagatgggacaagaagtaacaatgagatgtcagccaattttaggccacaatactgttttctggtacagacagaccatgatgcaaggactggagttgctggcttacttccgcaaccgggctcctctagatgattcggggatgccgaaggatcgattctcagcagagatgcctgatgcaactttagccactctgaagatccagccctcagaacccagggactcagctgtgtacttttgcagagaccttgcggccgtgtcttccgacgctgacagtgtagata

SEQ ID NO: 469 - DNA, 441 bp, Homo sapiens
  TRBV13_BB_1
gttcgaagctggcatgacacgaagacttgtacgccaccatgcttagtcctgacctgcctgactctgcctggaacaccaggctcctctgccatgtcatgctttgtctcctgggagcagtttcagtggctgctggagtcatccagtccccaagacatctgatcaaagaaaagagggaaacagccactctgaaatgctatcctatccctagacacgacactgtctactggtaccagcagggtccaggtcaggacccccagttcctcatttcgttttatgaaaagatgcagagcgataaaggaagcatccctgatcgattctcagctcaacagttcagtgactatcattctgaactgaacatgagctccttggagctgggggactcagccctgtacttttgcagagaccttgcggccgtgtcttccgacgctgacagtgtagata

SEQ ID NO: 470 - DNA, 414 bp, Homo sapiens
  TRBV14_BB_1
gttcgaagctggcatgacacgaagacttgtacgccaccatggtttccaggcttctcagtttagtgtccctttgtctcctgggagcaaagcacatagaagctggagttactcagttccccagccacagcgtaatagagaagggccagactgtgactctgagatgtgacccaatttctggacatgataatctttattggtatcgacgtgttatgggaaaagaaataaaatttctgttacattttgtgaaagagtctaaacaggatgagtccggtatgcccaacaatcgattcttagctgaaaggactggagggacgtattctactctgaaggtgcagcctgcagaactggaggattctggagtttacttttgcagagaccttgcggccgtgtcttccgacgctgacagtgtagata

SEQ ID NO: 471 - DNA, 411 bp, Homo sapiens
  TRBV15_BB_1
gttcgaagctggcatgacacgaagacttgtacgccaccatgggtcctgggcttctccactggatggccctttgtctccttggaacaggtcatggggatgccatggtcatccagaacccaagataccaggttacccagtttggaaagccagtgaccctgagttgttctcagactttgaaccataacgtcatgtactggtaccagcagaagtcaagtcaggccccaaagctgctgttccactactatgacaaagattttaacaatgaagcagacacccctgataacttccaatccaggaggccgaacacttctttctgctttcttgacatccgctcaccaggcctgggggacacagccatgtacctttgcagagaccttgcggccgtgtcttccgacgctgacagtgtagata

SEQ ID NO: 472 - DNA, 414 bp, Homo sapiens
  TRBV16_BB_1
gttcgaagctggcatgacacgaagacttgtacgccaccatgagcccaatattcacctgcatcacaatcctttgtctgctggctgcaggttctcctggtgaagaagtcgcccagactccaaaacatcttgtcagaggggaaggacagaaagcaaaattatattgtgccccaataaaaggacacagttatgttttttggtaccaacaggtcctgaaaaacgagttcaagttcttgatttccttccagaatgaaaatgtctttgatgaaacaggtatgcccaaggaaagattttcagctaagtgcctcccaaattcaccctgtagccttgagatccaggctacgaagcttgaggattcagcagtgtacttttgcagagaccttgcggccgtgtcttccgacgctgacagtgtagata

SEQ ID NO: 473 - DNA, 414 bp, Homo sapiens
  TRBV18_BB_1
gttcgaagctggcatgacacgaagacttgtacgccaccatggacaccagagtactctgctgtgcggtcatctgccttctgggggcaggactctcaaatgccggcgtcatgcagaacccaagacacctggtcaggaggaggggacaggaggcaagactgagatgcagcccaatgaaaggacacagtcatgtttactggtatcggcagctcccagaggaaggtctgaaattcatggtttatctccagaaagaaaatatcatagatgagtcaggaatgccaaaggaacgattttctgctgaatttcccaaagagggccccagcatcctgaggatccagcaggtagtgcgaggagattcggcagcttacttttgcagagaccttgcggccgtgtcttccgacgctgacagtgtagata

SEQ ID NO: 474 - DNA, 411 bp, Homo sapiens
  TRBV19_BB_1
gttcgaagctggcatgacacgaagacttgtacgccaccatgagcaaccaggtgctctgctgtgtggtcctttgtttcctgggagcaaacaccgtggatggtggaatcactcagtccccaaagtacctgttcagaaaggaaggacagaatgtgaccctgagttgtgaacagaatttgaaccacgatgccatgtactggtaccgacaggacccagggcaagggctgagattgatctactactcacagatagtaaatgactttcagaaaggagatatagctgaagggtacagcgtctctcgggagaagaaggaatcctttcctctcactgtgacatcggcccaaaagaacccgacagctttctatctttgcagagaccttgcggccgtgtcttccgacgctgacagtgtagata

SEQ ID NO: 475 - DNA, 405 bp, Homo sapiens
  TRBV20-1_BB_1
gttcgaagctggcatgacacgaagacttgtacgccaccatgctgctgcttctgctgcttctggggccaggctccgggcttggtgctgtcgtctctcaacatccgagctgggttatctgtaagagtggaacctctgtgaagatcgagtgccgttccctggactttcaggccacaactatgttttggtatcgtcagttcccgaaacagagtctcatgctgatggcaacttccaatgagggctccaaggccacatacgagcaaggcgtcgagaaggacaagtttctcatcaaccatgcaagcctgaccttgtccactctgacagtgaccagtgcccatcctgaagatagcagcttctacatttgcagagaccttgcggccgtgtcttccgacgctgacagtgtagata

SEQ ID NO: 476 - DNA, 411 bp, Homo sapiens
  TRBV24-1_BB_1
gttcgaagctggcatgacacgaagacttgtacgccaccatggcctccctgctcttcttctgtggggccttttatctcctgggaacagggtccatggatgctgatgttacccagaccccaaggaataggatcacaaagacaggaaagaggattatgctggaatgttctcagactaagggtcatgatagaatgtactggtatcgacaagacccaggactgggcctacggttgatctattactcctttgatgtcaaagatataaacaaaggagagatctctgatggatacagtgtctctcgacaggcacaggctaaattctccctgtccctagagtctgccatccccaaccagacagctctttacttttgcagagaccttgcggccgtgtcttccgacgctgacagtgtagata

SEQ ID NO: 477 - DNA, 411 bp, Homo sapiens
  TRBV25-1_BB_1
gttcgaagctggcatgacacgaagacttgtacgccaccatgactatcaggctcctctgctacatgggcttttattttctgggggcaggcctcatggaagctgacatctaccagaccccaagataccttgttatagggacaggaaagaagatcactctggaatgttctcaaaccatgggccatgacaaaatgtactggtatcaacaagatccaggaatggaactacacctcatccactattcctatggagttaattccacagagaagggagatctttcctctgagtcaacagtctccagaataaggacggagcattttcccctgaccctggagtctgccaggccctcacatacctctcagtacctttgcagagaccttgcggccgtgtcttccgacgctgacagtgtagata

SEQ ID NO: 478 - DNA, 411 bp, Homo sapiens
  TRBV27_BB_1
gttcgaagctggcatgacacgaagacttgtacgccaccatgggcccccagctccttggctatgtggtcctttgccttctaggagcaggccccctggaagcccaagtgacccagaacccaagatacctcatcacagtgactggaaagaagttaacagtgacttgttctcagaatatgaaccatgagtatatgtcctggtatcgacaagacccagggctgggcttaaggcagatctactattcaatgaatgttgaggtgactgataagggagatgttcctgaagggtacaaagtctctcgaaaagagaagaggaatttccccctgatcctggagtcgcccagccccaaccagacctctctgtacttttgcagagaccttgcggccgtgtcttccgacgctgacagtgtagata

SEQ ID NO: 479 - DNA, 411 bp, Homo sapiens
  TRBV28_BB_1
gttcgaagctggcatgacacgaagacttgtacgccaccatgggaatcaggctcctctgtcgtgtggccttttgtttcctggctgtaggcctcgtagatgtgaaagtaacccagagctcgagatatctagtcaaaaggacgggagagaaagtttttctggaatgtgtccaggatatggaccatgaaaatatgttctggtatcgacaagacccaggtctggggctacggctgatctatttctcatatgatgttaaaatgaaagaaaaaggagatattcctgaggggtacagtgtctctagagagaagaaggagcgcttctccctgattctggagtccgccagcaccaaccagacatctatgtacctttgcagagaccttgcggccgtgtcttccgacgctgacagtgtagata

SEQ ID NO: 480 - DNA, 405 bp, Homo sapiens
  TRBV29-1_BB_1
gttcgaagctggcatgacacgaagacttgtacgccaccatgctgagtctactgctccttctcctgggactaggctctgtgttcagtgctgtcatctctcaaaagccaagcagggatatctgtcaacgtggaacctccctgacgatccagtgtcaagtcgatagccaagtcaccatgatgttctggtaccgtcagcaacctggacagagcctgacactgatcgcaactgcaaatcagggctctgaggccacatatgagagtggatttgtcattgacaagtttcccatcagccgcccaaacctaacattctcaactctgactgtgagcaacatgagccctgaagatagcagcatatatctttgcagagaccttgcggccgtgtcttccgacgctgacagtgtagata

SEQ ID NO: 481 - DNA, 405 bp, Homo sapiens
  TRBV30_BB_1
gttcgaagctggcatgacacgaagacttgtacgccaccatgctctgctctctccttgcccttctcctgggcactttctttggggtcagatctcagactattcatcaatggccagcgaccctggtgcagcctgtgggcagcccgctctctctggagtgcactgtggagggaacatcaaaccccaacctatactggtaccgacaggctgcaggcaggggcctccagctgctcttctactccgttggtattggccagatcagctctgaggtgccccagaatctctcagcctccagaccccaggaccggcagttcatcctgagttctaagaagctccttctcagtgactctggcttctatctttgcagagaccttgcggccgtgtcttccgacgctgacagtgtagata

SEQ ID NO: 482 - DNA, 595 bp, Homo sapiens
  TRBC1 cloning fragment
cagcactcgtatgatcagaggaagactaggccgcataggtctcagtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctgactaggtgtcttcgctacactgatccgatggtc

SEQ ID NO: 483 - DNA, 601 bp, Homo sapiens
  TRBC2 cloning fragment
cagcactcgtatgatcagaggaagactaggccgcataggtctcagtgttcccacccgaggtcgctgtgtttgagccatcagaagcagagatctcccacacccaaaaggccacactggtatgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctagctaggtgtcttcgctacactgatccgatggtc

SEQ ID NO: 484 - DNA, 891 bp, Homo sapiens
  V-C entry TRBV2_TRBC2
gccaccatggatacctggctcgtatgctgggcaatttttagtctcttgaaagcaggactcacagaacctgaagtcacccagactcccagccatcaggtcacacagatgggacaggaagtgatcttgcgctgtgtccccatctctaatcacttatacttctattggtacagacaaatcttggggcagaaagtcgagtttctggtttccttttataataatgaaatctcagagaagtctgaaatattcgatgatcaattctcagttgaaaggcctgatggatcaaatttcactctgaagatccggtccacaaagctggaggactcagccatgtacttttgcagagaccttgcggccgcataggtctcagtgttcccacccgaggtcgctgtgtttgagccatcagaagcagagatctcccacacccaaaaggccacactggtatgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 485 - DNA, 888 bp, Homo sapiens
  V-C entry TRBV3-1_TRBC2
gccaccatgggctgcaggctcctctgctgtgtggtcttttgcctcctccaagcaggtcccttggacacagctgtttcccagactccaaaatacctggtcacacagatgggaaacgacaagtccattaaatgtgaacaaaatctgggccatgatactatgtattggtataaacaggactctaagaaatttctgaagataatgtttagctacaataataaggagctcattataaatgaaacagttccaaatcgcttctcacctaaatctccagacaaagctcacttaaatcttcacatcaattccctggagcttggtgactctgctgtgtatttttgcagagaccttgcggccgcataggtctcagtgttcccacccgaggtcgctgtgtttgagccatcagaagcagagatctcccacacccaaaaggccacactggtatgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 486 - DNA, 888 bp, Homo sapiens
  V-C entry TRBV4-1_TRBC2
gccaccatgggctgcaggctgctctgctgtgcggttctctgtctcctgggagcagttcccatagacactgaagttacccagacaccaaaacacctggtcatgggaatgacaaataagaagtctttgaaatgtgaacaacatatggggcacagggctatgtattggtacaagcagaaagctaagaagccaccggagctcatgtttgtctacagctatgagaaactctctataaatgaaagtgtgccaagtcgcttctcacctgaatgccccaacagctctctcttaaaccttcacctacacgccctgcagccagaagattcagccctgtatctttgcagagaccttgcggccgcataggtctcagtgttcccacccgaggtcgctgtgtttgagccatcagaagcagagatctcccacacccaaaaggccacactggtatgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 487 - DNA, 888 bp, Homo sapiens
  V-C entry TRBV4-2_TRBC2
gccaccatgggctgcaggctgctctgctgtgcggttctctgtctcctgggagcggtccccatggaaacgggagttacgcagacaccaagacacctggtcatgggaatgacaaataagaagtctttgaaatgtgaacaacatctggggcataacgctatgtattggtacaagcaaagtgctaagaagccactggagctcatgtttgtctacaactttaaagaacagactgaaaacaacagtgtgccaagtcgcttctcacctgaatgccccaacagctctcacttattccttcacctacacaccctgcagccagaagattcggccctgtatctttgcagagaccttgcggccgcataggtctcagtgttcccacccgaggtcgctgtgtttgagccatcagaagcagagatctcccacacccaaaaggccacactggtatgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 488 - DNA, 888 bp, Homo sapiens
  V-C entry TRBV4-3_TRBC2
gccaccatgggctgcaggctgctctgctgtgcggttctctgtctcctgggagcggtccccatggaaacgggagttacgcagacaccaagacacctggtcatgggaatgacaaataagaagtctttgaaatgtgaacaacatctgggtcataacgctatgtattggtacaagcaaagtgctaagaagccactggagctcatgtttgtctacagtcttgaagaacgggttgaaaacaacagtgtgccaagtcgcttctcacctgaatgccccaacagctctcacttattccttcacctacacaccctgcagccagaagattcggccctgtatctttgcagagaccttgcggccgcataggtctcagtgttcccacccgaggtcgctgtgtttgagccatcagaagcagagatctcccacacccaaaaggccacactggtatgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 489 - DNA, 888 bp, Homo sapiens
  V-C entry TRBV5-1_TRBC2
gccaccatgggctccaggctgctctgttgggtgctgctttgtctcctgggagcaggcccagtaaaggctggagtcactcaaactccaagatatctgatcaaaacgagaggacagcaagtgacactgagctgctcccctatctctgggcataggagtgtatcctggtaccaacagaccccaggacagggccttcagttcctctttgaatacttcagtgagacacagagaaacaaaggaaacttccctggtcgattctcagggcgccagttctctaactctcgctctgagatgaatgtgagcaccttggagctgggggactcggccctttatctttgcagagaccttgcggccgcataggtctcagtgttcccacccgaggtcgctgtgtttgagccatcagaagcagagatctcccacacccaaaaggccacactggtatgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 490 - DNA, 888 bp, Homo sapiens
  V-C entry TRBV5-4_TRBC2
gccaccatgggccctgggctcctctgctgggtgctgctttgtctcctgggagcaggctcagtggagactggagtcacccaaagtcccacacacctgatcaaaacgagaggacagcaagtgactctgagatgctcttctcagtctgggcacaacactgtgtcctggtaccaacaggccctgggtcaggggccccagtttatctttcagtattatagggaggaagagaatggcagaggaaacttccctcctagattctcaggactccagttccctaattatagctctgagctgaatgtgaacgccttggagctggacgactcggccctgtatctttgcagagaccttgcggccgcataggtctcagtgttcccacccgaggtcgctgtgtttgagccatcagaagcagagatctcccacacccaaaaggccacactggtatgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 491 - DNA, 888 bp, Homo sapiens
  V-C entry TRBV5-5_TRBC2
gccaccatgggccctgggctcctctgctgggtgctgctttgtctcctgggagcaggcccagtggacgctggagtcacccaaagtcccacacacctgatcaaaacgagaggacagcaagtgactctgagatgctctcctatctctgggcacaagagtgtgtcctggtaccaacaggtcctgggtcaggggccccagtttatctttcagtattatgagaaagaagagagaggaagaggaaacttccctgatcgattctcagctcgccagttccctaactatagctctgagctgaatgtgaacgccttgttgctgggggactcggccctgtatctttgcagagaccttgcggccgcataggtctcagtgttcccacccgaggtcgctgtgtttgagccatcagaagcagagatctcccacacccaaaaggccacactggtatgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 492 - DNA, 888 bp, Homo sapiens
  V-C entry TRBV5-6_TRBC2
gccaccatgggccccgggctcctctgctgggcactgctttgtctcctgggagcaggcttagtggacgctggagtcacccaaagtcccacacacctgatcaaaacgagaggacagcaagtgactctgagatgctctcctaagtctgggcatgacactgtgtcctggtaccaacaggccctgggtcaggggccccagtttatctttcagtattatgaggaggaagagagacagagaggcaacttccctgatcgattctcaggtcaccagttccctaactatagctctgagctgaatgtgaacgccttgttgctgggggactcggccctctatctttgcagagaccttgcggccgcataggtctcagtgttcccacccgaggtcgctgtgtttgagccatcagaagcagagatctcccacacccaaaaggccacactggtatgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 493 - DNA, 888 bp, Homo sapiens
  V-C entry TRBV5-7_TRBC2
gccaccatgggccccgggctcctctgctgggtgctgctttgtcccctaggagaaggcccagtggacgctggagtcacccaaagtcccacacacctgatcaaaacgagaggacagcacgtgactctgagatgctctcctatctctgggcacaccagtgtgtcctcgtaccaacaggccctgggtcaggggccccagtttatctttcagtattatgagaaagaagagagaggaagaggaaacttccctgatcaattctcaggtcaccagttccctaactatagctctgagctgaatgtgaacgccttgttgctaggggactcggccctctatctttgcagagaccttgcggccgcataggtctcagtgttcccacccgaggtcgctgtgtttgagccatcagaagcagagatctcccacacccaaaaggccacactggtatgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 494 - DNA, 888 bp, Homo sapiens
  V-C entry TRBV5-8_TRBC2
gccaccatgggacccaggctcctcttctgggcactgctttgtctcctcggaacaggcccagtggaggctggagtcacacaaagtcccacacacctgatcaaaacgagaggacagcaagcgactctgagatgctctcctatctctgggcacaccagtgtgtactggtaccaacaggccctgggtctgggcctccagttcctcctttggtatgacgagggtgaagagagaaacagaggaaacttccctcctagattttcaggtcgccagttccctaattatagctctgagctgaatgtgaacgccttggagctggaggactcggccctgtatctttgcagagaccttgcggccgcataggtctcagtgttcccacccgaggtcgctgtgtttgagccatcagaagcagagatctcccacacccaaaaggccacactggtatgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 495 - DNA, 888 bp, Homo sapiens
  V-C entry TRBV6-1_TRBC2
gccaccatgagcatcgggctcctgtgctgtgtggccttttctctcctgtgggcaagtccagtgaatgctggtgtcactcagaccccaaaattccaggtcctgaaaacaggacagagcatgacactgcagtgtgcccaggatatgaaccataactccatgtactggtatcgacaagacccaggcatgggactgaggctgatttattactcagcttctgagggtaccactgacaaaggagaagtccccaatggctacaatgtctccagattaaacaaacgggagttctcgctcaggctggagtcggctgctccctcccagacatctgtgtacttttgcagagaccttgcggccgcataggtctcagtgttcccacccgaggtcgctgtgtttgagccatcagaagcagagatctcccacacccaaaaggccacactggtatgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 496 - DNA, 888 bp, Homo sapiens
  V-C entry TRBV6-3_TRBC2
gccaccatgagcctcgggctcctgtgctgtggggtcttttctctcctgtgggcaggtccagtgaatgctggtgtcactcagaccccaaaattccgggtcctgaaaacaggacagagcatgacactgctgtgtgcccaggatatgaaccatgaatacatgtactggtatcgacaagacccaggcatggggctgaggctgattcattactcagttggtgagggtacaactgccaaaggagaggtccctgatggctacaatgtctccagattaaaaaaacagaatttcctgctggggttggagtcggctgctccctcccaaacatctgtgtacttttgcagagaccttgcggccgcataggtctcagtgttcccacccgaggtcgctgtgtttgagccatcagaagcagagatctcccacacccaaaaggccacactggtatgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 497 - DNA, 888 bp, Homo sapiens
  V-C entry TRBV6-4_TRBC2
gccaccatgagaatcaggctcctgtgctgtgtggccttttctctcctgtgggcaggtccagtgattgctgggatcacccaggcaccaacatctcagatcctggcagcaggacggcgcatgacactgagatgtacccaggatatgagacataatgccatgtactggtatagacaagatctaggactggggctaaggctcatccattattcaaatactgcaggtaccactggcaaaggagaagtccctgatggttatagtgtctccagagcaaacacagatgatttccccctcacgttggcgtctgctgtaccctctcagacatctgtgtacttttgcagagaccttgcggccgcataggtctcagtgttcccacccgaggtcgctgtgtttgagccatcagaagcagagatctcccacacccaaaaggccacactggtatgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 498 - DNA, 888 bp, Homo sapiens
  V-C entry TRBV6-5_TRBC2
gccaccatgagcatcggcctcctgtgctgtgcagccttgtctctcctgtgggcaggtccagtgaatgctggtgtcactcagaccccaaaattccaggtcctgaaaacaggacagagcatgacactgcagtgtgcccaggatatgaaccatgaatacatgtcctggtatcgacaagacccaggcatggggctgaggctgattcattactcagttggtgctggtatcactgaccaaggagaagtccccaatggctacaatgtctccagatcaaccacagaggatttcccgctcaggctgctgtcggctgctccctcccagacatctgtgtacttttgcagagaccttgcggccgcataggtctcagtgttcccacccgaggtcgctgtgtttgagccatcagaagcagagatctcccacacccaaaaggccacactggtatgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 499 - DNA, 888 bp, Homo sapiens
  V-C entry TRBV6-6_TRBC2
gccaccatgagcatcagcctcctgtgctgtgcagcctttcctctcctgtgggcaggtccagtgaatgctggtgtcactcagaccccaaaattccgcatcctgaagataggacagagcatgacactgcagtgtacccaggatatgaaccataactacatgtactggtatcgacaagacccaggcatggggctgaagctgatttattattcagttggtgctggtatcactgataaaggagaagtcccgaatggctacaacgtctccagatcaaccacagaggatttcccgctcaggctggagttggctgctccctcccagacatctgtgtacttttgcagagaccttgcggccgcataggtctcagtgttcccacccgaggtcgctgtgtttgagccatcagaagcagagatctcccacacccaaaaggccacactggtatgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 500 - DNA, 885 bp, Homo sapiens
  V-C entry TRBV6-8_TRBC2
gccaccatgagcctcgggctcctgtgctgtgcggccttttctctcctgtgggcaggtcccgtgaatgctggtgtcactcagaccccaaaattccacatcctgaaaacaggacagagcatgacactgcagtgtgcccaggatatgaaccatggatacatgtcctggtatcgacaagacccaggcatggggctgagactgatttactactcagctgctgctggtactactgacaaagaagtccccaatggctacaatgtctctagattaaacacagaggatttcccactcaggctggtgtcggctgctccctcccagacatctgtgtacctttgcagagaccttgcggccgcataggtctcagtgttcccacccgaggtcgctgtgtttgagccatcagaagcagagatctcccacacccaaaaggccacactggtatgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 501 - DNA, 888 bp, Homo sapiens
  V-C entry TRBV6-9_TRBC2
gccaccatgagcatcgggctcctgtgctgtgtggccttttctctcctgtgggcaggtccagtgaatgctggtgtcactcagaccccaaaattccacatcctgaaaacaggacagagcatgacactgcagtgtgcccaggatatgaaccatggatacttgtcctggtatcgacaagacccaggcatggggctgaggcgcattcattactcagttgctgctggtatcactgacaaaggagaagtccccgatggctacaatgtatccagatcaaacacagaggatttcccgctcaggctggagtcagctgctccctcccagacatctgtatacttttgcagagaccttgcggccgcataggtctcagtgttcccacccgaggtcgctgtgtttgagccatcagaagcagagatctcccacacccaaaaggccacactggtatgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 502 - DNA, 891 bp, Homo sapiens
  V-C entry TRBV7-2_TRBC2
gccaccatgggcaccaggctcctcttctgggtggccttctgtctcctgggggcagatcacacaggagctggagtctcccagtcccccagtaacaaggtcacagagaagggaaaggatgtagagctcaggtgtgatccaatttcaggtcatactgccctttactggtaccgacagagcctggggcagggcctggagtttttaatttacttccaaggcaacagtgcaccagacaaatcagggctgcccagtgatcgcttctctgcagagaggactgggggatccgtctccactctgacgatccagcgcacacagcaggaggactcggccgtgtatctttgcagagaccttgcggccgcataggtctcagtgttcccacccgaggtcgctgtgtttgagccatcagaagcagagatctcccacacccaaaaggccacactggtatgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 503 - DNA, 891 bp, Homo sapiens
  V-C entry TRBV7-3_TRBC2
gccaccatgggcaccaggctcctctgctgggcagccctgtgcctcctgggggcagatcacacaggtgctggagtctcccagacccccagtaacaaggtcacagagaagggaaaatatgtagagctcaggtgtgatccaatttcaggtcatactgccctttactggtaccgacaaagcctggggcagggcccagagtttctaatttacttccaaggcacgggtgcggcagatgactcagggctgcccaacgatcggttctttgcagtcaggcctgagggatccgtctctactctgaagatccagcgcacagagcggggggactcagccgtgtatctttgcagagaccttgcggccgcataggtctcagtgttcccacccgaggtcgctgtgtttgagccatcagaagcagagatctcccacacccaaaaggccacactggtatgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 504 - DNA, 891 bp, Homo sapiens
  V-C entry TRBV7-6_TRBC2
gccaccatgggcaccagtctcctatgctgggtggtcctgggtttcctagggacagatcacacaggtgctggagtctcccagtctcccaggtacaaagtcacaaagaggggacaggatgtagctctcaggtgtgatccaatttcgggtcatgtatccctttattggtaccgacaggccctggggcagggcccagagtttctgacttacttcaattatgaagcccaacaagacaaatcagggctgcccaatgatcggttctctgcagagaggcctgagggatccatctccactctgacgatccagcgcacagagcagcgggactcggccatgtatcgttgcagagaccttgcggccgcataggtctcagtgttcccacccgaggtcgctgtgtttgagccatcagaagcagagatctcccacacccaaaaggccacactggtatgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 505 - DNA, 891 bp, Homo sapiens
  V-C entry TRBV7-7_TRBC2
gccaccatgggtaccagtctcctatgctgggtggtcctgggtttcctagggacagatcacacaggtgctggagtctcccagtctcccaggtacaaagtcacaaagaggggacaggatgtaactctcaggtgtgatccaatttcgagtcatgcaaccctttattggtatcaacaggccctggggcagggcccagagtttctgacttacttcaattatgaagctcaaccagacaaatcagggctgcccagtgatcggttctctgcagagaggcctgagggatccatctccactctgacgattcagcgcacagagcagcgggactcagccatgtatcgttgcagagaccttgcggccgcataggtctcagtgttcccacccgaggtcgctgtgtttgagccatcagaagcagagatctcccacacccaaaaggccacactggtatgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 506 - DNA, 891 bp, Homo sapiens
  V-C entry TRBV7-8_TRBC2
gccaccatgggcaccaggctcctctgctgggtggtcctgggtttcctagggacagatcacacaggtgctggagtctcccagtcccctaggtacaaagtcgcaaagagaggacaggatgtagctctcaggtgtgatccaatttcgggtcatgtatcccttttttggtaccaacaggccctggggcaggggccagagtttctgacttatttccagaatgaagctcaactagacaaatcggggctgcccagtgatcgcttctttgcagaaaggcctgagggatccgtctccactctgaagatccagcgcacacagcaggaggactccgccgtgtatctttgcagagaccttgcggccgcataggtctcagtgttcccacccgaggtcgctgtgtttgagccatcagaagcagagatctcccacacccaaaaggccacactggtatgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 507 - DNA, 891 bp, Homo sapiens
  V-C entry TRBV7-9_TRBC2
gccaccatgggcaccagcctcctctgctggatggccctgtgtctcctgggggcagatcacgcagatactggagtctcccagaaccccagacacaagatcacaaagaggggacagaatgtaactttcaggtgtgatccaatttctgaacacaaccgcctttattggtaccgacagaccctggggcagggcccagagtttctgacttacttccagaatgaagctcaactagaaaaatcaaggctgctcagtgatcggttctctgcagagaggcctaagggatctttctccaccttggagatccagcgcacagagcagggggactcggccatgtatctttgcagagaccttgcggccgcataggtctcagtgttcccacccgaggtcgctgtgtttgagccatcagaagcagagatctcccacacccaaaaggccacactggtatgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 508 - DNA, 888 bp, Homo sapiens
  V-C entry TRBV9_TRBC2
gccaccatgggcttcaggctcctctgctgtgtggccttttgtctcctgggagcaggcccagtggattctggagtcacacaaaccccaaagcacctgatcacagcaactggacagcgagtgacgctgagatgctcccctaggtctggtgacctctctgtgtactggtaccaacagagcctggaccagggcctccagttcctcattcagtattataatggagaagagagagcaaaaggaaacattcttgaacgattctccgcacaacagttccctgacttgcactctgaactaaacctgagctctctggagctgggggactcagctttgtacttttgcagagaccttgcggccgcataggtctcagtgttcccacccgaggtcgctgtgtttgagccatcagaagcagagatctcccacacccaaaaggccacactggtatgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 509 - DNA, 888 bp, Homo sapiens
  V-C entry TRBV10-1_TRBC2
gccaccatgggcacgaggctcttcttctatgtggccctttgtctgctgtgggcaggacacagggatgctgaaatcacccagagcccaagacacaagatcacagagacaggaaggcaggtgaccttggcgtgtcaccagacttggaaccacaacaatatgttctggtatcgacaagacctgggacatgggctgaggctgatccattactcatatggtgttcaagacactaacaaaggagaagtctcagatggctacagtgtctctagatcaaacacagaggacctccccctcactctggagtctgctgcctcctcccagacatctgtatacttttgcagagaccttgcggccgcataggtctcagtgttcccacccgaggtcgctgtgtttgagccatcagaagcagagatctcccacacccaaaaggccacactggtatgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 510 - DNA, 888 bp, Homo sapiens
  V-C entry TRBV10-2_TRBC2
gccaccatgggcaccaggctcttcttctatgtggccctttgtctgctgtgggcaggacacagggatgctggaatcacccagagcccaagatacaagatcacagagacaggaaggcaggtgaccttgatgtgtcaccagacttggagccacagctatatgttctggtatcgacaagacctgggacatgggctgaggctgatctattactcagcagctgctgatattacagataaaggagaagtccccgatggctatgttgtctccagatccaagacagagaatttccccctcactctggagtcagctacccgctcccagacatctgtgtacttttgcagagaccttgcggccgcataggtctcagtgttcccacccgaggtcgctgtgtttgagccatcagaagcagagatctcccacacccaaaaggccacactggtatgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 511 - DNA, 888 bp, Homo sapiens
  V-C entry TRBV10-3_TRBC2
gccaccatgggcacaaggttgttcttctatgtggccctttgtctcctgtggacaggacacatggatgctggaatcacccagagcccaagacacaaggtcacagagacaggaacaccagtgactctgagatgtcaccagactgagaaccaccgctatatgtactggtatcgacaagacccggggcatgggctgaggctgatccattactcatatggtgttaaagatactgacaaaggagaagtctcagatggctatagtgtctctagatcaaagacagaggatttcctcctcactctggagtccgctaccagctcccagacatctgtgtacttttgcagagaccttgcggccgcataggtctcagtgttcccacccgaggtcgctgtgtttgagccatcagaagcagagatctcccacacccaaaaggccacactggtatgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 512 - DNA, 891 bp, Homo sapiens
  V-C entry TRBV11-1_TRBC2
gccaccatgagcaccaggcttctctgctggatggccctctgtctcctgggggcagaactctcagaagctgaagttgcccagtcccccagatataagattacagagaaaagccaggctgtggctttttggtgtgatcctatttctggccatgctaccctttactggtaccggcagatcctgggacagggcccggagcttctggttcaatttcaggatgagagtgtagtagatgattcacagttgcctaaggatcgattttctgcagagaggctcaaaggagtagactccactctcaagatccagcctgcagagcttggggactcggccatgtatctttgcagagaccttgcggccgcataggtctcagtgttcccacccgaggtcgctgtgtttgagccatcagaagcagagatctcccacacccaaaaggccacactggtatgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 513 - DNA, 891 bp, Homo sapiens
  V-C entry TRBV11-2_TRBC2
gccaccatgggcaccaggctcctctgctgggcggccctctgtctcctgggagcagaactcacagaagctggagttgcccagtctcccagatataagattatagagaaaaggcagagtgtggctttttggtgcaatcctatatctggccatgctaccctttactggtaccagcagatcctgggacagggcccaaagcttctgattcagtttcagaataacggtgtagtggatgattcacagttgcctaaggatcgattttctgcagagaggctcaaaggagtagactccactctcaagatccagcctgcaaagcttgaggactcggccgtgtatctttgcagagaccttgcggccgcataggtctcagtgttcccacccgaggtcgctgtgtttgagccatcagaagcagagatctcccacacccaaaaggccacactggtatgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 514 - DNA, 891 bp, Homo sapiens
  V-C entry TRBV11-3_TRBC2
gccaccatgggtaccaggctcctctgctgggtggccttctgtctcctggtggaagaactcatagaagctggagtggttcagtctcccagatataagattatagagaaaaaacagcctgtggctttttggtgcaatcctatttctggccacaataccctttactggtacctgcagaacttgggacagggcccggagcttctgattcgatatgagaatgaggaagcagtagacgattcacagttgcctaaggatcgattttctgcagagaggctcaaaggagtagactccactctcaagatccagcctgcagagcttggggactcggccgtgtatctttgcagagaccttgcggccgcataggtctcagtgttcccacccgaggtcgctgtgtttgagccatcagaagcagagatctcccacacccaaaaggccacactggtatgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 515 - DNA, 891 bp, Homo sapiens
  V-C entry TRBV12-3_TRBC2
gccaccatggactcctggaccttctgctgtgtgtccctttgcatcctggtagcgaagcatacagatgctggagttatccagtcaccccgccatgaggtgacagagatgggacaagaagtgactctgagatgtaaaccaatttcaggccacaactcccttttctggtacagacagaccatgatgcggggactggagttgctcatttactttaacaacaacgttccgatagatgattcagggatgcccgaggatcgattctcagctaagatgcctaatgcatcattctccactctgaagatccagccctcagaacccagggactcagctgtgtacttttgcagagaccttgcggccgcataggtctcagtgttcccacccgaggtcgctgtgtttgagccatcagaagcagagatctcccacacccaaaaggccacactggtatgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 516 - DNA, 891 bp, Homo sapiens
  V-C entry TRBV12-4_TRBC2
gccaccatggactcctggaccctctgctgtgtgtccctttgcatcctggtagcaaagcacacagatgctggagttatccagtcaccccggcacgaggtgacagagatgggacaagaagtgactctgagatgtaaaccaatttcaggacacgactaccttttctggtacagacagaccatgatgcggggactggagttgctcatttactttaacaacaacgttccgatagatgattcagggatgcccgaggatcgattctcagctaagatgcctaatgcatcattctccactctgaagatccagccctcagaacccagggactcagctgtgtacttttgcagagaccttgcggccgcataggtctcagtgttcccacccgaggtcgctgtgtttgagccatcagaagcagagatctcccacacccaaaaggccacactggtatgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 517 - DNA, 891 bp, Homo sapiens
  V-C entry TRBV12-5_TRBC2
gccaccatggccaccaggctcctctgctgtgtggttctttgtctcctgggagaagagcttatagatgctagagtcacccagacaccaaggcacaaggtgacagagatgggacaagaagtaacaatgagatgtcagccaattttaggccacaatactgttttctggtacagacagaccatgatgcaaggactggagttgctggcttacttccgcaaccgggctcctctagatgattcggggatgccgaaggatcgattctcagcagagatgcctgatgcaactttagccactctgaagatccagccctcagaacccagggactcagctgtgtacttttgcagagaccttgcggccgcataggtctcagtgttcccacccgaggtcgctgtgtttgagccatcagaagcagagatctcccacacccaaaaggccacactggtatgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 518 - DNA, 918 bp, Homo sapiens
  V-C entry TRBV13_TRBC2
gccaccatgcttagtcctgacctgcctgactctgcctggaacaccaggctcctctgccatgtcatgctttgtctcctgggagcagtttcagtggctgctggagtcatccagtccccaagacatctgatcaaagaaaagagggaaacagccactctgaaatgctatcctatccctagacacgacactgtctactggtaccagcagggtccaggtcaggacccccagttcctcatttcgttttatgaaaagatgcagagcgataaaggaagcatccctgatcgattctcagctcaacagttcagtgactatcattctgaactgaacatgagctccttggagctgggggactcagccctgtacttttgcagagaccttgcggccgcataggtctcagtgttcccacccgaggtcgctgtgtttgagccatcagaagcagagatctcccacacccaaaaggccacactggtatgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 519 - DNA, 891 bp, Homo sapiens
  V-C entry TRBV14_TRBC2
gccaccatggtttccaggcttctcagtttagtgtccctttgtctcctgggagcaaagcacatagaagctggagttactcagttccccagccacagcgtaatagagaagggccagactgtgactctgagatgtgacccaatttctggacatgataatctttattggtatcgacgtgttatgggaaaagaaataaaatttctgttacattttgtgaaagagtctaaacaggatgagtccggtatgcccaacaatcgattcttagctgaaaggactggagggacgtattctactctgaaggtgcagcctgcagaactggaggattctggagtttacttttgcagagaccttgcggccgcataggtctcagtgttcccacccgaggtcgctgtgtttgagccatcagaagcagagatctcccacacccaaaaggccacactggtatgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 520 - DNA, 888 bp, Homo sapiens
  V-C entry TRBV15_TRBC2
gccaccatgggtcctgggcttctccactggatggccctttgtctccttggaacaggtcatggggatgccatggtcatccagaacccaagataccaggttacccagtttggaaagccagtgaccctgagttgttctcagactttgaaccataacgtcatgtactggtaccagcagaagtcaagtcaggccccaaagctgctgttccactactatgacaaagattttaacaatgaagcagacacccctgataacttccaatccaggaggccgaacacttctttctgctttcttgacatccgctcaccaggcctgggggacacagccatgtacctttgcagagaccttgcggccgcataggtctcagtgttcccacccgaggtcgctgtgtttgagccatcagaagcagagatctcccacacccaaaaggccacactggtatgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 521 - DNA, 891 bp, Homo sapiens
  V-C entry TRBV16_TRBC2
gccaccatgagcccaatattcacctgcatcacaatcctttgtctgctggctgcaggttctcctggtgaagaagtcgcccagactccaaaacatcttgtcagaggggaaggacagaaagcaaaattatattgtgccccaataaaaggacacagttatgttttttggtaccaacaggtcctgaaaaacgagttcaagttcttgatttccttccagaatgaaaatgtctttgatgaaacaggtatgcccaaggaaagattttcagctaagtgcctcccaaattcaccctgtagccttgagatccaggctacgaagcttgaggattcagcagtgtacttttgcagagaccttgcggccgcataggtctcagtgttcccacccgaggtcgctgtgtttgagccatcagaagcagagatctcccacacccaaaaggccacactggtatgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 522 - DNA, 891 bp, Homo sapiens
  V-C entry TRBV18_TRBC2
gccaccatggacaccagagtactctgctgtgcggtcatctgccttctgggggcaggactctcaaatgccggcgtcatgcagaacccaagacacctggtcaggaggaggggacaggaggcaagactgagatgcagcccaatgaaaggacacagtcatgtttactggtatcggcagctcccagaggaaggtctgaaattcatggtttatctccagaaagaaaatatcatagatgagtcaggaatgccaaaggaacgattttctgctgaatttcccaaagagggccccagcatcctgaggatccagcaggtagtgcgaggagattcggcagcttacttttgcagagaccttgcggccgcataggtctcagtgttcccacccgaggtcgctgtgtttgagccatcagaagcagagatctcccacacccaaaaggccacactggtatgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 523 - DNA, 888 bp, Homo sapiens
  V-C entry TRBV19_TRBC2
gccaccatgagcaaccaggtgctctgctgtgtggtcctttgtttcctgggagcaaacaccgtggatggtggaatcactcagtccccaaagtacctgttcagaaaggaaggacagaatgtgaccctgagttgtgaacagaatttgaaccacgatgccatgtactggtaccgacaggacccagggcaagggctgagattgatctactactcacagatagtaaatgactttcagaaaggagatatagctgaagggtacagcgtctctcgggagaagaaggaatcctttcctctcactgtgacatcggcccaaaagaacccgacagctttctatctttgcagagaccttgcggccgcataggtctcagtgttcccacccgaggtcgctgtgtttgagccatcagaagcagagatctcccacacccaaaaggccacactggtatgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 524 - DNA, 882 bp, Homo sapiens
  V-C entry TRBV20-1_TRBC2
gccaccatgctgctgcttctgctgcttctggggccaggctccgggcttggtgctgtcgtctctcaacatccgagctgggttatctgtaagagtggaacctctgtgaagatcgagtgccgttccctggactttcaggccacaactatgttttggtatcgtcagttcccgaaacagagtctcatgctgatggcaacttccaatgagggctccaaggccacatacgagcaaggcgtcgagaaggacaagtttctcatcaaccatgcaagcctgaccttgtccactctgacagtgaccagtgcccatcctgaagatagcagcttctacatttgcagagaccttgcggccgcataggtctcagtgttcccacccgaggtcgctgtgtttgagccatcagaagcagagatctcccacacccaaaaggccacactggtatgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 525 - DNA, 888 bp, Homo sapiens
  V-C entry TRBV24-1_TRBC2
gccaccatggcctccctgctcttcttctgtggggccttttatctcctgggaacagggtccatggatgctgatgttacccagaccccaaggaataggatcacaaagacaggaaagaggattatgctggaatgttctcagactaagggtcatgatagaatgtactggtatcgacaagacccaggactgggcctacggttgatctattactcctttgatgtcaaagatataaacaaaggagagatctctgatggatacagtgtctctcgacaggcacaggctaaattctccctgtccctagagtctgccatccccaaccagacagctctttacttttgcagagaccttgcggccgcataggtctcagtgttcccacccgaggtcgctgtgtttgagccatcagaagcagagatctcccacacccaaaaggccacactggtatgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 526 - DNA, 888 bp, Homo sapiens
  V-C entry TRBV25-1_TRBC2
gccaccatgactatcaggctcctctgctacatgggcttttattttctgggggcaggcctcatggaagctgacatctaccagaccccaagataccttgttatagggacaggaaagaagatcactctggaatgttctcaaaccatgggccatgacaaaatgtactggtatcaacaagatccaggaatggaactacacctcatccactattcctatggagttaattccacagagaagggagatctttcctctgagtcaacagtctccagaataaggacggagcattttcccctgaccctggagtctgccaggccctcacatacctctcagtacctttgcagagaccttgcggccgcataggtctcagtgttcccacccgaggtcgctgtgtttgagccatcagaagcagagatctcccacacccaaaaggccacactggtatgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 527 - DNA, 888 bp, Homo sapiens
  V-C entry TRBV27_TRBC2
gccaccatgggcccccagctccttggctatgtggtcctttgccttctaggagcaggccccctggaagcccaagtgacccagaacccaagatacctcatcacagtgactggaaagaagttaacagtgacttgttctcagaatatgaaccatgagtatatgtcctggtatcgacaagacccagggctgggcttaaggcagatctactattcaatgaatgttgaggtgactgataagggagatgttcctgaagggtacaaagtctctcgaaaagagaagaggaatttccccctgatcctggagtcgcccagccccaaccagacctctctgtacttttgcagagaccttgcggccgcataggtctcagtgttcccacccgaggtcgctgtgtttgagccatcagaagcagagatctcccacacccaaaaggccacactggtatgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 528 - DNA, 888 bp, Homo sapiens
  V-C entry TRBV28_TRBC2
gccaccatgggaatcaggctcctctgtcgtgtggccttttgtttcctggctgtaggcctcgtagatgtgaaagtaacccagagctcgagatatctagtcaaaaggacgggagagaaagtttttctggaatgtgtccaggatatggaccatgaaaatatgttctggtatcgacaagacccaggtctggggctacggctgatctatttctcatatgatgttaaaatgaaagaaaaaggagatattcctgaggggtacagtgtctctagagagaagaaggagcgcttctccctgattctggagtccgccagcaccaaccagacatctatgtacctttgcagagaccttgcggccgcataggtctcagtgttcccacccgaggtcgctgtgtttgagccatcagaagcagagatctcccacacccaaaaggccacactggtatgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 529 - DNA, 882 bp, Homo sapiens
  V-C entry TRBV29-1_TRBC2
gccaccatgctgagtctactgctccttctcctgggactaggctctgtgttcagtgctgtcatctctcaaaagccaagcagggatatctgtcaacgtggaacctccctgacgatccagtgtcaagtcgatagccaagtcaccatgatgttctggtaccgtcagcaacctggacagagcctgacactgatcgcaactgcaaatcagggctctgaggccacatatgagagtggatttgtcattgacaagtttcccatcagccgcccaaacctaacattctcaactctgactgtgagcaacatgagccctgaagatagcagcatatatctttgcagagaccttgcggccgcataggtctcagtgttcccacccgaggtcgctgtgtttgagccatcagaagcagagatctcccacacccaaaaggccacactggtatgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 530 - DNA, 882 bp, Homo sapiens
  V-C entry TRBV30_TRBC2
gccaccatgctctgctctctccttgcccttctcctgggcactttctttggggtcagatctcagactattcatcaatggccagcgaccctggtgcagcctgtgggcagcccgctctctctggagtgcactgtggagggaacatcaaaccccaacctatactggtaccgacaggctgcaggcaggggcctccagctgctcttctactccgttggtattggccagatcagctctgaggtgccccagaatctctcagcctccagaccccaggaccggcagttcatcctgagttctaagaagctccttctcagtgactctggcttctatctttgcagagaccttgcggccgcataggtctcagtgttcccacccgaggtcgctgtgtttgagccatcagaagcagagatctcccacacccaaaaggccacactggtatgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 531 - DNA, 885 bp, Homo sapiens
  V-C entry TRBV2_TRBC1
gccaccatggatacctggctcgtatgctgggcaatttttagtctcttgaaagcaggactcacagaacctgaagtcacccagactcccagccatcaggtcacacagatgggacaggaagtgatcttgcgctgtgtccccatctctaatcacttatacttctattggtacagacaaatcttggggcagaaagtcgagtttctggtttccttttataataatgaaatctcagagaagtctgaaatattcgatgatcaattctcagttgaaaggcctgatggatcaaatttcactctgaagatccggtccacaaagctggaggactcagccatgtacttttgcagagaccttgcggccgcataggtctcagtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctga

SEQ ID NO: 532 - DNA, 882 bp, Homo sapiens
  V-C entry TRBV3-1_TRBC1
gccaccatgggctgcaggctcctctgctgtgtggtcttttgcctcctccaagcaggtcccttggacacagctgtttcccagactccaaaatacctggtcacacagatgggaaacgacaagtccattaaatgtgaacaaaatctgggccatgatactatgtattggtataaacaggactctaagaaatttctgaagataatgtttagctacaataataaggagctcattataaatgaaacagttccaaatcgcttctcacctaaatctccagacaaagctcacttaaatcttcacatcaattccctggagcttggtgactctgctgtgtatttttgcagagaccttgcggccgcataggtctcagtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctga

SEQ ID NO: 533 - DNA, 882 bp, Homo sapiens
  V-C entry TRBV4-1_TRBC1
gccaccatgggctgcaggctgctctgctgtgcggttctctgtctcctgggagcagttcccatagacactgaagttacccagacaccaaaacacctggtcatgggaatgacaaataagaagtctttgaaatgtgaacaacatatggggcacagggctatgtattggtacaagcagaaagctaagaagccaccggagctcatgtttgtctacagctatgagaaactctctataaatgaaagtgtgccaagtcgcttctcacctgaatgccccaacagctctctcttaaaccttcacctacacgccctgcagccagaagattcagccctgtatctttgcagagaccttgcggccgcataggtctcagtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctga

SEQ ID NO: 534 - DNA, 882 bp, Homo sapiens
  V-C entry TRBV4-2_TRBC1
gccaccatgggctgcaggctgctctgctgtgcggttctctgtctcctgggagcggtccccatggaaacgggagttacgcagacaccaagacacctggtcatgggaatgacaaataagaagtctttgaaatgtgaacaacatctggggcataacgctatgtattggtacaagcaaagtgctaagaagccactggagctcatgtttgtctacaactttaaagaacagactgaaaacaacagtgtgccaagtcgcttctcacctgaatgccccaacagctctcacttattccttcacctacacaccctgcagccagaagattcggccctgtatctttgcagagaccttgcggccgcataggtctcagtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctga

SEQ ID NO: 535 - DNA, 882 bp, Homo sapiens
  V-C entry TRBV4-3_TRBC1
gccaccatgggctgcaggctgctctgctgtgcggttctctgtctcctgggagcggtccccatggaaacgggagttacgcagacaccaagacacctggtcatgggaatgacaaataagaagtctttgaaatgtgaacaacatctgggtcataacgctatgtattggtacaagcaaagtgctaagaagccactggagctcatgtttgtctacagtcttgaagaacgggttgaaaacaacagtgtgccaagtcgcttctcacctgaatgccccaacagctctcacttattccttcacctacacaccctgcagccagaagattcggccctgtatctttgcagagaccttgcggccgcataggtctcagtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctga

SEQ ID NO: 536 - DNA, 882 bp, Homo sapiens
  V-C entry TRBV5-1_TRBC1
gccaccatgggctccaggctgctctgttgggtgctgctttgtctcctgggagcaggcccagtaaaggctggagtcactcaaactccaagatatctgatcaaaacgagaggacagcaagtgacactgagctgctcccctatctctgggcataggagtgtatcctggtaccaacagaccccaggacagggccttcagttcctctttgaatacttcagtgagacacagagaaacaaaggaaacttccctggtcgattctcagggcgccagttctctaactctcgctctgagatgaatgtgagcaccttggagctgggggactcggccctttatctttgcagagaccttgcggccgcataggtctcagtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctga

SEQ ID NO: 537 - DNA, 882 bp, Homo sapiens
  V-C entry TRBV5-4_TRBC1
gccaccatgggccctgggctcctctgctgggtgctgctttgtctcctgggagcaggctcagtggagactggagtcacccaaagtcccacacacctgatcaaaacgagaggacagcaagtgactctgagatgctcttctcagtctgggcacaacactgtgtcctggtaccaacaggccctgggtcaggggccccagtttatctttcagtattatagggaggaagagaatggcagaggaaacttccctcctagattctcaggactccagttccctaattatagctctgagctgaatgtgaacgccttggagctggacgactcggccctgtatctttgcagagaccttgcggccgcataggtctcagtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctga

SEQ ID NO: 538 - DNA, 882 bp, Homo sapiens
  V-C entry TRBV5-5_TRBC1
gccaccatgggccctgggctcctctgctgggtgctgctttgtctcctgggagcaggcccagtggacgctggagtcacccaaagtcccacacacctgatcaaaacgagaggacagcaagtgactctgagatgctctcctatctctgggcacaagagtgtgtcctggtaccaacaggtcctgggtcaggggccccagtttatctttcagtattatgagaaagaagagagaggaagaggaaacttccctgatcgattctcagctcgccagttccctaactatagctctgagctgaatgtgaacgccttgttgctgggggactcggccctgtatctttgcagagaccttgcggccgcataggtctcagtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctga

SEQ ID NO: 539 - DNA, 882 bp, Homo sapiens
  V-C entry TRBV5-6_TRBC1
gccaccatgggccccgggctcctctgctgggcactgctttgtctcctgggagcaggcttagtggacgctggagtcacccaaagtcccacacacctgatcaaaacgagaggacagcaagtgactctgagatgctctcctaagtctgggcatgacactgtgtcctggtaccaacaggccctgggtcaggggccccagtttatctttcagtattatgaggaggaagagagacagagaggcaacttccctgatcgattctcaggtcaccagttccctaactatagctctgagctgaatgtgaacgccttgttgctgggggactcggccctctatctttgcagagaccttgcggccgcataggtctcagtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctga

SEQ ID NO: 540 - DNA, 882 bp, Homo sapiens
  V-C entry TRBV5-7_TRBC1
gccaccatgggccccgggctcctctgctgggtgctgctttgtcccctaggagaaggcccagtggacgctggagtcacccaaagtcccacacacctgatcaaaacgagaggacagcacgtgactctgagatgctctcctatctctgggcacaccagtgtgtcctcgtaccaacaggccctgggtcaggggccccagtttatctttcagtattatgagaaagaagagagaggaagaggaaacttccctgatcaattctcaggtcaccagttccctaactatagctctgagctgaatgtgaacgccttgttgctaggggactcggccctctatctttgcagagaccttgcggccgcataggtctcagtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctga

SEQ ID NO: 541 - DNA, 882 bp, Homo sapiens
  V-C entry TRBV5-8_TRBC1
gccaccatgggacccaggctcctcttctgggcactgctttgtctcctcggaacaggcccagtggaggctggagtcacacaaagtcccacacacctgatcaaaacgagaggacagcaagcgactctgagatgctctcctatctctgggcacaccagtgtgtactggtaccaacaggccctgggtctgggcctccagttcctcctttggtatgacgagggtgaagagagaaacagaggaaacttccctcctagattttcaggtcgccagttccctaattatagctctgagctgaatgtgaacgccttggagctggaggactcggccctgtatctttgcagagaccttgcggccgcataggtctcagtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctga

SEQ ID NO: 542 - DNA, 882 bp, Homo sapiens
  V-C entry TRBV6-1_TRBC1
gccaccatgagcatcgggctcctgtgctgtgtggccttttctctcctgtgggcaagtccagtgaatgctggtgtcactcagaccccaaaattccaggtcctgaaaacaggacagagcatgacactgcagtgtgcccaggatatgaaccataactccatgtactggtatcgacaagacccaggcatgggactgaggctgatttattactcagcttctgagggtaccactgacaaaggagaagtccccaatggctacaatgtctccagattaaacaaacgggagttctcgctcaggctggagtcggctgctccctcccagacatctgtgtacttttgcagagaccttgcggccgcataggtctcagtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctga

SEQ ID NO: 543 - DNA, 882 bp, Homo sapiens
  V-C entry TRBV6-3_TRBC1
gccaccatgagcctcgggctcctgtgctgtggggtcttttctctcctgtgggcaggtccagtgaatgctggtgtcactcagaccccaaaattccgggtcctgaaaacaggacagagcatgacactgctgtgtgcccaggatatgaaccatgaatacatgtactggtatcgacaagacccaggcatggggctgaggctgattcattactcagttggtgagggtacaactgccaaaggagaggtccctgatggctacaatgtctccagattaaaaaaacagaatttcctgctggggttggagtcggctgctccctcccaaacatctgtgtacttttgcagagaccttgcggccgcataggtctcagtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctga

SEQ ID NO: 544 - DNA, 882 bp, Homo sapiens
  V-C entry TRBV6-4_TRBC1
gccaccatgagaatcaggctcctgtgctgtgtggccttttctctcctgtgggcaggtccagtgattgctgggatcacccaggcaccaacatctcagatcctggcagcaggacggcgcatgacactgagatgtacccaggatatgagacataatgccatgtactggtatagacaagatctaggactggggctaaggctcatccattattcaaatactgcaggtaccactggcaaaggagaagtccctgatggttatagtgtctccagagcaaacacagatgatttccccctcacgttggcgtctgctgtaccctctcagacatctgtgtacttttgcagagaccttgcggccgcataggtctcagtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctga

SEQ ID NO: 545 - DNA, 882 bp, Homo sapiens
  V-C entry TRBV6-5_TRBC1
gccaccatgagcatcggcctcctgtgctgtgcagccttgtctctcctgtgggcaggtccagtgaatgctggtgtcactcagaccccaaaattccaggtcctgaaaacaggacagagcatgacactgcagtgtgcccaggatatgaaccatgaatacatgtcctggtatcgacaagacccaggcatggggctgaggctgattcattactcagttggtgctggtatcactgaccaaggagaagtccccaatggctacaatgtctccagatcaaccacagaggatttcccgctcaggctgctgtcggctgctccctcccagacatctgtgtacttttgcagagaccttgcggccgcataggtctcagtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctga

SEQ ID NO: 546 - DNA, 882 bp, Homo sapiens
  V-C entry TRBV6-6_TRBC1
gccaccatgagcatcagcctcctgtgctgtgcagcctttcctctcctgtgggcaggtccagtgaatgctggtgtcactcagaccccaaaattccgcatcctgaagataggacagagcatgacactgcagtgtacccaggatatgaaccataactacatgtactggtatcgacaagacccaggcatggggctgaagctgatttattattcagttggtgctggtatcactgataaaggagaagtcccgaatggctacaacgtctccagatcaaccacagaggatttcccgctcaggctggagttggctgctccctcccagacatctgtgtacttttgcagagaccttgcggccgcataggtctcagtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctga

SEQ ID NO: 547 - DNA, 879 bp, Homo sapiens
  V-C entry TRBV6-8_TRBC1
gccaccatgagcctcgggctcctgtgctgtgcggccttttctctcctgtgggcaggtcccgtgaatgctggtgtcactcagaccccaaaattccacatcctgaaaacaggacagagcatgacactgcagtgtgcccaggatatgaaccatggatacatgtcctggtatcgacaagacccaggcatggggctgagactgatttactactcagctgctgctggtactactgacaaagaagtccccaatggctacaatgtctctagattaaacacagaggatttcccactcaggctggtgtcggctgctccctcccagacatctgtgtacctttgcagagaccttgcggccgcataggtctcagtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctga

SEQ ID NO: 548 - DNA, 882 bp, Homo sapiens
  V-C entry TRBV6-9_TRBC1
gccaccatgagcatcgggctcctgtgctgtgtggccttttctctcctgtgggcaggtccagtgaatgctggtgtcactcagaccccaaaattccacatcctgaaaacaggacagagcatgacactgcagtgtgcccaggatatgaaccatggatacttgtcctggtatcgacaagacccaggcatggggctgaggcgcattcattactcagttgctgctggtatcactgacaaaggagaagtccccgatggctacaatgtatccagatcaaacacagaggatttcccgctcaggctggagtcagctgctccctcccagacatctgtatacttttgcagagaccttgcggccgcataggtctcagtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctga

SEQ ID NO: 549 - DNA, 885 bp, Homo sapiens
  V-C entry TRBV7-2_TRBC1
gccaccatgggcaccaggctcctcttctgggtggccttctgtctcctgggggcagatcacacaggagctggagtctcccagtcccccagtaacaaggtcacagagaagggaaaggatgtagagctcaggtgtgatccaatttcaggtcatactgccctttactggtaccgacagagcctggggcagggcctggagtttttaatttacttccaaggcaacagtgcaccagacaaatcagggctgcccagtgatcgcttctctgcagagaggactgggggatccgtctccactctgacgatccagcgcacacagcaggaggactcggccgtgtatctttgcagagaccttgcggccgcataggtctcagtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctga

SEQ ID NO: 550 - DNA, 885 bp, Homo sapiens
  V-C entry TRBV7-3_TRBC1
gccaccatgggcaccaggctcctctgctgggcagccctgtgcctcctgggggcagatcacacaggtgctggagtctcccagacccccagtaacaaggtcacagagaagggaaaatatgtagagctcaggtgtgatccaatttcaggtcatactgccctttactggtaccgacaaagcctggggcagggcccagagtttctaatttacttccaaggcacgggtgcggcagatgactcagggctgcccaacgatcggttctttgcagtcaggcctgagggatccgtctctactctgaagatccagcgcacagagcggggggactcagccgtgtatctttgcagagaccttgcggccgcataggtctcagtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctga

SEQ ID NO: 551 - DNA, 885 bp, Homo sapiens
  V-C entry TRBV7-6_TRBC1
gccaccatgggcaccagtctcctatgctgggtggtcctgggtttcctagggacagatcacacaggtgctggagtctcccagtctcccaggtacaaagtcacaaagaggggacaggatgtagctctcaggtgtgatccaatttcgggtcatgtatccctttattggtaccgacaggccctggggcagggcccagagtttctgacttacttcaattatgaagcccaacaagacaaatcagggctgcccaatgatcggttctctgcagagaggcctgagggatccatctccactctgacgatccagcgcacagagcagcgggactcggccatgtatcgttgcagagaccttgcggccgcataggtctcagtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctga

SEQ ID NO: 552 - DNA, 885 bp, Homo sapiens
  V-C entry TRBV7-7_TRBC1
gccaccatgggtaccagtctcctatgctgggtggtcctgggtttcctagggacagatcacacaggtgctggagtctcccagtctcccaggtacaaagtcacaaagaggggacaggatgtaactctcaggtgtgatccaatttcgagtcatgcaaccctttattggtatcaacaggccctggggcagggcccagagtttctgacttacttcaattatgaagctcaaccagacaaatcagggctgcccagtgatcggttctctgcagagaggcctgagggatccatctccactctgacgattcagcgcacagagcagcgggactcagccatgtatcgttgcagagaccttgcggccgcataggtctcagtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctga

SEQ ID NO: 553 - DNA, 885 bp, Homo sapiens
  V-C entry TRBV7-8_TRBC1
gccaccatgggcaccaggctcctctgctgggtggtcctgggtttcctagggacagatcacacaggtgctggagtctcccagtcccctaggtacaaagtcgcaaagagaggacaggatgtagctctcaggtgtgatccaatttcgggtcatgtatcccttttttggtaccaacaggccctggggcaggggccagagtttctgacttatttccagaatgaagctcaactagacaaatcggggctgcccagtgatcgcttctttgcagaaaggcctgagggatccgtctccactctgaagatccagcgcacacagcaggaggactccgccgtgtatctttgcagagaccttgcggccgcataggtctcagtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctga

SEQ ID NO: 554 - DNA, 885 bp, Homo sapiens
  V-C entry TRBV7-9_TRBC1
gccaccatgggcaccagcctcctctgctggatggccctgtgtctcctgggggcagatcacgcagatactggagtctcccagaaccccagacacaagatcacaaagaggggacagaatgtaactttcaggtgtgatccaatttctgaacacaaccgcctttattggtaccgacagaccctggggcagggcccagagtttctgacttacttccagaatgaagctcaactagaaaaatcaaggctgctcagtgatcggttctctgcagagaggcctaagggatctttctccaccttggagatccagcgcacagagcagggggactcggccatgtatctttgcagagaccttgcggccgcataggtctcagtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctga

SEQ ID NO: 555 - DNA, 882 bp, Homo sapiens
  V-C entry TRBV9_TRBC1
gccaccatgggcttcaggctcctctgctgtgtggccttttgtctcctgggagcaggcccagtggattctggagtcacacaaaccccaaagcacctgatcacagcaactggacagcgagtgacgctgagatgctcccctaggtctggtgacctctctgtgtactggtaccaacagagcctggaccagggcctccagttcctcattcagtattataatggagaagagagagcaaaaggaaacattcttgaacgattctccgcacaacagttccctgacttgcactctgaactaaacctgagctctctggagctgggggactcagctttgtacttttgcagagaccttgcggccgcataggtctcagtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctga

SEQ ID NO: 556 - DNA, 882 bp, Homo sapiens
  V-C entry TRBV10-1_TRBC1
gccaccatgggcacgaggctcttcttctatgtggccctttgtctgctgtgggcaggacacagggatgctgaaatcacccagagcccaagacacaagatcacagagacaggaaggcaggtgaccttggcgtgtcaccagacttggaaccacaacaatatgttctggtatcgacaagacctgggacatgggctgaggctgatccattactcatatggtgttcaagacactaacaaaggagaagtctcagatggctacagtgtctctagatcaaacacagaggacctccccctcactctggagtctgctgcctcctcccagacatctgtatacttttgcagagaccttgcggccgcataggtctcagtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctga

SEQ ID NO: 557 - DNA, 882 bp, Homo sapiens
  V-C entry TRBV10-2_TRBC1
gccaccatgggcaccaggctcttcttctatgtggccctttgtctgctgtgggcaggacacagggatgctggaatcacccagagcccaagatacaagatcacagagacaggaaggcaggtgaccttgatgtgtcaccagacttggagccacagctatatgttctggtatcgacaagacctgggacatgggctgaggctgatctattactcagcagctgctgatattacagataaaggagaagtccccgatggctatgttgtctccagatccaagacagagaatttccccctcactctggagtcagctacccgctcccagacatctgtgtacttttgcagagaccttgcggccgcataggtctcagtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctga

SEQ ID NO: 558 - DNA, 882 bp, Homo sapiens
  V-C entry TRBV10-3_TRBC1
gccaccatgggcacaaggttgttcttctatgtggccctttgtctcctgtggacaggacacatggatgctggaatcacccagagcccaagacacaaggtcacagagacaggaacaccagtgactctgagatgtcaccagactgagaaccaccgctatatgtactggtatcgacaagacccggggcatgggctgaggctgatccattactcatatggtgttaaagatactgacaaaggagaagtctcagatggctatagtgtctctagatcaaagacagaggatttcctcctcactctggagtccgctaccagctcccagacatctgtgtacttttgcagagaccttgcggccgcataggtctcagtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctga

SEQ ID NO: 559 - DNA, 885 bp, Homo sapiens
  V-C entry TRBV11-1_TRBC1
gccaccatgagcaccaggcttctctgctggatggccctctgtctcctgggggcagaactctcagaagctgaagttgcccagtcccccagatataagattacagagaaaagccaggctgtggctttttggtgtgatcctatttctggccatgctaccctttactggtaccggcagatcctgggacagggcccggagcttctggttcaatttcaggatgagagtgtagtagatgattcacagttgcctaaggatcgattttctgcagagaggctcaaaggagtagactccactctcaagatccagcctgcagagcttggggactcggccatgtatctttgcagagaccttgcggccgcataggtctcagtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctga

SEQ ID NO: 560 - DNA, 885 bp, Homo sapiens
  V-C entry TRBV11-2_TRBC1
gccaccatgggcaccaggctcctctgctgggcggccctctgtctcctgggagcagaactcacagaagctggagttgcccagtctcccagatataagattatagagaaaaggcagagtgtggctttttggtgcaatcctatatctggccatgctaccctttactggtaccagcagatcctgggacagggcccaaagcttctgattcagtttcagaataacggtgtagtggatgattcacagttgcctaaggatcgattttctgcagagaggctcaaaggagtagactccactctcaagatccagcctgcaaagcttgaggactcggccgtgtatctttgcagagaccttgcggccgcataggtctcagtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctga

SEQ ID NO: 561 - DNA, 885 bp, Homo sapiens
  V-C entry TRBV11-3_TRBC1
gccaccatgggtaccaggctcctctgctgggtggccttctgtctcctggtggaagaactcatagaagctggagtggttcagtctcccagatataagattatagagaaaaaacagcctgtggctttttggtgcaatcctatttctggccacaataccctttactggtacctgcagaacttgggacagggcccggagcttctgattcgatatgagaatgaggaagcagtagacgattcacagttgcctaaggatcgattttctgcagagaggctcaaaggagtagactccactctcaagatccagcctgcagagcttggggactcggccgtgtatctttgcagagaccttgcggccgcataggtctcagtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctga

SEQ ID NO: 562 - DNA, 885 bp, Homo sapiens
  V-C entry TRBV12-3_TRBC1
gccaccatggactcctggaccttctgctgtgtgtccctttgcatcctggtagcgaagcatacagatgctggagttatccagtcaccccgccatgaggtgacagagatgggacaagaagtgactctgagatgtaaaccaatttcaggccacaactcccttttctggtacagacagaccatgatgcggggactggagttgctcatttactttaacaacaacgttccgatagatgattcagggatgcccgaggatcgattctcagctaagatgcctaatgcatcattctccactctgaagatccagccctcagaacccagggactcagctgtgtacttttgcagagaccttgcggccgcataggtctcagtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctga

SEQ ID NO: 563 - DNA, 885 bp, Homo sapiens
  V-C entry TRBV12-4_TRBC1
gccaccatggactcctggaccctctgctgtgtgtccctttgcatcctggtagcaaagcacacagatgctggagttatccagtcaccccggcacgaggtgacagagatgggacaagaagtgactctgagatgtaaaccaatttcaggacacgactaccttttctggtacagacagaccatgatgcggggactggagttgctcatttactttaacaacaacgttccgatagatgattcagggatgcccgaggatcgattctcagctaagatgcctaatgcatcattctccactctgaagatccagccctcagaacccagggactcagctgtgtacttttgcagagaccttgcggccgcataggtctcagtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctga

SEQ ID NO: 564 - DNA, 885 bp, Homo sapiens
  V-C entry TRBV12-5_TRBC1
gccaccatggccaccaggctcctctgctgtgtggttctttgtctcctgggagaagagcttatagatgctagagtcacccagacaccaaggcacaaggtgacagagatgggacaagaagtaacaatgagatgtcagccaattttaggccacaatactgttttctggtacagacagaccatgatgcaaggactggagttgctggcttacttccgcaaccgggctcctctagatgattcggggatgccgaaggatcgattctcagcagagatgcctgatgcaactttagccactctgaagatccagccctcagaacccagggactcagctgtgtacttttgcagagaccttgcggccgcataggtctcagtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctga

SEQ ID NO: 565 - DNA, 912 bp, Homo sapiens
  V-C entry TRBV13_TRBC1
gccaccatgcttagtcctgacctgcctgactctgcctggaacaccaggctcctctgccatgtcatgctttgtctcctgggagcagtttcagtggctgctggagtcatccagtccccaagacatctgatcaaagaaaagagggaaacagccactctgaaatgctatcctatccctagacacgacactgtctactggtaccagcagggtccaggtcaggacccccagttcctcatttcgttttatgaaaagatgcagagcgataaaggaagcatccctgatcgattctcagctcaacagttcagtgactatcattctgaactgaacatgagctccttggagctgggggactcagccctgtacttttgcagagaccttgcggccgcataggtctcagtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctga

SEQ ID NO: 566 - DNA, 885 bp, Homo sapiens
  V-C entry TRBV14_TRBC1
gccaccatggtttccaggcttctcagtttagtgtccctttgtctcctgggagcaaagcacatagaagctggagttactcagttccccagccacagcgtaatagagaagggccagactgtgactctgagatgtgacccaatttctggacatgataatctttattggtatcgacgtgttatgggaaaagaaataaaatttctgttacattttgtgaaagagtctaaacaggatgagtccggtatgcccaacaatcgattcttagctgaaaggactggagggacgtattctactctgaaggtgcagcctgcagaactggaggattctggagtttacttttgcagagaccttgcggccgcataggtctcagtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctga

SEQ ID NO: 567 - DNA, 882 bp, Homo sapiens
  V-C entry TRBV15_TRBC1
gccaccatgggtcctgggcttctccactggatggccctttgtctccttggaacaggtcatggggatgccatggtcatccagaacccaagataccaggttacccagtttggaaagccagtgaccctgagttgttctcagactttgaaccataacgtcatgtactggtaccagcagaagtcaagtcaggccccaaagctgctgttccactactatgacaaagattttaacaatgaagcagacacccctgataacttccaatccaggaggccgaacacttctttctgctttcttgacatccgctcaccaggcctgggggacacagccatgtacctttgcagagaccttgcggccgcataggtctcagtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctga

SEQ ID NO: 568 - DNA, 885 bp, Homo sapiens
  V-C entry TRBV16_TRBC1
gccaccatgagcccaatattcacctgcatcacaatcctttgtctgctggctgcaggttctcctggtgaagaagtcgcccagactccaaaacatcttgtcagaggggaaggacagaaagcaaaattatattgtgccccaataaaaggacacagttatgttttttggtaccaacaggtcctgaaaaacgagttcaagttcttgatttccttccagaatgaaaatgtctttgatgaaacaggtatgcccaaggaaagattttcagctaagtgcctcccaaattcaccctgtagccttgagatccaggctacgaagcttgaggattcagcagtgtacttttgcagagaccttgcggccgcataggtctcagtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctga

SEQ ID NO: 569 - DNA, 885 bp, Homo sapiens
  V-C entry TRBV18_TRBC1
gccaccatggacaccagagtactctgctgtgcggtcatctgccttctgggggcaggactctcaaatgccggcgtcatgcagaacccaagacacctggtcaggaggaggggacaggaggcaagactgagatgcagcccaatgaaaggacacagtcatgtttactggtatcggcagctcccagaggaaggtctgaaattcatggtttatctccagaaagaaaatatcatagatgagtcaggaatgccaaaggaacgattttctgctgaatttcccaaagagggccccagcatcctgaggatccagcaggtagtgcgaggagattcggcagcttacttttgcagagaccttgcggccgcataggtctcagtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctga

SEQ ID NO: 570 - DNA, 882 bp, Homo sapiens
  V-C entry TRBV19_TRBC1
gccaccatgagcaaccaggtgctctgctgtgtggtcctttgtttcctgggagcaaacaccgtggatggtggaatcactcagtccccaaagtacctgttcagaaaggaaggacagaatgtgaccctgagttgtgaacagaatttgaaccacgatgccatgtactggtaccgacaggacccagggcaagggctgagattgatctactactcacagatagtaaatgactttcagaaaggagatatagctgaagggtacagcgtctctcgggagaagaaggaatcctttcctctcactgtgacatcggcccaaaagaacccgacagctttctatctttgcagagaccttgcggccgcataggtctcagtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctga

SEQ ID NO: 571 - DNA, 876 bp, Homo sapiens
  V-C entry TRBV20-1_TRBC1
gccaccatgctgctgcttctgctgcttctggggccaggctccgggcttggtgctgtcgtctctcaacatccgagctgggttatctgtaagagtggaacctctgtgaagatcgagtgccgttccctggactttcaggccacaactatgttttggtatcgtcagttcccgaaacagagtctcatgctgatggcaacttccaatgagggctccaaggccacatacgagcaaggcgtcgagaaggacaagtttctcatcaaccatgcaagcctgaccttgtccactctgacagtgaccagtgcccatcctgaagatagcagcttctacatttgcagagaccttgcggccgcataggtctcagtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctga

SEQ ID NO: 572 - DNA, 882 bp, Homo sapiens
  V-C entry TRBV24-1_TRBC1
gccaccatggcctccctgctcttcttctgtggggccttttatctcctgggaacagggtccatggatgctgatgttacccagaccccaaggaataggatcacaaagacaggaaagaggattatgctggaatgttctcagactaagggtcatgatagaatgtactggtatcgacaagacccaggactgggcctacggttgatctattactcctttgatgtcaaagatataaacaaaggagagatctctgatggatacagtgtctctcgacaggcacaggctaaattctccctgtccctagagtctgccatccccaaccagacagctctttacttttgcagagaccttgcggccgcataggtctcagtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctga

SEQ ID NO: 573 - DNA, 882 bp, Homo sapiens
  V-C entry TRBV25-1_TRBC1
gccaccatgactatcaggctcctctgctacatgggcttttattttctgggggcaggcctcatggaagctgacatctaccagaccccaagataccttgttatagggacaggaaagaagatcactctggaatgttctcaaaccatgggccatgacaaaatgtactggtatcaacaagatccaggaatggaactacacctcatccactattcctatggagttaattccacagagaagggagatctttcctctgagtcaacagtctccagaataaggacggagcattttcccctgaccctggagtctgccaggccctcacatacctctcagtacctttgcagagaccttgcggccgcataggtctcagtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctga

SEQ ID NO: 574 - DNA, 882 bp, Homo sapiens
  V-C entry TRBV27_TRBC1
gccaccatgggcccccagctccttggctatgtggtcctttgccttctaggagcaggccccctggaagcccaagtgacccagaacccaagatacctcatcacagtgactggaaagaagttaacagtgacttgttctcagaatatgaaccatgagtatatgtcctggtatcgacaagacccagggctgggcttaaggcagatctactattcaatgaatgttgaggtgactgataagggagatgttcctgaagggtacaaagtctctcgaaaagagaagaggaatttccccctgatcctggagtcgcccagccccaaccagacctctctgtacttttgcagagaccttgcggccgcataggtctcagtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctga

SEQ ID NO: 575 - DNA, 882 bp, Homo sapiens
  V-C entry TRBV28_TRBC1
gccaccatgggaatcaggctcctctgtcgtgtggccttttgtttcctggctgtaggcctcgtagatgtgaaagtaacccagagctcgagatatctagtcaaaaggacgggagagaaagtttttctggaatgtgtccaggatatggaccatgaaaatatgttctggtatcgacaagacccaggtctggggctacggctgatctatttctcatatgatgttaaaatgaaagaaaaaggagatattcctgaggggtacagtgtctctagagagaagaaggagcgcttctccctgattctggagtccgccagcaccaaccagacatctatgtacctttgcagagaccttgcggccgcataggtctcagtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctga

SEQ ID NO: 576 - DNA, 876 bp, Homo sapiens
  V-C entry TRBV29-1_TRBC1
gccaccatgctgagtctactgctccttctcctgggactaggctctgtgttcagtgctgtcatctctcaaaagccaagcagggatatctgtcaacgtggaacctccctgacgatccagtgtcaagtcgatagccaagtcaccatgatgttctggtaccgtcagcaacctggacagagcctgacactgatcgcaactgcaaatcagggctctgaggccacatatgagagtggatttgtcattgacaagtttcccatcagccgcccaaacctaacattctcaactctgactgtgagcaacatgagccctgaagatagcagcatatatctttgcagagaccttgcggccgcataggtctcagtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctga

SEQ ID NO: 577 - DNA, 876 bp, Homo sapiens
  V-C entry TRBV30_TRBC1
gccaccatgctctgctctctccttgcccttctcctgggcactttctttggggtcagatctcagactattcatcaatggccagcgaccctggtgcagcctgtgggcagcccgctctctctggagtgcactgtggagggaacatcaaaccccaacctatactggtaccgacaggctgcaggcaggggcctccagctgctcttctactccgttggtattggccagatcagctctgaggtgccccagaatctctcagcctccagaccccaggaccggcagttcatcctgagttctaagaagctccttctcagtgactctggcttctatctttgcagagaccttgcggccgcataggtctcagtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctga

SEQ ID NO: 578 - DNA, 64 bp, Artificial Sequence
  TRBJ-C1_receiving_F1
aattcggtctcgaagtcttctgcggccgctgaagacacaggacctgaacaaggtgttgagaccc

SEQ ID NO: 579 - DNA, 64 bp, Artificial Sequence
  TRBJ-C2_receiving_F1
aattcggtctcgaagtcttctgcggccgctgaagacacaggacctgaaaaacgtgttgagaccc

SEQ ID NO: 580 - DNA, 64 bp, Artificial Sequence
  TRBJ-C1_receiving_R1
tcgagggtctcaacaccttgttcaggtcctgtgtcttcagcggccgcagaagacttcgagaccg

SEQ ID NO: 581 - DNA, 64 bp, Artificial Sequence
  TRBJ-C2_receiving_R1
tcgagggtctcaacacgtttttcaggtcctgtgtcttcagcggccgcagaagacttcgagaccg

SEQ ID NO: 582 - DNA, 2366 bp, Artificial Sequence
  TRB J_C1 receiving cassette vector
gccagggttttcccagtcacgacgttgtaaaacgacggccagtgagcgcgacgtaatacgactcactatagggcgaattggcggaaggccgtcaaggccgcatgaattcggtctcgaagtcttctgcggccgctgaagacacaggacctgaacaaggtgttgagaccctcgagctgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaattcgtccagcatcagatgaaattgcagtttgttcatgtccgggttatcaataccatatttctggaacagacgtttctgcaggctcgggctaaattcacccagacaattccacagaattgccagatcctgataacgatctgcaataccaacacgaccaacatcaatgcagccaatcagtttaccctcatcaaaaatcaggttatccaggctaaaatcaccatgggtaacaacgctatccggactaaacggcagcagtttatgcatttctttccaaacctgttcaacaggccaaccattacgttcatcatcaaaatcgcttgcatcaaccagaccattattcatacggctctgtgcctgtgccagacgaaaaacacgatcgctattaaacggacaattacaaaccggaatgctatgcagacgacgcagaaaaactgccagtgcatcaacaatattttcgcctgaatccggatattcttccagaacctgaaatgcggttttacccggaattgcggtggtcagcagccatgcatcatccggtgtacgaataaaatgtttaatggtcggcagcggcataaattcggtcagccaattcagacgaaccatttcatcggtcacatcatttgcaacgctacctttaccatgtttcagaaacagttccggtgcatccggtttaccatacagacgataaatggttgcaccgctctgaccaacattatcacgtgcccatttatagccatacagatctgcatccatattgctattcagacgcggacggctacagctggtttcacgctgaatatggctcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaac

SEQ ID NO: 583 - DNA, 2366 bp, Artificial Sequence
  TRB J_C2 receiving cassette vector
gccagggttttcccagtcacgacgttgtaaaacgacggccagtgagcgcgacgtaatacgactcactatagggcgaattggcggaaggccgtcaaggccgcatgaattcggtctcgaagtcttctgcggccgctgaagacacaggacctgaaaaacgtgttgagaccctcgagctgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaattcgtccagcatcagatgaaattgcagtttgttcatgtccgggttatcaataccatatttctggaacagacgtttctgcaggctcgggctaaattcacccagacaattccacagaattgccagatcctgataacgatctgcaataccaacacgaccaacatcaatgcagccaatcagtttaccctcatcaaaaatcaggttatccaggctaaaatcaccatgggtaacaacgctatccggactaaacggcagcagtttatgcatttctttccaaacctgttcaacaggccaaccattacgttcatcatcaaaatcgcttgcatcaaccagaccattattcatacggctctgtgcctgtgccagacgaaaaacacgatcgctattaaacggacaattacaaaccggaatgctatgcagacgacgcagaaaaactgccagtgcatcaacaatattttcgcctgaatccggatattcttccagaacctgaaatgcggttttacccggaattgcggtggtcagcagccatgcatcatccggtgtacgaataaaatgtttaatggtcggcagcggcataaattcggtcagccaattcagacgaaccatttcatcggtcacatcatttgcaacgctacctttaccatgtttcagaaacagttccggtgcatccggtttaccatacagacgataaatggttgcaccgctctgaccaacattatcacgtgcccatttatagccatacagatctgcatccatattgctattcagacgcggacggctacagctggtttcacgctgaatatggctcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaac

SEQ ID NO: 584 - DNA, 35 bp, Homo sapiens
  TRBJ1-1*01_BB-S_F1
ctcgtttggacaaggcaccagactcacagttgtag

SEQ ID NO: 585 - DNA, 35 bp, Homo sapiens
  TRBJ1-2*01_BB-S_F1
ctcgtttggttcggggaccaggttaaccgttgtag

SEQ ID NO: 586 - DNA, 35 bp, Homo sapiens
  TRBJ1-3*01_BB-S_F1
ctcgtttggagagggaagttggctcactgttgtag

SEQ ID NO: 587 - DNA, 35 bp, Homo sapiens
  TRBJ1-4*01_BB-S_F1
ctcgtttggcagtggaacccagctctctgtcttgg

SEQ ID NO: 588 - DNA, 35 bp, Homo sapiens
  TRBJ1-5*01_BB-S_F1
ctcgtttggtgatgggactcgactctccatcctag

SEQ ID NO: 589 - DNA, 35 bp, Homo sapiens
  TRBJ1-6*01_BB-S_F1
ctcgtttgggaacgggaccaggctcactgtgacag

SEQ ID NO: 590 - DNA, 35 bp, Homo sapiens
  TRBJ2-1*01_BB-S_F1
ctcgtttgggccagggacacggctcaccgtgctag

SEQ ID NO: 591 - DNA, 35 bp, Homo sapiens
  TRBJ2-2*01_BB-S_F1
ctcgtttggagaaggctctaggctgaccgtactgg

SEQ ID NO: 592 - DNA, 35 bp, Homo sapiens
  TRBJ2-3*01_BB-S_F1
ctcgtttggcccaggcacccggctgacagtgctcg

SEQ ID NO: 593 - DNA, 35 bp, Homo sapiens
  TRBJ2-4*01_BB-S_F1
ctcgtttggcgccgggacccggctctcagtgctgg

SEQ ID NO: 594 - DNA, 35 bp, Homo sapiens
  TRBJ2-5*01_BB-S_F1
ctcgtttgggccaggcacgcggctcctggtgctcg

SEQ ID NO: 595 - DNA, 35 bp, Homo sapiens
  TRBJ2-6*01_BB-S_F1
ctcgtttggggccggcagcaggctgaccgtgctgg

SEQ ID NO: 596 - DNA, 35 bp, Homo sapiens
  TRBJ2-7*01_BB-S_F1
ctcgtttgggccgggcaccaggctcacggtcacag

SEQ ID NO: 597 - DNA, 35 bp, Homo sapiens
  TRBJ1-1*01_BB-S_R1
tcctctacaactgtgagtctggtgccttgtccaaa

SEQ ID NO: 598 - DNA, 35 bp, Homo sapiens
  TRBJ1-2*01_BB-S_R1
tcctctacaacggttaacctggtccccgaaccaaa

SEQ ID NO: 599 - DNA, 35 bp, Homo sapiens
  TRBJ1-3*01_BB-S_R1
tcctctacaacagtgagccaacttccctctccaaa

SEQ ID NO: 600 - DNA, 35 bp, Homo sapiens
  TRBJ1-4*01_BB-S_R1
tcctccaagacagagagctgggttccactgccaaa

SEQ ID NO: 601 - DNA, 35 bp, Homo sapiens
  TRBJ1-5*01_BB-S_R1
tcctctaggatggagagtcgagtcccatcaccaaa

SEQ ID NO: 602 - DNA, 35 bp, Homo sapiens
  TRBJ1-6*01_BB-S_R1
tcctctgtcacagtgagcctggtcccgttcccaaa

SEQ ID NO: 603 - DNA, 35 bp, Homo sapiens
  TRBJ2-1*01_BB-S_R1
tcctctagcacggtgagccgtgtccctggcccaaa

SEQ ID NO: 604 - DNA, 35 bp, Homo sapiens
  TRBJ2-2*01_BB-S_R1
tcctccagtacggtcagcctagagccttctccaaa

SEQ ID NO: 605 - DNA, 35 bp, Homo sapiens
  TRBJ2-3*01_BB-S_R1
tcctcgagcactgtcagccgggtgcctgggccaaa

SEQ ID NO: 606 - DNA, 35 bp, Homo sapiens
  TRBJ2-4*01_BB-S_R1
tcctccagcactgagagccgggtcccggcgccaaa

SEQ ID NO: 607 - DNA, 35 bp, Homo sapiens
  TRBJ2-5*01_BB-S_R1
tcctcgagcaccaggagccgcgtgcctggcccaaa

SEQ ID NO: 608 - DNA, 35 bp, Homo sapiens
  TRBJ2-6*01_BB-S_R1
tcctccagcacggtcagcctgctgccggccccaaa

SEQ ID NO: 609 - DNA, 35 bp, Homo sapiens
  TRBJ2-7*01_BB-S_R1
tcctctgtgaccgtgagcctggtgcccggcccaaa

SEQ ID NO: 610 - DNA, 44 bp, Homo sapiens
  TRBJ1-1*01_BB-L_F1
ctcggaagctttctttggacaaggcaccagactcacagttgtag

SEQ ID NO: 611 - DNA, 44 bp, Homo sapiens
  TRBJ1-2*01_BB-L_F1
ctcgggctacacctttggttcggggaccaggttaaccgttgtag

SEQ ID NO: 612 - DNA, 44 bp, Homo sapiens
  TRBJ1-3*01_BB-L_F1
ctcgaccatatattttggagagggaagttggctcactgttgtag

SEQ ID NO: 613 - DNA, 47 bp, Homo sapiens
  TRBJ1-4*01_BB-L_F1
ctcggaaaagctgttctttggcagtggaacccagctctctgtcttgg

SEQ ID NO: 614 - DNA, 44 bp, Homo sapiens
  TRBJ1-5*01_BB-L_F1
ctcgccccagcattttggtgatgggactcgactctccatcctag

SEQ ID NO: 615 - DNA, 47 bp, Homo sapiens
  TRBJ1-6*01_BB-L_F1
ctcgtcacccctccactttgggaacgggaccaggctcactgtgacag

SEQ ID NO: 616 - DNA, 44 bp, Homo sapiens
  TRB J Long segment part
ctcggagcagttctttgggccagggacacggctcaccgtgctag

SEQ ID NO: 617 - DNA, 47 bp, Homo sapiens
  TRB J Long segment part
ctcgggggagctgttctttggagaaggctctaggctgaccgtactgg

SEQ ID NO: 618 - DNA, 44 bp, Homo sapiens
  TRB J Long segment part
ctcgacgcagtattttggcccaggcacccggctgacagtgctcg

SEQ ID NO: 619 - DNA, 44 bp, Homo sapiens
  TRB J Long segment part
ctcgattcagtactttggcgccgggacccggctctcagtgctgg

SEQ ID NO: 620 - DNA, 44 bp, Homo sapiens
  TRB J Long segment part
ctcgacccagtactttgggccaggcacgcggctcctggtgctcg

SEQ ID NO: 621 - DNA, 47 bp, Homo sapiens
  TRB J Long segment part
ctcgaacgtcctgacttttggggccggcagcaggctgaccgtgctgg

SEQ ID NO: 622 - DNA, 41 bp, Homo sapiens
  TRB J Long segment part
ctcgcagtactttgggccgggcaccaggctcacggtcacag

SEQ ID NO: 623 - DNA, 44 bp, Homo sapiens
  TRBJ1-1*01_BB-L_R1
tcctctacaactgtgagtctggtgccttgtccaaagaaagcttc

SEQ ID NO: 624 - DNA, 44 bp, Homo sapiens
  TRBJ1-2*01_BB-L_R1
tcctctacaacggttaacctggtccccgaaccaaaggtgtagcc

SEQ ID NO: 625 - DNA, 44 bp, Homo sapiens
  TRBJ1-3*01_BB-L_R1
tcctctacaacagtgagccaacttccctctccaaaatatatggt

SEQ ID NO: 626 - DNA, 47 bp, Homo sapiens
  TRBJ1-4*01_BB-L_R1
tcctccaagacagagagctgggttccactgccaaagaacagcttttc

SEQ ID NO: 627 - DNA, 44 bp, Homo sapiens
  TRBJ1-5*01_BB-L_R1
tcctctaggatggagagtcgagtcccatcaccaaaatgctgggg

SEQ ID NO: 628 - DNA, 47 bp, Homo sapiens
  TRBJ1-6*01_BB-L_R1
tcctctgtcacagtgagcctggtcccgttcccaaagtggaggggtga

SEQ ID NO: 629 - DNA, 44 bp, Homo sapiens
  TRBJ2-1*01_BB-L_R1
tcctctagcacggtgagccgtgtccctggcccaaagaactgctc

SEQ ID NO: 630 - DNA, 47 bp, Homo sapiens
  TRBJ2-2*01_BB-L_R1
tcctccagtacggtcagcctagagccttctccaaagaacagctcccc

SEQ ID NO: 631 - DNA, 44 bp, Homo sapiens
  TRBJ2-3*01_BB-L_R1
tcctcgagcactgtcagccgggtgcctgggccaaaatactgcgt

SEQ ID NO: 632 - DNA, 44 bp, Homo sapiens
  TRBJ2-4*01_BB-L_R1
tcctccagcactgagagccgggtcccggcgccaaagtactgaat

SEQ ID NO: 633 - DNA, 44 bp, Homo sapiens
  TRBJ2-5*01_BB-L_R1
tcctcgagcaccaggagccgcgtgcctggcccaaagtactgggt

SEQ ID NO: 634 - DNA, 47 bp, Homo sapiens
  TRBJ2-6*01_BB-L_R1
tcctccagcacggtcagcctgctgccggccccaaaagtcaggacgtt

SEQ ID NO: 635 - DNA, 41 bp, Homo sapiens
  TRBJ2-7*01_BB-L_R1
tcctctgtgaccgtgagcctggtgcccggcccaaagtactg

SEQ ID NO: 636 - DNA, 63 bp, Homo sapiens
  J Donor_Short_C1_TRBJ1-1
ggtctcgtttggacaaggcaccagactcacagttgtagaggacctgaacaaggtgttgagacc

SEQ ID NO: 637 - DNA, 63 bp, Homo sapiens
  J Donor_Short_C1_TRBJ1-2
ggtctcgtttggttcggggaccaggttaaccgttgtagaggacctgaacaaggtgttgagacc

SEQ ID NO: 638 - DNA, 63 bp, Homo sapiens
  J Donor_Short_C1_TRBJ1-3
ggtctcgtttggagagggaagttggctcactgttgtagaggacctgaacaaggtgttgagacc

SEQ ID NO: 639 - DNA, 63 bp, Homo sapiens
  J Donor_Short_C1_TRBJ1-4
ggtctcgtttggcagtggaacccagctctctgtcttggaggacctgaacaaggtgttgagacc

SEQ ID NO: 640 - DNA, 63 bp, Homo sapiens
  J Donor_Short_C1_TRBJ1-5
ggtctcgtttggtgatgggactcgactctccatcctagaggacctgaacaaggtgttgagacc

SEQ ID NO: 641 - DNA, 63 bp, Homo sapiens
  J Donor_Short_C1_TRBJ1-6
ggtctcgtttgggaacgggaccaggctcactgtgacagaggacctgaacaaggtgttgagacc

SEQ ID NO: 642 - DNA, 63 bp, Homo sapiens
  J Donor_Short_C1_TRBJ2-1
ggtctcgtttgggccagggacacggctcaccgtgctagaggacctgaacaaggtgttgagacc

SEQ ID NO: 643 - DNA, 63 bp, Homo sapiens
  J Donor_Short_C1_TRBJ2-2
ggtctcgtttggagaaggctctaggctgaccgtactggaggacctgaacaaggtgttgagacc

SEQ ID NO: 644 - DNA, 63 bp, Homo sapiens
  J Donor_Short_C1_TRBJ2-3
ggtctcgtttggcccaggcacccggctgacagtgctcgaggacctgaacaaggtgttgagacc

SEQ ID NO: 645 - DNA, 63 bp, Homo sapiens
  J Donor_Short_C1_TRBJ2-4
ggtctcgtttggcgccgggacccggctctcagtgctggaggacctgaacaaggtgttgagacc

SEQ ID NO: 646 - DNA, 63 bp, Homo sapiens
  J Donor_Short_C1_TRBJ2-5
ggtctcgtttgggccaggcacgcggctcctggtgctcgaggacctgaacaaggtgttgagacc

SEQ ID NO: 647 - DNA, 63 bp, Homo sapiens
  J Donor_Short_C1_TRBJ2-6
ggtctcgtttggggccggcagcaggctgaccgtgctggaggacctgaacaaggtgttgagacc

SEQ ID NO: 648 - DNA, 63 bp, Homo sapiens
  J Donor_Short_C1_TRBJ2-7
ggtctcgtttgggccgggcaccaggctcacggtcacagaggacctgaacaaggtgttgagacc

SEQ ID NO: 649 - DNA, 63 bp, Homo sapiens
  J Donor_Short_C2_TRBJ1-1
ggtctcgtttggacaaggcaccagactcacagttgtagaggacctgaaaaacgtgttgagacc

SEQ ID NO: 650 - DNA, 63 bp, Homo sapiens
  J Donor_Short_C2_TRBJ1-2
ggtctcgtttggttcggggaccaggttaaccgttgtagaggacctgaaaaacgtgttgagacc

SEQ ID NO: 651 - DNA, 63 bp, Homo sapiens
  J Donor_Short_C2_TRBJ1-3
ggtctcgtttggagagggaagttggctcactgttgtagaggacctgaaaaacgtgttgagacc

SEQ ID NO: 652 - DNA, 63 bp, Homo sapiens
  J Donor_Short_C2_TRBJ1-4
ggtctcgtttggcagtggaacccagctctctgtcttggaggacctgaaaaacgtgttgagacc

SEQ ID NO: 653 - DNA, 63 bp, Homo sapiens
  J Donor_Short_C2_TRBJ1-5
ggtctcgtttggtgatgggactcgactctccatcctagaggacctgaaaaacgtgttgagacc

SEQ ID NO: 654 - DNA, 63 bp, Homo sapiens
  J Donor_Short_C2_TRBJ1-6
ggtctcgtttgggaacgggaccaggctcactgtgacagaggacctgaaaaacgtgttgagacc

SEQ ID NO: 655 - DNA, 63 bp, Homo sapiens
  J Donor_Short_C2_TRBJ2-1
ggtctcgtttgggccagggacacggctcaccgtgctagaggacctgaaaaacgtgttgagacc

SEQ ID NO: 656 - DNA, 63 bp, Homo sapiens
  J Donor_Short_C2_TRBJ2-2
ggtctcgtttggagaaggctctaggctgaccgtactggaggacctgaaaaacgtgttgagacc

SEQ ID NO: 657 - DNA, 63 bp, Homo sapiens
  J Donor_Short_C2_TRBJ2-3
ggtctcgtttggcccaggcacccggctgacagtgctcgaggacctgaaaaacgtgttgagacc

SEQ ID NO: 658 - DNA, 63 bp, Homo sapiens
  J Donor_Short_C2_TRBJ2-4
ggtctcgtttggcgccgggacccggctctcagtgctggaggacctgaaaaacgtgttgagacc

SEQ ID NO: 659 - DNA, 63 bp, Homo sapiens
  J Donor_Short_C2_TRBJ2-5
ggtctcgtttgggccaggcacgcggctcctggtgctcgaggacctgaaaaacgtgttgagacc

SEQ ID NO: 660 - DNA, 63 bp, Homo sapiens
  TRB C2 J Short donor vector
ggtctcgtttggggccggcagcaggctgaccgtgctggaggacctgaaaaacgtgttgagacc

SEQ ID NO: 661 - DNA, 63 bp, Homo sapiens
  J Donor_Short_C2_TRBJ2-7
ggtctcgtttgggccgggcaccaggctcacggtcacagaggacctgaaaaacgtgttgagacc

SEQ ID NO: 662 - DNA, 72 bp, Homo sapiens
  J Donor_Long_C1_TRBJ1-1
ggtctcggaagctttctttggacaaggcaccagactcacagttgtagaggacctgaacaaggtgttgagacc

SEQ ID NO: 663 - DNA, 72 bp, Homo sapiens
  J Donor_Long_C1_TRBJ1-2
ggtctcgggctacacctttggttcggggaccaggttaaccgttgtagaggacctgaacaaggtgttgagacc

SEQ ID NO: 664 - DNA, 72 bp, Homo sapiens
  J Donor_Long_C1_TRBJ1-3
ggtctcgaccatatattttggagagggaagttggctcactgttgtagaggacctgaacaaggtgttgagacc

SEQ ID NO: 665 - DNA, 75 bp, Homo sapiens
  J Donor_Long_C1_TRBJ1-4
ggtctcggaaaagctgttctttggcagtggaacccagctctctgtcttggaggacctgaacaaggtgttgagacc

SEQ ID NO: 666 - DNA, 72 bp, Homo sapiens
  J Donor_Long_C1_TRBJ1-5
ggtctcgccccagcattttggtgatgggactcgactctccatcctagaggacctgaacaaggtgttgagacc

SEQ ID NO: 667 - DNA, 75 bp, Homo sapiens
  J Donor_Long_C1_TRBJ1-6
ggtctcgtcacccctccactttgggaacgggaccaggctcactgtgacagaggacctgaacaaggtgttgagacc

SEQ ID NO: 668 - DNA, 72 bp, Homo sapiens
  J Donor_Long_C1_TRBJ2-1
ggtctcggagcagttctttgggccagggacacggctcaccgtgctagaggacctgaacaaggtgttgagacc

SEQ ID NO: 669 - DNA, 75 bp, Homo sapiens
  J Donor_Long_C1_TRBJ2-2
ggtctcgggggagctgttctttggagaaggctctaggctgaccgtactggaggacctgaacaaggtgttgagacc

SEQ ID NO: 670 - DNA, 72 bp, Homo sapiens
  J Donor_Long_C1_TRBJ2-3
ggtctcgacgcagtattttggcccaggcacccggctgacagtgctcgaggacctgaacaaggtgttgagacc

SEQ ID NO: 671 - DNA, 72 bp, Homo sapiens
  J Donor_Long_C1_TRBJ2-4
ggtctcgattcagtactttggcgccgggacccggctctcagtgctggaggacctgaacaaggtgttgagacc

SEQ ID NO: 672 - DNA, 72 bp, Homo sapiens
  J Donor_Long_C1_TRBJ2-5
ggtctcgacccagtactttgggccaggcacgcggctcctggtgctcgaggacctgaacaaggtgttgagacc

SEQ ID NO: 673 - DNA, 75 bp, Homo sapiens
  J Donor_Long_C1_TRBJ2-6
ggtctcgaacgtcctgacttttggggccggcagcaggctgaccgtgctggaggacctgaacaaggtgttgagacc

SEQ ID NO: 674 - DNA, 69 bp, Homo sapiens
  J Donor_Long_C1_TRBJ2-7
ggtctcgcagtactttgggccgggcaccaggctcacggtcacagaggacctgaacaaggtgttgagacc

SEQ ID NO: 675 - DNA, 72 bp, Homo sapiens
  J Donor_Long_C2_TRBJ1-1
ggtctcggaagctttctttggacaaggcaccagactcacagttgtagaggacctgaaaaacgtgttgagacc

SEQ ID NO: 676 - DNA, 72 bp, Homo sapiens
  J Donor_Long_C2_TRBJ1-2
ggtctcgggctacacctttggttcggggaccaggttaaccgttgtagaggacctgaaaaacgtgttgagacc

SEQ ID NO: 677 - DNA, 72 bp, Homo sapiens
  J Donor_Long_C2_TRBJ1-3
ggtctcgaccatatattttggagagggaagttggctcactgttgtagaggacctgaaaaacgtgttgagacc

SEQ ID NO: 678 - DNA, 75 bp, Homo sapiens
  J Donor_Long_C2_TRBJ1-4
ggtctcggaaaagctgttctttggcagtggaacccagctctctgtcttggaggacctgaaaaacgtgttgagacc

SEQ ID NO: 679 - DNA, 72 bp, Homo sapiens
  J Donor_Long_C2_TRBJ1-5
ggtctcgccccagcattttggtgatgggactcgactctccatcctagaggacctgaaaaacgtgttgagacc

SEQ ID NO: 680 - DNA, 75 bp, Homo sapiens
  J Donor_Long_C2_TRBJ1-6
ggtctcgtcacccctccactttgggaacgggaccaggctcactgtgacagaggacctgaaaaacgtgttgagacc

SEQ ID NO: 681 - DNA, 72 bp, Homo sapiens
  J Donor_Long_C2_TRBJ2-1
ggtctcggagcagttctttgggccagggacacggctcaccgtgctagaggacctgaaaaacgtgttgagacc

SEQ ID NO: 682 - DNA, 75 bp, Homo sapiens
  J Donor_Long_C2_TRBJ2-2
ggtctcgggggagctgttctttggagaaggctctaggctgaccgtactggaggacctgaaaaacgtgttgagacc

SEQ ID NO: 683 - DNA, 72 bp, Homo sapiens
  J Donor_Long_C2_TRBJ2-3
ggtctcgacgcagtattttggcccaggcacccggctgacagtgctcgaggacctgaaaaacgtgttgagacc

SEQ ID NO: 684 - DNA, 72 bp, Homo sapiens
  J Donor_Long_C2_TRBJ2-4
ggtctcgattcagtactttggcgccgggacccggctctcagtgctggaggacctgaaaaacgtgttgagacc

SEQ ID NO: 685 - DNA, 72 bp, Homo sapiens
  J Donor_Long_C2_TRBJ2-5
ggtctcgacccagtactttgggccaggcacgcggctcctggtgctcgaggacctgaaaaacgtgttgagacc

SEQ ID NO: 686 - DNA, 75 bp, Homo sapiens
  J Donor_Long_C2_TRBJ2-6
ggtctcgaacgtcctgacttttggggccggcagcaggctgaccgtgctggaggacctgaaaaacgtgttgagacc

SEQ ID NO: 687 - DNA, 69 bp, Homo sapiens
  J Donor_Long_C2_TRBJ2-7
ggtctcgcagtactttgggccgggcaccaggctcacggtcacagaggacctgaaaaacgtgttgagacc

SEQ ID NO: 688 - DNA, 2471 bp, Artificial Sequence
  V-C entry backbone F14/F15
tctagacgaagttcctattccgaagttcctattcttataggagtataggaacttcctcgagctgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcatgaattcgctaccgggaagttcctattccgaagttcctattctatcagaagtataggaacttcaggtacc

SEQ ID NO: 689 - DNA, 2471 bp, Artificial Sequence
  V-C entry backbone FRT/F3
tctagacgaagttcctattccgaagttcctattcttcaaatagtataggaacttcctcgagctgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcatgaattcgctaccgggaagttcctattccgaagttcctattctctagaaagtataggaacttcaggtacc

SEQ ID NO: 690 - DNA, 38 bp, Artificial Sequence
  Generic V cloning fragment (left part)
  misc_feature 38: /note=“The last nucleotide of SEQ ID 690 is linked to the first nucleotide of XNn (described in specification), and the last nucleotide of XNn is then linked to the first nucleotide of SEQ ID 745.”
gtcagatactccatgagcacgaagacttgtacgccacc

SEQ ID NO: 691 - DNA, 44 bp, Artificial Sequence
  Generic C cloning fragment (left part)
  misc_feature 44: /note=“The last nucleotide of SEQ ID 691 is linked to the first nucleotide of YNn (described in specification), and the last nucleotide of YNn is then linked to the first nucleotide of SEQ ID 746.”
gtgcactcctatgactaacggaagactaggccgcataggtctca

SEQ ID NO: 692 - DNA, 592 bp, Artificial Sequence
  Generic transient V-C entry vector (left part)
  misc_feature 592: /note=“The last nucleotide of SEQ ID 692 is linked to the first nucleotide of XNn (described in specification), and the last nucleotide of XNn is then linked to the first nucleotide of SEQ ID 747.”
atagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcaggtacgccacc

SEQ ID NO: 693 - DNA, 60 bp, Artificial Sequence
  Generic F14-F15 V-C entry vector (left part)
  misc_feature 60: /note=“The last nucleotide of SEQ ID 693 is linked to the first nucleotide of XNn (described in specification), and the last nucleotide of XNn is then linked to the first nucleotide of SEQ ID 749.”
gaagttcctattccgaagttcctattctatcagaagtataggaacttcaggtacgccacc

SEQ ID NO: 694 - DNA, 60 bp, Artificial Sequence
  Generic FRT-F3 V-C entry vector (left part)
  misc_feature 60: /note=“The last nucleotide of SEQ ID 694 is linked to the first nucleotide of XNn (described in specification), and the last nucleotide of XNn is then linked to the first nucleotide of SEQ ID 751.”
gaagttcctattccgaagttcctattctctagaaagtataggaacttcaggtacgccacc

SEQ ID NO: 695 - DNA, 38 bp, Artificial Sequence
  Generic J receiving cassette F (left part)
  misc_feature 38: /note=“The last nucleotide of SEQ ID 695 is linked to the first nucleotide of Y′Nn (described in specification), and the last nucleotide of Y′Nn is then linked to the first nucleotide of TGAGACCC.”
aattcggtctcgaagtcttctgcggccgctgaagacac

SEQ ID NO: 696 - DNA, 12 bp, Artificial Sequence
  Generic J receiving cassette R (left part)
  misc_feature 12: /note=“The last nucleotide of SEQ ID 696 is linked to the first nucleotide of Y′Nn (described in specification), and the last nucleotide of Y′Nn is then linked to the first nucleotide of SEQ ID 753.”
tcgagggtctca

SEQ ID NO: 697 - DNA, 38 bp, Artificial Sequence; identical to SEQ ID NO: 695
  Generic J receiving cassette vector (left part)
  misc_feature 38: /note=“The last nucleotide of SEQ ID 697 is linked to the first nucleotide of Y′Nn (described in specification), and the last nucleotide of Y′Nn is then linked to the first nucleotide of SEQ ID 754.”

SEQ ID NO: 698 - DNA, 10 bp, Artificial Sequence
  Generic J segment F (dummy, see specification for description)
aaaaaaaaaa

SEQ ID NO: 699 - DNA, 10 bp, Artificial Sequence
  Generic J segment R (dummy, see specification for description))
aaaaaaaaaa

SEQ ID NO: 700 - DNA, 13 bp, Artificial Sequence
  Generic J donor vector (left part)
  misc_feature 13: /note=“The last nucleotide of SEQ ID 700 is linked to the first nucleotide of ZNnY′Nn (described in specification), and the last nucleotide of ZNnY′Nn is then linked to the first nucleotide of SEQ ID 755.”
gaattcggtctcg

SEQ ID NO: 701 - DNA, 816 bp, Homo sapiens
  JG9 TRA FL
atgctccttgaacatttattaataatcttgtggatgcagctgacatgggtcagtggtcaacagctgaatcagagtcctcaatctatgtttatccaggaaggagaagatgtctccatgaactgcacttcttcaagcatatttaacacctggctatggtacaagcaggaccctggggaaggtcctgtcctcttgatagccttatataaggctggtgaattgacctcaaatggaagactgactgctcagtttggtataaccagaaaggacagcttcctgaatatctcagcatccatacccagtgatgtaggcatctacttctgtgctggacccatgaaaacctcctacgacaaggtgatatttgggccagggacaagcttatcagtcattccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 702 - DNA, 927 bp, Homo sapiens
  JG9 TRB FL
atggactcctggaccttctgctgtgtgtccctttgcatcctggtagcaaagcacacagatgctggagttatccagtcaccccggcacgaggtgacagagatgggacaagaagtgactctgagatgtaaaccaatttcaggacacgactaccttttctggtacagacagaccatgatgcggggactggagttgctcatttactttaacaacaacgttccgatagatgattcagggatgcccgaggatcgattctcagctaagatgcctaatgcatcattctccactctgaagatccagccctcagaacccagggactcagctgtgtacttctgtgccagcagttcggcaaactatggctacaccttcggttcggggaccaggttaaccgttgtagaggacctgaacaaggtgttcccacccgaggtcgctgtgtttgagccatcagaagcagagatctcccacacccaaaaggccacactggtgtgcctggccacaggcttcttccctgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctga

SEQ ID NO: 703 - DNA, 40 bp, Homo sapiens
  JG9 TRA odeCDR3 F
ctgcgctggacccatgaaaacctcctacgacaaggtgata

SEQ ID NO: 704 - DNA, 40 bp, Homo sapiens
  JG9 TRA odeCDR3 R
caaatatcaccttgtcgtaggaggttttcatgggtccagc

SEQ ID NO: 705 - DNA, 34 bp, Homo sapiens
  JG9 TRB odeCDR3 F
ttgcgccagcagttccgcaaactatggctacacc

SEQ ID NO: 706 - DNA, 34 bp, Homo sapiens
  JG9 TRB odeCDR3 R
caaaggtgtagccatagtttgcggaactgctggc

SEQ ID NO: 707 - DNA, 257 bp, Homo sapiens
  TRA HBD4.1.S9-1.D5.a
gaagacagaaagtccagcaccttgatcctgccccacgctacgctgagagacactgctgtgtactattgcatcgtcagggatcgttataacaccggtaaccagttctattttgggacagggacaagtttgacggtcattccaaatatccagaaccctgaccctgccgtgtaccagctgagagactctaaatccagtgacaagtctgtctgcctattcaccgattttgattctcaaacaaatgtgtcacaaagtaagga

SEQ ID NO: 708 - DNA, 257 bp, Homo sapiens
  TRB HBD4.1.S9-1.D5.b
cagggttttcccagtcacgactagaaaagagaagaggaatttccccctgatcctggagtcgcccagccccaaccagacctctctgtacttctgtgccagcagtttgggcccaagctcctacgagcagtacttcgggccgggcaccaggctcacggtcacagaggacctgaaaaacgtgttcccacccgaggtcgctgtgtttgagccatcagaagcagagatcttccacacccaaaaggccacactggtgtgcctgg

SEQ ID NO: 709 - DNA, 298 bp, Homo sapiens
  TRA HBD4.1.S9-2.A6.a
gttttcccagtcacgactccagaaggcaagaaaatccgccaaccttgtcatctccgcttcacaactgggggactcagcaatgtatttctgtgcaatgagagagggcatggatagcagctataaattgatcttcgggagtgggaccagactgctggtcaggcctgatatccagaaccctgaccctgccgtgtaccagctgagagactctaaatccagtgacaagtctgtctgcctattcaccgattttgattctcaaacaaatgtgtcacaaagtaaggattctgtactgagtgagtat

SEQ ID NO: 710 - DNA, 235 bp, Homo sapiens
  TRA HBD4.1.S9-2.A6.a
ctttctccactttggagatccagcgcacagagcagggggactcggccatgtatctctgtgccagcagtttgcacgaccggggggcgcggactgaagctttctttggacaaggcaccagactcacagttgtagaggacctgaacaaggtgttcccacccgaggtcgctgtgtttgagccatcagaagcagagatctcccacacccaaaaggccacactggtgtgcctggccacagg

SEQ ID NO: 711 - DNA, 273 bp, Homo sapiens
  TRA HBD4.1.S9-3.A1.a
cgacactaacctttcagtttggtgatgcaagaaaggacagttctctccacatcactgcagcccagcctggtgatacaggcctctacctctgtgcaggagctcgaggaggcttcaaaactatctttggagcaggaacaagactatttgttaaagcaaatatccagaaccctgaccctgccgtgtaccagctgagagactctaaatccagtgacaagtctgtctgcctattcaccgattttgattctcaaacaaatgtgtcacaaagtaaggatt

SEQ ID NO: 712 - DNA, 221 bp, Homo sapiens
  TRB HBD4.1.S9-3.A1.b
ccccctgatcctggagtcgcccaaccccaaccaagcctctctgtacttctgtgccagcarwttatcaccatctactgggaactatggctacaccttcggttcggggaccaggttaaccgttgtagaggacctgaacaaggtgttcccacccgaggtcgctgtgtttgagccatcagaagcagagatctcccacacccaaaaggcccaccactggtgkgcct

SEQ ID NO: 713 - DNA, 286 bp, Homo sapiens
  TRA HBD4.1.S9-7.E5.a
aaggcaagaaaatccgccaaccttgtcatmtccgcttcacaactgggggactcagcaatgtatttctgtgcaatgaggccttatactggaggcttcaaaactatctttggagcaggaacaagactatttgttaaagcaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactktgc

SEQ ID NO: 714 - DNA, 238 bp, Homo sapiens
  TRA HBD4.1.S9-7.E5.a
cgaaaagagaagaggaatttccccmtgatcctggagtcgcccagccccawccagacctctctgtacttctgtgccagctscactagcccctcgmcagctcgctatggctacwccttcggttcggggaccaggttaaccgttgtagaggacctgaacaaggtgttcccacccgaggtcgctgtgtttgagccatcagaagcagmswtctcccacacccaaaaggccacactgagkgkgc

SEQ ID NO: 715 - DNA, 306 bp, Homo sapiens
  TRA HBD4.1.S9-7.C5.a
tttgaacccgaatttaacaagacacaaacctccttccccctgaagaaaccatctgcccttgtgagcgattccgctttgtacttctgtgctgtgagatcccggatggatagcagctataaattgatcttcgggagtgggaccagactactggtcaggcctgatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtg

SEQ ID NO: 716 - DNA, 251 bp, Homo sapiens
  TRB HBD4.1.S9-7.C5.b
acgacctccagggttttctaagggatgtttctccaccttgaagatccagcgcacagagcagggggactcggccatgtatctctgtgccagcagcccccccgacgcggcctacaatgagcagttcttcgggccagggacacggctcaccgtgctagaggacctgaaaaacgtgttcccacccgaggtcgctgtgtttgagccatcagaagcagagatctcccacacccaaaaggccacactggtgtgcctgg

SEQ ID NO: 717 - DNA, 319 bp, Homo sapiens
  TRA HBD4.1.S9-5.E3.a
agggttttcccagtcacgacttaagagtcacgcttgacacttccaagaaaagcagttccttgttgatcacggcttcccgggcagcagacactgcttcttacttctgtgctacggagctccggatggatagcagctataaattgatcttcgggagtgggaccagactgctggtcaggcctgatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaa

SEQ ID NO: 718 - DNA, 270 bp, Homo sapiens
  TRA HBD4.1.S9-5.E3.b
agggttttcccagtcacgacctccagagaggcctaagggatgtttctccactttggagatccagcgcacagagcagggggactcggccatgtatctctgtgccagcagcttaagggaaggacgactcccagagacccagtacttcgggccaggcacgcggctcctggtgctcgaggacctgaaaaacgtgttcccacccgaggtcgctgtgtttgagccatcagaagcagagatctcccacacccaaaaggccacactggtgtgcctggc

SEQ ID NO: 719 - DNA, 43 bp, Homo sapiens
  Forward TRA HBD4.1.S9-1.D5
ctgcatcgtcagggatcgttataacaccggtaaccagttctat

SEQ ID NO: 720 - DNA, 43 bp, Homo sapiens
  Reverse TRA HBD4.1.S9-1.D5
caaaatagaactggttaccggtgttataacgatccctgacgat

SEQ ID NO: 721 - DNA, 40 bp, Homo sapiens
  Forward TRB HBD4.1.S9-1.D5
ttgcgccagcagtttgggcccaagctcctacgagcagtac

SEQ ID NO: 722 - DNA, 40 bp, Homo sapiens
  Reverse TRB HBD4.1.S9-1.D5
caaagtactgctcgtaggagcttgggcccaaactgctggc

SEQ ID NO: 723 - DNA, 43 bp, Homo sapiens
  Forward TRA HBD4.1.S9-2.A6
ctgcgcaatgagagagggcatggatagcagctataaattgatc

SEQ ID NO: 724 - DNA, 43 bp, Homo sapiens
  Reverse TRA HBD4.1.S9-2.A6
caaagatcaatttatagctgctatccatgccctctctcattgc

SEQ ID NO: 725 - DNA, 46 bp, Homo sapiens
  Forward TRB HBD4.1.S9-2.A6
ttgcgccagcagtttgcacgaccgtggtgcgcggactgaagctttc

SEQ ID NO: 726 - DNA, 46 bp, Homo sapiens
  Reverse TRB HBD4.1.S9-2.A6
caaagaaagcttcagtccgcgcaccacggtcgtgcaaactgctggc

SEQ ID NO: 727 - DNA, 34 bp, Homo sapiens
  Forward TRA HBD4.1.S9-3.A1
ctgcgcaggagctcgaggaggcttcaaaactatc

SEQ ID NO: 728 - DNA, 34 bp, Homo sapiens
  Reverse TRA HBD4.1.S9-3.A1
caaagatagttttgaagcctcctcgagctcctgc

SEQ ID NO: 729 - DNA, 46 bp, Homo sapiens
  Forward TRB HBD4.1.S9-3.A1
ttgcgccagcagtttatcaccatctactgggaactatggctacacc

SEQ ID NO: 730 - DNA, 46 bp, Homo sapiens
  Reverse TRB HBD4.1.S9-3.A1
caaaggtgtagccatagttcccagtagatggtgataaactgctggc

SEQ ID NO: 731 - DNA, 40 bp, Homo sapiens
  Forward TRA HBD4.1.S9-7.E5
ctgcgcaatgaggccttatactggaggcttcaaaactatc

SEQ ID NO: 732 - DNA, 40 bp, Homo sapiens
  Reverse TRA HBD4.1.S9-7.E5
caaagatagttttgaagcctccagtataaggcctcattgc

SEQ ID NO: 733 - DNA, 46 bp, Homo sapiens
  Forward TRB HBD4.1.S9-7.E5
ttgcgccagctccactagcccctcgacagctcgctatggctacacc

SEQ ID NO: 734 - DNA, 46 bp, Homo sapiens
  Reverse TRB HBD4.1.S9-7.E5
caaaggtgtagccatagcgagctgtcgaggggctagtggagctggc

SEQ ID NO: 735 - DNA, 43 bp, Homo sapiens
  Forward TRA HBD4.1.S9-7.C5
ctgcgctgtgagatcccggatggatagcagctataaattgatc

SEQ ID NO: 736 - DNA, 43 bp, Homo sapiens
  Reverse TRA HBD4.1.S9-7.C5
caaagatcaatttatagctgctatccatccgggatctcacagc

SEQ ID NO: 737 - DNA, 43 bp, Homo sapiens
  Forward TRB HBD4.1.S9-7.C5
ttgcgccagcagccctcctgacgcggcctacaatgagcagttc

SEQ ID NO: 738 - DNA, 43 bp, Homo sapiens
  Reverse TRB HBD4.1.S9-7.C5
caaagaactgctcattgtaggccgcgtcaggagggctgctggc

SEQ ID NO: 739 - DNA, 43 bp, Homo sapiens
  Forward TRA HBD4.1.S9-5.E3
ctgcgctacggagctccggatggatagcagctataaattgatc

SEQ ID NO: 740 - DNA, 43 bp, Homo sapiens
  Reverse TRA HBD4.1.S9-5.E3
caaagatcaatttatagctgctatccatccggagctccgtagc

SEQ ID NO: 741 - DNA, 46 bp, Homo sapiens
  Forward TRB HBD4.1.S9-5.E3
ttgcgccagcagcttaagggaaggacgactcccagagacacagtac

SEQ ID NO: 742 - DNA, 46 bp, Homo sapiens
  Reverse TRB HBD4.1.S9-5.E3
caaagtactgtgtctctgggagtcgtccttcccttaagctgctggc

SEQ ID NO: 743 - DNA, 40 bp, Artificial Sequence
  Forward TRA odeCDR3 - 3 positions degenerate
  misc_feature 15: /note=“n = a, t, c or g”
  misc_feature 20: /note=“n = a, t, c or g”
  misc_feature 26: /note=“n = a, t, c or g”
ctgcgctggacccangaaancctccnacgacaaggtgata

SEQ ID NO: 744 - DNA, 40 bp, Artificial Sequence
  Reverse TRA odeCDR3 - 3 positions degenerate
  misc_feature 19: /note=“n = a, t, c or g”
  misc_feature 25: /note=“n = a, t, c or g”
  misc_feature 30: /note=“n = a, t, c or g”
caaatatcaccttgtcgtnggaggntttcntgggtccagc

SEQ ID NO: 745 - DNA, 43 bp, Artificial Sequence
  Generic V cloning fragment (right part)
  misc_feature 1: /note=“The first nucleotide of SEQ ID 745 is linked to the last nucleotide of XNn (described in specification), and the first nucleotide of XNn is then linked to the last nucleotide of SEQ ID 690. ”
agagaccttgcggccgtgtcttcgactagtagctcacctacga

SEQ ID NO: 746 - DNA, 32 bp, Artificial Sequence
  Generic C cloning fragment (right part)
  misc_feature 1: /note=“The first nucleotide of SEQ ID 746 is linked to the last nucleotide of YNn (described in specification), and the first nucleotide of YNn is then linked to the last nucleotide of SEQ ID 691.”
ctaggtgtcttccctatgctgaatcgatggtc

SEQ ID NO: 747 - DNA, 27 bp, Artificial Sequence
  Generic transient V-C entry vector (middle part)
  misc_feature 1: /note=“The first nucleotide of SEQ ID 747 is linked to the last nucleotide of XNn (described in specification), and the first nucleotide of XNn is then linked to the last nucleotide of SEQ ID 692.”
  misc_feature 27: /note=“The last nucleotide of SEQ ID 747 is linked to the first nucleotide of YNn (described in specification), and the last nucleotide of YNn is then linked to the first nucleotide of SEQ ID 748.”
agagaccttgcggccgcataggtctca

SEQ ID NO: 748 - DNA, 2622 bp, Artificial Sequence
  Generic transient V-C entry vector (right part)
  misc_feature 1: /note=“The first nucleotide of SEQ ID 748 is linked to the last nucleotide of YNn (described in specification), and the first nucleotide of YNn is then linked to the last nucleotide of SEQ ID 747.”
ctagacctgatcataatcaagccatatcacatctgtagaggtttacttgctttaaaaaacctccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgcggatccaatctcgagctgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcatgaattcgctaccggt

SEQ ID NO: 749 - DNA, 27 bp, Artificial Sequence
  Generic F14-F15 V-C entry vector (middle part)
  misc_feature 1: /note=“The first nucleotide of SEQ ID 749 is linked to the last nucleotide of XNn (described in specification), and the first nucleotide of XNn is then linked to the last nucleotide of SEQ ID 693.”
  misc_feature 27: /note=“The last nucleotide of SEQ ID 749 is linked to the first nucleotide of YNn (described in specification), and the last nucleotide of YNn is then linked to the first nucleotide of SEQ ID 750.”
agagaccttgcggccgcataggtctca

SEQ ID NO: 750 - DNA, 2415 bp, Artificial Sequence
  Generic F14-F15 V-C entry vector (right part)
  misc_feature 1: /note=“The first nucleotide of SEQ ID 750 is linked to the last nucleotide of YNn (described in specification), and the first nucleotide of YNn is then linked to the last nucleotide of SEQ ID 749.”
ctagacgaagttcctattccgaagttcctattcttataggagtataggaacttcctcgagctgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcatgaattcgctaccgg

SEQ ID NO: 751 - DNA, 27 bp, Artificial Sequence
  Generic FRT-F3 V-C entry vector (middle part)
  misc_feature 1: /note=“The first nucleotide of SEQ ID 751 is linked to the last nucleotide of XNn (described in specification), and the first nucleotide of XNn is then linked to the last nucleotide of SEQ ID 694.”
  misc_feature 27: /note=“The last nucleotide of SEQ ID 751 is linked to the first nucleotide of YNn (described in specification), and the last nucleotide of YNn is then linked to the first nucleotide of SEQ ID 752.”
agagaccttgcggccgcataggtctca

SEQ ID NO: 752 - DNA, 2415 bp, Artificial Sequence
  Generic FRT-F3 V-C entry vector (middle part)
  misc_feature 1: /note=“The first nucleotide of SEQ ID 752 is linked to the last nucleotide of YNn (described in specification), and the first nucleotide of YNn is then linked to the last nucleotide of SEQ ID 751.”
ctagacgaagttcctattccgaagttcctattcttcaaatagtataggaacttcctcgagctgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcatgaattcgctaccgg

SEQ ID NO: 753 - DNA, 34 bp, Artificial Sequence
  Generic J receiving cassette R (right part)
  misc_feature 1: /note=“The first nucleotide of SEQ ID 753 is linked to the last nucleotide of Y′Nn (described in specification), and the first nucleotide of Y′Nn is then linked to the last nucleotide of SEQ ID 696.”
gtgtcttcagcggccgcagaagacttcgagaccg

SEQ ID NO: 754 - DNA, 2310 bp, Artificial Sequence
  Generic J receiving cassette vector (right part)
  misc_feature 1: /note=“The first nucleotide of SEQ ID 754 is linked to the last nucleotide of Y′Nn (described in specification), and the first nucleotide of Y′Nn is then linked to the last nucleotide of SEQ ID 697.”
tgagaccctcgagctgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaattcgtccagcatcagatgaaattgcagtttgttcatgtccgggttatcaataccatatttctggaacagacgtttctgcaggctcgggctaaattcacccagacaattccacagaattgccagatcctgataacgatctgcaataccaacacgaccaacatcaatgcagccaatcagtttaccctcatcaaaaatcaggttatccaggctaaaatcaccatgggtaacaacgctatccggactaaacggcagcagtttatgcatttctttccaaacctgttcaacaggccaaccattacgttcatcatcaaaatcgcttgcatcaaccagaccattattcatacggctctgtgcctgtgccagacgaaaaacacgatcgctattaaacggacaattacaaaccggaatgctatgcagacgacgcagaaaaactgccagtgcatcaacaatattttcgcctgaatccggatattcttccagaacctgaaatgcggttttacccggaattgcggtggtcagcagccatgcatcatccggtgtacgaataaaatgtttaatggtcggcagcggcataaattcggtcagccaattcagacgaaccatttcatcggtcacatcatttgcaacgctacctttaccatgtttcagaaacagttccggtgcatccggtttaccatacagacgataaatggttgcaccgctctgaccaacattatcacgtgcccatttatagccatacagatctgcatccatattgctattcagacgcggacggctacagctggtttcacgctgaatatggctcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcatg

SEQ ID NO: 755 - DNA, 2309 bp, Artificial Sequence
  Generic J donor vector (right part)
  misc_feature 1: /note=“The first nucleotide of SEQ ID 755 is linked to the last nucleotide of ZNnY′Nn (described in specification), and the first nucleotide of ZNnY′Nn is then linked to the last nucleotide of SEQ ID 700.”
tgagaccctcgagctgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaattcgtccagcatcagatgaaattgcagtttgttcatgtccgggttatcaataccatatttctggaacagacgtttctgcaggctcgggctaaattcacccagacaattccacagaattgccagatcctgataacgatctgcaataccaacacgaccaacatcaatgcagccaatcagtttaccctcatcaaaaatcaggttatccaggctaaaatcaccatgggtaacaacgctatccggactaaacggcagcagtttatgcatttctttccaaacctgttcaacaggccaaccattacgttcatcatcaaaatcgcttgcatcaaccagaccattattcatacggctctgtgcctgtgccagacgaaaaacacgatcgctattaaacggacaattacaaaccggaatgctatgcagacgacgcagaaaaactgccagtgcatcaacaatattttcgcctgaatccggatattcttccagaacctgaaatgcggttttacccggaattgcggtggtcagcagccatgcatcatccggtgtacgaataaaatgtttaatggtcggcagcggcataaattcggtcagccaattcagacgaaccatttcatcggtcacatcatttgcaacgctacctttaccatgtttcagaaacagttccggtgcatccggtttaccatacagacgataaatggttgcaccgctctgaccaacattatcacgtgcccatttatagccatacagatctgcatccatattgctattcagacgcggacggctacagctggtttcacgctgaatatggctcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcat

SEQ ID NO: 756 - DNA, 2510 bp, Artificial Sequence
  V-Ca entry backbone FRT/F3
ctagctagagacgttgtcgacatcgtctcttacctagacgaagttcctattccgaagttcctattcttcaaatagtataggaacttcctcgagctgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcatgaattcgctaccgggaagttcctattccgaagttcctattctctagaaagtataggaacttcaggtactcaggtac

SEQ ID NO: 757 - DNA, 785 bp, Artificial Sequence
  V-C entry TRAV7_TRAC_FRT-F3
gccaccatggagaagatgcgtagacctgtcctaattatattttgtctatgtcttggctgggcaaatggagaaaaccaggtggagcacagccctcattttctgggaccccagcagggagatgttgcctccatgagctgcacgtactctgtcagtcgttttaacaatttgcagtggtacaggcaaaatacagggatgggtcccaaacacctattatccatgtattcagctggatatgagaagcagaaaggaaggctaaatgctacattactgaagaatggaagcagcttgtacattacagccgtgcagcctgaagattcagccacctatttctgcagagaccttgcggccgcataggtctcaccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaggacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctgactag

SEQ ID NO: 758 - DNA, 788 bp, Artificial Sequence
  V-C entry TRAV8-2_TRAC_FRT-F3
gccaccatgctcctgctgctcgtcccagtgctcgaggtgatttttactctgggaggaaccagagcccagtcggtgacccagcttgacagccacgtttctgtctctgaaggaaccccggtgctgctgaggtgcaactactcatcttcttattcaccatctctcttctggtatgtgcaacaccccaacaaaggactccagcttctcctgaagtacacatcagcggccaccctggttaaaggcatcaacggttttgaggctgaatttaagaagagtgaaacctccttccacctgacgaaaccctcagcccatatgagcgacgcggctgagtacttctgcagagaccttgcggccgcataggtctcaccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaggacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctgactag

SEQ ID NO: 759 - DNA, 784 bp, Artificial Sequence
  V-C entry TRAV8-4_TRAC_FRT-F3
gccaccatgctcctgctgctcgtcccagtgctcgaggtgatttttaccctgggaggaaccagagcccagtcggtgacccagcttggcagccacgtttctgtctctgaaggagccctggttctgctgaggtgcaactactcatcgtctgttccaccatatctcttctggtatgtgcaataccccaaccaaggactccagcttctcctgaagtacacatcagcggccaccctggttaaaggcatcaacggttttgaggctgaatttaagaagagtgaaacctccttccacctgacgaaaccctcagcccatatgagcgacgcggctgagtacttctgcagagaccttgcggccgcataggtctcaccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaggacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 760 - DNA, 778 bp, Artificial Sequence
  V-C entry TRAV18_TRAC_FRT-F3
gccaccatgctgtctgcttcctgctcaggacttgtgatcttgttgatattcagaaggaccagtggagactcggttacccagacagaaggcccagttaccctccctgagagggcagctctgacattaaactgcacttatcagtccagctattcaacttttctattctggtatgtccagtatctaaacaaagagcctgagctcctcctgaaaagttcagaaaaccaggagactgacagcagaggttttcaggccagtcctatcaagagtgacagttccttccacctggagaagccctcggtgcagctgtcggactctgccgtgtactactgcagagaccttgcggccgcataggtctcaccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaggacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 761 - DNA, 790 bp, Artificial Sequence
  V-C entry TRAV38-2DV8_TRAC_FRT-F3
gccaccatggcatgccctggcttcctgtgggcacttgtgatctccacctgtcttgaatttagcatggctcagacagtcactcagtctcaaccagagatgtctgtgcaggaggcagaaacggtgaccctgagctgcacatatgacaccagtgagagtgattattatttattctggtacaagcagcctcccagcaggcagatgattctcgttattcgccaagaagcttataagcaacagaatgcaacagagaatcgtttctctgtgaacttccagaaagcagccaaatccttcagtctcaagatctcagactcacagctgggggatgccgcgatgtatttctgcagagaccttgcggccgcataggtctcaccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaggacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 762 - DNA, 775 bp, Artificial Sequence
  V-C entry TRAV39_TRAC_FRT-F3
gccaccatgaagaagctactagcaatgattctgtggcttcaactagaccggttaagtggagagctgaaagtggaacaaaaccctctgttcctgagcatgcaggagggaaaaaactataccatctactgcaattattcaaccacttcagacagactgtattggtacaggcaggatcctgggaaaagtctggaatctctgtttgtgttgctatcaaatggagcagtgaagcaggagggacgattaatggcctcacttgataccaaagcccgactcagcaccctccacatcacagctgccgtgcatgacctctctgccacctacttctgcagagaccttgcggccgcataggtctcaccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaggacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 763 - DNA, 760 bp, Artificial Sequence
  V-C entry TRAV40_TRAC_FRT-F3
gccaccatgaactcctctctggactttctaattctgatcttaatgtttggaggaaccagcagcaattcagtcaagcagacgggccaaataaccgtatcggagggagcatctgtgactatgaactgcacatacacatccacggggtaccctacccttttctggtatgtggaataccccagcaaacctctgcagcttcttcagagagagacaatggaaaacagcaaaaacttcggaggcggaaatattaaagacaaaaactcccccattgtgaaatattcagtccaggtatcagactcagccgtgtactactgcagagaccttgcggccgcataggtctcaccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaggacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 764 - DNA, 2501 bp, Artificial Sequence
  V-Cb entry backbone F14/F15
ctagacctggagacgacgcgtctagacgaagttcctattccgaagttcctattcttataggagtataggaacttcctcgagctgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcatgaattcgctaccgggaagttcctattccgaagttcctattctatcagaagtataggaacttcaggtacgtctcaggtac

SEQ ID NO: 765 - DNA, 888 bp, Artificial Sequence
  V-Cb entry TRBV6-6_TRBC2_F14-F15
gccaccatgagcatcagcctcctgtgctgtgcagcctttcctctcctgtgggcaggtccagtgaatgctggtgtcactcagaccccaaaattccgcatcctgaagataggacagagcatgacactgcagtgtacccaggatatgaaccataactacatgtactggtatcgacaagacccaggcatggggctgaagctgatttattattcagttggtgctggtatcactgataaaggagaagtcccgaatggctacaacgtgtccagatcaaccacagaggatttcccgctcaggctggagttggctgctccctcccagacatctgtgtacttttgcagagaccttgcggccgcataggtctcagtgttcccacccgaggtcgctgtgtttgagccatcagaagcagagatctcccacacccaaaaggccacactggtatgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 766 - DNA, 891 bp, Artificial Sequence
  V-Cb entry TRBV7-2_TRBC2_F14-F15
gccaccatgggcaccaggctcctcttctgggtggccttctgtctcctgggggcagatcacacaggagctggagtctcccagtcccccagtaacaaggtcacagagaagggaaaggatgtagagctcaggtgtgatccaatttcaggtcatactgccctttactggtaccgacagagcctggggcagggcctggagtttttaatttacttccaaggcaacagtgcaccagacaaatcagggctgcccagtgatcgcttctctgcagagaggactgggggatccgtgtccactctgacgatccagcgcacacagcaggaggactcggccgtgtatctttgcagagaccttgcggccgcataggtctcagtgttcccacccgaggtcgctgtgtttgagccatcagaagcagagatctcccacacccaaaaggccacactggtatgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 767 - DNA, 891 bp, Artificial Sequence
  V-Cb entry TRBV7-3_TRBC2_F14-F15
gccaccatgggcaccaggctcctctgctgggcagccctgtgcctcctgggggcagatcacacaggtgctggagtctcccagacccccagtaacaaggtcacagagaagggaaaatatgtagagctcaggtgtgatccaatttcaggtcatactgccctttactggtaccgacaaagcctggggcagggcccagagtttctaatttacttccaaggcacgggtgcggcagatgactcagggctgcccaacgatcggttctttgcagtcaggcctgagggatccgtgtctactctgaagatccagcgcacagagcggggggactcagccgtgtatctttgcagagaccttgcggccgcataggtctcagtgttcccacccgaggtcgctgtgtttgagccatcagaagcagagatctcccacacccaaaaggccacactggtatgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 768 - DNA, 891 bp, Artificial Sequence
  V-Cb entry TRBV7-8_TRBC2_F14-F15
gccaccatgggcaccaggctcctctgctgggtggtcctgggtttcctagggacagatcacacaggtgctggagtctcccagtcccctaggtacaaagtcgcaaagagaggacaggatgtagctctcaggtgtgatccaatttcgggtcatgtatcccttttttggtaccaacaggccctggggcaggggccagagtttctgacttatttccagaatgaagctcaactagacaaatcggggctgcccagtgatcgcttctttgcagaaaggcctgagggatctgtctccactctgaagatccagcgcacacagcaggaggactccgccgtgtatctttgcagagaccttgcggccgcataggtctcagtgttcccacccgaggtcgctgtgtttgagccatcagaagcagagatctcccacacccaaaaggccacactggtatgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 769 - DNA, 888 bp, Artificial Sequence
  V-Cb entry TRBV19_TRBC2_F14-F15
gccaccatgagcaaccaggtgctctgctgtgtggtcctttgtttcctgggagcaaacaccgtggatggtggaatcactcagtccccaaagtacctgttcagaaaggaaggacagaatgtgaccctgagttgtgaacagaatttgaaccacgatgccatgtactggtaccgacaggacccagggcaagggctgagattgatctactactcacagatagtaaatgactttcagaaaggagatatagctgaagggtacagcgtgtctcgggagaagaaggaatcctttcctctcactgtgacatcggcccaaaagaacccgacagctttctatctttgcagagaccttgcggccgcataggtctcagtgttcccacccgaggtcgctgtgtttgagccatcagaagcagagatctcccacacccaaaaggccacactggtatgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 770 - DNA, 882 bp, Artificial Sequence
  V-Cb entry TRBV20-1_TRBC2_F14-F15
gccaccatgctgctgcttctgctgcttctggggccaggctccgggcttggtgctgtcgtgtctcaacatccgagctgggttatctgtaagagtggaacctctgtgaagatcgagtgccgttccctggactttcaggccacaactatgttttggtatcgtcagttcccgaaacagagtctcatgctgatggcaacttccaatgagggctccaaggccacatacgagcaaggcgtcgagaaggacaagtttctcatcaaccatgcaagcctgaccttgtccactctgacagtgaccagtgcccatcctgaagatagcagcttctacatttgcagagaccttgcggccgcataggtctcagtgttcccacccgaggtcgctgtgtttgagccatcagaagcagagatctcccacacccaaaaggccacactggtatgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 771 - DNA, 882 bp, Artificial Sequence
  V-Cb entry TRBV6-6_TRBC1_F14-F15
gccaccatgagcatcagcctcctgtgctgtgcagcctttcctctcctgtgggcaggtccagtgaatgctggtgtcactcagaccccaaaattccgcatcctgaagataggacagagcatgacactgcagtgtacccaggatatgaaccataactacatgtactggtatcgacaagacccaggcatggggctgaagctgatttattattcagttggtgctggtatcactgataaaggagaagtcccgaatggctacaacgtgtccagatcaaccacagaggatttcccgctcaggctggagttggctgctccctcccagacatctgtgtacttttgcagagaccttgcggccgcataggtctcagtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctga

SEQ ID NO: 772 - DNA, 885 bp, Artificial Sequence
  V-Cb entry TRBV7-2_TRBC1_F14-F15
gccaccatgggcaccaggctcctcttctgggtggccttctgtctcctgggggcagatcacacaggagctggagtctcccagtcccccagtaacaaggtcacagagaagggaaaggatgtagagctcaggtgtgatccaatttcaggtcatactgccctttactggtaccgacagagcctggggcagggcctggagtttttaatttacttccaaggcaacagtgcaccagacaaatcagggctgcccagtgatcgcttctctgcagagaggactgggggatccgtgtccactctgacgatccagcgcacacagcaggaggactcggccgtgtatctttgcagagaccttgcggccgcataggtctcagtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctga

SEQ ID NO: 773 - DNA, 885 bp, Artificial Sequence
  V-Cb entry TRBV7-3_TRBC1_F14-F15
gccaccatgggcaccaggctcctctgctgggcagccctgtgcctcctgggggcagatcacacaggtgctggagtctcccagacccccagtaacaaggtcacagagaagggaaaatatgtagagctcaggtgtgatccaatttcaggtcatactgccctttactggtaccgacaaagcctggggcagggcccagagtttctaatttacttccaaggcacgggtgcggcagatgactcagggctgcccaacgatcggttctttgcagtcaggcctgagggatccgtgtctactctgaagatccagcgcacagagcggggggactcagccgtgtatctttgcagagaccttgcggccgcataggtctcagtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctga

SEQ ID NO: 774 - DNA, 885 bp, Artificial Sequence
  V-Cb entry TRBV7-8_TRBC1_F14-F15
gccaccatgggcaccaggctcctctgctgggtggtcctgggtttcctagggacagatcacacaggtgctggagtctcccagtcccctaggtacaaagtcgcaaagagaggacaggatgtagctctcaggtgtgatccaatttcgggtcatgtatcccttttttggtaccaacaggccctggggcaggggccagagtttctgacttatttccagaatgaagctcaactagacaaatcggggctgcccagtgatcgcttctttgcagaaaggcctgagggatctgtctccactctgaagatccagcgcacacagcaggaggactccgccgtgtatctttgcagagaccttgcggccgcataggtctcagtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctga

SEQ ID NO: 775 - DNA, 882 bp, Artificial Sequence
  V-Cb entry TRBV19_TRBC1_F14-F15
gccaccatgagcaaccaggtgctctgctgtgtggtcctttgtttcctgggagcaaacaccgtggatggtggaatcactcagtccccaaagtacctgttcagaaaggaaggacagaatgtgaccctgagttgtgaacagaatttgaaccacgatgccatgtactggtaccgacaggacccagggcaagggctgagattgatctactactcacagatagtaaatgactttcagaaaggagatatagctgaagggtacagcgtgtctcgggagaagaaggaatcctttcctctcactgtgacatcggcccaaaagaacccgacagctttctatctttgcagagaccttgcggccgcataggtctcagtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctga

SEQ ID NO: 776 - DNA, 876 bp, Artificial Sequence
  V-Cb entry TRBV20-1_TRBC1_F14-F15
gccaccatgctgctgcttctgctgcttctggggccaggctccgggcttggtgctgtcgtgtctcaacatccgagctgggttatctgtaagagtggaacctctgtgaagatcgagtgccgttccctggactttcaggccacaactatgttttggtatcgtcagttcccgaaacagagtctcatgctgatggcaacttccaatgagggctccaaggccacatacgagcaaggcgtcgagaaggacaagtttctcatcaaccatgcaagcctgaccttgtccactctgacagtgaccagtgcccatcctgaagatagcagcttctacatttgcagagaccttgcggccgcataggtctcagtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctga

SEQ ID NO: 777 - DNA, 519 bp, Artificial Sequence
  Bidirectional terminator element (Esp3I to Esp3I site)
cgtctcggactccgctgatcataatcaagccatatcacatctgtagaggtttacttgctttaaaaaacctccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgctgaagagaagccttcttcaccatagagcccaccgcatccccagcatgcctgctattgtctacccaatcctcccccttgctgtcctgccccaccccaccccccagaatagaatgacacctactcagacaatgcgttgcaatttcctcattttattaggaaaggacagtgggagtggcaccttccagggtcaaggaaggcacgggggaggggcaaacaacagatggctggcaactagaaggcacagtcgcaaggtcgagacg

SEQ ID NO: 778 - DNA, 825 bp, Artificial Sequence
  TRA ORF
atggagaccctcttgggcctgcttatcctttggctgcagctgcaatgggttagcagcaaacaggaggtgacgcagattcctgcagctctgagtgtcccagaaggagaaaacttggttctcaactgcagtttcactgatagcgctatttacaacctccagtggtttaggcaggaccctgggaaaggactcacatctctgttgcttattcagtcaagtcagagagagcaaacaagtggacgccttaatgcctcgctggataaatcatcaggacgtagtactttatacattgcagcttctcagcctggtgactcagccacctacctctgcgccgtgaggccaaccagcggcggcagctacatcccaacctttggaagaggaaccagccttattgttcatccgtatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaggacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 779 - DNA, 936 bp, Artificial Sequence
  TRB ORF
atgagcatcggcctcctgtgctgtgcagccttgtctctcctgtgggcaggtccagtgaatgctggtgtcactcagaccccaaaattccaggtcctgaaaacaggacagagcatgacactgcagtgtgcccaggatatgaaccatgaatacatgtcctggtatcgacaagacccaggcatggggctgaggctgattcattactcagttggtgctggtatcactgaccaaggagaagtccccaatggctacaatgtctccagatcaaccacagaggatttcccgctcaggctgctgtcggctgctccctcccagacatctgtgtacttttgcgccagcagctacgtgggcaacaccggcgaactgttctttggagaaggctctaggctgaccgtactggaggacctgaaaaacgtgttcccacccgaggtcgctgtgtttgagccatcagaagcagagatctcccacacccaaaaggccacactggtatgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag